(19) United States
(12) Patent Application Publication
(10) Pub. No.: US 2014/0215653 A1
(43) Pub. Date: Jul. 31, 2014

(54) IDENTIFICATION AND USE OF EARLY EMBRYO AND/OR EARLY ENDOSPERM SPECIFIC PROMOTERS FOR GENE EXPRESSION IN MAIZE

(71) Applicant: TARGETED GROWTH, INC., Seattle, WA (US)

(72) Inventors: Jean Paul OLIVIER, Seattle, WA (US); James Michael Roberts, Seattle, WA (US); Fred Cross, New York, NY (US); Carolyn Hutcheon, Kirkland, WA (US); Asha Palta, Carmel, IN (US)

(73) Assignee: TARGETED GROWTH, INC., Seattle, WA (US)

(21) Appl. No.: 14/061,275

(22) Filed: Oct. 23, 2013

Related U.S. Application Data
(60) Provisional application No. 61/718,015, filed on Oct. 24, 2012.

(51) Int. Cl. C12N 15/82; C12Q 1/68

(52) U.S. Cl. CPC C12N 15/8216; C12Q 1/6895
USPC 800/275; 435/320.1; 435/419; 800/298; 800/320.1; 435/412; 800/278; 435/468; 435/6.11; 800/260

(57) ABSTRACT

The invention provides early embryo and early endosperm specific or preferred promoters, which are capable of driving and/or regulating the expression of an operably linked nucleic acid in a maize plant during early development of embryo and/or endosperm, and methods of isolating them. The promoters are derived from Zea mays or other plant species. The invention also provides compositions comprising one or more said promoters and methods of using the same.

### CROSS-REFERENCE TO RELATED APPLICATIONS

This application claims the benefit of U.S. Provisional Application Ser. No. 61/718,015, filed Oct. 24, 2012, which is hereby incorporated by reference in its entirety for all purposes.

### TECHNICAL FIELD

The invention generally relates to compositions and methods for gene expression. More specifically, the present invention relates to plant promoters and compositions and methods using the same.

### BACKGROUND OF THE INVENTION

The timing of expression of a transgene may be critical to the outcome. Early embryo- or endosperm-specific or preferred promoters can be particularly important and useful for improving crop performance, including for yield.

In looking for an early embryo- or endosperm-specific or preferred promoter, a thorough search of published literature revealed that very few genes expressed specifically in early embryo or endosperm development have been identified and characterized in plants, such as monocotyledonous plants, e.g., maize. Thus, there is a great need to identify early embryo- and early endosperm-specific genes.

### SUMMARY OF THE INVENTION

The present invention provides methods of identifying early embryo and/or early endosperm specific or preferred promoters from a maize plant, e.g., Zea mays. In some embodiments, the methods comprise (1) searches of public sequence databases, and/or (2) Illumina®/Solexa® sequencing of embryo and endosperm mRNA 7, 8, and 9 days after pollination (DAP).

The present invention also provides identification of promoters which can drive gene expression in an embryo- and/or endosperm-specific or preferred manner in maize. In some embodiments, the promoters can drive gene expression in an early embryo- and/or endosperm-specific or preferred manner in maize. In some embodiments, the promoters are derived from maize. In some embodiments, the promoters are derived from a plant other than maize, such as barley. In some embodiments, the promoters can drive gene expression in an early embryo-specific or preferred manner. In some embodiments, the promoters can drive gene expression in an early endosperm-specific or preferred manner. In some embodiments, the promoters can drive gene expression in an early embryo- and endosperm-specific or preferred manner. In some embodiments, the promoters can drive gene expression at different levels in an early embryo- and/or endosperm-specific or preferred manner in maize. For example, some of the promoters described herein can be used to achieve low level expression for genes that are toxic when highly expressed, while some of the promoters described herein can be used to achieve high level expression for genes that are naturally weakly expressed.

In some embodiments, the promoters are associated with genes including, but not limited to, MaizeSequence Nos. GRMZM2G124663 (also called TGI ZmLEC1 or ZmEmb2 herein), GRMZM2G040517, GRMZM2G066546, GRMZM2G046532, GRMZM2G087413 (also called ZmBETL-9 herein), GRMZM2G091054, GRMZM2G120008 (also called ZmEmb1 herein), GRMZM2G158407 (also called ZmSh-2 herein), GRMZM2G132162, GRMZM2G006585 (also called ZmEndosperm1 herein), GRMZM2G700896, GRMZM2G091445 (also called ZmBETL-10), GRMZM2G059620, GRMZM2G088896, GRMZM2G175976 (also called ZmBETL-3 herein), GRMZM2G046086, GRMZM2G175912, GRMZM2G152655 (also called ZmBETL-2 herein), GRMZM2G138727 (also called ZmEndosperm2 herein), GRMZM2G025763, GRMZM2G090264, GRMZM2G157806 (also called ZmEmb3 herein), GRMZM2G472234 (also called ZmEmb5 herein), GRMZM2G409372, GRMZM2G065157, GRMZM2G169149, GRMZM2G701926, GRMZM2G174883, GRMZM2G369799, GRMZM2G140302, GRMZM2G176390 (also called ZmEmb4 herein) and genes encoding PER1 (e.g., Hordeum vulgare PER1), Hordein (e.g., Hordeum vulgare B1 Hordein), ASP (e.g., ZmASP1, OsASP), CLV1 (e.g., ZmCLV1), and MRP (e.g., ZmMRP).

In some embodiments, such promoters comprise a polynucleotide sequence selected from the group consisting of SEQ ID NOs. 1-37.

In some embodiments, such promoters comprise a polynucleotide sequence having at least 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or higher nucleotide sequence identity to any one of SEQ ID NOs: 1-37, wherein such promoters can drive gene expression in a maize plant in an embryo- and/or endosperm-specific or preferred manner.

In some embodiments, such promoters comprise a polynucleotide sequence having a fragment of the sequence described herein, such as having at least about 50 nt, 100 nt, 150 nt, 200 nt, 250 nt, 300 nt, 350 nt, 400 nt, 450 nt, 500 nt, 600 nt, 700 nt, 800 nt, 900 nt, 1100 nt, 1200 nt, 1300 nt, 1400 nt, 1500 nt, 1600 nt, 1700 nt, 1800 nt, 1900 nt, 2000 nt or more of any one of SEQ ID NOs 1-37, wherein such promoters can drive gene expression in a maize plant in an embryo- and/or endosperm-specific or preferred manner.

In some embodiments, such promoters comprise one or more conserved regions. In some embodiments, the conserved regions comprise about 5, about 10, about 15, about 20, about 25, about 30, about 35, about 40, about 45, about 50, about 55, about 60, about 65, about 70, about 75, about 80, about 85, about 90, about 95, about 100, or more nucleic acids. For example, the conserved motifs can be about 6-29 bp long, or longer.

In some embodiments, the promoters are embryo and/or endosperm expressed, wherein the promoters comprise one or more conserved regions of about 6-29 bp nucleic acids derived from motif 1, motif 2, or motif 3 of FIG. 19A. For example, the conserved regions can be any one of the regions identified in FIGS. 19B to 19E.

In some embodiments, the promoters are embryo expressed, wherein the promoters comprise one or more conserved regions of about 6-41 bp nucleic acids derived from motif 1, motif 2, motif 3, motif 4, or motif 5 of FIG. 20A. For example, the conserved regions can be any one of the regions identified in FIGS. 20B to 20G.

In some embodiments, the promoters are endosperm expressed, wherein the promoters comprise one or more conserved regions of about 6-41 bp nucleic acids derived from motif 1, motif 2, motif 3, motif 4, or motif 5 of FIG. 21A. For example, the conserved regions can be any one of the regions identified in FIGS. 21B to 21G.

In some embodiments, the promoters are embryo and/or endosperm expressed, comprising at least two, three, four, five or more motifs selected from motifs 1-3 of FIG. 19A. In some embodiments, the embryo and/or endosperm expressed promoters comprise at least two, three, four, five or more conserved regions of about 6-29 bp derived from motifs 1-3 of FIG. 19A, such as any one of the regions identified in FIGS. 19B to 19E.

In some embodiments, the promoters are embryo expressed, comprising at least two, three, four, five or more motifs selected from motifs 1-5 of FIG. 20A. In some embodiments, the embryo expressed promoters comprise at least two, three, four, five or more conserved regions of about 6-41 bp derived from motifs 1-5 of FIG. 20A, such as any one of the regions identified in FIGS. 20B to 20G.

In some embodiments, the promoters are endosperm expressed, comprising at least two, three, four, five or more motifs selected from motifs 1-5 of FIG. 21A. In some embodiments, the endosperm expressed promoters comprise at least two, three, four, five or more conserved regions of about 6-41 bp derived from motifs 1-5 of FIG. 21A, such as any one of the regions identified in FIGS. 21B to 21G.

In some embodiments, the promoters are associated with the genes identified in Takacs et al. (Ontogeny of the Maize Shoot Apical Meristem, The Plant Cell, 24:3219-3234, August 2012), which is incorporated herein by reference in its entirety.

In some embodiments, synthetic promoters are produced by multimerizing the conservative regions described above. In some embodiments, the synthetic promoters are plant promoters. In some embodiments, the synthetic plant promoters have the same, greater, or weaker strength and/or tissue-specificity compared to the natural promoters of any one of SEQ ID NOs 1-37.

In some embodiments, the invention provides expression cassettes containing one or more promoter sequences associated with the early embryo- and/or endosperm-specific or preferred genes disclosed herein.

In some embodiments, the invention provides expression cassettes containing one or more promoter sequences having at least 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or higher nucleotide sequence identity to the promoter sequence associated with the early embryo- and/or endosperm-specific or preferred genes disclosed herein.

In some embodiments, the invention provides expression cassettes containing one or more promoter sequences associated with a gene encoding a polypeptide having at least 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or higher amino acid sequence identity to the polypeptide encoded by the early embryo- and/or endosperm-specific or preferred genes disclosed herein.

In some embodiments, the invention provides expression cassettes containing one or more promoter sequences that can drive gene expression in an early embryo- and/or endosperm-specific or preferred manner, wherein the promoter sequence can hybridize to any one of the promoter sequences disclosed herein under stringent conditions. In some embodiments, said stringent conditions are hybridization in 0.25 M Na₂HPO₄ buffer (pH 7.2) containing 1 mM Na₂EDTA, 0.5-20% sodium dodecyl sulfate at 45° C., such as 0.5%, 1%, 2%, 3%, 4%, 5%, 6%, 7%, 8%, 9%, 10%, 11%, 12%, 13%, 14%, 15%, 16%, 17%, 18%, 19% or 20%, followed by a wash in 5×SSC, containing 0.1% (w/v) sodium dodecyl sulfate, at 55° C. to 65° C.

In some embodiments, the invention provides expression cassettes containing one or more fragments of any one or more of the promoter sequences disclosed herein. In some embodiments, the one or more fragments by itself is capable of driving the expression of an operably linked gene of interest in an embryo- and/or endosperm-specific or preferred manner in a maize plant. In some embodiments, the fragment comprises about 50 bp, 100 bp, 150 bp, 200 bp, 300 bp, 400 bp, 500 bp, 600 bp, 700 bp, 800 bp, 900 bp, 1 kb, 1.5 kb, 2.0 kb, 2.5 kb, 3 kb, 4 kb, 5, kb, 6 kb, 7 kb, 8 kb, 9 kb, 10 kb, 15 kb, 20 kb, 25 kb, 30 kb, or more of the promoter sequences upstream of the coding sequence of the embryo- and/or endosperm-specific or preferred genes disclosed herein.

In some embodiments, the expression cassettes further comprise at least one enhancer sequence. In some embodiments, the enhancer sequence is a transcriptional enhancer sequence and/or a translation enhancer sequence. In some embodiments, the enhancer sequence is derived from a virus or a plant. In some embodiments, the enhancer derived from a plant is an intron sequence. In some embodiments, the enhancer derived from a plant is an exon sequence. In some embodiments, the enhancer derived from a plant is an intron sequence combined with an exon sequence. In some embodiments, the intron sequence is associated with a plant alcohol dehydrogenase gene. In some embodiments, the intron sequence is associated with the maize alcohol dehydrogenase gene.

In some embodiments, the expression cassettes further comprises at least one gene of interest. In some embodiments, the gene of interest is operably linked to the promoter. In some embodiments, the gene of interest is a gene associated with certain agronomically important traits. In some embodiments, the gene of interest is encoding a polypeptide. In some embodiments, the gene is selected from the group consisting of REVOLUTA genes and dominant negative cyclin-dependent kinase Inhibitor Proteins or KIP-Related Proteins (KRP). In some embodiments, the gene of interest is encoding an interference RNA. In some embodiments, the interfering polynucleotide is an inverted repeat sequence, antisense sequence, dsRNA sequence, RNAi sequence, siRNA sequence, shRNA sequence, or microRNA sequence. In some embodiments, the interference RNA is targeting one or more endogenous genes in the maize plant. In some embodiments, the interference RNA is targeting one or more REVOLUTA genes or KRP genes in maize. In some embodiments, the gene of interest is a gene naturally driven by a promoter of the present invention in a plant. In some embodiments, the gene naturally driven by a promoter of the present invention in plant contributes to one or more favorable agronomic phenotype.

In some embodiments, the expression cassettes are isolated from a plant species. In some other embodiments, the expression cassettes do not occur naturally. In some embodiments, the expression cassettes comprise a promoter of the present application, and a gene of interest, wherein the promoter and the gene of interest do not link to each other under natural conditions, e.g., the linkage between the promoter and the gene of interest does not exist in nature. For example, in some embodiments, the promoter and the gene of interest are derived from a same plant species, but are not linked to each other under natural conditions. In some embodiments, the promoter and the gene of interest are derived from two different species, e.g., the promoter and the gene of interest are heterologous to each other. In some embodiments, the gene of interest is derived from a different plant species, a bacteria species, a fungal species, a viral species, an algae species, or an animal species. In some embodiments, the expression cassettes comprise synthetic sequences.

In some embodiments, the expression cassettes further comprise a terminator sequence, wherein the terminator sequence does not naturally occur together with the promoter and/or the gene of interest.

The present invention also provides expression vectors for expressing a gene of interest in a maize plant in an embryo- and/or endosperm-specific or preferred manner. In some embodiments, the expression vectors comprise one or more of the expression cassettes as described herein. In some embodiments, the vector comprising: (a) a promoter sequence, (b) a DNA sequence of interest; and (c) a gene termination sequence, wherein the promoter comprises at least one embryo- and/or endosperm-specific or preferred promoter described herein. In some embodiments, the elements (a)-(c) above are arranged in the 5′-3′ direction. In some embodiments, the expression vectors comprise an open reading frame encoding a polypeptide of interest. In some embodiments, the expression vectors comprise an interference polynucleotide which when expressed can suppress the expression of one or more endogenous genes in the embryo and/or the endosperm.

The present invention also provides non-human transgenic cells. In some embodiments, the non-human transgenic cell comprises the expression cassettes described herein. In some embodiments, the transgenic cell is a maize plant cell, an animal cell, a bacterial cell, and a fungal cell.

The present invention also provides organisms comprising the expression cassettes described herein. In some embodiments, the organism is a maize plant.

The present invention also provides parts of the organisms comprising the expression cassettes described herein. In some embodiments, the part is a seed, wherein the seed comprises the expression cassette.

The present invention also provides methods for expressing a gene of interest in a maize plant, plant part or plant cell. In some embodiments, the methods comprise incorporating into a maize plant, plant part, or plant cell a nucleic acid molecule operably linked to a gene of interest, wherein the nucleic acid molecule comprises at least one promoter described herein. In some embodiments, the expression cassette is incorporated into the maize plant, plant part or plant cell by transformation or by homologous recombination. In some embodiments, the expression cassette is stably incorporated into the genome of the maize plant, plant part, or plant cell. In some embodiments, the gene of interest encodes a polypeptide. In some embodiments, the gene of interest is an interfering polynucleotide targeting one or more endogenous genes. In some embodiments, the gene of interest is a gene naturally associated with an embryo- and/or endosperm-specific or preferred promoter of the present application.

The present invention also provides maize progeny plants of the transgenic plants described herein. In some embodiments, the progeny plants have the expression cassette described herein.

The present invention also provides methods for producing maize transgenic plants, plant parts, or plant cells. In some embodiments, the methods are used to produce maize seeds. In some embodiments, the methods comprise crossing a transgenic maize plant or the progeny of the transgenic plant described herein as a donor with a recipient plant. In some embodiments, the donor maize plant and the recipient plant belong to the same or different varieties.

The present invention also provides methods for expressing a gene of interest in an embryo- and/or endosperm-specific or preferred manner in a maize plant, plant part, or plant cell. In some embodiments, the methods comprise introducing one or more expression cassettes of the present invention into a maize plant, plant part, or plant cell.

The present invention also provides methods for producing a plant having a recombinant gene under the control of an embryo- and/or endosperm-specific or preferred promoter. In some embodiments, the methods comprise transforming a plant cell with an expression cassette of the present invention. In some embodiments, the methods further comprise cultivating the transgenic cell under conditions conducive to regeneration and mature plant growth of a plant having the expression cassette.

The present invention also provides methods for modifying a phenotype of a target organism, comprising stably incorporating into the genome of the target organism an expression cassette of the present invention.

The present invention also provides processes of determining the presence or absence of an expression cassette of the present invention, and fragments and variations thereof in a plant. In some embodiments, the processes comprise at least one of:
(a) isolating nucleic acid molecules from said plant and amplifying sequences homologous to the expression cassette;
(b) isolating nucleic acid molecules from said plant and performing a Southern hybridization to detect the expression cassette;
(c) isolating proteins from said plant and performing a Western Blot using antibodies to a protein encoded by the expression cassette; and/or
(d) demonstrating the presence of mRNA sequences derived from a polynucleotide mRNA transcript and unique to the expression cassette.

The present invention also provides methods of breeding plants to produce a plant having an expression cassette of the present invention. In some embodiments, the methods comprise making a cross between a plant with an expression cassette of the present invention with a second plant to produce an F1 plant. In some embodiments, the methods further comprise backcrossing the F1 plant to the second plant. In some embodiments, the methods further comprise repeating the backcrossing step to generate a near isogenic or isogenic line, wherein the expression cassette of the present invention is integrated into the genome of the second plant and the near isogenic or isogenic line derived from the second plant with the expression cassette.

The invention also provides products derived from the transgenic plants described herein. Any and all products made using the seeds, plants and parts thereof obtained from the transgenic plants or from any line produced using the transgenic plants described herein as a direct or indirect parent are also part of the invention. Examples of such products include but are not limited to corn meal, corn flour, corn starch, corn syrup, corn sweetener and corn oil. The origin of the corn used in such corn products can be determined by tracking the source of the corn used to make the products and/or by using protein (isozyme, ELISA, etc.) and/or DNA (RFLP, PCR, SSR, SNP, EST, etc.) testing.

The invention also provides molecular markers. In some embodiments, the molecular markers are derived from the early embryo- and/or endosperm-specific or preferred promoters of the present application. In some embodiments, the molecular markers are used in molecular marker assisted breeding. In some embodiments, the molecular markers can be utilized to monitor the transfer of specific genetic material.

### DESCRIPTION OF THE TEXT FILE SUBMITTED ELECTRONICALLY

The contents of the text file submitted electronically are incorporated herein by reference in their entirety: A computer readable format copy of the Sequence Listing (filename: TARG02401US.txt, date recorded: Oct. 21, 2013, file size 152 kilobytes).

### BRIEF DESCRIPTION OF THE DRAWINGS

FIG. 1: FIG. 1A depicts mRNA abundance in 8 days after pollination (DAP) embryo/mRNA abundance in endosperm+leaf vs. mRNA abundance in 8 DAP embryo (Group A). FIG. 1B depicts mRNA abundance of 8 DAP embryo/mRNA abundance in leaf vs. abundance in 8 DAP embryo (Group B). FIG. 1C depicts mRNA abundance in 9 DAP embryo/mRNA abundance in leaf vs. abundance in 9 DAP embryo (Group C). FIG. 1D depicts mRNA abundance in 9 DAP embryo vs. mRNA abundance in leaf (Groups D and E). Candidate genes in each group are described in further detail in Example 1. As used herein, the term “abundance” refers to mRNA-Seq count of the gene in a specified tissue, as measured by Illumina sequencing, normalized to the total mRNA-Seq count in that tissue.

FIG. 2 depicts expression of candidate embryo-specific or preferred genes, determined by qPCR: GRMZM2G120008 (2A), GRMZM2G124663 (2B), GRMZM2G472234 (2C), GRMZM2G046086 (2D and 2E), GRMZM2G040517 (2F and 2G), GRMZM2G065157 (2H AND 2I), GRMZM2G157806 (2J and 2K), GRMZM2G176390 (2L AND 2M).

FIG. 3 depicts expression of candidate embryo and endosperm specific or preferred genes, determined by qPCR: GRMZM2G066546 (3A), GRMZM2G087413 (3B), GRMZM2G158407 (3C), GRMZM2G132162 (3D), GRMZM2G700896 (3E), GRMZM2G091445 (3F), GRMZM2G175976 (3G and 3H), GRMZM2G175912 (3I and 3J), GRMZM2G152655 (3K and 3L), GRMZM2G090264 (3M), GRMZM2G174883 (3N and 3O), GRMZM2G369799 (3P and 3Q), GRMZM2G140302 (3R and 3S), GRMZM2G088896 (3T and 3U), GRMZM2G046532 (3V and 3W).

FIG. 4 depicts expression of candidate endosperm specific or preferred genes, determined by qPCR: GRMZM2G091054 (4A), GRMZM2G006585 (4B), GRMZM2G025763 (4C), GRMZM2G138727 (4D and 4E), GRMZM2G059620 (4F and 4G).

FIG. 5 depicts expression results due to poor signal/non-specific amplification in qPCR assay: GRMZM2G169149 (5A and 5B), GRMZM2G409372 (5C and 5D), GRMZM2G701926 (5E and 5F).

FIG. 6 depicts expression pattern of GUS gene under the control of maize ASP1 promoter: qualitative summary of GUS expression (FIG. 6A), 1=weak GUS expression to 5=strong GUS expression; expression in transverse section (TS) of corn ear at 3 DAP (FIG. 6B); expression in basal endosperm transfer layer at 14 DAP (FIG. 6C).

FIG. 7 depicts GUS expression in control non-transformed kernels at 9, 14, and 21 DAP.

FIG. 8 depicts expression pattern of GUS gene under the control of maize CLV1 promoter: qualitative summary of GUS expression (FIG. 8A), 1=weak GUS expression to 5=strong GUS expression; expression in the transverse section (TS) of corn ear at 3 DAP or 5 DAP (FIGS. 8B and 8C); expression in endosperm at 21 DAP (FIG. 8D).

FIG. 9 depicts expression pattern of GUS gene under the control of Hordeum vulgare PER1 promoter: qualitative summary of GUS expression (FIG. 9A), 1=weak GUS expression to 5=strong GUS expression; expression in the transverse section (TS) of corn ear at 3 DAP (FIG. 9B); expression in zygotic embryo, endosperm and aleurone at 14 DAP (FIG. 9C); expression in zygotic embryo and endosperm and aleurone at 21 DAP (FIGS. 9D and 9E).

FIG. 10 depicts expression pattern of GUS gene under the control of Hordeum vulgare B1 Hordein promoter: qualitative summary of GUS expression (FIG. 10A), 1=weak GUS expression to 5=strong GUS expression; expression in zygotic endosperm and embryo at 9 DAP (FIG. 10B) and 21 DAP (FIG. 10C).

FIG. 11 depicts expression pattern of GUS gene under the control of maize Emb1v1 promoter: qualitative summary of GUS expression (FIG. 11A), 1=weak GUS expression to 5=strong GUS expression; expression in the transverse section (TS) of corn ear at 6 DAP (FIG. 11B); expression in endosperm at 14 DAP (FIG. 11C); expression in zygotic embryo, endosperm and basal endosperm transfer layer for 21 DAP (FIG. 11D).

FIG. 12 depicts expression pattern of GUS gene under the control of maize Emb4v1 promoter: qualitative summary of GUS expression (FIG. 12A), 1=weak GUS expression to 5=strong GUS expression; expression in the transverse section (TS) of corn ear at 6 DAP (FIG. 12B); expression in embryo and endosperm at 9 DAP (FIG. 12C).

FIG. 13 depicts expression pattern of GUS gene under the control of maize Emb2v1 promoter: qualitative summary of GUS expression (FIG. 13A), 1=weak GUS expression to 5=strong GUS expression; expression in the transverse section (TS) of corn ear at 5 DAP (FIG. 13B); expression in basal endosperm transfer layer and endosperm at 9, 14 and 21 DAP (FIG. 13C).

FIG. 14 depicts expression pattern of GUS gene under the control of maize Endosperm 1v 1 promoter: qualitative summary of GUS expression (FIG. 14A), 1=weak GUS expression to 5=strong GUS expression; expression in the transverse section (TS) of corn ear at 5 DAP (FIG. 14B); expression in basal endosperm transfer layer at 9, 14 and 21 DAP (FIG. 14C).

FIG. 15 depicts expression pattern of GUS gene under the control of rice ASP1v2 promoter: qualitative summary of GUS expression (FIG. 15A), 1=weak GUS expression to 5=strong GUS expression; expression in the transverse section (TS) of corn ear at 3 and 5 DAP (FIG. 15B); expression in basal endosperm transfer layer at 9 and 21 DAP (FIG. 15C).

FIG. 16 depicts comparison of histochemical GUS expression for 9 promoters in T1 zygotic embryo and endosperm. 1=weak GUS expression to 5=strong GUS expression.

FIG. 17 depicts comparison of gene expression for 9 promoters in other tissue types. 1=weak GUS expression to 5=strong GUS expression.

FIG. 18 depicts sequence comparison of maize LEC1 promoter disclosed in U.S. Pat. No. 7,432,418 and the maize LEC1 promoter disclosed in the present application. The comparison shows several conserved promoter consensus fragments.

FIG. 19: FIG. 19A depicts the top three conserved motifs observed in the 31 corn embryo and/or endosperm promoter sequences identified from Illumina®/Solexa® sequencing of embryo and endosperm mRNA 7, 8, and 9 days after pollination (DAP). FIG. 19B depicts a summary of the locations of all three motifs on 27 of the 31 promoter sequences. FIG. 19C-E give the nucleotide start positions and DNA strand (+=sense strand, −=antisense strand) on which each motif occurs for motifs 1, 2 and 3, respectively, for relevant embryo and/or endosperm promoter sequences, sorted according to their p-values. Each motif site is flanked by the 5′ ten nucleotides and 3′ ten nucleotides found in each promoter sequence.

FIG. 20: FIG. 20A depicts the top five conserved motifs observed in the 26 corn embryo-specific and embryo+endosperm promoter sequences identified from Illumina®/Solexa® sequencing of embryo and endosperm mRNA 7, 8, and 9 days after pollination (DAP). FIG. 20B depicts a summary of the locations of all five motifs on 24 of the 26 promoter sequences. FIG. 20C-G give the nucleotide start positions and DNA strand (+=sense strand, −=antisense strand) on which each motif occurs for motifs 1, 2, 3, 4 and 5, respectively, for relevant embryo and/or endosperm promoter sequences, sorted according to their p-values. Each motif site is flanked by the 5′ ten nucleotides and 3′ ten nucleotides found in each promoter sequence.

FIG. 21: FIG. 21A depicts the top five conserved motifs observed in the 23 corn endosperm-specific and embryo+endosperm promoter sequences identified from Illumina®/Solexa® sequencing of embryo and endosperm mRNA 7, 8, and 9 days after pollination (DAP). FIG. 21B depicts a summary of the locations of all five motifs on 21 of the 23 promoter sequences. FIG. 21C-G give the nucleotide start positions and DNA strand (+sense strand, −=antisense strand) on which each motif occurs for motifs 1, 2, 3, 4 and 5, respectively, for relevant embryo and/or endosperm promoter sequences, sorted according to their p-values. Each motif site is flanked by the 5′ ten nucleotides and 3′ ten nucleotides found in each promoter sequence.

### DESCRIPTION OF THE TEXT FILE SUBMITTED ELECTRONICALLY

The contents of the text file submitted electronically are incorporated herein by reference in their entirety: A computer readable format copy of the Sequence Listing (filename: TARG02401US.txt, date recorded: Oct. 21, 2013, file size 152 kilobytes).

### DETAILED DESCRIPTION OF THE INVENTION

All publications, patents and patent applications, including any drawings and appendices, and all nucleic acid sequences and polypeptide sequences identified by MaizeSequence numbers, herein are incorporated by reference to the same extent as if each individual publication or patent application was specifically and individually indicated to be incorporated by reference.

The following description includes information that may be useful in understanding the present invention. It is not an admission that any of the information provided herein is prior art or relevant to the presently claimed inventions, or that any publication specifically or implicitly referenced is prior art.

### DEFINITION

As used herein, the verb “comprise” as is used in this description and in the claims and its conjugations are used in its non-limiting sense to mean that items following the word are included, but items not specifically mentioned are not excluded.

The invention provides compositions and methods to produce maize plants comprising an early embryo- or endosperm-specific or preferred promoter. As used herein, the term “plant” refers to corn (i.e., Zea mays), unless specified otherwise.

The invention provides plant parts. As used herein, the term “plant part” refers to any part of a plant including but not limited to the embryo, shoot, root, stem, seed, stipule, leaf, petal, flower, ovule, bract, branch, petiole, internode, bark, pubescence, tiller, rhizome, frond, blade, ovule, pollen, stamen, and the like. The two main parts of plants grown in some sort of media, such as soil, are often referred to as the “above-ground” part, also often referred to as the “shoots”, and the “below-ground” part, also often referred to as the “roots”.

The term “a” or “an” refers to one or more of that entity; for example, “a gene” refers to one or more genes or at least one gene. As such, the terms “a” (or “an”), “one or more” and “at least one” are used interchangeably herein. In addition, reference to “an element” by the indefinite article “a” or “an” does not exclude the possibility that more than one of the elements are present, unless the context clearly requires that there is one and only one of the elements.

The invention provides isolated, chimeric, recombinant or synthetic polynucleotide sequences. As used herein, the terms “polynucleotide”, “polynucleotide sequence”, “nucleic acid sequence”, “nucleic acid fragment”, and “isolated nucleic acid fragment” are used interchangeably herein. These terms encompass nucleotide sequences and the like. A polynucleotide may be a polymer of RNA or DNA that is single- or double-stranded, that optionally contains synthetic, non-natural or altered nucleotide bases. A polynucleotide in the form of a polymer of DNA may be comprised of one or more segments of cDNA, genomic DNA, synthetic DNA, or mixtures thereof. Nucleotides (usually found in their 5′-monophosphate form) are referred to by a single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

As used herein, the term “chimeric” or “recombinant” when describing a nucleic acid sequence or a protein sequence refers to a nucleic acid or a protein sequence that links at least two heterologous polynucleotides or two heterologous polypeptides into a single macromolecule, or that re-arranges one or more elements of at least one natural nucleic acid or protein sequence. For example, the term “recombinant” can refer to an artificial combination of two otherwise separated segments of sequence, e.g., by chemical synthesis or by the manipulation of isolated segments of nucleic acids by genetic engineering techniques.

As used herein, a “synthetic nucleotide sequence” or “synthetic polynucleotide sequence” is a nucleotide sequence that is not known to occur in nature or that is not naturally occurring. Generally, such a synthetic nucleotide sequence will comprise at least one nucleotide difference when compared to any other naturally occurring nucleotide sequence. It is recognized that a genetic regulatory element of the present invention comprises a synthetic nucleotide sequence. In some embodiments, the synthetic nucleotide sequence shares little or no extended homology to natural sequences. Extended homology in this context generally refers to 100% sequence identity extending beyond about 25 nucleotides of contiguous sequence. A synthetic genetic regulatory element of the present invention comprises a synthetic nucleotide sequence.

As used herein, an “isolated” or “purified” nucleic acid molecule or polynucleotide, or biologically active portion thereof, is substantially or essentially free from components that normally accompany or interact with the nucleic acid molecule or polynucleotide as found in its naturally occurring environment. Thus, an isolated or purified nucleic acid molecule or polynucleotide is substantially free of other cellular material or culture medium when produced by recombinant techniques, or substantially free of chemical precursors or other chemicals when chemically synthesized.

The invention provides isolated, chimeric, recombinant or synthetic nucleic acids. As used herein, the term “nucleic acid” refers to a polymeric form of nucleotides of any length, either ribonucleotides or deoxyribonucleotides, or analogs thereof. This term refers to the primary structure of the molecule, and thus includes double- and single-stranded DNA, as well as double- and single-stranded RNA. It also includes modified nucleic acids such as methylated and/or capped nucleic acids, nucleic acids containing modified bases, backbone modifications, and the like. The terms “nucleic acid” and “nucleotide sequence” are used interchangeably.

The invention provides genes comprising the isolated, chimeric, recombinant or synthetic genes. As used herein, the term “gene” refers to any segment of DNA associated with a biological function. Thus, genes include, but are not limited to, coding sequences and/or the regulatory sequences required for their expression. Genes can also include nonexpressed DNA segments that, for example, form recognition sequences for other proteins. Genes can be obtained from a variety of sources, including cloning from a source of interest or synthesizing from known or predicted sequence information, and may include sequences designed to have desired parameters.

The invention provides homologous and orthologous polynucleotides and polypeptides. As used herein, the term “homologous” or “homologue” or “ortholog” is known in the art and refers to related sequences that share a common ancestor or family member and are determined based on the degree of sequence identity. The terms “homology”, “homologous”, “substantially similar” and “corresponding substantially” are used interchangeably herein. They refer to nucleic acid fragments wherein changes in one or more nucleotide bases do not affect the ability of the nucleic acid fragment to mediate gene expression or produce a certain phenotype. These terms also refer to modifications of the nucleic acid fragments of the instant invention such as deletion or insertion of one or more nucleotides that do not substantially alter the functional properties of the resulting nucleic acid fragment relative to the initial, unmodified fragment. It is therefore understood, as those skilled in the art will appreciate, that the invention encompasses more than the specific exemplary sequences. These terms describe the relationship between a gene found in one species, subspecies, variety, cultivar or strain and the corresponding or equivalent gene in another species, subspecies, variety, cultivar or strain. For purposes of this invention homologous sequences are compared. “Homologous sequences” or “homologues” or “orthologs” are thought, believed, or known to be functionally related. A functional relationship may be indicated in any one of a number of ways, including, but not limited to: (a) degree of sequence identity and/or (b) the same or similar biological function. Preferably, both (a) and (b) are indicated. The degree of sequence identity may vary, but in some embodiments, is at least 50% (when using standard sequence alignment programs known in the art), at least 60%, at least 65%, at least 70%, at least 75%, at least 80%, at least 85%, at least 90%, at least about 91%, at least about 92%, at least about 93%, at least about 94%, at least about 95%, at least about 96%, at least about 97%, at least about 98%, or at least 98.5%, or at least about 99%, or at least 99.5%, or at least 99.8%, or at least 99.9%. Homology can be determined using software programs readily available in the art, such as those discussed in Current Protocols in Molecular Biology (F. M. Ausubel et al., eds., 1987) Supplement 30, section 7.718, Table 7.71. Some alignment programs are MacVector (Oxford Molecular Ltd, Oxford, U.K.), ALIGN Plus (Scientific and Educational Software, Pennsylvania) and AlignX (Vector NTI, Invitrogen, Carlsbad, Calif.). Another alignment program is Sequencher (Gene Codes, Ann Arbor, Mich.), using default parameters.

The invention provides polynucleotides with nucleotide change when compared to a wild-type reference sequence. As used herein, the term “nucleotide change” refers to, e.g., nucleotide substitution, deletion, and/or insertion, as is well understood in the art. For example, mutations contain alterations that produce silent substitutions, additions, or deletions, but do not alter the properties or activities of the encoded protein or how the proteins are made.

The invention provides polypeptides with protein modification when compared to a wild-type reference sequence. As used herein, the term “protein modification” refers to, e.g., amino acid substitution, amino acid modification, deletion, and/or insertion, as is well understood in the art.

The invention provides polynucleotides and polypeptides derived from wild-type reference sequences. As used herein, the term “derived from” refers to the origin or source, and may include naturally occurring, recombinant, unpurified, or purified molecules. A nucleic acid or an amino acid derived from an origin or source may have all kinds of nucleotide changes or protein modification as defined elsewhere herein.

The invention provides agents to make and use the biological materials of the present invention. As used herein, the term “agent”, as used herein, means a biological or chemical compound such as a simple or complex organic or inorganic molecule, a peptide, a protein or an oligonucleotide that modulates the function of a nucleic acid or polypeptide. A vast array of compounds can be synthesized, for example oligomers, such as oligopeptides and oligonucleotides, and synthetic organic and inorganic compounds based on various core structures, and these are also included in the term “agent”. In addition, various natural sources can provide compounds for screening, such as plant or animal extracts, and the like. Compounds can be tested singly or in combination with one another.

The invention provides portions or fragments of the nucleic acid sequences and polypeptide sequences of the present invention. As used herein, the term “at least a portion” or “fragment” of a nucleic acid or polypeptide means a portion having the minimal size characteristics of such sequences, or any larger fragment of the full length molecule, up to and including the full length molecule. For example, Thus, fragments of a nucleotide sequence may range from at least about 10 nucleotides, at least about 15 nucleotides, at least about 20 nucleotides, at least about 30 nucleotides, at least about 40 nucleotides, at least about 50 nucleotides, at least about 60 nucleotides, at least about 70 nucleotides, at least about 80 nucleotides, at least about 90 nucleotides, at least about 100 nucleotides, at least about 150 nucleotides, at least about 200 nucleotides, at least about 250 nucleotides, at least about 300 nucleotides, at least about 350 nucleotides, at least about 400 nucleotides, at least about 450 nucleotides, at least about 500 nucleotides, at least about 550 nucleotides, at least about 600 nucleotides, and up to the full-length polynucleotide of the invention. A fragment of a polynucleotide of the invention may encode a biologically active portion of a genetic regulatory element. A biologically active portion of a genetic regulatory element can be prepared by isolating a portion of one of the polynucleotides of the invention that comprises the genetic regulatory element and assessing activity as described herein.

Similarly, a portion of a polypeptide may be 4 amino acids, 5 amino acids, 6 amino acids, 7 amino acids, and so on, going up to the full length polypeptide. The length of the portion to be used will depend on the particular application. A portion of a nucleic acid useful as hybridization probe may be as short as 12 nucleotides; in some embodiments, it is 20 nucleotides. A portion of a polypeptide useful as an epitope may be as short as 4 amino acids. A portion of a polypeptide that performs the function of the full-length polypeptide would generally be longer than 4 amino acids.

The invention provides sequences having high similarity or identity to the nucleic acid sequences and polypeptide sequences of the present invention. As used herein, “sequence identity” or “identity” in the context of two nucleic acid or polypeptide sequences includes reference to the residues in the two sequences which are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are said to have “sequence similarity” or “similarity.” Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, Computer Applic. Biol. Sci., 4:11-17 (1988).

The invention provides sequences substantially complementary to the nucleic acid sequences of the present invention. As used herein, the term “substantially complementary” means that two nucleic acid sequences have at least about 65%, 70%, 75%, 80%, 85%, 90%, 91%, 92%, 93%, 94%, 95%, 96%, 97%, 98%, 99%, 99.5% or more sequence complementarities to each other. This means that primers and probes must exhibit sufficient complementarity to their template and target nucleic acid, respectively, to hybridize under stringent conditions. Therefore, the primer and probe sequences need not reflect the exact complementary sequence of the binding region on the template and degenerate primers can be used. For example, a non-complementary nucleotide fragment may be attached to the 5′-end of the primer, with the remainder of the primer sequence being complementary to the strand. Alternatively, non-complementary bases or longer sequences can be interspersed into the primer, provided that the primer has sufficient complementarity with the sequence of one of the strands to be amplified to hybridize therewith, and to thereby form a duplex structure which can be extended by polymerizing means. The non-complementary nucleotide sequences of the primers may include restriction enzyme sites. Appending a restriction enzyme site to the end(s) of the target sequence would be particularly helpful for cloning of the target sequence. A substantially complementary primer sequence is one that has sufficient sequence complementarity to the amplification template to result in primer binding and second-strand synthesis. The skilled person is familiar with the requirements of primers to have sufficient sequence complementarity to the amplification template.

The invention provides biologically active variants or functional variants of the nucleic acid sequences and polypeptide sequences of the present invention. As used herein, the phrase “a biologically active variant” or “functional variant” with respect to a protein refers to an amino acid sequence that is altered by one or more amino acids with respect to a reference sequence, while still maintains substantial biological activity of the reference sequence. The variant can have “conservative” changes, wherein a substituted amino acid has similar structural or chemical properties, e.g., replacement of leucine with isoleucine. Alternatively, a variant can have “nonconservative” changes, e.g., replacement of a glycine with a tryptophan. Analogous minor variations can also include amino acid deletion or insertion, or both. Guidance in determining which amino acid residues can be substituted, inserted, or deleted without eliminating biological or immunological activity can be found using computer programs well known in the art, for example, DNASTAR software. For polynucleotides, a variant comprises a polynucleotide having deletions (i.e., truncations) at the 5′ and/or 3′ end; deletion and/or addition of one or more nucleotides at one or more internal sites in the reference polynucleotide; and/or substitution of one or more nucleotides at one or more sites in the reference polynucleotide. As used herein, a “reference” polynucleotide comprises a nucleotide sequence produced by the methods disclosed herein. Variant polynucleotides also include synthetically derived polynucleotides, such as those generated, for example, by using site directed mutagenesis but which still comprise genetic regulatory element activity. Generally, variants of a particular polynucleotide or nucleic acid molecule of the invention will have at least about 60%, 65%, 70%, 75%, 80%, 85%, 90%, 91%, 91.5%, 92%, 92.5%, 93%, 93.5%, 94%, 94.5%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or more sequence identity to that particular polynucleotide as determined by sequence alignment programs and parameters as described elsewhere herein.

Variant polynucleotides also encompass sequences derived from a mutagenic and recombinogenic procedure such as DNA shuffling. Strategies for such DNA shuffling are known in the art. See, for example, Stemmer (1994) PNAS 91:10747-10751; Stemmer (1994) Nature 370:389-391; Crameri et al. (1997) Nature Biotech. 15:436-438; Moore et al. (1997) J. Mol. Biol. 272:336-347; Zhang et al. (1997) PNAS 94:4504-4509; Crameri et al. (1998) Nature 391:288-291; and U.S. Pat. Nos. 5,605,793 and 5,837,458. For PCR amplifications of the polynucleotides disclosed herein, oligonucleotide primers can be designed for use in PCR reactions to amplify corresponding DNA sequences from cDNA or genomic DNA extracted from any plant of interest. Methods for designing PCR primers and PCR cloning are generally known in the art and are disclosed in Sambrook et al. (1989) Molecular Cloning: A Laboratory Manual (2nd ed., Cold Spring Harbor Laboratory Press, Plainview, N.Y.). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Known methods of PCR include, but are not limited to, methods using paired primers, nested primers, single specific primers, degenerate primers, gene-specific primers, vector-specific primers, partially-mismatched primers, and the like.

The invention provides primers that are derived from the nucleic acid sequences and polypeptide sequences of the present invention. The term “primer” as used herein refers to an oligonucleotide which is capable of annealing to the amplification target allowing a DNA polymerase to attach, thereby serving as a point of initiation of DNA synthesis when placed under conditions in which synthesis of primer extension product is induced, i.e., in the presence of nucleotides and an agent for polymerization such as DNA polymerase and at a suitable temperature and pH. The (amplification) primer is preferably single stranded for maximum efficiency in amplification. Preferably, the primer is an oligodeoxyribonucleotide. The primer must be sufficiently long to prime the synthesis of extension products in the presence of the agent for polymerization. The exact lengths of the primers will depend on many factors, including temperature and composition (A/T vs. G/C content) of primer. A pair of bi-directional primers consists of one forward and one reverse primer as commonly used in the art of DNA amplification such as in PCR amplification.

The invention provides polynucleotide sequences that can hybridize with the nucleic acid sequences of the present invention. The terms “stringency” or “stringent hybridization conditions” refer to hybridization conditions that affect the stability of hybrids, e.g., temperature, salt concentration, pH, formamide concentration and the like. These conditions are empirically optimized to maximize specific binding and minimize non-specific binding of primer or probe to its target nucleic acid sequence. The terms as used include reference to conditions under which a probe or primer will hybridize to its target sequence, to a detectably greater degree than other sequences (e.g. at least 2-fold over background). Stringent conditions are sequence dependent and will be different in different circumstances. Longer sequences hybridize specifically at higher temperatures. Generally, stringent conditions are selected to be about 5° C. lower than the thermal melting point (Tm) for the specific sequence at a defined ionic strength and pH. The Tm is the temperature (under defined ionic strength and pH) at which 50% of a complementary target sequence hybridizes to a perfectly matched probe or primer. Typically, stringent conditions will be those in which the salt concentration is less than about 1.0 M Na⁺ion, typically about 0.01 to 1.0 M Na+ion concentration (or other salts) at pH 7.0 to 8.3 and the temperature is at least about 30° C. for short probes or primers (e.g. 10 to 50 nucleotides) and at least about 60° C. for long probes or primers (e.g. greater than 50 nucleotides). Stringent conditions may also be achieved with the addition of destabilizing agents such as formamide. Exemplary low stringent conditions or “conditions of reduced stringency” include hybridization with a buffer solution of 30% formamide, 1 M NaCl, 1% SDS at 37° C. and a wash in 2×SSC at 40° C. Exemplary high stringency conditions include hybridization in 50% formamide, 1M NaCl, 1% SDS at 37° C., and a wash in 0.1×SSC at 60° C. Hybridization procedures are well known in the art and are described by e.g. Ausubel et al., 1998 and Sambrook et al., 2001.

The invention provides promoter sequences. As used herein, “promoter” refers to a DNA sequence capable of controlling the expression of a coding sequence or functional RNA. The promoter sequence consists of proximal and more distal upstream elements, the latter elements often referred to as enhancers. Accordingly, an “enhancer” is a DNA sequence that can stimulate promoter activity, and may be an innate element of the promoter or a heterologous element inserted to enhance the level or tissue specificity of a promoter. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. It is further recognized that since in most cases the exact boundaries of regulatory sequences have not been completely defined, DNA fragments of some variation may have identical promoter activity.

In some embodiments, the invention provides plant promoters. As used herein, a “plant promoter” is a promoter capable of initiating transcription in plant cells whether or not its origin is a plant cell, e.g. it is well known that Agrobacterium promoters are functional in plant cells. Thus, plant promoters include promoter DNA obtained from plants, plant viruses and bacteria such as Agrobacterium and Bradyrhizobium bacteria. A plant promoter can be a constitutive promoter or a non-constitutive promoter.

In some embodiments, the invention provides constitutive promoters. As used herein, a “constitutive promoter” is a promoter which is active under most conditions and/or during most development stages. There are several advantages to using constitutive promoters in expression vectors used in plant biotechnology, such as: high level of production of proteins used to select transgenic cells or plants; high level of expression of reporter proteins or storable markers, allowing easy detection and quantification; high level of production of a transcription factor that is part of a regulatory transcription system; production of compounds that requires ubiquitous activity in the plant; and production of compounds that are required during all stages of plant development. Non-limiting exemplary constitutive promoters include, CaMV 35S promoter, opine promoters, ubiquitin promoter, alcohol dehydrogenase promoter, etc. For example, in some embodiments, the embryo- and/or endosperm-specific or preferred promoters of the present invention can be constitutive promoters which are active not only during the early development stage of the embryo and/or the endosperm, but also active throughout the whole development of the embryo and/or the endosperm.

In some embodiments, the invention provides non-constitutive promoters. As used herein, a “non-constitutive promoter” is a promoter which is active under certain conditions, in certain types of cells, and/or during certain developmental stages. For example, tissue specific, tissue preferred, cell type specific, cell type preferred, inducible promoters, and promoters under development control are non-constitutive promoters. Examples of promoters under developmental control include promoters that preferentially initiate transcription in certain tissues, such as stems, leaves, roots, or seeds. For example, in some embodiments, the embryo- and/or endosperm-specific or preferred promoters can be non-constitutive promoters which are active only during the early development stage of the embryo and/or the endosperm. For example, the promoters are active for no more than one week, two weeks, three weeks, or four weeks after pollination.

In some embodiments, the invention provides inducible promoters. As used herein, “inducible” or “repressible” promoter is a promoter which is under chemical or environmental factors control. Examples of environmental conditions that may effect transcription by inducible promoters include anaerobic conditions, or certain chemicals, or the presence of light.

In some embodiments, the invention provides tissue specific promoters. As used herein, a “tissue specific” promoter is a promoter that initiates transcription only in certain tissues. Unlike constitutive expression of genes, tissue-specific expression is the result of several interacting levels of gene regulation. As such, in the art sometimes it is preferable to use promoters from homologous or closely related plant species to achieve efficient and reliable expression of transgenes in particular tissues. This is one of the main reasons for the large amount of tissue-specific promoters isolated from particular plants and tissues found in both scientific and patent literature.

In some embodiments, the invention provides tissue-preferred promoters. As used herein, a “tissue preferred” promoter is a promoter that initiates transcription mostly, but not necessarily entirely or solely in certain tissues.

In some embodiments, the invention provides cell type specific promoters. As used herein, a “cell type specific” promoter is a promoter that primarily drives expression in certain cell types in one or more organs, for example, vascular cells in roots, leaves, stalk cells, and stem cells.

In some embodiments, the invention provides cell type preferred promoters. As used herein, a “cell type preferred” promoter is a promoter that primarily drives expression mostly, but not necessarily entirely or solely in certain cell types in one or more organs, for example, vascular cells in roots, leaves, stalk cells, and stem cells.

In some embodiments, the invention provides seed specific, embryo specific, and/or endosperm specific promoters. As used herein, a “seed specific”, an “embryo specific”, or an “endosperm specific promoter” is a promoter that initiates transcription only in seed tissues, embryo tissue, or endosperm tissue.

In some embodiments, the invention provides seed preferred promoters. As used herein, a “seed preferred” promoter is a promoter that primarily drives expression mostly, but not necessarily entirely or solely in seed tissue.

In some embodiments, the invention provides embryo preferred promoters. As used herein, an “embryo preferred” promoter is a promoter that primarily drives expression mostly, but not necessarily entirely or solely in embryo tissue.

In some embodiments, the invention provides endosperm preferred promoters. As used herein, an “endosperm preferred” promoter is a promoter that primarily drives expression mostly, but not necessarily entirely or solely in endosperm tissue.

In some embodiments, the invention provides embryo and endosperm specific promoters. As used herein, an “embryo and endosperm specific” promoter is a promoter that initiates transcription only in embryo and endosperm tissue.

In some embodiments, the invention provides embryo and endosperm preferred promoters. As used herein, an “embryo and endosperm preferred” promoter is a promoter that primarily drives expression mostly, but not necessarily entirely or solely in embryo and endosperm tissue.

In some embodiments, the invention provides early embryo and/or early endosperm promoters. As used herein, an “early embryo and/or endosperm” promoter with respect to maize plant refers to a promoter that can drive gene expression in maize embryo and/or endosperm tissue in early developmental stage, such as 1 day, 2 days, 3 days, 4 days, 5 days, 6 days, 7 days, 8 days, 9 days, or 10 days after pollination (DAP). In some embodiments, the promoters can drive gene expression only during this period of time. In some embodiments, the promoters can drive gene expression even after this period of time.

The invention provides recombinant genes comprising 3′ non-coding sequences or 3′ untranslated regions. As used herein, the “3′ non-coding sequences” or “3′ untranslated regions” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht, I. L., et al. (1989) Plant Cell 1:671-680.

The invention provides RNA transcripts. As used herein, “RNA transcript” refers to the product resulting from RNA polymerase-catalyzed transcription of a DNA sequence. When the RNA transcript is a perfect complementary copy of the DNA sequence, it is referred to as the primary transcript. An RNA transcript is referred to as the mature RNA when it is an RNA sequence derived from post-transcriptional processing of the primary transcript. “Messenger RNA (mRNA)” refers to the RNA that is without introns and that can be translated into protein by the cell. “cDNA” refers to a DNA that is complementary to and synthesized from an mRNA template using the enzyme reverse transcriptase. The cDNA can be single-stranded or converted into the double-stranded form using the Klenow fragment of DNA polymerase I. “Sense” RNA refers to RNA transcript that includes the mRNA and can be translated into protein within a cell or in vitro. “Antisense RNA” refers to an RNA transcript that is complementary to all or part of a target primary transcript or mRNA, and that blocks the expression of a target gene (U.S. Pat. No. 5,107,065). The complementarity of an antisense RNA may be with any part of the specific gene transcript, i.e., at the 5′ non-coding sequence, 3′ non-coding sequence, introns, or the coding sequence. “Functional RNA” refers to antisense RNA, ribozyme RNA, or other RNA that may not be translated but yet has an effect on cellular processes. The terms “complement” and “reverse complement” are used interchangeably herein with respect to mRNA transcripts, and are meant to define the antisense RNA of the message.

The invention provides recombinant genes in which a gene of interest is operably linked to a promoter sequence. As used herein, the term “operably linked” refers to the association of nucleic acid sequences on a single nucleic acid fragment so that the function of one is regulated by the other. For example, a promoter is operably linked with a coding sequence when it is capable of regulating the expression of that coding sequence (i.e., that the coding sequence is under the transcriptional control of the promoter). Coding sequences can be operably linked to regulatory sequences in a sense or antisense orientation. In another example, the complementary RNA regions of the invention can be operably linked, either directly or indirectly, 5′ to the target mRNA, or 3′ to the target mRNA, or within the target mRNA, or a first complementary region is 5′ and its complement is 3′ to the target mRNA.

The invention provides recombinant expression cassettes and recombinant constructs. As used herein, the phrases “recombinant construct”, “expression construct”, “chimeric construct”, “construct”, and “recombinant DNA construct” are used interchangeably herein. A recombinant construct comprises an artificial combination of nucleic acid fragments, e.g., regulatory and coding sequences that are not found together in nature. For example, a chimeric construct may comprise regulatory sequences and coding sequences that are derived from different sources, or regulatory sequences and coding sequences derived from the same source, but arranged in a manner different than that found in nature. Such construct may be used by itself or may be used in conjunction with a vector. If a vector is used then the choice of vector is dependent upon the method that will be used to transform host cells as is well known to those skilled in the art. For example, a plasmid vector can be used. The skilled artisan is well aware of the genetic elements that must be present on the vector in order to successfully transform, select and propagate host cells comprising any of the isolated nucleic acid fragments of the invention. The skilled artisan will also recognize that different independent transformation events will result in different levels and patterns of expression (Jones et al., (1985) EMBO J. 4:2411-2418; De Almeida et al., (1989) Mol. Gen. Genetics 218:78-86), and thus that multiple events must be screened in order to obtain lines displaying the desired expression level and pattern. Such screening may be accomplished by Southern analysis of DNA, Northern analysis of mRNA expression, immunoblotting analysis of protein expression, or phenotypic analysis, among others. Vectors can be plasmids, viruses, bacteriophages, pro-viruses, phagemids, transposons, artificial chromosomes, and the like, that replicate autonomously or can integrate into a chromosome of a host cell. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that is not autonomously replicating. As used herein, the term “expression” refers to the production of a functional end-product e.g., an mRNA or a protein (precursor or mature).

In some embodiments, the expression cassettes or recombinant constructs comprise at least one selectable or screenable marker. In some embodiments, the selectable or screenable marker is a plant selectable or screenable marker. As used herein, the phrase “plant selectable or screenable marker” refers to a genetic marker functional in a plant cell. A selectable marker allows cells containing and expressing that marker to grow under conditions unfavorable to growth of cells not expressing that marker. A screenable marker facilitates identification of cells which express that marker.

The invention provides inbred plants comprising recombinant sequences. As used herein, the term “inbred”, “inbred plant” is used in the context of the present invention. This also includes any single gene conversions of that inbred. The term single allele converted plant as used herein refers to those plants which are developed by a plant breeding technique called backcrossing wherein essentially all of the desired morphological and physiological characteristics of an inbred are recovered in addition to the single allele transferred into the inbred via the backcrossing technique.

The invention provides samples comprising recombinant sequences. As used herein, the term “sample” includes a sample from a plant, a plant part, a plant cell, or from a transmission vector, or a soil, water or air sample.

The invention provides offsprings comprising recombinant sequences. As used herein, the term “offspring” refers to any plant resulting as progeny from a vegetative or sexual reproduction from one or more parent plants or descendants thereof. For instance an offspring plant may be obtained by cloning or selling of a parent plant or by crossing two parent plants and include selfings as well as the F1 or F2 or still further generations. An F1 is a first-generation offspring produced from parents at least one of which is used for the first time as donor of a trait, while offspring of second generation (F2) or subsequent generations (F3, F4, etc.) are specimens produced from selfings of F1's, F2's etc. An F1 may thus be (and usually is) a hybrid resulting from a cross between two true breeding parents (true-breeding is homozygous for a trait), while an F2 may be (and usually is) an offspring resulting from self-pollination of said F1 hybrids.

The invention provides methods for crossing a first plant comprising recombinant sequences with a second plant. As used herein, the term “cross”, “crossing”, “cross pollination” or “cross-breeding” refer to the process by which the pollen of one flower on one plant is applied (artificially or naturally) to the ovule (stigma) of a flower on another plant.

The invention provides plant cultivars comprising recombinant sequences. As used herein, the term “cultivar” refers to a variety, strain or race of plant that has been produced by horticultural or agronomic techniques and is not normally found in wild populations.

In some embodiments, the present invention provides methods for obtaining plant genotypes comprising recombinant genes. As used herein, the term “genotype” refers to the genetic makeup of an individual cell, cell culture, tissue, organism (e.g., a plant), or group of organisms.

In some embodiments, the present invention provides homozygotes comprising recombinant genes. As used herein, the term “homozygote” refers to an individual cell or plant having the same alleles at one or more loci.

In some embodiments, the present invention provides homozygous plants comprising recombinant genes. As used herein, the term “homozygous” refers to the presence of identical alleles at one or more loci in homologous chromosomal segments.

In some embodiments, the transgenic cell or organism is hemizygous for the gene of interest which is under control of promoters of the present invention. As used herein, the term “hemizygous” refers to a cell, tissue or organism in which a gene is present only once in a genotype, as a gene in a haploid cell or organism, a sex-linked gene in the heterogametic sex, or a gene in a segment of chromosome in a diploid cell or organism where its partner segment has been deleted.

In some embodiments, the present invention provides heterozygotes comprising recombinant genes. As used herein, the terms “heterozygote” and “heterozygous” refer to a diploid or polyploid individual cell or plant having different alleles (forms of a given gene) present at least at one locus. In some embodiments, the cell or organism is heterozygous for the gene of interest which is under control of the synthetic regulatory element.

In some embodiments, the present invention provides heterologous polynucleotide. As used herein, the terms “heterologous polynucleotide” or a “heterologous nucleic acid” or an “exogenous DNA segment” refer to a polynucleotide, nucleic acid or DNA segment that originates from a source foreign to the particular host cell, or, if from the same source, is modified from its original form. Thus, a heterologous gene in a host cell can include a gene that is endogenous to the particular host cell, but has been modified. The terms also refer to a DNA segment which is foreign or heterologous to the cell, or homologous to the cell but in a position within the host cell nucleic acid in which the element is not ordinarily found. A heterologous gene can refer to a gene not normally found in the host organism, but that is introduced into the host organism by gene transfer. A heterologous gene can include a native coding region, or portion thereof, that is reintroduced into the source organism in a form that is different from the corresponding native gene. For example, a heterologous gene may include a native coding region that is a portion of a chimeric gene including non-native regulatory regions that is reintroduced into the native host. Also a foreign gene can comprise native genes inserted into a non-native organism, or chimeric genes. Exogenous DNA segments are expressed to yield exogenous polypeptides.

In some embodiments, the cell or organism has at least one heterologous trait. As used herein, the term “heterologous trait” refers to a phenotype imparted to a transformed host cell or transgenic organism by an exogenous DNA segment, heterologous polynucleotide or heterologous nucleic acid. Various changes in phenotype are of interest to the present invention, including but not limited to modifying the fatty acid composition in a plant, altering the amino acid content of a plant, altering a plant's pathogen defense mechanism, increasing a plant's yield of an economically important trait (e.g., grain yield, forage yield, etc.) and the like. These results can be achieved by providing expression of heterologous products or increased expression of endogenous products in plants using the methods and compositions of the present invention.

The invention provides methods for obtaining plant lines comprising recombinant genes. As used herein, the term “line” is used broadly to include, but is not limited to, a group of plants vegetatively propagated from a single parent plant, via tissue culture techniques or a group of inbred plants which are genetically very similar due to descent from a common parent(s). A plant is said to “belong” to a particular line if it (a) is a primary transformant (T0) plant regenerated from material of that line; (b) has a pedigree comprised of a T0 plant of that line; or (c) is genetically very similar due to common ancestry (e.g., via inbreeding or selfing). In this context, the term “pedigree” denotes the lineage of a plant, e.g. in terms of the sexual crosses affected such that a gene or a combination of genes, in heterozygous (hemizygous) or homozygous condition, imparts a desired trait to the plant.

The invention provides open-pollinated populations comprising recombinant genes. As used herein, the terms “open-pollinated population” or “open-pollinated variety” refer to plants normally capable of at least some cross-fertilization, selected to a standard, that may show variation but that also have one or more genotypic or phenotypic characteristics by which the population or the variety can be differentiated from others. A hybrid, which has no barriers to cross-pollination, is an open-pollinated population or an open-pollinated variety.

The invention provides self-pollination populations comprising recombinant genes. As used herein, the term “self-crossing”, “self pollinated” or “self-pollination” means the pollen of one flower on one plant is applied (artificially or naturally) to the ovule (stigma) of the same or a different flower on the same plant.

The invention provides ovules and pollens comprising recombinant genes. As used herein when discussing plants, the term “ovule” refers to the female gametophyte, whereas the term “pollen” means the male gametophyte.

In some embodiments, the transgenic plants comprising recombinant genes have one or more preferred phenotypes. As used herein, the term “phenotype” refers to the observable characters of an individual cell, cell culture, organism (e.g., a plant), or group of organisms which results from the interaction between that individual's genetic makeup (i.e., genotype) and the environment.

The invention provides plant tissue comprising recombinant genes. As used herein, the term “plant tissue” refers to any part of a plant. Examples of plant organs include, but are not limited to the leaf, stem, root, tuber, seed, branch, pubescence, nodule, leaf axil, flower, pollen, stamen, pistil, petal, peduncle, stalk, stigma, style, bract, fruit, trunk, carpel, sepal, anther, ovule, pedicel, needle, cone, rhizome, stolon, shoot, pericarp, endosperm, placenta, berry, stamen, and leaf sheath.

The invention provides methods for obtaining plants comprising recombinant genes through transformation. As used herein, the term “transformation” refers to the transfer of nucleic acid (i.e., a nucleotide polymer) into a cell. As used herein, the term “genetic transformation” refers to the transfer and incorporation of DNA, especially recombinant DNA, into a cell.

The invention provides transformants comprising recombinant genes. As used herein, the term “transformant” refers to a cell, tissue or organism that has undergone transformation. The original transformant is designated as “T0” or “T₀.” Selfing the T0 produces a first transformed generation designated as “T1” or “T₁.”

The present invention provides transgenes comprising recombinant promoters. As used herein, the term “transgene” refers to a nucleic acid that is inserted into an organism, host cell or vector in a manner that ensures its function.

The invention provides transgenic plants comprising recombinant promoters. As used herein, the term “transgenic” refers to cells, cell cultures, organisms (e.g., plants), and progeny which have received a foreign or modified gene by one of the various methods of transformation, wherein the foreign or modified gene is from the same or different species than the species of the organism receiving the foreign or modified gene.

The invention provides transgenic events comprising recombinant promoters. As used herein, the term “transposition event” refers to the movement of a transposon from a donor site to a target site.

In some embodiments, the present invention provides plant varieties comprising the recombinant genes. As used herein, the term “variety” refers to a subdivision of a species, consisting of a group of individuals within the species that are distinct in form or function from other similar arrays of individuals.

The invention provides novel nucleotide sequences of genetic regulatory elements. It is recognized that from such nucleotide sequences, a nucleic acid molecule can be synthesized or produced using a number of methods known in the art. As used herein “producing a nucleic acid molecule” is intended to comprise the making of a nucleic acid molecule by any known method including, but not limited to, chemical synthesis of the entire nucleic acid molecule or part or parts thereof, modification of a pre-existing nucleic acid molecule, such as, for example, a DNA molecule comprising a genetic regulatory element of the present invention, by molecular biology methods such as, for example, restriction endonuclease digestion, DNA amplification by polymerase and ligation, and the combination of chemical synthesis and modification.

In some embodiments, the present invention provides organisms with recombinant genes. As used herein, an “organism” refers any life form that has genetic material comprising nucleic acids including, but not limited to, prokaryotes, eukaryotes, and viruses. Organisms of the present invention include, for example, plants, animals, fungi, bacteria, and viruses, and cells and parts thereof.

The invention provides coding sequences of a gene of interest that are operably linked with the promoters of the present invention. As used herein, “coding sequence” refers to a DNA sequence that codes for a specific amino acid sequence. By “gene of interest” is intended any nucleotide sequence, either coding sequence or non-coding sequence that can be expressed when operably linked to a promoter. A gene of interest of the present invention may, but need not, encode a protein. Unless stated otherwise or readily apparent from the context, when a gene of interest of the present invention is said to be operably linked to a promoter of the invention, the gene of interest does not by itself comprise a functional promoter. “Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. As used herein, “regulatory sequences” may include, but are not limited to, promoters, translation leader sequences, introns, and polyadenylation recognition sequences. As used herein, the term “operably linked” refers to the association of nucleic acid sequences on a single nucleic acid fragment so that the function of one is regulated by the other. For example, a promoter is operably linked with a coding sequence when it is capable of regulating the expression of that coding sequence (i.e., that the coding sequence is under the transcriptional control of the promoter). Coding sequences can be operably linked to regulatory sequences in a sense or antisense orientation. In another example, the complementary RNA regions of the invention can be operably linked, either directly or indirectly, 5′ to the target mRNA, or 3′ to the target mRNA, or within the target mRNA, or a first complementary region is 5′ and its complement is 3′ to the target mRNA.

In some embodiments, the transgenes of the present invention comprise at least one reporter gene. As used herein a “reporter” or a “reporter gene” refers to a nucleic acid molecule encoding a detectable marker. The reporter gene can be, for example, luciferase (e.g., firefly luciferase or Renilla luciferase), β-glucuronidase (GUS), β-galactosidase, chloramphenicol acetyl transferase (CAT), or a fluorescent protein (e.g., green fluorescent protein (GFP), red fluorescent protein (DsRed), yellow fluorescent protein, blue fluorescent protein, cyan fluorescent protein, or variants thereof, including enhanced variants such as enhanced GFP (eGFP). Reporter genes are detectable by a reporter assay. Reporter assays can measure the level of reporter gene expression or activity by any number of means, including, for example, measuring the level of reporter mRNA, the level of reporter protein, or the amount of reporter protein activity. Reporter assays are known in the art or otherwise disclosed herein.

Identification of Embryo- and/or Endosperm-Specific or Preferred Promoters

There has been little interest in the scientific community in isolating early embryo-specific promoters from corn. There are instances of using microarray technology (for instance, Lee et al Funct Integr Genomics (2002) 2:13-27); however to our knowledge, the genes identified have not been validated and their promoter regions isolated.

The invention provides methods to identify embryo- and endosperm-specific or preferred promoters from plant species. In some embodiments, the plant species is a maize species.

In some embodiments, to search for early embryo- and endosperm-specific or preferred promoters in maize, two different approaches can be taken: (1) public sequence databases are queried for potential genes expressed only in embryo tissue, and (2) Illumina® next-generation sequencing is performed on mRNA from early embryo and endosperm maize tissue.

In some embodiments, the public maize EST database are the full length cDNA libraries available online, e.g., the “unique contigs” in the PAVE EST assemblies database. In some embodiments, the cDNA libraries are the libraries of embryo or endosperm.

In some embodiments, candidate genes are tested by qRT-PCR or other suitable methods to confirm their expression patterns and the ones that show embryo- and/or endosperm-specific or preferred expression pattern are selected and subjected to further investigation. In some embodiments, a reporter gene is operably linked to the promoter sequence of a candidate gene and transformed into a plant, plant part or plant cell, and the expression of the reporter gene is monitored. Promoters that can drive the expression of the reporter gene in an embryo- and/or endosperm-specific or preferred manner are identified.

Embryo- and/or Endosperm-Specific or Preferred Promoters

The present invention provides promoters that can be used to express a gene of interest in an embryo- and/or endosperm-specific or preferred manner in a maize plant.

In some embodiments, the promoters are the ones associated with the genes listed in Table 2. In some embodiments, the promoters are selected from the group consisting of the promoters associated with the genes having MaizeSequence Nos. GRMZM2G124663 (also called TGI ZmLEC1 or ZmEmb2 herein), GRMZM2G040517, GRMZM2G066546, GRMZM2G046532, GRMZM2G087413 (also called ZmBETL-9 herein), GRMZM2G091054, GRMZM2G120008 (also called ZmEmb1 herein), GRMZM2G158407 (also called ZmSh-2 herein), GRMZM2G132162, GRMZM2G006585 (also called ZmEndosperm1 herein), GRMZM2G700896, GRMZM2G091445 (also called ZmBETL-10), GRMZM2G059620, GRMZM2G088896, GRMZM2G175976 (also called ZmBETL-3 herein), GRMZM2G046086, GRMZM2G175912, GRMZM2G152655 (also called ZmBETL-2 herein), GRMZM2G138727 (also called ZmEndosperm2 herein), GRMZM2G025763, GRMZM2G090264, GRMZM2G157806 (also called ZmEmb3 herein), GRMZM2G472234 (also called ZmEmb5 herein), GRMZM2G409372, GRMZM2G065157, GRMZM2G169149, GRMZM2G701926, GRMZM2G174883, GRMZM2G369799, GRMZM2G140302, GRMZM2G176390 (also called ZmEmb4 herein) and genes encoding PER1 (e.g., HvPER1), Hordein (e.g., Hv B1 Hordein), ASP (e.g., ZmASP1, OsASP), CLV1 (e.g., ZmCLV1), and MRP (e.g., ZmMRP1). The MaizeSequence accession numbers referred above are assigned by the Maize Genome Sequencing Project, MaizeSequence Release 5b.60, February 2011, which is available through the website of MaizeSequence organization, sponsored by NSF, USDA, DOE, and Cold Spring Harbor Laboratory. See Schnable et al. (“The B73 maize genome: complexity, diversity and dynamics.” Science, 2009; 326:1112-1115), and Taber et al. (“MaizeGDB becomes ‘sequence-centric’”, Database (Oxford). 2009; 2009: bap020.), each of which is incorporated by reference in its entirety.

In some embodiments, the invention provides promoter variants having one or more alterations when compared to any one of SEQ ID NOs: 1-37. For example, the mutant promoters are the ones that share at least 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or more to any one of SEQ ID NOs: 1-37, while still maintaining the ability to drive gene expression in an embryo- and/or endosperm-specific or preferred manner. In some embodiments, the promoter variants are generated by silent substitutions, additions, or deletions, but do not alter the properties or activities of the promoters. Nucleotide variants can be produced for a variety of reasons, e.g., to optimize codon expression for a particular host.

In some embodiments, the invention provides promoter variants having one or more fragments of one or more promoters defined by SEQ ID NOs: 1-37. For example, the fragment can comprise about 50 bp, 100 bp, 150 bp, 200 bp, 300 bp, 400 bp, 500 bp, 600 bp, 700 bp, 800 pb, 900 bp, 1 kb, 1.5 kb, 2.0 kb, 2.5 kb, 3 kb, 4 kb, 5, kb, 6 kb, 7 kb, 8 kb, 9 kb, 10 kb, 15 kb, 20 kb, 25 kb, 30 kb, or more of the promoter sequences disclosed herein, while still being able to drive gene expression in an embryo- and/or endosperm-specific or preferred manner in a plant, such as a maize plant.

Also provided are isolated homologous promoters having similar function of the promoters described above from difference maize varieties or different species in the genus of Zea (e.g., Zea diploperennis, Zea luxurians, Zea mays L., Zea mays huehuetenangensis, Zea mays mays, Zea mays mexicana, Zea mays parviglumis, Zea nicaraguensis, and Zea perennis). Such promoters can also be used to express a gene of interest in maize plants. In some embodiments, homologous promoters from other species can be cloned by the classical approach. For example, the homologous promoters can be identified by using any part of any one of the maize promoters described herein as a probe to a genomic library of a different maize varieties or different species in the genus of Zea. In some embodiments, a homologous promoter can be isolated based on hybridization of two nucleic acid molecules under stringent conditions. More detailed methods of cloning homologous genes based on a known gene is described in “Gene Cloning and DNA Analysis: An Introduction”, (Publisher: John Wiley and Sons, 2010, ISBN 1405181737, 9781405181730), and “Gene cloning: principles and applications” (Publisher: Nelson Thornes, 2006). In some embodiments, the homologous promoters share at least 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or more sequence identity to any one of SEQ ID NOs: 1-37.

Alternatively, one can first use any part of the coding sequence of any one of the maize embryo- and/or endosperm-specific or preferred genes described herein to isolate the coding sequence of homologous genes in other maize varieties or other species in the genus of Zea, then isolate the promoters associated with said the coding sequence of the homologous genes. In some embodiments, the homologous genes isolated from other maize varieties or other species in the genus of Zea share at least 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or more sequence identity to any one of the embryo and/or endosperm specific or preferred genes disclosed herein. Accordingly, promoter sequences associate with those homologous genes can be isolated and used to drive the expression of a gene of interest in an embryo- and/or endosperm-specific or preferred manner in maize plants.

GRMZM2G124663 gene is also called TGI ZmLEC1 or ZmEmb2 herein. This gene is different from the maize LEC1 gene described in U.S. Pat. No. 7,432,418, or WO 2002/042424, each of which is incorporated herein by reference in its entirety. The promoter of the maize LEC1 gene described in the U.S. Pat. No. 7,432,418 (SEQ ID NO: 6 in that patent) is included in the present application as SEQ ID NO: 39. Arabidopsis LEC1 gene promoter is described in U.S. Pat. No. 6,545,201 (e.g., positions 1-1998 of SEQ ID NO: 3 in the U.S. Pat. No. 6,545,201, which is included in the present application as SEQ ID NO: 40, or the first 4.4 kb of SEQ ID NO: 4 in the U.S. Pat. No. 6,545,201, which is included in the present application as SEQ ID NO: 41).

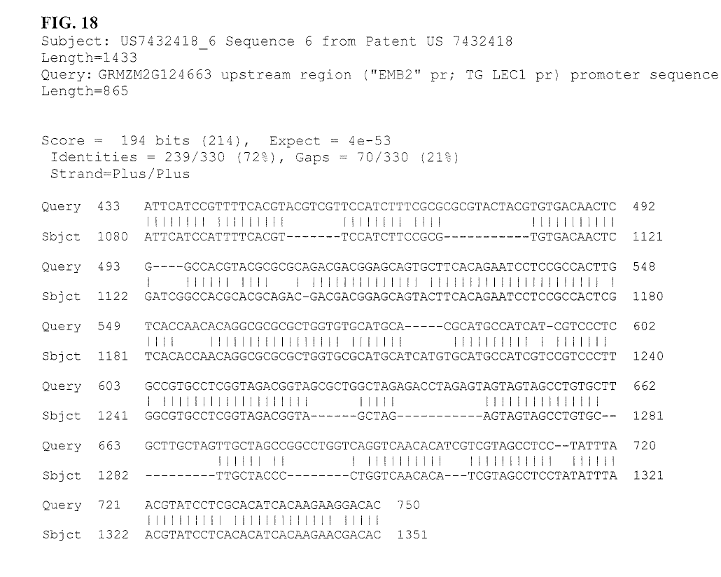

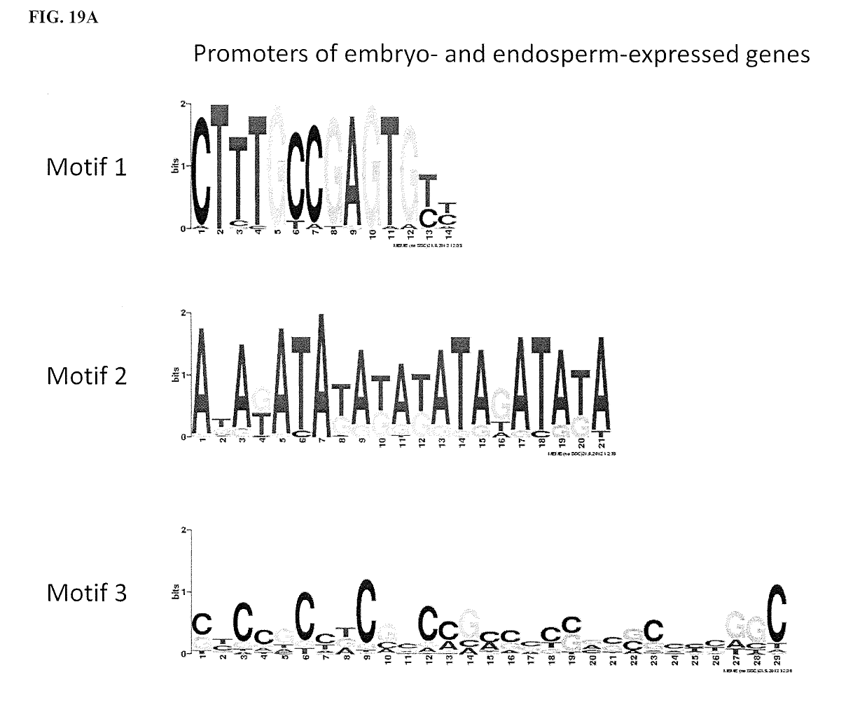

The sequence alignment in FIG. 18 shows that there are several conserved consensus fragments between the LEC1 gene promoter described in U.S. Pat. No. 7,432,418 and the maize LEC1 promoter of the present application. Therefore, the present invention also include promoter sequences comprising one, or two, or three, or more conserved consensus fragments of the specific promoter sequences disclosed herein, wherein the promoters can drive gene expression in an embryo- and/or endosperm-specific or preferred manner. Such conserved consensus fragments can be arranged in the same order as those in the specific promoter sequences disclosed herein, or re-arranged. The conserved consensus fragments can be identified by comparing a specific promoter sequence of the embryo- and/or endosperm genes provided herein to a promoter sequence of an orthologous or paralogous embryo- and/or endosperm gene derived from a different source, such as a difference plant species. Alternatively, the conservative consensus fragments can be identified with the help of computational software, such as Promoter 2.0 Prediction Server provided by Center for Biological Sequence Analysis, Promoter Prediction by Neural Network provided by Lawrence Berkeley Laboratory, USA, PromScan provided by Queen's University, Canada, Virtual Footprint provided by R. Munch et al. (2005, Bioinformatics 2005, 21:4187-4189), Suite for Computational Identification of Promoter Elements provided by J. M. Carlson et al. (2007, Nucl. Acid Res. 35:W259-W264), Neural Network Promoter Prediction (M. G. Reese 2001. Comput. Chem. 26: 51-6), and Promoter and gene expression regulatory motifs search provided by Softberry, U.S.A.

Conserved consensus fragments in embryo- and/or endosperm-specific or preferred promoters can also be discovered with the help of computational software such as those found in the MEME Suite Motif-based sequence analysis tools (Timothy L. Bailey, Mikael Bodén, Fabian A. Buske, Martin Frith, Charles E. Grant, Luca Clementi, Jingyuan Ren, Wilfred W. Li, William S. Noble, “MEME SUITE: tools for motif discovery and searching”, Nucleic Acids Research, 37:W202-W208, 2009). Such conserved consensus fragments can be arranged in the same order as those in the specific promoter sequences disclosed herein, or re-arranged. Such conserved consensus fragments can be multimerized or used individually. Such conserved consensus fragments can be combined with a heterologous promoter to alter expression of the heterologous promoter to be embryo- and/or endosperm-specific or preferred.

The promoters of the present invention can drive gene expression at different levels in an early embryo- and/or endosperm-specific or preferred manner in maize. The promoters disclosed herein have different activities, allowing expression of a gene interest at a desired or pre-determined level. This is very important in regulating gene expression. Some genes can be toxic or bring detrimental effects to the host when highly expressed, but beneficial to the host when expressed at a lower level. On the other hand, some genes are naturally weakly expressed at a low level, and can only bring beneficial effects to the host when highly expressed. The promoters of the present invention provide a broad spectrum of expression levels which can be elected based on the purpose to achieve the most ideal results.

### Expression Cassettes and Vectors

The present invention provides expression cassettes and expression vectors comprising one or more embryo- and/or endosperm-specific or preferred promoters of the present application.

The backbone of the expression vectors can be any expression vectors suitable for producing transgenic plant, which are well known in the art. In some embodiments, the expression vector is suitable for expressing transgene in maize.

In some embodiments, the expression vector is an Agrobacterium binary vector (see, Karimi et al., Plant Physiol 145: 1183-1191; Komari et al., Methods Mol Biol 343: 15-42; Bevan M W (1984) Nucleic Acids Res 12: 1811-1821; Becker (1992), Plant Mol Biol 20: 1195-1197; Datla et al., (1992), Gene 122: 383-384; Hajdukiewicz (1994) Plant Mol Biol 25:989-994; Xiang (1999), Plant Mol Biol 40: 711-717; Chen et al., (2003) Mol Breed 11: 287-293; Weigel et al., (2000) Plant Physiol 122: 1003-1013). In another embodiment, the expression vector is a co-integrated vector (also called hybrid Ti plasmids). More expression vectors and methods of using them can be found in U.S. Pat. Nos. 4,940,838, 5,464,763, 5,149,645, 5,501,967, 6,265,638, 4,693,976, 5,635,381, 5,731,179, 5,693,512, 6,162,965, 5,693,512, 5,981,840, 6,420,630, 6,919,494, 6,329,571, 6,215,051, 6,369,298, 5,169,770, 5,376,543, 5,416,011, 5,569,834, 5,824,877, 5,959,179, 5,563,055, and 5,968,830. Each of the references mentioned herein is incorporated by reference in its entirety.

In some embodiments, the expression cassettes or the expression vectors comprise at least one nucleic acid sequence encoding a gene of interest. In some embodiments, the gene of interest is operably linked to one or more embryo- and/or endosperm-specific or preferred promoters described herein.

In some embodiments, the gene of interest is associated with one or more agronomically important traits. As used herein, “agronomically important traits” include any phenotype in a plant or plant part that is useful or advantageous for human use. Examples of agronomically important traits include but are not limited to those that result in increased biomass production, increased food production, improved food quality, decrease in cracking, quicker color change when the fruit matures etc. Additional examples of agronomically important traits includes pest resistance, vigor, development time (time to harvest), enhanced nutrient content, novel growth patterns, flavors or colors, salt, heat, drought and cold tolerance, and the like.

Other agronomically important traits include resistance to biotic and/or abiotic stresses. As used herein, the phrase “biotic stress” or “biotic pressure” refers to a situation where damage is done to plants by other living organisms, such as bacteria, viruses, fungi, parasites, insects, weeds, animals and human. As used herein, the phrase “abiotic stress” or “abiotic pressure” refers to the negative impact of non-living factors on plants in a specific environment. The non-living variable must influence the environment beyond its normal range of variation to adversely affect the population performance or individual physiology of plants in a significant way. Non-limiting examples of stressors are high winds, extreme temperatures, drought, flood, and other natural disasters, such as tornados and wildfires.

In some embodiments, the trait is associated with increased biomass production, production of specific biofuels, increased food production, improved food quality, increased seed oil content, altered fatty acid composition, etc. In some embodiments, the gene of interest is a REVOLUTA gene or a dominant negative KRP gene, as described in WO 2007/016319 and WO 2007/079353, each of which is incorporated herein by reference in its entirety. In some embodiments, the gene of interest is an acyl-acyl carrier protein (acyl-ACP) thioesterase (Hawkins and Kridl 1998 The Plant Journal 13: 743-752; Facciotti 1999 Nature Biotechnology 17: 593-597; WO 1996/06936), an acyl-acyl carrier protein (acyl-ACP) desaturase (Knutzon 1992 PNAS 89: 2624-2628; Liu 2002 Plant Physiology 129: 1732-1743; Rousselin 2002 Plant Breeding 121: 108-116), a fatty acid elongase (Millar and Kunst 1997 The Plant Journal 12: 121-131), a fatty acid desaturase (Okuley 1994 The Plant Cell 6: 147-158; Hitz 1994 Plant Phys. 105: 635-641; U.S. Pat. No. 5,846,784; Jaworski and Cahoon 2003 Current Opinion in Plant Biology 6: 178-184), an acyl-acyl carrier protein synthase III (Verwoert 1995 Plant Molecular Biology 27: 875-886), a leafy cotyledon 2 (Mendoza 2005 FEBS Letters 579: 4666-4670), a glycerol-3-phosphate dehydrogenase (Vigeolas 2007 Plant Biotechnology Journal 5:431-441), a diacylglycerol acyltransferase (Lardizabal 2008 Plant Physiology 148: 89-96), a lysophosphatidic acid acyltransferase (Zou 1997 The Plant Cell, 9:909-923; Lassner 1995 Plant Physiology 109: 1389-1394), a seed storage protein (WO 1997/47731; WO 92/14822; Kohno-Murase 1994 Plant Molecular Biology 26: 1115-1124; Pickardt 1995 Molecular Breeding 1: 295-301; De Clercq 1990 Plant Physiol 94: 970-979), an aspartate kinase (Karchi 1993 The Plant Journal 3: 721-727; Falco 1995 Nature Biotechnology 13, 577-582), a dihydrodipicolinic acid synthase (Falco 1995 Nature Biotechnology 13, 577-582; WO 1989/11789; Shaul and Galili 1992 Plant J. 2: 203-209; Lee 2001 Molecular Breeding 8: 75-84), an Sb401 gene from potato (Yu 2004 Molecular Breeding 14: 1-7), a phosphoenolpyruvate carboxylase (Rolletschek 2004 Plant Biotechnology Journal 2: 211-219), puroindoline genes (Zhang Plant Biotechnology Journal (2009) 7: 733-743), a phytoene synthase (Shewmaker 1999 The Plant Journal 20: 401-412; Ravanello 2003 Metabolic Engineering 5: 255-263), an abscisic acid biosynthesis gene (Frey 1999 Plant Molecular Biology 39: 1267-1274), an isopentenyl transferase (Ma 1998 Australian Journal of Plant Physiology 25: 53-59; U.S. Pat. No. 6,992,237; U.S. Pat. No. 7,531,723), a sucrose symporter (Rosche 2002 The Plant Journal 30: 165-175), a starch branching enzyme gene (WO 1997/22703), a levansucrase (Caimi 1996 Plant Physiol. 110: 355-363), an invertase (Hirose 2002 Plant Cell Physiol 43: 452-459; Weber 1998 The Plant Journal 16: 163-172; Cheng 1996 The Plant Cell 8: 971-983), a sucrose synthase (Ruan 2003 The Plant Cell 15: 952-964; Ruan 2010 Mol. Plant. 3: 942-955), a sucrose isomerase (PCT/US2012/027750; Wu 2007 Plant Biotechnol J. 5:109-17), a phytase (Chiera 2004 Plant Molecular Biology 56: 895-904), DREB1A (Kasuga 1999 Nature Biotech 17: 287-291), ABI3 (Tamminen 2001 The Plant Journal 25: 1-8), ADP-glucose pyrophosphorylase (Smidansky 2002 PNAS 99: 1724-1729; Hannah 2012 The Plant Cell 24: 2352-2363), recombinant proteins for molecular farming applications (Boothe 2010 Plant Biotechnology Journal 8: 588-606), each of which is incorporated by reference in its entirety.

In some embodiments, the gene of interest encodes a polypeptide. In some other embodiments, the gene of interest encodes a microRNA. In some other embodiments, the gene of interest encodes an interference RNA. The agronomically important traits are achieved by expression of the polypeptide, and/or the expression of the microRNA or the interference RNA in the plant.

RNA interference (RNAi) is the process of sequence-specific, post-transcriptional gene silencing or transcriptional gene silencing in animals and plants, initiated by double-stranded RNA (dsRNA) that is homologous in sequence to the silenced gene. The term “dsRNA” or “dsRNA molecule” or “double-strand RNA effector molecule” refers to an at least partially double-strand ribonucleic acid molecule containing a region of at least about 19 or more nucleotides that are in a double-strand conformation. The double-stranded RNA effector molecule may be a duplex double-stranded RNA formed from two separate RNA strands or it may be a single RNA strand with regions of self-complementarity capable of assuming an at least partially double-stranded hairpin conformation (i.e., a hairpin dsRNA or stem-loop dsRNA). In various embodiments, the dsRNA consists entirely of ribonucleotides or consists of a mixture of ribonucleotides and deoxynucleotides, such as RNA/DNA hybrids. The dsRNA may be a single molecule with regions of self-complementarity such that nucleotides in one segment of the molecule base pair with nucleotides in another segment of the molecule. In one aspect, the regions of self-complementarity are linked by a region of at least about 3-4 nucleotides, or about 5, 6, 7, 9 to 15 nucleotides or more, which lacks complementarity to another part of the molecule and thus remains single-stranded (i.e., the “loop region”). Such a molecule will assume a partially double-stranded stem-loop structure, optionally, with short single stranded 5′ and/or 3′ ends. In one aspect the regions of self-complementarity of the hairpin dsRNA or the double-stranded region of a duplex dsRNA will comprise an Effector Sequence and an Effector Complement (e.g., linked by a single-stranded loop region in a hairpin dsRNA). The Effector Sequence or Effector Strand is that strand of the double-stranded region or duplex which is incorporated in or associates with RISC. In one aspect the double-stranded RNA effector molecule will comprise an at least 19 contiguous nucleotide effector sequence, preferably 19 to 29, 19 to 27, or 19 to 21 nucleotides, which is a reverse complement to the targeted gene, or an opposite strand replication intermediate, or the anti-genomic plus strand or non-mRNA plus strand sequences of the targeted gene. In one embodiment, said double-stranded RNA effector molecules are provided by providing to a plant, plant tissue, or plant cell an expression construct comprising one or more double-stranded RNA effector molecules. One skilled in the art will be able to design suitable double-strand RNA effector molecule based on the nucleotide sequences of the targeted gene.

In some embodiments, the dsRNA effector molecule of the invention is a “hairpin dsRNA”, a “dsRNA hairpin”, “short-hairpin RNA” or “shRNA”, i.e., an RNA molecule of less than approximately 400 to 500 nucleotides (nt), or less than 100 to 200 nt, in which at least one stretch of at least 15 to 100 nucleotides (e.g., 17 to 50 nt, 19 to 29 nt) is based paired with a complementary sequence located on the same RNA molecule (single RNA strand), and where said sequence and complementary sequence are separated by an unpaired region of at least about 4 to 7 nucleotides (or about 9 to about 15 nt, about 15 to about 100 nt, about 100 to about 1000 nt) which forms a single-stranded loop above the stem structure created by the two regions of base complementarity. The shRNA molecules comprise at least one stein-loop structure comprising a double-stranded stem region of about 17 to about 100 bp; about 17 to about 50 bp; about 40 to about 100 bp; about 18 to about 40 bp; or from about 19 to about 29 bp; homologous and complementary to a target sequence to be inhibited; and an unpaired loop region of at least about 4 to 7 nucleotides, or about 9 to about 15 nucleotides, about 15 to about 100 nt, about 100 to about 1000 nt, which forms a single-stranded loop above the stem structure created by the two regions of base complementarity. It will be recognized, however, that it is not strictly necessary to include a “loop region” or “loop sequence” because an RNA molecule comprising a sequence followed immediately by its reverse complement will tend to assume a stem-loop conformation even when not separated by an irrelevant “stuffer” sequence.

Optionally, the nucleic acid sequence encoding the gene of interest is also operably linked to a plant 3′ non-translated region (3′ UTR). A plant 3′ non-translated sequence is not necessarily derived from a plant gene. For example, it can be a terminator sequence derived from viral or bacterium gene, or T-DNA. The 3′ non-translated regulatory DNA sequence can include from about 20 to 50, about 50 to 100, about 100 to 500, or about 500 to 1,000 nucleotide base pairs and may contain plant transcriptional and translational termination sequences in addition to a polyadenylation signal and any other regulatory signals capable of effecting mRNA processing or gene expression. Non-limiting examples of suitable 3′ non-translated sequences are the 3′ transcribed non-translated regions containing a polyadenylation signal from the nopaline synthase (NOS) gene of Agrobacterium tumefaciens (Bevan et al., 1983, Nucl. Acid Res., 11:369), or terminator for the T7 transcript from the octopine synthase gene of Agrobacterium tumefaciens. More suitable 3′ non-translated sequences include, 3′UTR of the potato cathepsin D inhibitor gene (GenBank Ace. No.: X74985), 3′UTR of the field bean storage protein gene VfLEIB3 (GenBank Ace. No.: Z26489), 3′UTR of pea E9 small subunit of the ribulose-1,5-bisphosphate carboxylase (ssRUBISCO) gene, 3′UTR of pea bcs, the tm1 terminator, the AHAS large and small subunit terminators, and OCS gene (octopene synthase) terminator. Each of the publications on plant 3′ non-translated region mentioned herein is incorporated by reference in its entirety. The plant 3′ non-translated regions and plant promoters mentioned herein can be used in vectors for both monocotyledon and dicotyledon transformations.

The expression cassettes or the expression vectors of the present invention further comprise nucleic acids encoding one or more selection markers. The selection marker can be a positive selectable marker, a negative selectable marker, or combination thereof. A “positive selectable marker gene” encodes a protein that allows growth on selective medium of cells that carry the marker gene, but not of cells that do not carry the marker gene. Selection is for cells that grow on the selective medium (showing acquisition of the marker) and is used to identify transformants. A common example is a drug-resistance marker such as NPT (neomycin phosphotransferase), whose gene product detoxifies kanamycin by phosphorylation and thus allows growth on media containing the drug. Other positive selectable marker genes for use in connection with the present invention include, but are not limited to, a Neo gene (Potrykus et al., 1985), which codes for kanamycin resistance and can be selected for using kanamycin, G418, etc.; a bar gene, which codes for bialaphos (basta) resistance; a mutant aroA gene, which encodes an altered EPSP synthase protein (Hinchee et al., 1988), thus conferring glyphosate resistance; a nitrilase gene such as bxn from Klebsiella ozaenae, which confers resistance to bromoxynil (Stalker et al., 1988); a mutant acetolactate synthase gene (ALS), which confers resistance to imidazolinone, sulfonylurea or other ALS inhibiting chemicals (European Patent Application 154,204,1985); a methotrexate resistant DHFR gene (Thillet et al., 1988), or a dalapon dehalogenase gene that confers resistance to the herbicide dalapon; the pat gene from Streptomyces viridochromogenes, which encodes the enzyme phosphinothricin acetyl transferase (PAT) and inactivates the active ingredient in the herbicide bialaphos, phosphinothricin (PPT); or a mutated anthranilate synthase gene that confers resistance to 5-methyl tryptophan. Additional positive selectable marker genes include those genes that provide resistance to environmental factors such as excess moisture, chilling, freezing, high temperature, salt, and oxidative stress. Of course, when it is desired to introduce such a trait into a plant as a “gene of interest”, the selectable marker cannot be one that provides for resistance to an environmental factor.

Markers useful in the practice of the claimed invention include: an “antifreeze” protein such as that of the winter flounder (Cutler et al., 1989) or synthetic gene derivatives thereof; genes which provide improved chilling tolerance, such as that conferred through increased expression of glycerol-3-phosphate acetyltransferase in chloroplasts (Murata et al., 1992; Wolter et al., 1992); resistance to oxidative stress conferred by expression of superoxide dismutase (Gupta et al., 1993), and may be improved by glutathione reductase (Bowler et al., 1992); genes providing “drought resistance” and “drought tolerance”, such as genes encoding for mannitol dehydrogenase (Lee and Saier, 1982) and trehalose-6-phosphate synthase (Kaasen et al., 1992).

A “negative selectable marker gene” encodes a protein that prevents the growth of a plant or plant cell on selective medium of plants that carry the marker gene, but not of plants that do not carry the marker gene. Selection of plants that grow on the selective medium provides for the identification of plants that have eliminated or evicted the selectable marker genes. An example is CodA (Escherichia coli cytosine deaminase), whose gene product deaminates 5-fluorocytosine (which is normally non-toxic as plants do not metabolize cytosine) to the toxic 5-fluorouracil. Other negative selectable markers include the haloalkane dehalogenase (dhlA) gene of Xanthobacter autotrophicus GJ10 which encodes a dehalogenase, which hydrolyzes dihaloalkanes, such as 1,2-dichloroethane (DCE), to a halogenated alcohol and an inorganic halide (Naested et al., 1999, Plant J. 18 (5): 571-6). Each of the publications on selectable markers mentioned herein is incorporated by reference in its entirety.

Optionally, additional nucleic acid sequence can be included into the expression vectors of the present invention to facilitate the transcription, translation, and post-translational modification, so that expression and accumulation of a gene of interest in a plant cell are increased. Such additional nucleic acid sequence can enhance either the expression, or the stability of the protein. In some embodiments, such nucleic acid is an intron that has positive effect on gene expression, which has been also known as intron-mediated enhancement (IME, see Mascarenhas et al., (1990). Plant Mol. Biol. 15: 913-920). IME has been observed in a wide range of eukaryotes, including vertebrates, invertebrates, fungi, and plants (see references 17-26), suggesting that it reflects a fundamental feature of gene expression. In many cases, introns have a larger influence than do promoters in determining the level and pattern of expression. Non-limiting IME in plants have been described in Rose et al. (The Plant Cell 20:543-551 (2008)); Lee et al. (Plant Physiology 145:1294-1300 (2007)); Casas-Mollano et al. (Journal of Experimental Botany Volume 57, Number 12 Pp. 3301-3311); Jeong et al. (Plant Physiology 140:196-209 (2006)); Clancy et al. (Plant Physiol, October 2002, Vol. 130, pp. 918-929); Jeon et al. (Plant Physiol, July 2000, Vol. 123, pp. 1005-1014); Rose et al. (Plant Physiol, February 2000, Vol. 122, pp. 535-542); Kim et al. (Plant Physiol. (1999) 121: 225-236), and Callis et al. (Genes Dev. 1987 1: 1183-1200). Each of the publications on IMEs mentioned herein is incorporated by reference in its entirety. Thus, in some embodiments, any one of the IME described herein can be included in the expression vectors of the present invention. For example, an intron (SEQ ID NO: 38, 43 or 44) of ADH1 (Alcohol Dehydrogenase 1) (see Callis et al., Genes Dev. 1987 1: 1183-1200; Mascarenhas et al., Plant Mol Biol 1990 15: 913-920) gene or an intron from the gene associated with the promoter, such as the Embryo4 intron (SEQ ID NO: 42) of GRMZM2G176390, can be included upstream of the initiator methionine to increase expression.

In some embodiments, the expression cassettes or the expression vectors comprise at least one enhancer sequence. In some embodiments, the enhancer sequence is a transcriptional enhancer sequence and/or a translation enhancer sequence. In some embodiments, the enhancer is a non-translated leader sequence. In some embodiments, the enhancer sequence is the intron sequence associated with an alcohol dehydrogenase (ADH) gene, the Heat Shock Protein 70 leader sequence (U.S. Pat. No. 5,659,122), or the petE enhancer sequence (WO 97/20056). In some embodiments, the alcohol dehydrogenase gene is a plant gene. In some embodiments, the plant alcohol dehydrogenase gene is the ADH gene in rice or maize. In some embodiments, the intron of the plant ADH gene comprises the sequence of SEQ ID NO. 38, 43 or 44. In some embodiments, the enhancer is associated with a viral gene. For example, the enhancer sequence is the 5′proximal region of the genome of a potyvirus (e.g., WO/1998/044097), or viral non-translated leader sequence associated with virus selected from the group consisting of Tobacco Mosaic Virus (TMV), Maize Chlorotic Mottle Virus (MCMV), Alfalfa Mosaic Virus (AMV), Picornavirus, Potyvirus, and AMV RNA4. In some embodiments, the viral enhancer sequence can suppress gene silencing, such as the sequence associated with the gene encoding P1\HC-Pro, the 2b protein of cucumber mosaic virus (CMV), the enhancer sequences derived from the CaMV (U.S. Pat. Nos. 5,164,316; 5,196,525; 5,322,938; 5,530,196; 5,352,605; 5,359,142; and 5,858,742 for example), and HC-Pro of potato virus Y (PVY) (e.g., U.S. Pat. No. 6,395,962). Other non-limiting examples of enhancer sequences include shrunken-1 (Sh1) intron/exon enhancer sequence (U.S. Pat. No. 5,955,330), the second intron of the AGAMOUS gene (Deyholos Plant Cell. 2000 October; 12(10):1799-1810), the enhancer associated with maize tb1 gene (Richard et al., Nature Genetics—38, 594-597, 2006), and the enhancer sequence associated with a polyubiquitin gene (e.g., Sivamani and Qu, Plant Molecular Biology, 2006, 60:225-239). Each of the references mentioned above is incorporated herein by reference in its entirety.

The expression cassettes or the expression vectors of the present invention can be transformed into a maize plant to increase the seed weight, seed number, and/or seed size thereof, using the transformation methods described separately below. Thus, the present invention provides transgenic maize plants transformed with the expression vectors as described herein. The plant can be any maize plant in which an increased seed weight and/or seed size is preferred by breeders for any reasons, e.g., for economical/agricultural interests.

Methods of Using the Embryo- and/or Endosperm-Specific or Preferred Promoters

The present invention provides methods of making and using the expression cassettes comprising one or more embryo- and/or endosperm-specific or preferred promoters described herein.

In some embodiments, the expression cassettes are used to express a gene of interest in a plant or plant cell, said method comprising incorporating into a plant cell a polynucleotide construct comprising a nucleic acid molecule of the present invention. In some embodiments, the nucleic acid molecule is operably linked to a gene of interest. In some embodiments, the gene of interest is a gene which when expressed will lead to one or more agronomically important traits.

The present invention also provides methods of expressing a gene of interest in an embryo- and/or endosperm-specific or preferred manner in a maize plant. In some embodiments, the methods comprise incorporating an expression cassette of the present invention into a maize plant. The present invention also provides methods of making and using a plant comprising the expression cassettes described herein.

Any transgenic plant incorporated with the expression cassette generated from the present invention can be used as a donor to produce more transgenic plants through plant breeding methods well known to those skilled in the art. The goal in general is to develop new, unique and superior varieties and hybrids. In some embodiments, selection methods, e.g., molecular marker assisted selection, can be combined with breeding methods to accelerate the process. Additional breeding methods have been known to one of ordinary skill in the art, e.g., methods discussed in Chahal and Gosal (Principles and procedures of plant breeding: biotechnological and conventional approaches, CRC Press, 2002, ISBN 084931321X, 9780849313219), Taji et al. (In vitro plant breeding, Routledge, 2002, ISBN 156022908X, 9781560229087), Richards (Plant breeding systems, Taylor & Francis U S, 1997, ISBN 0412574500, 9780412574504), Hayes (Methods of Plant Breeding, Publisher: READ BOOKS, 2007, ISBN1406737062, 9781406737066), each of which is incorporated by reference in its entirety.

In some embodiments, said method comprises (i) crossing any one of the plants of the present invention comprising the expression cassette as a donor to a recipient plant line to create a F1 population; (ii) selecting offsprings that have expression cassette. Optionally, the offsprings can be further selected by testing the expression of the gene of interest.

In some embodiments, complete chromosomes of the donor plant are transferred. For example, the transgenic plant with the expression cassette can serve as a male or female parent in a cross pollination to produce offspring plants, wherein by receiving the transgene from the donor plant, the offspring plants have the expression cassette.

In a method for producing plants having the expression cassette, protoplast fusion can also be used for the transfer of the transgene from a donor plant to a recipient plant. Protoplast fusion is an induced or spontaneous union, such as a somatic hybridization, between two or more protoplasts (cells in which the cell walls are removed by enzymatic treatment) to produce a single bi- or multi-nucleate cell. The fused cell, that may even be obtained with plant species that cannot be interbred in nature, is tissue cultured into a hybrid plant exhibiting the desirable combination of traits. More specifically, a first protoplast can be obtained from a plant having the expression cassette. A second protoplast can be obtained from a second plant line, optionally from another plant species or variety, preferably from the same plant species or variety, that comprises commercially desirable characteristics, such as, but not limited to disease resistance, insect resistance, valuable grain characteristics (e.g., increased seed weight and/or seed size) etc. The protoplasts are then fused using traditional protoplast fusion procedures, which are known in the art to produce the cross.

Alternatively, embryo rescue may be employed in the transfer of the expression cassette from a donor plant to a recipient plant. Embryo rescue can be used as a procedure to isolate embryo's from crosses wherein plants fail to produce viable seed. In this process, the fertilized ovary or immature seed of a plant is tissue cultured to create new plants (see Pierik, 1999, In vitro culture of higher plants, Springer, ISBN 079235267x, 9780792352679, which is incorporated herein by reference in its entirety).

In some embodiments, the recipient plant is an elite line having one or more certain agronomically important traits. Examples of agronomically important traits include but are not limited to those that result in increased biomass production, production of specific biofuels, increased food production, improved food quality, increased seed oil content, etc. Additional examples of agronomically important traits includes pest resistance, vigor, development time (time to harvest), enhanced nutrient content, novel growth patterns, flavors or colors, salt, heat, drought and cold tolerance, and the like. Agronomically important traits do not include selectable marker genes (e.g., genes encoding herbicide or antibiotic resistance used only to facilitate detection or selection of transformed cells), hormone biosynthesis genes leading to the production of a plant hormone (e.g., auxins, gibberellins, cytokinins, abscisic acid and ethylene that are used only for selection), or reporter genes (e.g. luciferase, β-glucuronidase, chloramphenicol acetyl transferase (CAT, etc.). For example, the recipient plant can be a plant with increased seed weight and/or seed size which is due to a trait not related to the expression cassette in the donor plant. The recipient plant can also be a plant with preferred carbohydrate composition, e.g., composition preferred for nutritional or industrial applications, especially those plants in which the preferred composition is present in seeds.

In some embodiments, molecular markers are designed and made, based on the early embryo- and/or endosperm-specific or preferred promoters of the present application. In some embodiments, the molecular markers are selected from Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARS). Amplified Fragment Length Polymorphisms (AFLPs), and Simple Sequence Repeats (SSRs) which are also referred to as Microsatellites, etc. Methods of developing molecular markers and their applications are described by Avise (Molecular markers, natural history, and evolution, Publisher: Sinauer Associates, 2004, ISBN 0878930418, 9780878930418), Srivastava et al. (Plant biotechnology and molecular markers, Publisher: Springer, 2004, ISBN1402019114, 9781402019111), and Vienne (Molecular markers in plant genetics and biotechnology, Publisher: Science Publishers, 2003), each of which is incorporated by reference in its entirety.

The molecular markers can be used in molecular marker assisted breeding. For example, the molecular markers can be utilized to monitor the transfer of the genetic material. In some embodiments, the transferred genetic material is a gene of interest, such as genes that contribute to one or more favorable agronomic phenotypes when expressed in a plant cell, a plant part, or a plant. In some embodiments, the gene of interest is driven by a promoter of the present invention naturally in a plant.

In some embodiments, the early embryo- and/or endosperm-specific or preferred promoters of the present application can be analyzed and molecules that bind to one of the promoters can be designed. In some embodiments, the molecule can be small molecule compounds (e.g., polyamides), nucleic acid molecules or polypeptide molecules, or combination thereof. In some embodiments, such molecule can be used to influence the activity of the promoter, either positively or negatively. In some embodiments, the molecule is designed with the help of the TouCH Platform of Cropomics (division of NeoVentures Biotechnology Inc.). In some embodiments, the promoter activity can be altered with use of gene editing nucleases (review in Baker 2012 Nature Methods 9:23-34).

Transgenic Plants with a Recombinant Embryo- and/or Endosperm-Specific or Preferred Promoter

The present invention provides transgenic plants having a recombinant embryo- and/or endosperm-specific or preferred promoter, biologically active variants, or fragments thereof, wherein the recombinant promoter is driving the expression of a gene of interest.

The present invention also provides a seed, a fruit, a plant population, a plant part, a plant cell and/or a plant tissue culture derived from the transgenic plants as described herein.

Modern plant tissue culture is performed under aseptic conditions under filtered air. Living plant materials from the environment are naturally contaminated on their surfaces (and sometimes interiors) with microorganisms, so surface sterilization of starting materials (explants) in chemical solutions (usually alcohol or bleach) is required. Explants are then usually placed on the surface of a solid culture medium, but are sometimes placed directly into a liquid medium, particularly when cell suspension cultures are desired. Solid and liquid media are generally composed of inorganic salts plus a few organic nutrients, vitamins and plant hormones. Solid media are prepared from liquid media with the addition of a gelling agent, usually purified agar.

The composition of the medium, particularly the plant hormones and the nitrogen source (nitrate versus ammonium salts or amino acids) have profound effects on the morphology of the tissues that grow from the initial explant. For example, an excess of auxin will often result in a proliferation of roots, while an excess of cytokinin may yield shoots. A balance of both auxin and cytokinin will often produce an unorganized growth of cells, or callus, but the morphology of the outgrowth will depend on the plant species as well as the medium composition. As cultures grow, pieces are typically sliced off and transferred to new media (subcultured) to allow for growth or to alter the morphology of the culture. The skill and experience of the tissue culturist are important in judging which pieces to culture and which to discard. As shoots emerge from a culture, they may be sliced off and rooted with auxin to produce plantlets which, when mature, can be transferred to potting soil for further growth in the greenhouse as normal plants.

The transgenic plants of the present invention can be used for many purposes. In some embodiments, the transgenic plant is used as a donor plant of genetic material which can be transferred to a recipient plant to produce a plant which has the transferred genetic material. Any suitable method known in the art can be applied to transfer genetic material from a donor plant to a recipient plant. In most cases, such genetic material is genomic material.

In some embodiments, the whole genome of the transgenic plants of the present invention is transferred into a recipient plant. This can be done by crossing the transgenic plants to a recipient plant to create a F1 plant. The F1 plant can be further selfed and selected for one, two, three, four, or more generations to give plants with the recombinant embryo- and/or endosperm-specific or preferred promoter.

In another embodiment, at least the parts containing the transgene of the donor plant's genome are transferred. This can be done by crossing the transgenic plants to a recipient plant to create a F1 plant, followed with one or more backcrosses to one of the parent plants to plants with the desired genetic background. The progeny resulting from the backcrosses can be further selfed and selected to give plants with increased seed weight and/or seed size. In some embodiments, the recipient plant is an elite line having one or more certain agronomically important traits.

In some embodiments, the transgenic plants have increased seed weight, seed number, and/or seed size compared to a wild type plant. In some embodiments, the transgenic plants of the present invention may have altered metabolic profiles compared to a wild type plant. A plant with altered metabolic profiles can be selected by methods well known to one skilled in the art. For example, metabolic profiles can be screened using quantitative chemical analysis methods, e.g., using gas chromatography (GC) analysis, or high performance liquid chromatography analysis (or high pressure liquid chromatography, HPLC).

### Plant Transformation

The expression cassettes of the present invention can be transformed into a plant. The most common method for the introduction of new genetic material into a plant genome involves the use of living cells of the bacterial pathogen Agrobacterium tumefaciens to literally inject a piece of DNA, called transfer or T-DNA, into individual plant cells (usually following wounding of the tissue) where it is targeted to the plant nucleus for chromosomal integration. There are numerous patents governing Agrobacterium mediated transformation and particular DNA delivery plasmids designed specifically for use with Agrobacterium—for example, U.S. Pat. No. 4,536,475, EP0265556, EP0270822, WO8504899, WO8603516, U.S. Pat. No. 5,591,616, EP0604662, EP0672752, WO8603776, WO9209696, WO9419930, WO9967357, U.S. Pat. No. 4,399,216, WO8303259, U.S. Pat. No. 5,731,179, EP068730, WO9516031, U.S. Pat. No. 5,693,512, U.S. Pat. No. 6,051,757 and EP904362A1. Agrobacterium-mediated plant transformation involves as a first step the placement of DNA fragments cloned on plasmids into living Agrobacterium cells, which are then subsequently used for transformation into individual plant cells. Agrobacterium-mediated plant transformation is thus an indirect plant transformation method. Methods of Agrobacterium-mediated plant transformation that involve using vectors with no T-DNA are also well known to those skilled in the art and can have applicability in the present invention. See, for example, U.S. Pat. No. 7,250,554, which utilizes P-DNA instead of T-DNA in the transformation vector.

Direct plant transformation methods using DNA have also been reported. The first of these to be reported historically is electroporation, which utilizes an electrical current applied to a solution containing plant cells (M. E. Fromm et al., Nature, 319, 791 (1986); H. Jones et al., Plant Mol. Biol., 13, 501 (1989) and H. Yang et al., Plant Cell Reports, 7, 421 (1988). Another direct method, called “biolistic bombardment”, uses ultrafine particles, usually tungsten or gold, that are coated with DNA and then sprayed onto the surface of a plant tissue with sufficient force to cause the particles to penetrate plant cells, including the thick cell wall, membrane and nuclear envelope, but without killing at least some of them (U.S. Pat. No. 5,204,253, U.S. Pat. No. 5,015,580). A third direct method uses fibrous forms of metal or ceramic consisting of sharp, porous or hollow needle-like projections that literally impale the cells, and also the nuclear envelope of cells. Both silicon carbide and aluminum borate whiskers have been used for plant transformation (Mizuno et al., 2004; Petolino et al., 2000; U.S. Pat. No. 5,302,523 US Application 20040197909) and also for bacterial and animal transformation (Kaepler et al., 1992; Raloff, 1990; Wang, 1995). There are other methods reported, and undoubtedly, additional methods will be developed. However, the efficiencies of each of these indirect or direct methods in introducing foreign DNA into plant cells are invariably extremely low, making it necessary to use some method for selection of only those cells that have been transformed, and further, allowing growth and regeneration into plants of only those cells that have been transformed.

For efficient plant transformation, a selection method must be employed such that whole plants are regenerated from a single transformed cell and every cell of the transformed plant carries the DNA of interest. These methods can employ positive selection, whereby a foreign gene is supplied to a plant cell that allows it to utilize a substrate present in the medium that it otherwise could not use, such as mannose or xylose (for example, refer U.S. Pat. No. 5,767,378; U.S. Pat. No. 5,994,629). More typically, however, negative selection is used because it is more efficient, utilizing selective agents such as herbicides or antibiotics that either kill or inhibit the growth of nontransformed plant cells and reducing the possibility of chimeras. Resistance genes that are effective against negative selective agents are provided on the introduced foreign DNA used for the plant transformation. For example, one of the most popular selective agents used is the antibiotic kanamycin, together with the resistance gene neomycin phosphotransferase (nptII), which confers resistance to kanamycin and related antibiotics (see, for example, Messing & Vierra, Gene 19: 259-268 (1982); Bevan et al., Nature 304:184-187 (1983)). However, many different antibiotics and antibiotic resistance genes can be used for transformation purposes (refer U.S. Pat. No. 5,034,322, U.S. Pat. No. 6,174,724 and U.S. Pat. No. 6,255,560). In addition, several herbicides and herbicide resistance genes have been used for transformation purposes, including the bar gene, which confers resistance to the herbicide phosphinothricin (White et al., Nucl Acids Res 18: 1062 (1990), Spencer et al., Theor Appl Genet. 79: 625-631 (1990), U.S. Pat. No. 4,795,855, U.S. Pat. No. 5,378,824 and U.S. Pat. No. 6,107,549). In addition, the dhfr gene, which confers resistance to the anticancer agent methotrexate, has been used for selection (Bourouis et al., EMBO J. 2(7): 1099-1104 (1983).

Genes can be introduced in a site directed fashion using homologous recombination. Homologous recombination permits site specific modifications in endogenous genes and thus inherited or acquired mutations may be corrected, and/or novel alterations may be engineered into the genome. Homologous recombination and site-directed integration in plants are discussed in, for example, U.S. Pat. Nos. 5,451,513; 5,501,967 and 5,527,695.

Methods of producing transgenic plants are well known to those of ordinary skill in the art. Transgenic plants can now be produced by a variety of different transformation methods including, but not limited to, electroporation; microinjection; microprojectile bombardment, also known as particle acceleration or biolistic bombardment; viral-mediated transformation; and Agrobacterium-mediated transformation. See, for example, U.S. Pat. Nos. 5,405,765; 5,472,869; 5,538,877; 5,538,880; 5,550,318; 5,641,664; 5,736,369 and 5,736,369; International Patent Application Publication Nos. WO2002/038779 and WO/2009/117555; Lu et al., (Plant Cell Reports, 2008, 27:273-278); Watson et al., Recombinant DNA, Scientific American Books (1992); Hinchee et al., Bio/Tech. 6:915-922 (1988); McCabe et al., Bio/Tech. 6:923-926 (1988); Toriyama et al., Bio/Tech. 6:1072-1074 (1988); Fromm et al., Bio/Tech. 8:833-839 (1990); Mullins et al., Bio/Tech. 8:833-839 (1990); Hiei et al., Plant Molecular Biology 35:205-218 (1997); Ishida et al., Nature Biotechnology 14:745-750 (1996); Zhang et al., Molecular Biotechnology 8:223-231 (1997); Ku et al., Nature Biotechnology 17:76-80 (1999); and, Raineri et al., Bio/Tech. 8:33-38 (1990)), each of which is expressly incorporated herein by reference in their entirety.

Microprojectile bombardment is also known as particle acceleration, biolistic bombardment, and the gene gun (Biolistic® Gene Gun). The gene gun is used to shoot pellets that are coated with genes (e.g., for desired traits) into plant seeds or plant tissues in order to get the plant cells to then express the new genes. The gene gun uses an actual explosive (.22 caliber blank) to propel the material. Compressed air or steam may also be used as the propellant. The Biolistic® Gene Gun was invented in 1983-1984 at Cornell University by John Sanford, Edward Wolf, and Nelson Allen. It and its registered trademark are now owned by E. I. du Pont de Nemours and Company. Most species of plants have been transformed using this method.

Agrobacterium tumefaciens is a naturally occurring bacterium that is capable of inserting its DNA (genetic information) into plants, resulting in a type of injury to the plant known as crown gall. Most species of plants can now be transformed using this method, including cucurbitaceous species. A transgenic plant formed using Agrobacterium transformation methods typically contains a single gene on one chromosome, although multiple copies are possible. Such transgenic plants can be referred to as being hemizygous for the added gene. A more accurate name for such a plant is an independent segregant, because each transformed plant represents a unique T-DNA integration event (U.S. Pat. No. 6,156,953). A transgene locus is generally characterized by the presence and/or absence of the transgene. A heterozygous genotype in which one allele corresponds to the absence of the transgene is also designated hemizygous (U.S. Pat. No. 6,008,437).

General transformation methods, and specific methods for transforming certain plant species (e.g., maize) are described in U.S. Pat. Nos. 4,940,838, 5,464,763, 5,149,645, 5,501,967, 6,265,638, 4,693,976, 5,635,381, 5,731,179, 5,693,512, 6,162,965, 5,693,512, 5,981,840, 6,420,630, 6,919,494, 6,329,571, 6,215,051, 6,369,298, 5,169,770, 5,376,543, 5,416,011, 5,569,834, 5,824,877, 5,959,179, 5,563,055, and 5,968,830, each of which is incorporated herein by reference in its entirety.

In some embodiments, the expression cassettes can be introduced into an expression vector suitable for corn transformation, such as the vectors described by Sidorov and Duncan, 2008 (Agrobacterium-Mediated Maize Transformation: Immature Embryos Versus Callus, Methods in Molecular Biology, 526:47-58), Frame et al., 2002 (Agrobacterium tumefaciens-Mediated Transformation of Maize Embryos Using a Standard Binary Vector System, Plant Physiology, May 2002, Vol. 129, pp. 13-22), Ahmadabadi et al., 2007 (A leaf-based regeneration and transformation system for maize (Zea mays L.), TransgenicRes. 16, 437-448), U.S. Pat. Nos. 6,420,630, 6,919,494 and 7,682,829, or similar experimental procedures well known to those skilled in the art. Each of the references above is incorporated herein by reference in its entirety.

### Breeding Methods

Classic breeding methods can be included in the present invention to introduce one or more recombinant expression cassettes of the present invention into other plant varieties, or other close-related species that are compatible to be crossed with the transgenic plant of the present invention.

Open-Pollinated Populations.

The improvement of open-pollinated populations of such crops as rye, many maizes and sugar beets, herbage grasses, legumes such as alfalfa and clover, and tropical tree crops such as cacao, coconuts, oil palm and some rubber, depends essentially upon changing gene-frequencies towards fixation of favorable alleles while maintaining a high (but far from maximal) degree of heterozygosity. Uniformity in such populations is impossible and trueness-to-type in an open-pollinated variety is a statistical feature of the population as a whole, not a characteristic of individual plants. Thus, the heterogeneity of open-pollinated populations contrasts with the homogeneity (or virtually so) of inbred lines, clones and hybrids.

Population improvement methods fall naturally into two groups, those based on purely phenotypic selection, normally called mass selection, and those based on selection with progeny testing. Interpopulation improvement utilizes the concept of open breeding populations; allowing genes to flow from one population to another. Plants in one population (cultivar, strain, ecotype, or any germplasm source) are crossed either naturally (e.g., by wind) or by hand or by bees (commonly Apis mellifera L. or Megachile rotundata F.) with plants from other populations. Selection is applied to improve one (or sometimes both) population(s) by isolating plants with desirable traits from both sources.

There are basically two primary methods of open-pollinated population improvement. First, there is the situation in which a population is changed en masse by a chosen selection procedure. The outcome is an improved population that is indefinitely propagable by random-mating within itself in isolation. Second, the synthetic variety attains the same end result as population improvement but is not itself propagable as such; it has to be reconstructed from parental lines or clones. These plant breeding procedures for improving open-pollinated populations are well known to those skilled in the art and comprehensive reviews of breeding procedures routinely used for improving cross-pollinated plants are provided in numerous texts and articles, including: Allard, Principles of Plant Breeding, John Wiley & Sons, Inc. (1960); Simmonds, Principles of Crop Improvement, Longman Group Limited (1979); Hallauer and Miranda, Quantitative Genetics in Maize Breeding, Iowa State University Press (1981); and, Jensen, Plant Breeding Methodology, John Wiley & Sons, Inc. (1988).

Mass Selection.

In mass selection, desirable individual plants are chosen, harvested, and the seed composited without progeny testing to produce the following generation. Since selection is based on the maternal parent only, and there is no control over pollination, mass selection amounts to a form of random mating with selection. As stated herein, the purpose of mass selection is to increase the proportion of superior genotypes in the population.

Synthetics.

A synthetic variety is produced by crossing inter se a number of genotypes selected for good combining ability in all possible hybrid combinations, with subsequent maintenance of the variety by open pollination. Whether parents are (more or less inbred) seed-propagated lines, as in some sugar beet and beans (Vicia) or clones, as in herbage grasses, clovers and alfalfa, makes no difference in principle. Parents are selected on general combining ability, sometimes by test crosses or topcrosses, more generally by polycrosses. Parental seed lines may be deliberately inbred (e.g. by selfing or sib crossing). However, even if the parents are not deliberately inbred, selection within lines during line maintenance will ensure that some inbreeding occurs. Clonal parents will, of course, remain unchanged and highly heterozygous.

Whether a synthetic can go straight from the parental seed production plot to the farmer or must first undergo one or two cycles of multiplication depends on seed production and the scale of demand for seed. In practice, grasses and clovers are generally multiplied once or twice and are thus considerably removed from the original synthetic.

While mass selection is sometimes used, progeny testing is generally preferred for polycrosses, because of their operational simplicity and obvious relevance to the objective, namely exploitation of general combining ability in a synthetic.

The number of parental lines or clones that enter a synthetic vary widely. In practice, numbers of parental lines range from 10 to several hundred, with 100-200 being the average. Broad based synthetics formed from 100 or more clones would be expected to be more stable during seed multiplication than narrow based synthetics.

Pedigreed Varieties.

A pedigreed variety is a superior genotype developed from selection of individual plants out of a segregating population followed by propagation and seed increase of self pollinated offspring and careful testing of the genotype over several generations. This is an open pollinated method that works well with naturally self pollinating species. This method can be used in combination with mass selection in variety development. Variations in pedigree and mass selection in combination are the most common methods for generating varieties in self pollinated crops.

Hybrids.

A hybrid is an individual plant resulting from a cross between parents of differing genotypes. Commercial hybrids are now used extensively in many crops, including corn (maize), sorghum, sugarbeet, sunflower and broccoli. Hybrids can be formed in a number of different ways, including by crossing two parents directly (single cross hybrids), by crossing a single cross hybrid with another parent (three-way or triple cross hybrids), or by crossing two different hybrids (four-way or double cross hybrids).

Strictly speaking, most individuals in an out breeding (i.e., open-pollinated) population are hybrids, but the term is usually reserved for cases in which the parents are individuals whose genomes are sufficiently distinct for them to be recognized as different species or subspecies. Hybrids may be fertile or sterile depending on qualitative and/or quantitative differences in the genomes of the two parents. Heterosis, or hybrid vigor, is usually associated with increased heterozygosity that results in increased vigor of growth, survival, and fertility of hybrids as compared with the parental lines that were used to form the hybrid. Maximum heterosis is usually achieved by crossing two genetically different, highly inbred lines.

The production of hybrids is a well-developed industry, involving the isolated production of both the parental lines and the hybrids which result from crossing those lines. For a detailed discussion of the hybrid production process, see, e.g., Wright, Commercial Hybrid Seed Production 8:161-176, In Hybridization of Crop Plants.

Corn is used as human food, livestock feed, and as raw material in industry. The food uses of corn, in addition to human consumption of corn kernels, include both products of dry- and wet-milling industries. The principal products of corn dry-milling are grits, meal and flour.

Corn meal is flour ground to fine, medium, and coarse consistencies from dried corn. In the United States, the finely ground corn meal is also referred to as corn flour. However, the term “corn flour” denotes corn starch in the United Kingdom. Corn meal has a long shelf life and is used to produce an assortment of products, including but not limited to tortillas, taco shells, bread, cereal and muffins.

The corn wet-milling industry can provide corn starch, corn syrups, corn sweeteners and dextrose for food use. Corn syrup is used in foods to soften texture, add volume, prevent crystallization of sugar and enhance flavor. Corn syrup is distinct from high-fructose corn syrup (HFCS), which is created when corn syrup undergoes enzymatic processing, producing a sweeter compound that contains higher levels of fructose.

Corn oil is recovered from corn germ, which is a by-product of both dry- and wet-milling industries. Corn oil which is high in mono and poly unsaturated fats, is used for reducing fat and trans fat in numerous food products.

Corn, including both grain and non-grain portions of the plant, is also used extensively as livestock feed, primarily for beef cattle, dairy cattle, hogs and poultry.

Industrial uses of corn include production of ethanol, corn starch in the wet-milling industry and corn flour in the dry-milling industry. Corn ethanol is ethanol produced from corn as a biomass through industrial fermentation, chemical processing and distillation. Corn is the main feedstock used for producing ethanol fuel in the United States. The industrial applications of corn starch and flour are based on functional properties, such as viscosity, film formation, adhesive properties, and ability to suspend particles. Corn starch and flour also have application in the paper and textile industries. Other industrial uses include applications in adhesives, building materials, foundry binders, laundry starches, explosives, oil-well muds and other mining applications.

This invention is further illustrated by the following examples which should not be construed as limiting. The contents of all references, patents and published patent applications cited throughout this application, as well as the Figures and the Sequence Listing, are incorporated herein by reference.

### EXAMPLES

### Example 1

### Identification of Embryo- and Endosperm-Specific or Preferred Promoters Using Illumina® Next-Generation Sequencing

For Illumina® next-generation sequencing, total RNA was isolated from 7, 8, and 9 days after pollination (DAP) maize embryo and endosperm tissue, as well as from young leaf tissue. Illumina® sequencing was performed on all 7 mRNA-Sequencing libraries generated, and the reads aligned to the maize cDNA sequence available in the official web portal for the Maize Genome Sequence Project (Sen et al., Database, Vol. 2009, Article ID bap020, doi:10.1093/database/bap020).

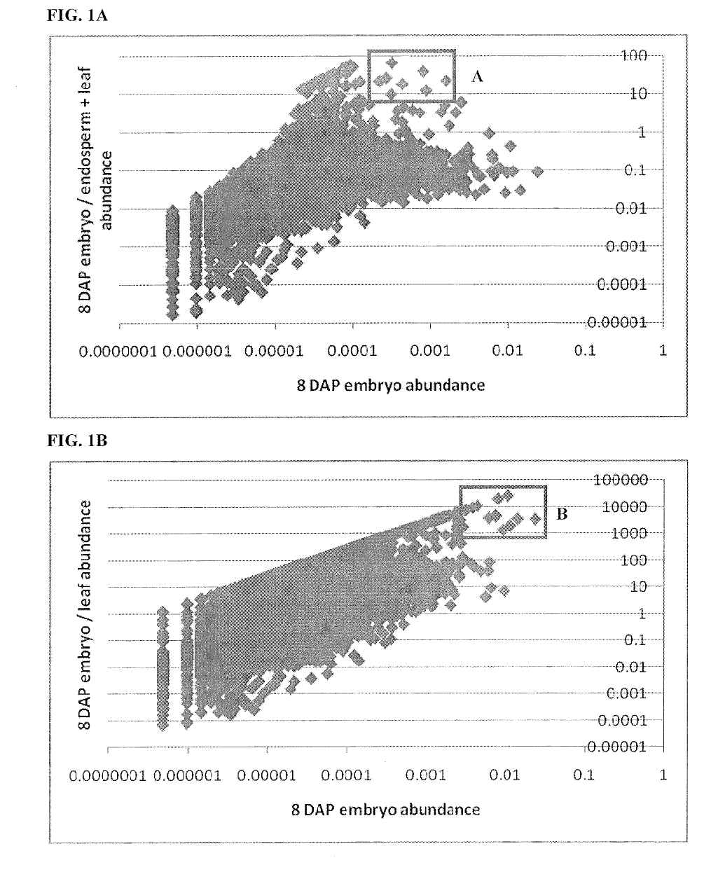

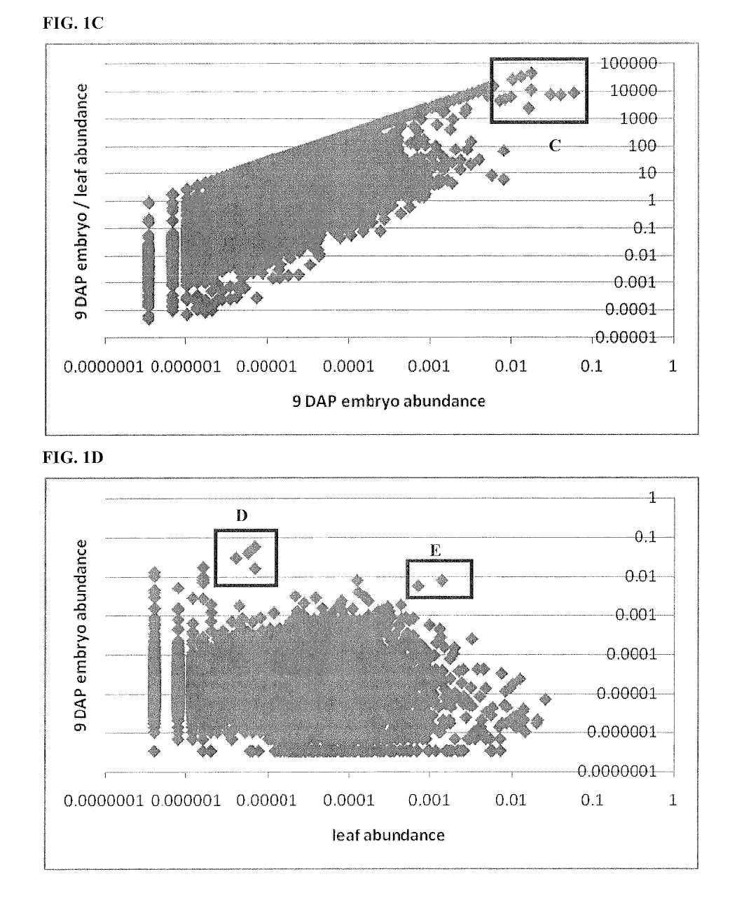

The resulting file was run through a count parsing program, which allowed for the following comparisons:
a. Abundance in 8 DAP embryo/abundance in endosperm+leaf vs. abundance in 8 DAP embryo. From this analysis 8 genes were identified which should be abundant in 8 DAP embryo and specific to embryo tissue. (FIG. 1A)
b. Abundance of 8 DAP embryo/abundance in leaf vs. abundance in 8 DAP embryo. From this analysis 13 genes were identified which should be abundant in 8 DAP embryo tissue, but have low expression in leaf tissue. They may have either high or low expression in endosperm tissue. (FIG. 1B)
c. Abundance in 9 DAP embryo/abundance in leaf vs. abundance in 9 DAP embryo. From this analysis 10 genes (were identified which should be abundant in 9 DAP embryo tissue, but have low expression in leaf tissue. They may have either high or low expression in endosperm tissue. (FIG. 1C)
d. Abundance in 9 DAP embryo abundance vs. abundance in leaf. From this analysis 4 genes were identified which should be abundant in 9 DAP embryo tissue, but have low expression in leaf tissue. Two genes were identified which should be abundant in both 9 DAP embryo tissue and in leaf tissue. They may have either high or low expression in endosperm tissue. (FIG. 1D)

A second, similar analysis was performed using a program written for MatLab and the following analyses:
a. Abundance in 8 DAP and 9 DAP embryo vs. abundance in 8 and 9 DAP embryo/abundance in leaf and endosperm.
b. Abundance in 8 DAP embryo vs. abundance in 8 DAP embryo/abundance in leaf.
c. Abundance in 8 DAP embryo vs. abundance in 8 DAP embryo/abundance in leaf and endosperm.
d. Abundance in 8 DAP embryo vs. abundance in 8 and 9 DAP embryo/abundance in leaf and endosperm.
e. Abundance in 8 DAP embryo vs. abundance in 8 and 9 DAP embryo/abundance in leaf.

This analysis resulted in the identification of 47 genes, of which 6 overlapped with the previous analysis. A further analysis in MatLab resulted in the identification of 22 embryo-specific genes, of which 3 overlapped with previous findings, and 3 endosperm-specific or preferred genes.

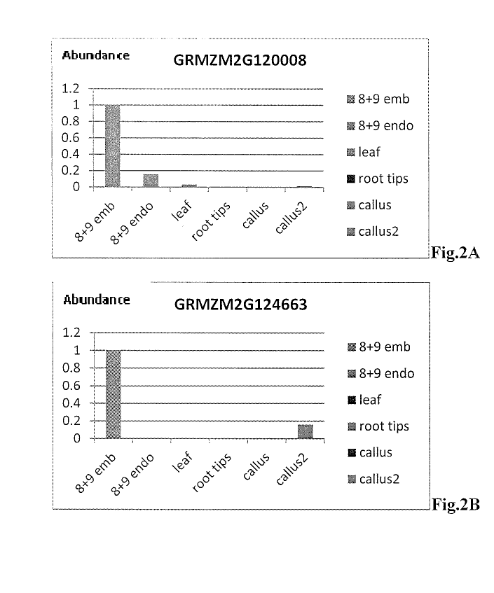

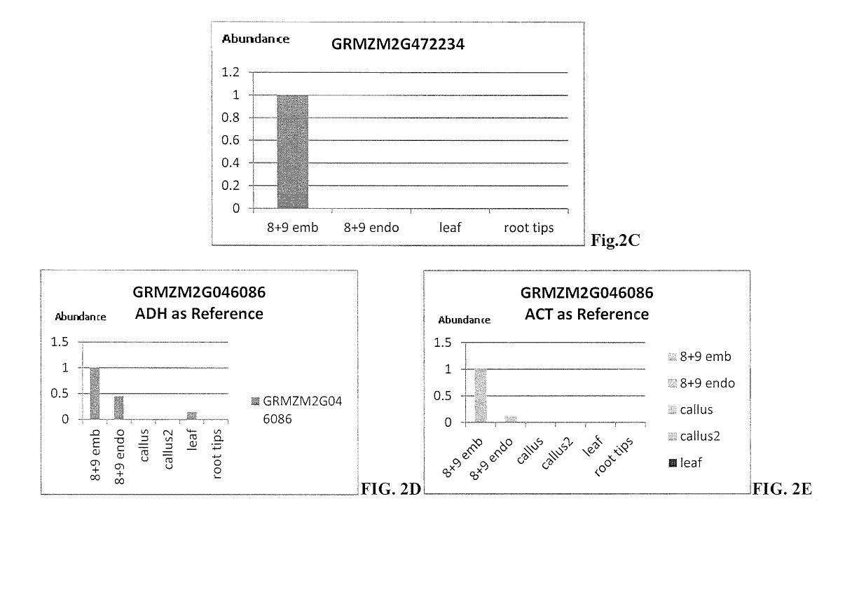

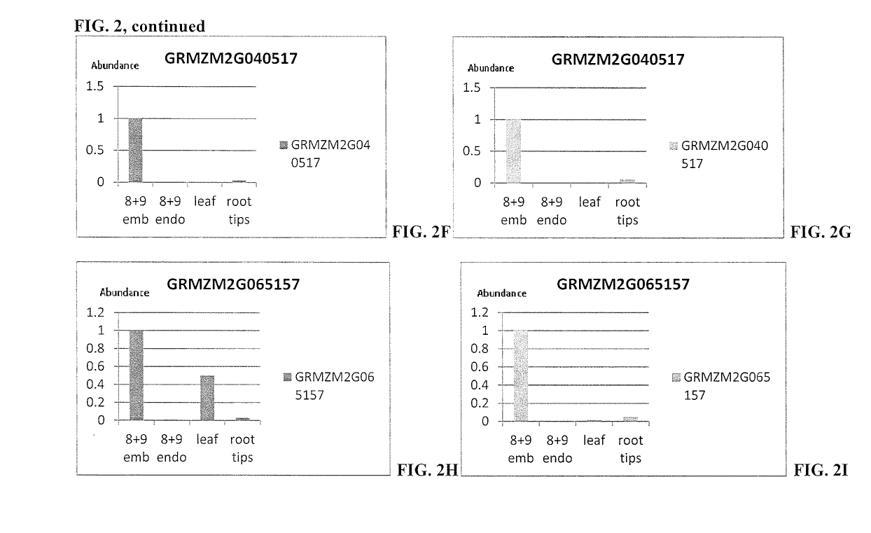

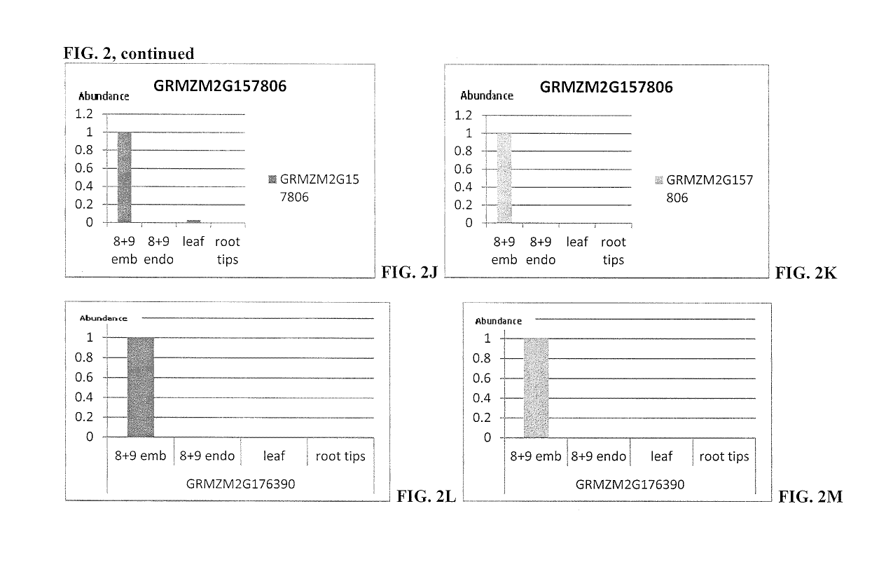

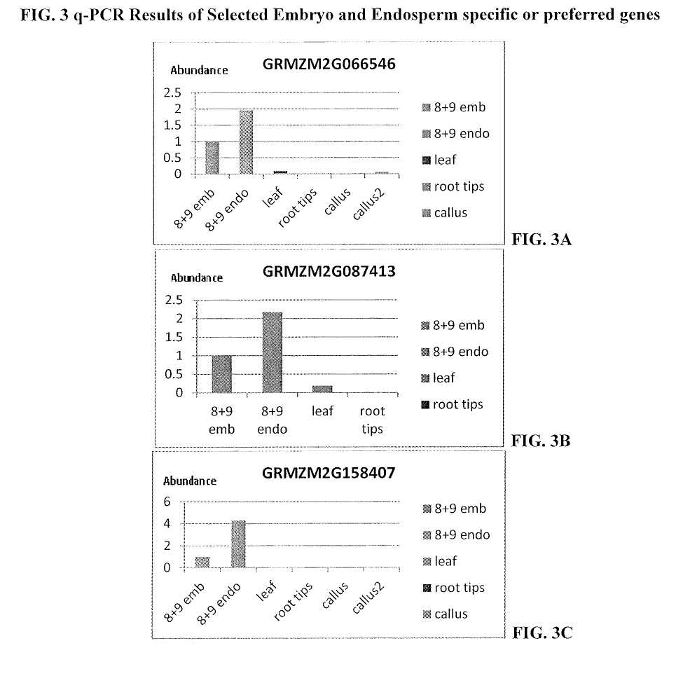

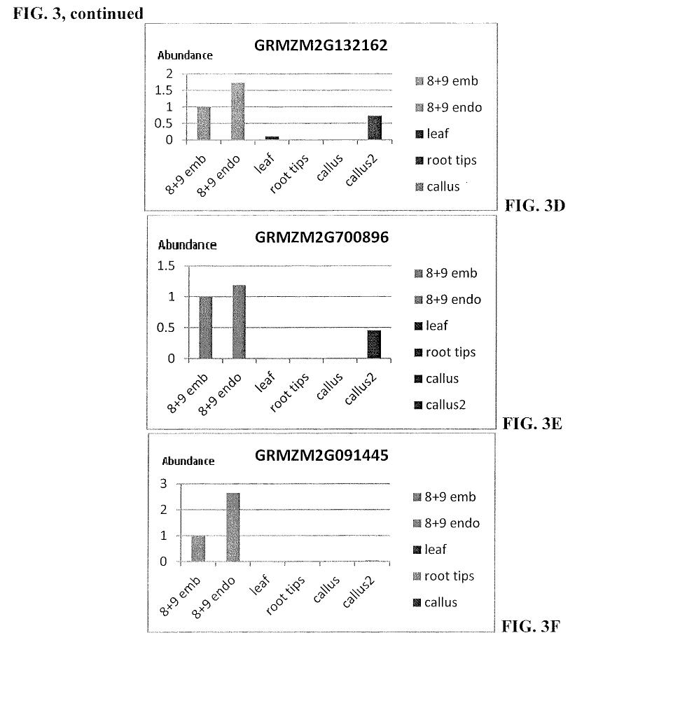

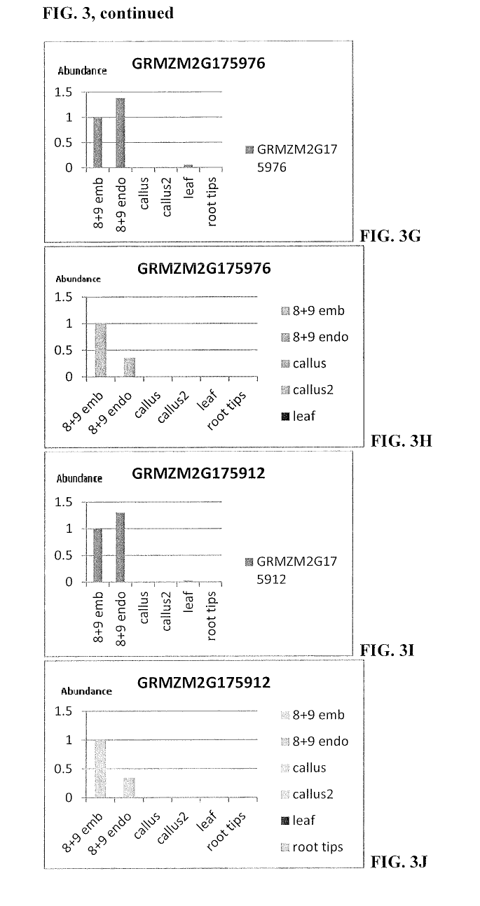

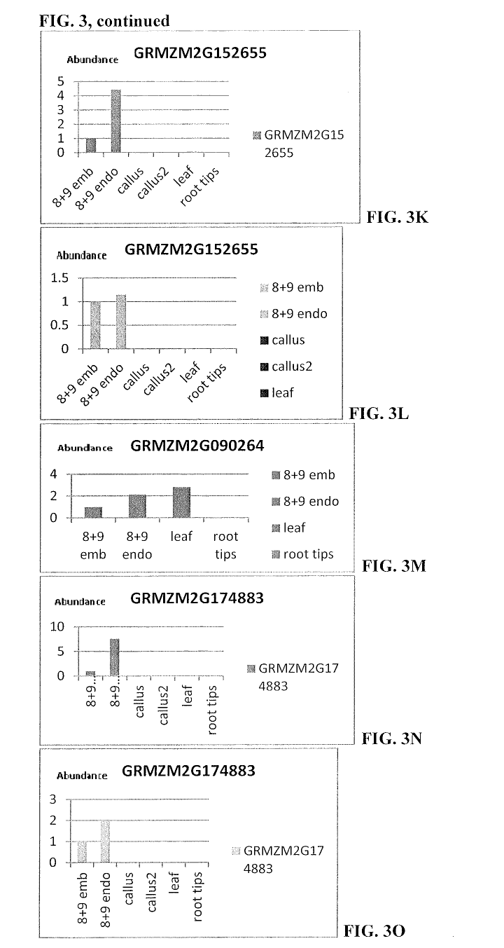

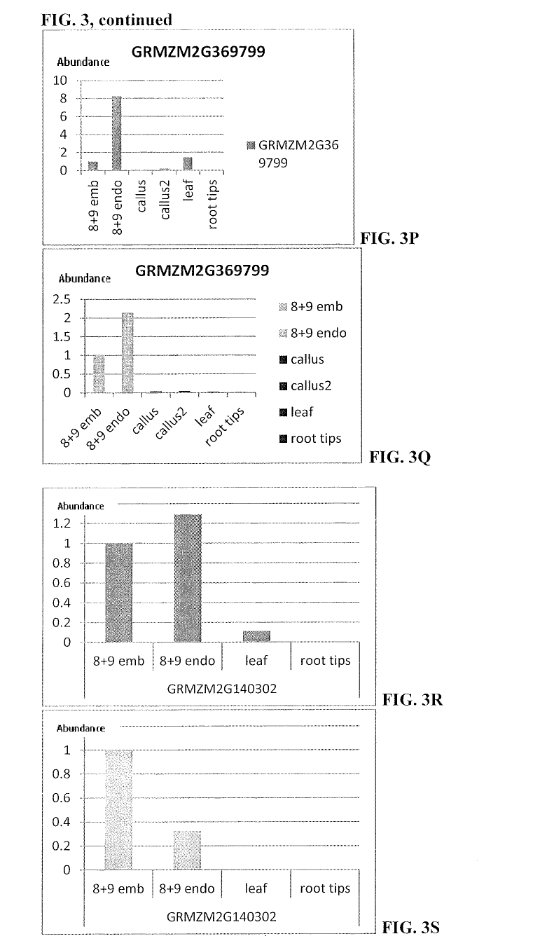

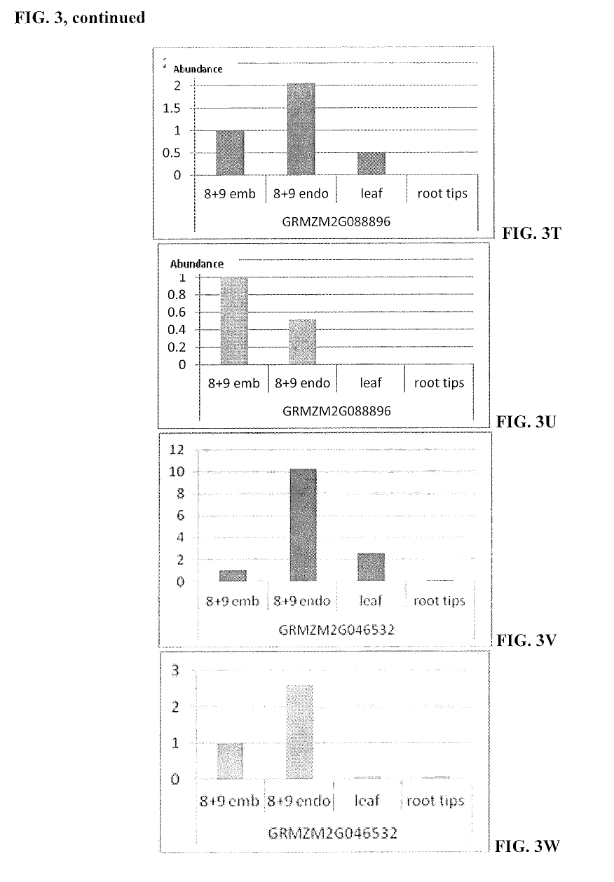

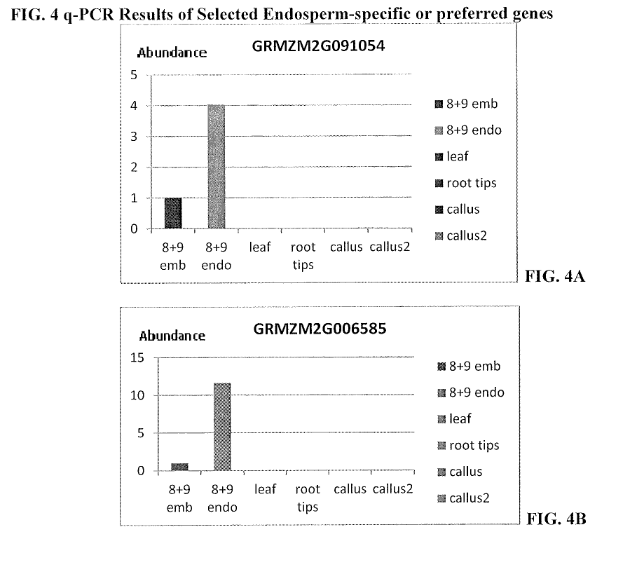

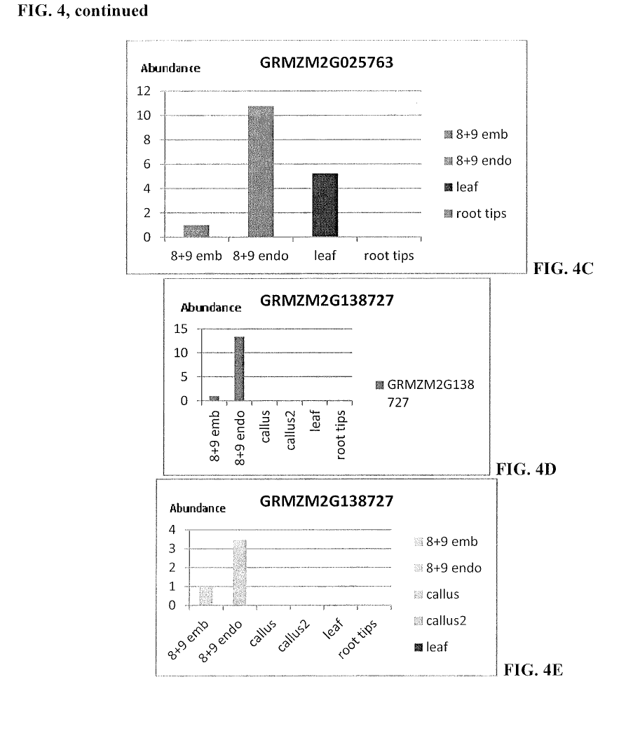

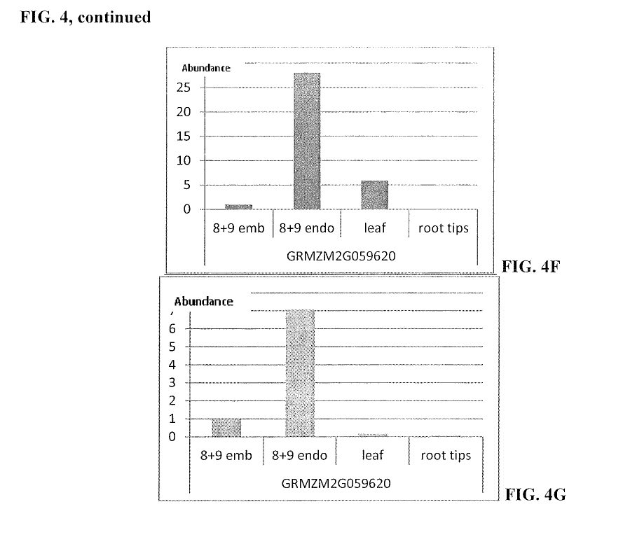

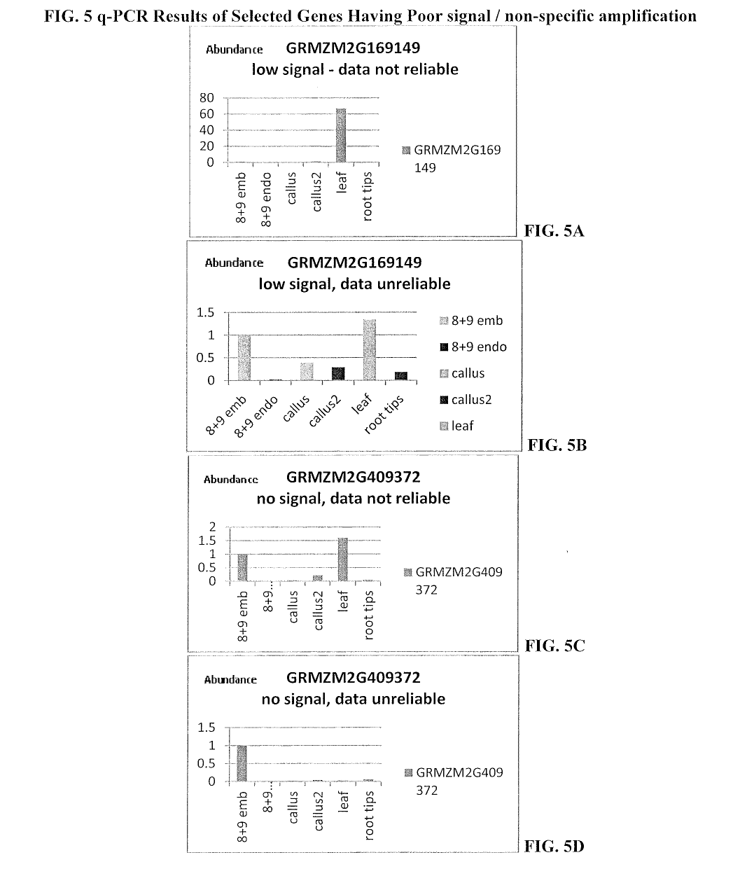

For all genes identified in these analyses, a BLAST search was performed against the full length cDNA libraries available online and maintained by the Maize cDNA organization, e.g., the “unique contigs” in the PAVE EST assemblies database (e.g., the libraries in Table 1). Genes that were confirmed to be embryo and/or endosperm-specific or preferred were further analyzed by qPCR (FIGS. 2, 3, 4, and 5). From these analyses, 31 potential early embryo and/or endosperm-specific or preferred genes were identified. The Illumina sequencing data for these 31 genes is shown in Table 2. The PAVE EST search results in shown in Table 3.

TABLE 1
PAVE EST assemblies database libraries

| Library | Title | Cultivar | Tissue |
|---|---|---|---|
| L04 | UGA-ZmSAM-XZ2 | inbred B73 | Vegetative Shoot Apical Meristem |
| | | | (SAM) and leaf primordia staged P1-P4 |
| | Src: Schnable, P. S. (2005) | | |
| L05 | Maize Endosperm cDNA Library | F-352 near | endosperm |
| | | isogenic line | |
| | Src: Arruda, P. (2005) | | |
| L06 | 3530 - Full length cDNA library created by | B73 | multiple |
| | Invitrogen from multiple tissues | | |
| | Src: Walbot, V. (2004) | | |
| L07 | ISUM5-RN | B73 | mixed |
| | Src: Schnable, P. S. (2003) | | |
| L08 | 946 - tassel primordium prepared by Schmidt | OH43 | tassels |
| | lab | | |
| | Src: Walbot, V. (2002) | | |
| L09 | 3529 - 2 mm ear tissue from Schmidt and Hake | B73 | ear |
| | labs | | |
| | Src: Walbot, V. (2003) | | |
| L10 | Normalized cDNA library from mix tissues of | (none) | mix tissues (leaf, stem, floral bud) |
| | maize | | |
| | Src: Li, Y. (2006) | | |
| L11 | GeneTag2 | mixture | (none) |
| | Src: Genoplante. (2003) | | |
| L12 | 952 - BMS tissue from Walbot Lab (reduced | BMS (Black | suspension culture |
| | rRNA) | Mexican Sweet) | |
| | Src: Walbot, V. (2002) | | |
| L13 | Zea mays embryo sac cDNA library | A188 | (none) |
| | Src: McCormick, S. (2007) | | |
| L14 | Endosperm_4 | W22 | Endosperm of 7-23DAP |
| | Src: Messing, J. (2003) | | |
| L15 | 949 - Juvenile leaf and shoot cDNA from Steve | W64A | immature leaf primordium and |
| | Moose | | vegetative meristem |
| | Src: Walbot, V. (2002) | | |
| L16 | ISUM4-TN | B73 | Seedling and silk |
| | Src: Schnable, P. S. (2006) | | |
| L17 | 1091 - Immature ear with common ESTs | OH43 | Inflorescence meristem - floral organ |
| | screened by Schmidt lab | | primordia |
| | Src: Walbot, V. (2002) | | |
| L18 | 947 - 2 week shoot from Barkan lab | B73 | leaf and stem, including leaf base |
| | Src: Walbot, V. (2002) | | |
| L19 | Endosperm_3 | W22 | Endosperm of 7-23DAP |
| | Src: Messing, J. (2003) | | |
| L20 | 605 - Endosperm cDNA library from Schmidt | Ohio43 | nucellar, embryo, and endosperm |
| | lab | | |
| | Src: Walbot, V. (2000) | | |
| L21 | zmrsub1 | (none) | (none) |
| | Src: Nguyen, H. T. (2006) | | |
| L22 | zmrws48 | (none) | (none) |
| | Src: Nguyen, H. T. (2003) | | |
| L23 | 606 - Ear tissue cDNA library from Schmidt lab | Ohio43 | mixed |
| | Src: Walbot, V. (2000) | | |
| L24 | zmrws05 | (none) | (none) |
| | Src: Nguyen, H. T. (2004) | | |
| L25 | Zea mays sperm cell cDNA library | A188 | (none) |
| | Src: McCormick, S. (2006) | | |
| L26 | Zea mays egg cell cDNA library | A188 | (none) |
| | Src: McCormick, S. (2007) | | |
| L27 | zmrww00 | (none) | (none) |
| | Src: Nguyen, H. T. (2004) | | |
| L28 | 660 - Mixed stages of anther and pollen | Ohio43 | whole premieotic anthers to pollen shed |
| | Src: Walbot, V. (2000) | | |
| L29 | 687 - Early embryo from Delaware | Illinois High Oil | embryo |
| | Src: Walbot, V. (2000) | | |
| L30 | Endosperm 5 | W22 | Endosperm of 7-23DAP |
| | Src: Messing, J. (2003) | | |
| L31 | QAE | F2 | pericarp |
| | Src: Genoplante. (2004) | | |
| L32 | GeneTag1 | mixture | (none) |
| | Src: Genoplante. (2003) | | |
| L33 | 945 - Mixed adult tissues from Walbot lab, | W23 | tassel, kernal, silk, husk, root, leaf |
| | same as 707 (SK) | | |
| | Src: Walbot, V. (2000) | | |
| L34 | 3524 - Mature pollen from Sheila McCormick's | B73 | pollen |
| | lab | | |
| | Src: Walbot, V. (2002) | | |
| L35 | 707 - Mixed adult tissues from Walbot lab (SK) | W23 | tassel, kernel, silk, husk, root, leaf |
| | Src: Walbot, V. (2000) | | |
| L36 | ISUM6 | B73 | mixed |
| | Src: Schnable, P. S. (2005) | | |
| L37 | QBJ | F2 | pollen |
| | Src: Genoplante. (2004) | | |
| L38 | QAF | F2 | pericarp |
| | Src: Genoplante. (2004) | | |
| L39 | QBI | F2 | pedicel |
| | Src: Genoplante. (2004) | | |
| L40 | QBH | F2 | sheath (in) |
| | Src: Genoplante. (2004) | | |
| L41 | QCG | F2 | embryo |
| | Src: Genoplante. (2005) | | |
| L42 | QCN | F2 | apex |
| | Src: Genoplante. (2005) | | |
| L43 | QCI | F2 | embryo |
| | Src: Genoplante. (2005) | | |
| L44 | QCO | F2 | apex |
| | Src: Genoplante. (2005) | | |
| L45 | QCL | F2 | apex |
| | Src: Genoplante. (2004) | | |
| L46 | QCM | F2 | apex |
| | Src: Genoplante. (2004) | | |
| L47 | QBB | F2 | embryo |
| | Src: Genoplante. (2003) | | |
| L48 | QCH | F2 | embryo |
| | Src: Genoplante. (2004) | | |
| L49 | QBM | F2 | apex |
| | Src: Genoplante. (2003) | | |
| L50 | QBN | F2 | pedicel, whole kernel |
| | Src: Genoplante. (2004) | | |
| L51 | QBA | F2 | endosperm |
| | Src: Genoplante. (2003) | | |
| L52 | QCD | F2 | 3rd adult leaf |
| | Src: Genoplante. (2004) | | |
| L53 | E7PCR | A188 | embryo |
| | Src: Rogowsky, P. M. (2005) | | |
| L54 | QBG | F2 | aleuron layer |
| | Src: Genoplante. (2004) | | |
| L55 | Maize Glume cDNAs Library | (none) | (none) |
| | Src: McCombie, W. 3. (2002) | | |
| L56 | QCK | F2 | cell division part of the 6th leaf |
| | Src: Genoplante. (2004) | | |
| L57 | QBK | F2 | embryo |
| | Src: Genoplante. (2003) | | |
| L58 | QBL | F2 | seedling minus kernel |
| | Src: Genoplante. (2004) | | |
| L59 | QCT | f333orf334 | seedling minus kernel |
| | Src: Genoplante. (2003) | | |
| L60 | QAN | F2 | pericarp |
| | Src: Genoplante. (2004) | | |
| L61 | QAM | F2 | ear leaf |
| | Src: Genoplante. (2003) | | |
| L62 | QCF | F2 | seedling minus kernel |
| | Src: Genoplante. (2004) | | |
| L63 | QCJ | F2 | cell lignification part of the 6th leaf |
| | Src: Genoplante. (2004) | | |
| L64 | QCU | f333orf334 | Seedling minus kernel |
| | Src: Genoplante. (2005) | | |
| L65 | QCB | f334 | 3rd adult leaf |
| | Src: Genoplante. (2004) | | |
| L66 | QAC | F2 | aerial, root, whole plant |
| | Src: Genoplante. (2004) | | |
| L67 | QBF | F2 | ear leaf |
| | Src: Genoplante. (2004) | | |
| L68 | QCA | f334 | 3rd adult leaf |
| | Src: Genoplante. (2004) | | |
| L69 | QBQ | F2 | endosperm |
| | Src: Genoplante. (2003) | | |
| L70 | 3528 - Positive selection of MADS-box genes | OH43 | ear |
| | from ear library 946 | | |
| | Src: Walbot, V. (2002) | | |
| L71 | QAD | F2 | aerial, root, whole plant |
| | Src: Genoplante. (2004) | | |
| L72 | Pioneer AF-1 array | (none) | (none) |
| | Src: Jung, R. (2003) | | |
| L73 | zmrws055 | (none) | (none) |
| | Src: Nguyen, H. T. (2003) | | |
| L74 | UGIV-3524-Reseq | (none) | (none) |
| | Src: Schnable, P. S. (2006) | | |
| L75 | zmrww005 | (none) | (none) |
| | Src: Nguyen, H. T. (2003) | | |
| L76 | 1021 - Unigene II from Maize Genome Project | (none) | (none) |
| | Src: Walbot, V. (2004) | | |
| L77 | QBS | F2 | roots extremities |
| | Src: Genoplante. (2004) | | |
| L78 | Endosperm 1 | W22 | Endosperm of 7-23DAP |
| | Src: Messing, J. (2003) | | |
| L79 | Zea mays central cell cDNA library | A188 | (none) |
| | Src: Scholten, S. (2006) | | |
| L80 | QCS | F2 | whole plant |
| | Src: Genoplante. (2004) | | |
| L81 | zmrws485 | (none) | (none) |
| | Src: Nguyen, H. T. (2003) | | |
| L82 | ZmEC | inbred A188 | (none) |
| | Src: Kranz, E. (2006) | | |
| L83 | QAI | F2 | ear leaf |
| | Src: Genoplante. (2004) | | |

| | |
|---|---|
| L99 | All other reads >150 bp submitted to Genbank |
| | since January 2000 (from small libraries) |

TABLE 2

| | young leaf | | 7 DAP emb | | 7 DAP endo | | 8 DAP emb | |
|---|---|---|---|---|---|---|---|---|
| Gene | s_1 | N¹ | s_2 | N | s_3 | N | s_4 | N |
| GRMZM2G124663 | 0 | 0 | 0 | 0 | 0 | 0 | 552 | 0.000268 |
| GRMZM2G040517 | 0 | 0 | 0 | 0 | 7 | 2.86E−06 | 1817 | 0.000881 |
| GRMZM2G066546 | 17 | 6.9E−06 | 191 | 0.048428 | 25927 | 0.010594 | 48819 | 0.023669 |
| GRMZM2G046532 | 10 | 4.06E−06 | 190 | 0.048174 | 52270 | 0.021358 | 29403 | 0.014255 |
| GRMZM2G087413 | 14 | 5.69E−06 | 105 | 0.026623 | 12092 | 0.004941 | 23617 | 0.01145 |
| GRMZM2G091054 | 4 | 1.62E−06 | 17 | 0.00431 | 20173 | 0.008243 | 15377 | 0.007455 |
| GRMZM2G120008 | 4 | 1.62E−06 | 37 | 0.009381 | 2726 | 0.001114 | 12681 | 0.006148 |
| GRMZM2G158407 | 0 | 0 | 54 | 0.013692 | 8870 | 0.003624 | 16498 | 0.007999 |
| GRMZM2G132162 | 1 | 4.06E−07 | 73 | 0.018509 | 4708 | 0.001924 | 8974 | 0.004351 |
| GRMZM2G006585 | 1 | 4.06E−07 | 22 | 0.005578 | 25761 | 0.010526 | 7837 | 0.0038 |
| GRMZM2G700896 | 0 | 0 | 12 | 0.003043 | 1377 | 0.000563 | 6473 | 0.003138 |
| GRMZM2G091445 | 4 | 1.62E−06 | 126 | 0.031947 | 7165 | 0.002928 | 15167 | 0.007353 |
| GRMZM2G059620 | 0 | 0 | 0 | 0 | 32 | 1.31E−05 | 655 | 0.000318 |
| GRMZM2G088896 | 0 | 0 | 3 | 0.000761 | 726 | 0.000297 | 897 | 0.000435 |
| GRMZM2G175976 | 0 | 0 | 40 | 0.010142 | 1518 | 0.00062 | 2547 | 0.001235 |
| GRMZM2G046086 | 0 | 0 | 5 | 0.001268 | 442 | 0.000181 | 1509 | 0.000732 |
| GRMZM2G175912 | 0 | 0 | 1 | 0.000254 | 1876 | 0.000767 | 2837 | 0.001375 |
| GRMZM2G152655 | 1 | 4.06E−07 | 7 | 0.001775 | 1211 | 0.000495 | 1825 | 0.000885 |
| GRMZM2G138727 | 2 | 8.12E−07 | 4 | 0.001014 | 2639 | 0.001078 | 1851 | 0.000897 |
| GRMZM2G025763 | 0 | 0 | 0 | 0 | 61 | 2.49E−05 | 781 | 0.000379 |
| GRMZM2G090264 | 0 | 0 | 0 | 0 | 657 | 0.000268 | 726 | 0.000352 |
| GRMZM2G157806 | 0 | 0 | 0 | 0 | 0 | 0 | 206 | 9.99E−05 |
| GRMZM2G472234 | 0 | 0 | 0 | 0 | 0 | 0 | 195 | 9.45E−05 |
| GRMZM2G409372 | 0 | 0 | 0 | 0 | 0 | 0 | 48 | 2.33E−05 |
| GRMZM2G065157 | 0 | 0 | 0 | 0 | 0 | 0 | 45 | 2.18E−05 |
| GRMZM2G176390 | 0 | 0 | 0 | 0 | 1 | 4.09E−07 | 1654 | 0.000802 |
| GRMZM2G174883 | 5 | 2.03E−06 | 5 | 0.001268 | 7088 | 0.002896 | 2507 | 0.001215 |
| GRMZM2G369799 | 0 | 0 | 0 | 0 | 687 | 0.000281 | 288 | 0.00014 |
| GRMZM2G169149 | 1 | 4.01E−07 | 0 | 0 | 1 | 4.04E−07 | 11 | 5.24E−06 |
| GRMZM2G701926 | 0 | 0 | 0 | 0 | 0 | 0 | 8 | 3.81E−06 |
| GRMZM2G140302 | 0 | 0 | 8 | 0.002017 | 280 | 0.000113 | 1197 | 0.000571 |

| | 8 DAP endo | | 9 DAP emb | | 9 DAP endo | |
|---|---|---|---|---|---|---|
| Gene | s_6 | N | s_7 | N | s_8 | N |
| GRMZM2G124663 | 1 | 6E−07 | 72 | 2.5E−05 | 4 | 1.93E−06 |
| GRMZM2G040517 | 21 | 1.26E−05 | 179 | 6.22E−05 | 5 | 2.41E−06 |
| GRMZM2G066546 | 13257 | 0.007959 | 166628 | 0.057941 | 95806 | 0.04627 |
| GRMZM2G046532 | 38533 | 0.023133 | 86397 | 0.030042 | 155799 | 0.075244 |
| GRMZM2G087413 | 7050 | 0.004232 | 115189 | 0.040054 | 43615 | 0.021064 |
| GRMZM2G091054 | 4885 | 0.002933 | 28501 | 0.009911 | 8877 | 0.004287 |
| GRMZM2G120008 | 1646 | 0.000988 | 24233 | 0.008426 | 8246 | 0.003982 |
| GRMZM2G158407 | 3341 | 0.002006 | 50432 | 0.017536 | 25413 | 0.012273 |
| GRMZM2G132162 | 1601 | 0.000961 | 29869 | 0.010386 | 26802 | 0.012944 |
| GRMZM2G006585 | 33619 | 0.020183 | 37994 | 0.013211 | 21594 | 0.010429 |
| GRMZM2G700896 | 848 | 0.000509 | 12467 | 0.004335 | 3868 | 0.001868 |
| GRMZM2G091445 | 3343 | 0.002007 | 50233 | 0.017467 | 23393 | 0.011298 |
| GRMZM2G059620 | 2691 | 0.001616 | 1831 | 0.000637 | 1914 | 0.000924 |
| GRMZM2G088896 | 339 | 0.000204 | 2986 | 0.001038 | 1124 | 0.000543 |
| GRMZM2G175976 | 444 | 0.000267 | 6339 | 0.002204 | 4714 | 0.002277 |
| GRMZM2G046086 | 166 | 9.97E−05 | 1579 | 0.000549 | 168 | 8.11E−05 |
| GRMZM2G175912 | 475 | 0.000285 | 7376 | 0.002565 | 2201 | 0.001063 |
| GRMZM2G152655 | 526 | 0.000316 | 11067 | 0.003848 | 4645 | 0.002243 |
| GRMZM2G138727 | 10572 | 0.006347 | 4098 | 0.001425 | 10012 | 0.004835 |
| GRMZM2G025763 | 6532 | 0.003921 | 3494 | 0.001215 | 2095 | 0.001012 |
| GRMZM2G090264 | 186 | 0.000112 | 1911 | 0.000665 | 330 | 0.000159 |
| GRMZM2G157806 | 0 | 0 | 30 | 1.04E−05 | 1 | 4.83E−07 |
| GRMZM2G472234 | 0 | 0 | 16 | 5.56E−06 | 0 | 0 |
| GRMZM2G409372 | 0 | 0 | 16 | 5.56E−06 | 0 | 0 |
| GRMZM2G065157 | 0 | 0 | 27 | 9.39E−06 | 0 | 0 |
| GRMZM2G176390 | 7 | 4.2E−06 | 82 | 2.85E−05 | 1 | 4.83E−07 |
| GRMZM2G174883 | 10485 | 0.006295 | 5431 | 0.001888 | 8419 | 0.004066 |
| GRMZM2G369799 | 560 | 0.000336 | 480 | 0.000167 | 1004 | 0.000485 |
| GRMZM2G169149 | 0 | 0 | 12 | 4.12E−06 | 0 | 0 |
| GRMZM2G701926 | 0 | 0 | 10 | 3.44E−06 | 0 | 0 |
| GRMZM2G140302 | 156 | 9.27E−05 | 1737 | 0.000597 | 64 | 3.07E−05 |

¹Normalized

TABLE 3

| | | | | | Search |
|---|---|---|---|---|---|
| Gene | TGI name | Orthologues | Rice Orthologue | PAVE EST tissue libraries | Identified in |
| GRMZM2G124663 | ZmEmbryo2 | Homolog of AtLEC1 | LOC_Os02g49370 | endosperm | Excel |
| GRMZM2G176390 | ZmEmbryo4 | At TPD1 (TAPETUM | LOC_Os10g14020 | endosperm | Excel and Matlab |
| | | DETERMINANT 1) | | | |
| GRMZM2G040517 | Unnamed | Os POEI52 - Pollen Ole e I | LOC_Os05g45460 | embryo, suspension cells, | Excel |
| | | allergen and extensin | | seedling | |
| | | family protein precursor, | | | |
| | | expressed | | | |
| GRMZM2G066546 | Unnamed | 0 | LOC_Os01g63310 | endosperm | Excel |
| GRMZM2G046532 | Unnamed | At LOW-MOLECULAR-WEIGHT | no clear orthologue | endosperm | Excel |
| | | CYSTEINE-RICH family | | | |
| GRMZM2G087413 | ZmBETL-9 | LTP family protein | LOC_Os03g25350 | pediciel, embryo, endosperm | Excel |
| GRMZM2G091054 | Unnamed | LTP family protein | LOC_Os01g58660 | pedicel, endosperm | Excel and Matlab |
| GRMZM2G120008 | ZmEmbryo1 | 0 | no clear orthologue | embryo | Excel and Matlab |
| GRMZM2G158407 | ZmSh-2 | 0 | no clear orthologue | endosperm, pedicel, embryo | Excel and Matlab |
| GRMZM2G132162 | Unnamed | 0 | LOC_Os01g07430 | pedicel, embryo | Excel and Matlab |
| GRMZM2G006585 | ZmEndosperm1 | Os zinc-binding protein, | LOC_Os01g33350 | endosperm, embryo, nucellar, | Excel and Matlab |
| | | putative, expressed | | pericarp | |
| GRMZM2G700896 | Unnamed | 0 | no clear orthologue | embryo | Excel and Matlab |
| GRMZM2G091445 | ZmBETL-10 | 0 | no clear orthologue | embryo, pedicel | Excel and Matlab |
| GRMZM2G059620 | Unnamed | 0 | LOC_Os07g29794 | endosperm | Matlab |
| GRMZM2G088896 | Unnamed | 0 | LOC_Os12g39950 | pedicel, embryo, endosperm | MatLab |
| GRMZM2G175976 | ZmBETL-3 | Orthologues in sorghum but | no hits | embryo, pedicel | MatLab |
| | | no description | | | |
| GRMZM2G046086 | Unnamed | 0 | no hits | pedicel, embryo | MatLab |
| GRMZM2G175912 | Unnamed | no Orthologues | LOC_Os04g05410 | endosperm, pedicle | MatLab |
| GRMZM2G152655 | ZmBETL-2 | 0 | LOC_Os08g21690 | pedicel, endosperm, embryo | MatLab |
| GRMZM2G138727 | ZmEndosperm2 | 0 | LOC_Os08g32370 | endosperm | MatLab |
| GRMZM2G025763 | Unnamed | 0 | LOC_Os03g55734 | endosperm, aleurone, embryo | MatLab |
| GRMZM2G090264 | Unnamed | Auxin response regulator | LOC_Os08g26990 | endosperm | MatLab |
| GRMZM2G157806 | ZmEmbryo3 | At TPD1 (TAPETUM | LOC_Os10g14020 | early embryo | MatLab |
| | | DETERMINANT 1) | | | |
| GRMZM2G472234 | ZmEmbryo5 | At hydroxyproline-rich | no Os homologue (but | no good hits | MatLab |
| | | glycoprotein family | indica and other oryza) | | |
| | | protein | | | |
| GRMZM2G409372 | Unnamed | Os zinc finger, C3HC4 type | LOC_Os02g43120 | no good hits | MatLab |
| | | domain containing protein, | | | |
| | | expressed | | | |
| GRMZM2G065157 | Unnamed | Os hypothetical protein | LOC_Os04g38440 | egg cell | MatLab |
| GRMZM2G169149 | Unnamed | OsWRKY62 - Superfamily of | LOC_Os09g25070 | ear | MatLab |
| | | TFs having WRKY and zinc | | | |
| | | finger domains, expressed | | | |
| GRMZM2G701926 | Unnamed | no orthologues | 0 | no hits | Matlab |
| GRMZM2G174883 | Unnamed | A.t. CRA1 (CRUCIFERINA); | LOC_Os02g15178 | endosperm | Matlab |
| | | nutrient reservoir; Encodes | glutelin, putative, | | |
| | | a 12S seed storage protein | expressed | | |
| GRMZM2G369799 | Unnamed | MYB family transcription | LOC_Os01g74590 | endosperm | Matlab |
| | | factor, putative | | | |
| GRMZM2G140302 | Unnamed | no orthologues | No clear rice | embryo; pedicel and whole | Matlab |
| | | | orthologue | kernel; nucellar, embryo | |
| | | | | and endsoperm; pedicel | |

### Example 2

Validation of Gene Expression by qPCR

Primers were designed for each of the selected genes and expression analysed in 4-6 different tissue types: pooled 8 and 9 DAP embryo tissue, pooled 8 and 9 DAP endosperm tissue, leaves, root tips, and in certain assays tissue from either one or two different calli.

The maize LEC1 gene (GRMZM2G011789, ZmLEC1) was used as a positive control for expression in embryo tissue, and an MCM licensing factor (GRMZM2G021069) was used as a positive control for proliferating tissue. Two different reference genes were used: ZmADH1-1 and ZmActin.

The Relative Quantification (RQ) or Delta Delta Ct (ddCt) method was used to analyze the expression of each gene, with ADH (or Actin) used as reference genes, and the 8+9 DAP embryo tissue used as the calibrator sample. For each reaction, a dissociation curve was run to determine the specificity of the PCR product.

PCR amplifications of embryo-specific genes, embryo and endosperm specific or preferred genes, endosperm-specific or preferred genes, and genes with poor signal/non-specific amplification are shown in FIGS. 2, 3, 4, and 5, respectively.

### Example 3

### Corn Promoter Characterization

A separate effort was conducted to characterize the expression pattern of several candidate embryo and/or endosperm promoters. The goal is to evaluate different promoters in stably transformed corn for expression in zygotic embryos, endosperm, and other tissue types using GUS as the reporter gene, and to enable selection of promoters to drive genes of interest for yield enhancement.

Table 4 lists all the constructs that have been or will be made and tested.

TABLE 4

| | | | | | Expected Tissue |
|---|---|---|---|---|---|
| Construct | Promoter | Intron | Gene | 3′ end | expression* |
| TGZM156 | OsASP1v1 | ADH | GUS | nos | embryo and nucellin |
| TGZM157 | ZmASP1 | ADH | GUS | nos | embryo and nucellin |
| TGZM158 | ZmCLV1 | ADH | GUS | nos | inflorescence meristem |
| TGZM159 | HvPER1 | ADH | GUS | nos | embryo and aleurone |
| TGZM160 | Hv B1 Hordein | ADH | GUS | nos | endosperm |
| TGZM161 | Emb1v1 (−2031 to +15 from ATG of the | ADH | GUS | nos | embryo |
| | maize gene GRMZM2G120008) | | | | |
| TGZM162 | Emb2v1 (−866 to −42 from ATG of the | ADH | GUS | nos | embryo |
| | maize gene GRMZM2G124663) | | | | |
| TGZM163 | Emb3 GRMZM2G157806 | ADH | GUS | nos | embryo |
| TGZM164 | Emb4v1 (−1527 to −52 from ATG of the | ADH | GUS | nos | embryo |
| | maize gene GRMZM2G176390) | | | | |
| TGZM165 | Emb5 (−1965 to −399 from ATG of the | ADH | GUS | nos | embryo |
| | maize gene GRMZM2G472234) | | | | |
| TGZM166 | Endosperm1v1 (−1902 to −43 from ATG | ADH | GUS | nos | endosperm |
| | of the maize gene GRMZM2G006585) | | | | |
| TGZM167 | Endosperm2 GRMZM2G138727 | ADH | GUS | nos | endosperm |
| TGZM181 | OsASP1v2 (containing deletion of base | ADH | GUS | nos | embryo and nucellin |
| | pairs 568 to 639) | | | | |
| TGZM212 | Emb2v2 (−866 to −1 from ATG of the | none | GUS | nos | embryo |
| | maize gene GRMZM2G124663) | | | | |
| TGZM213 | Emb2v3 (−600 to −1 from ATG of the | none | GUS | nos | embryo |
| | maize gene GRMZM2G124663) | | | | |
| TGZM214 | Emb4v2 (−1527 to −1 from ATG of the | Emb4 | GUS | nos | embryo |
| | maize gene GRMZM2G176390) | | | | |
| TGZM215 | Emb4v3 (−900 to −1 from ATG of the | none | GUS | nos | embryo |
| | maize gene GRMZM2G176390) | | | | |
| TGZM216 | Emb4v4 (−818 to −1 from ATG of the | none | GUS | nos | embryo |
| | maize gene GRMZM2G176390) | | | | |
| | Zm MRP-1 | ADH | GUS | nos | Basal endosperm |
| | | | | | transfer layer |
| | Zm BETL-3 | ADH | GUS | nos | Basal endosperm |
| | | | | | transfer layer, |
| | | | | | endosperm, or embryo |

*For ZmCLV1, HvPER1, Hv B1 Hordein, OsASP1, and ZmMRP-1, the tissue expression is expected from what has been reported in the literature. The references for each of these promoters are listed in Table 8.

Corn transformations were completed or in progress with constructs (TGZm157, 158, 159, 160, 161, 164, 162, 166, and 181) representing 9 different promoters driving the GUS reporter gene. A total of 10-15 plant events for each construct were regenerated and advanced to the greenhouse. The following independent transgenic events have been evaluated for promoter constructs:
TGZm157 (ZmAsp1)—8 events
TGZm158 (ZmCLV1)—7 events
TGZm159 (HvPER1)—8 events
TGZm160 (Hv B1 Hordein)—4 events
TGZm161 (ZmEmb1v1)—7 events
TGZm162 (ZmEmb2v1)—8 events
TGZm164 (ZmEmb4v1)—4 events
TGZm166 (ZmEndo1v1)—8 events
TGZm181 (OsAsp1v2)—8 events

At 9, 14, 21 day-after-pollination (DAP), T1 kernels and other tissues were evaluated from T0 plants. At 3, 5 and 7 DAP, developing ears were characterized from T0 or T1 plants.

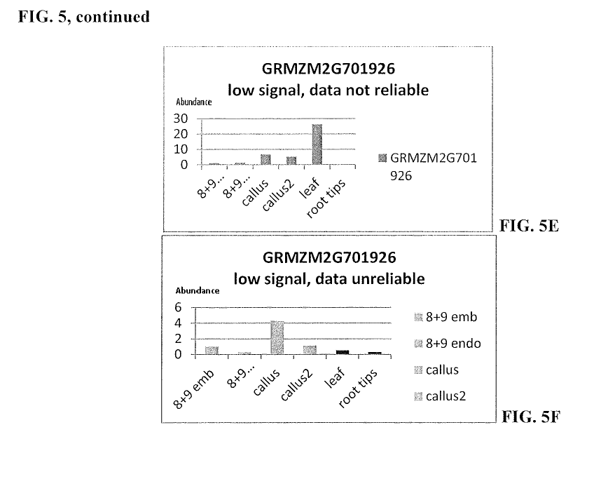

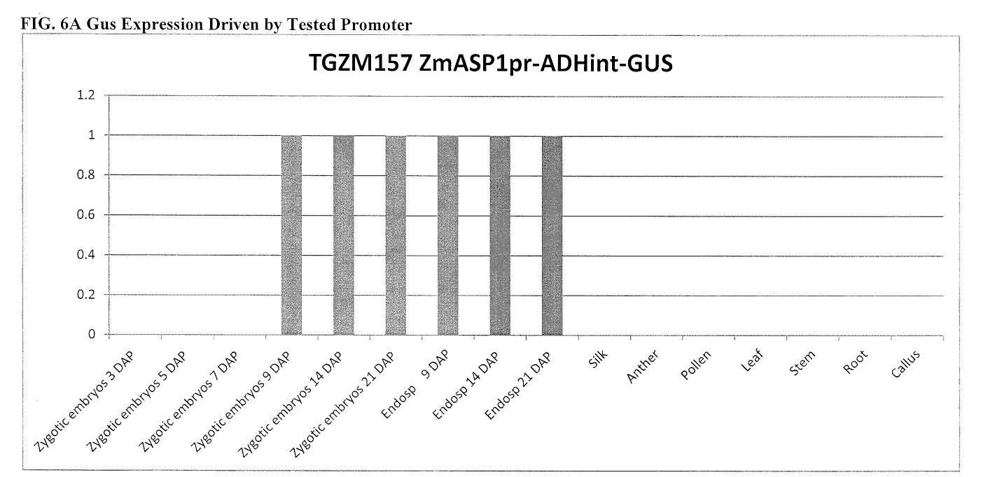

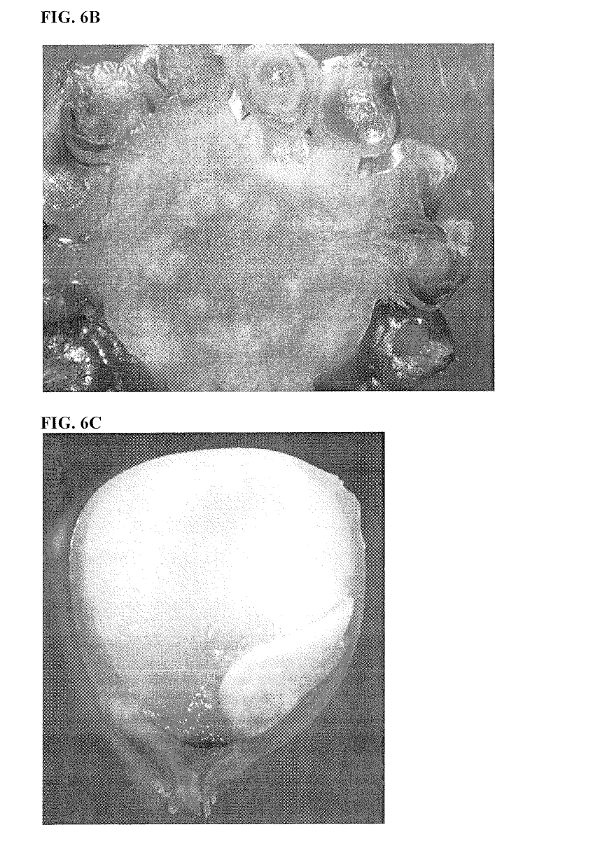

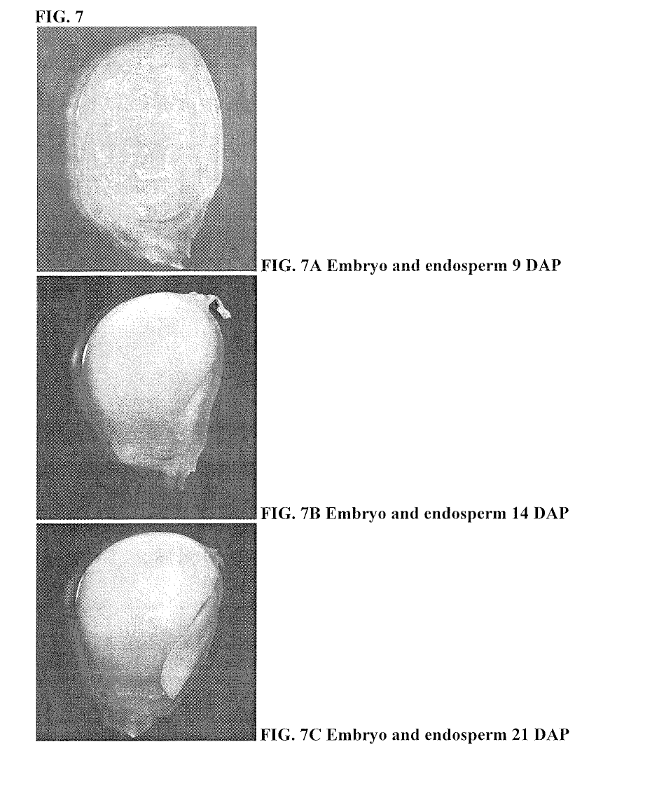

The expression pattern of GUS gene under the control of maize ASP1 promoter is shown in FIG. 6A. ZmASP1 promoter gave weak GUS expression in zygotic embryo and endosperm at 9-21 DAP but none at 3-7 DAP and other tissue types. There was no expression in any part of the transverse section (TS) of corn ear at 3 DAP (FIG. 6B), but GUS gene expression in basal endosperm transfer layer at 14 DAP was observed (FIG. 6C), showing tissue-specific GUS expression. No expression was observed in control non-transformed kernels (FIG. 7).

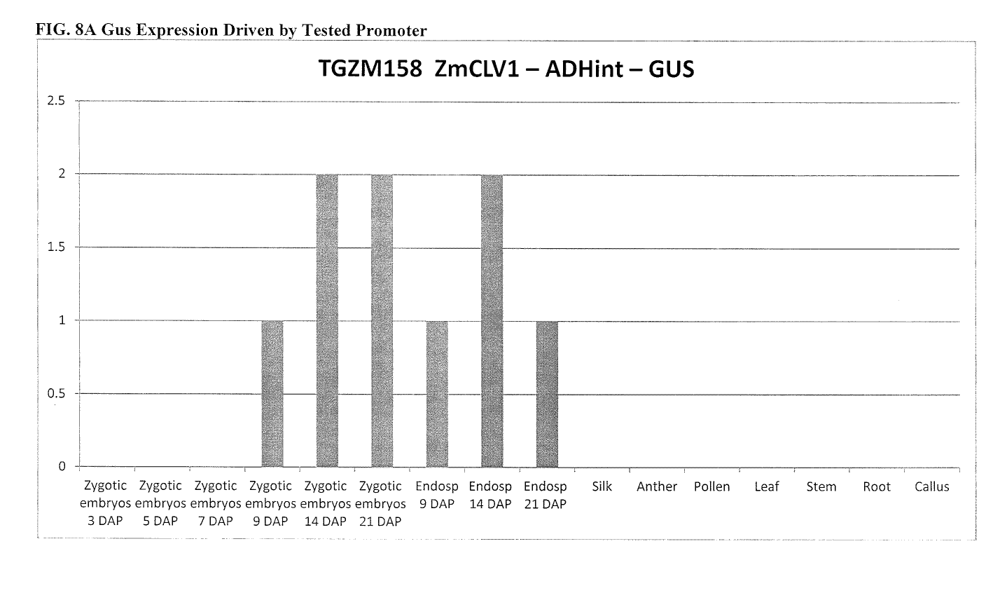

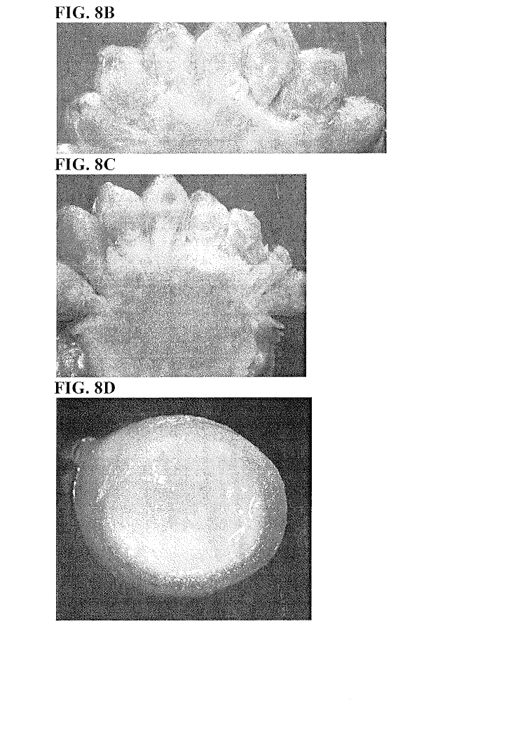

The expression pattern of GUS gene under the control of maize CLV1 promoter is shown in FIG. 8A. ZmCLV1 promoter gave weak GUS expression in zygotic embryo and endosperm at 9, 14, 21 DAP but none at 3-7 DAP and other tissues. There was no expression in any part of the transverse section (TS) of corn ear at 3 DAP or 5 DAP (FIGS. 8B and 8C). There was weak GUS expression in endosperm at 21 DAP (FIG. 8D).

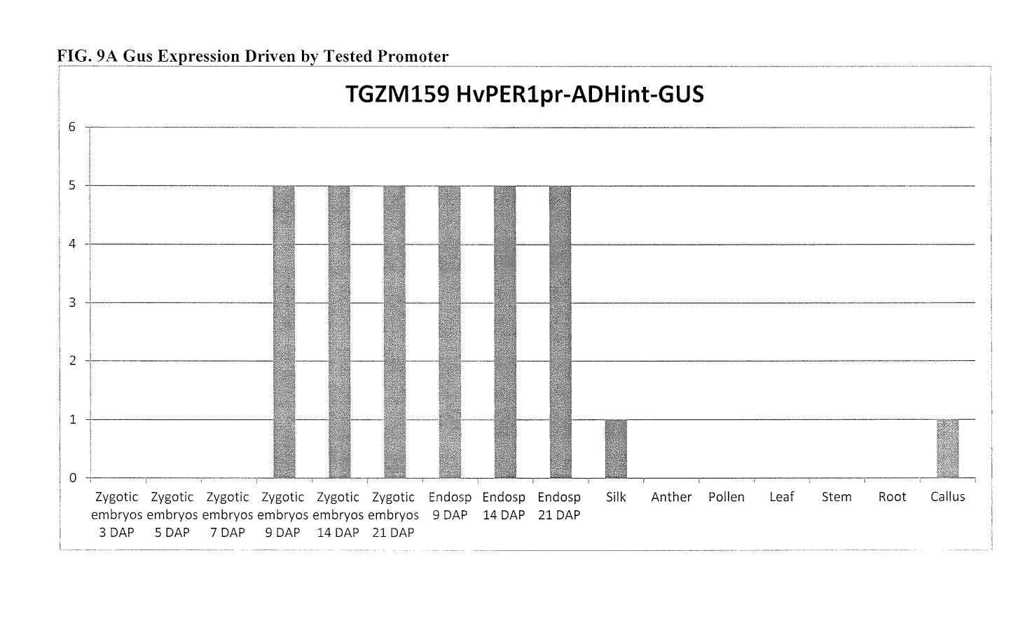

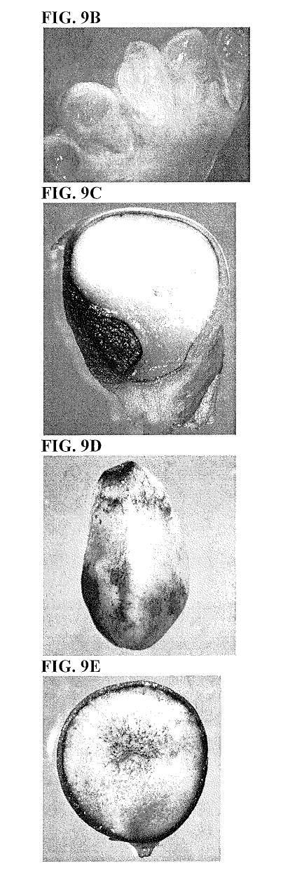

The expression pattern of GUS gene under the control of HvPER1 promoter is shown in FIG. 9A. HvPER1 promoter showed strong gene expression in embryo and endosperm at 9-21 DAP, none at 3-7 DAP and weak expression in callus and silk. There was no expression in any part of the transverse section (TS) of corn ear at 3 DAP (FIG. 9B). HvPER1 promoter gave strong GUS expression in zygotic embryo, endosperm and aleurone at 14 DAP (FIG. 9C). Strong expression was also observed in zygotic embryo and endosperm and aleurone at 21 DAP (FIGS. 9D and 9E).

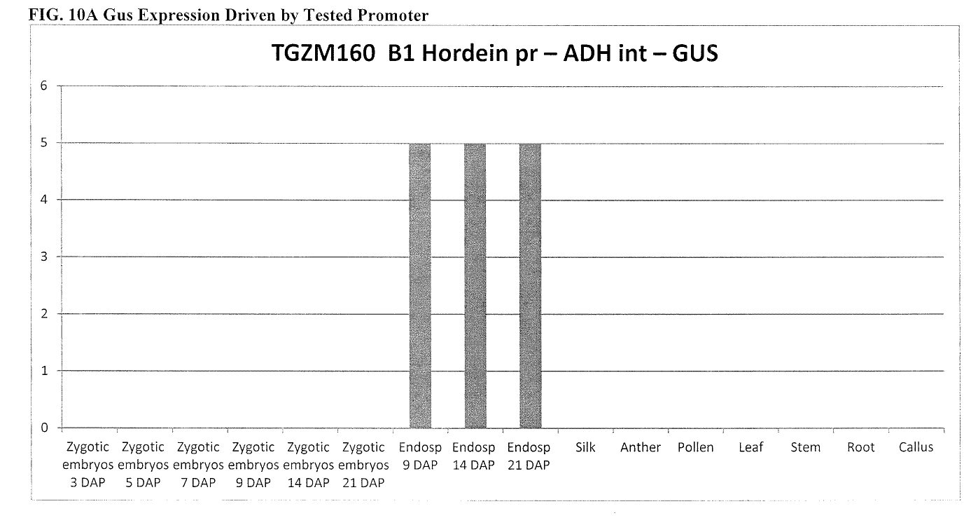

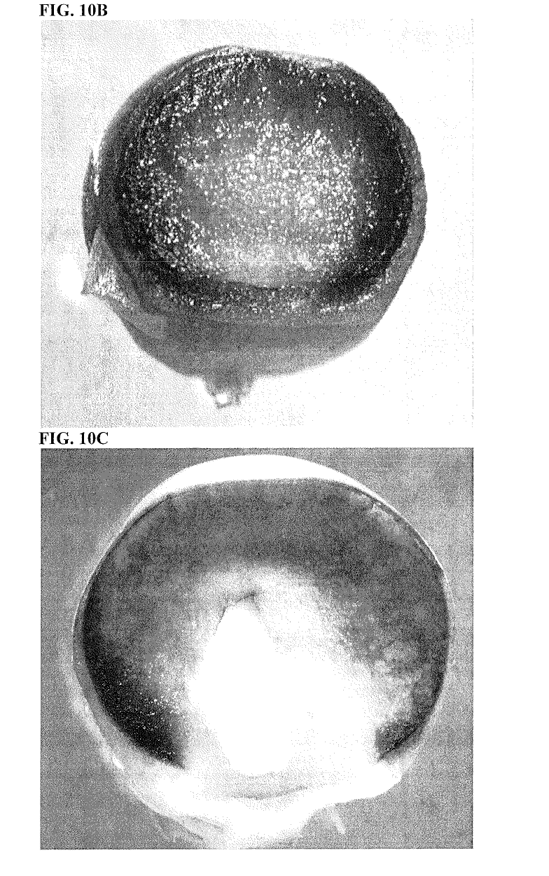

The expression pattern of GUS gene under the control of Hv B1 Hordein promoter is shown in FIG. 10A. The Hv B1 Hordein promoter has strong gene expression in the endosperm at 9, 14 and 21 DAP but none in other tissues. The Hv B1 Hordein promoter gave strong GUS expression in zygotic endosperm but none in zygotic embryo (FIGS. 10B and 10C).

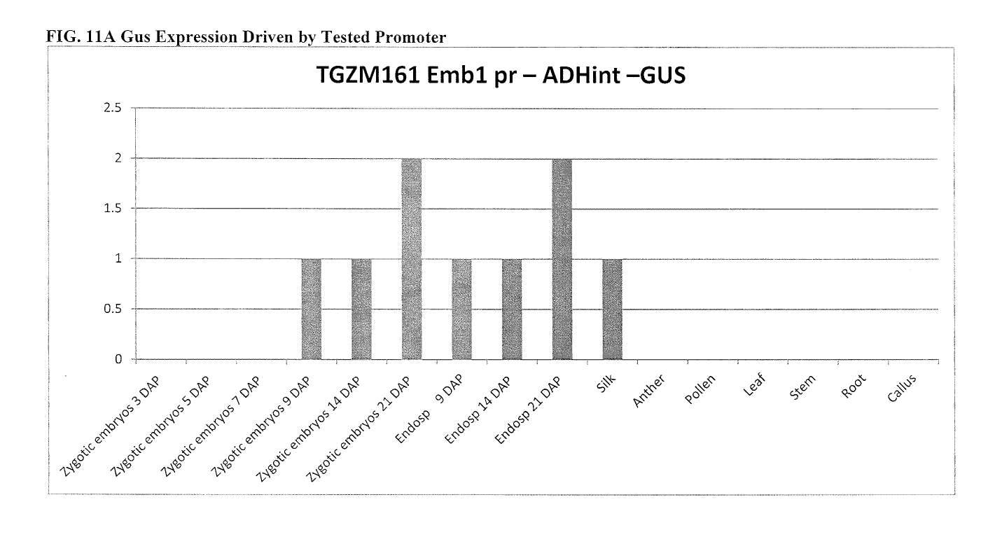

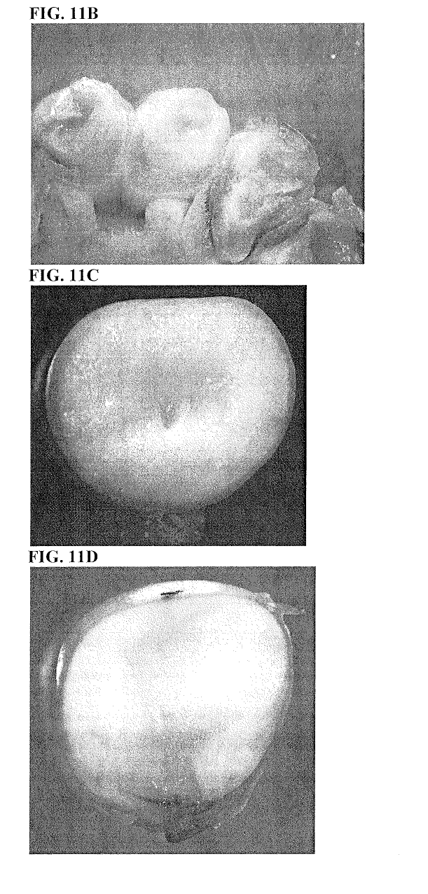

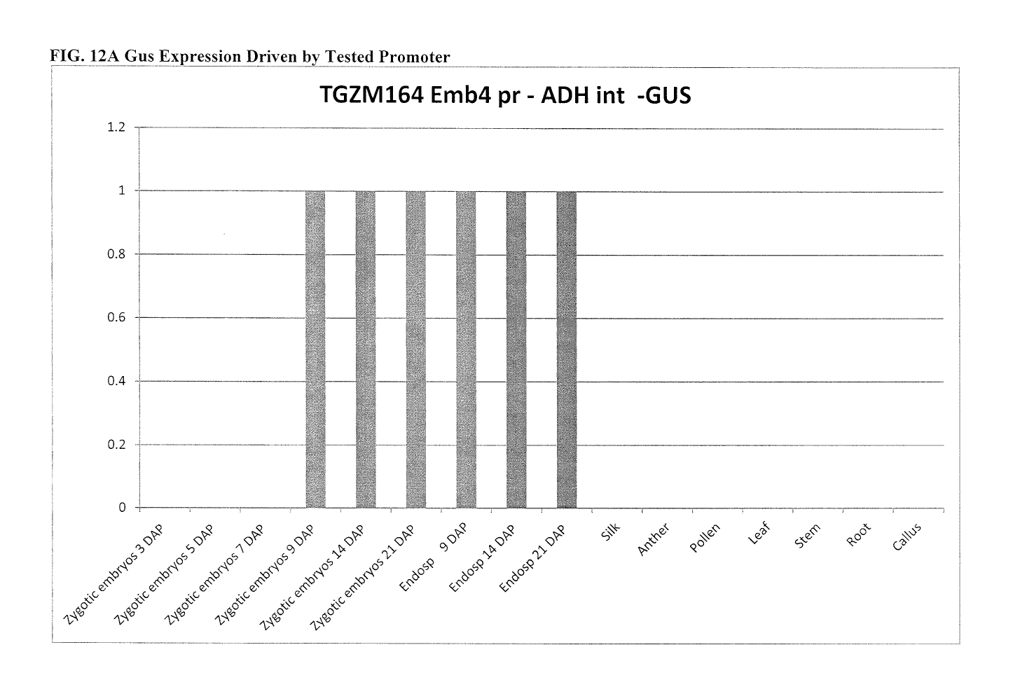

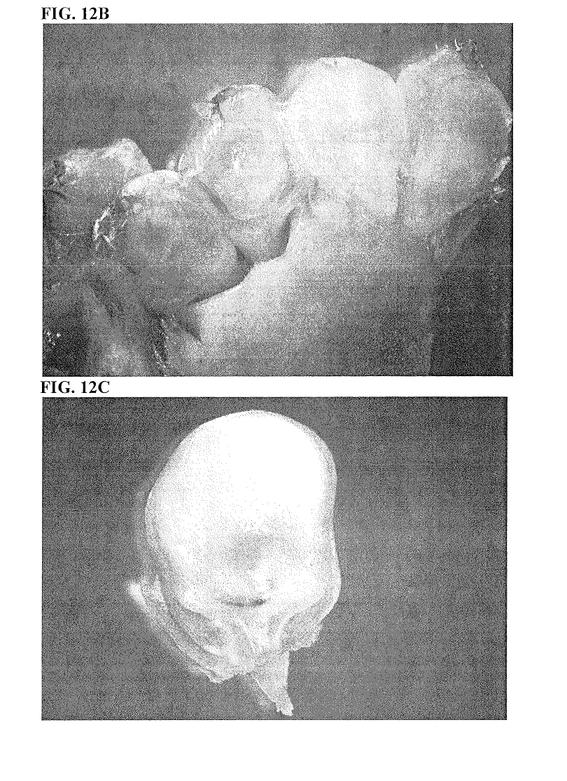

The expression pattern of GUS gene under the control of maize Emb1v1 promoter is shown in FIG. 11A. Emb1v1 promoter shows weak amorphous GUS expression in zygotic embryo and endosperm at 9-21 DAP but none at 3-7 DAP and other tissue types. There was no expression in any part of the transverse section (TS) of corn ear at 6 DAP (FIG. 11B). Weak GUS gene expression in endosperm was observed at 14 DAP (FIG. 11C). Emb1v1 promoter gave weak GUS expression in zygotic embryo, endosperm and basal endosperm transfer layer at 21 DAP (FIG. 11D) and also weak GUS expression in silk.

The expression pattern of GUS gene under the control of maize Emb4v1 promoter is shown in FIG. 12A. Emb4v1 promoter shows weak gene expression in zygotic embryo and endosperm at 9-21 DAP but none at 3-7 DAP, and other tissue types. There was no expression in any part of the transverse section (TS) of corn ear at 6 DAP (FIG. 12B). Embryo and endosperm at 9 DAP have weak gene expression that was specific in the basal endosperm transfer layer (FIG. 12C). The Emb4v1 promoter showed a highly tissue-specific response only in the basal transfer layer of the endosperm.

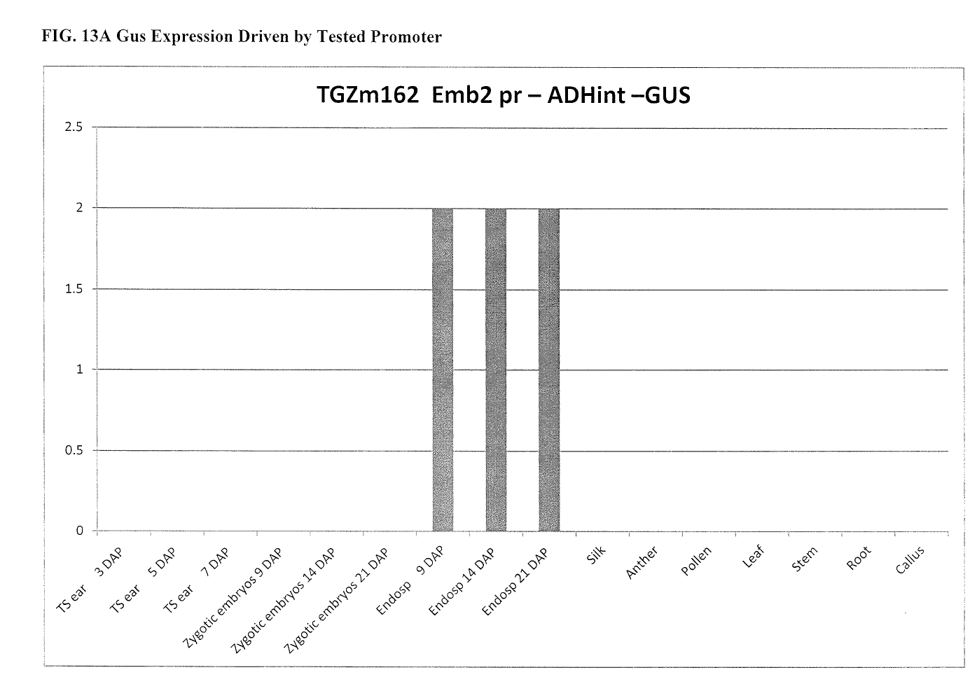

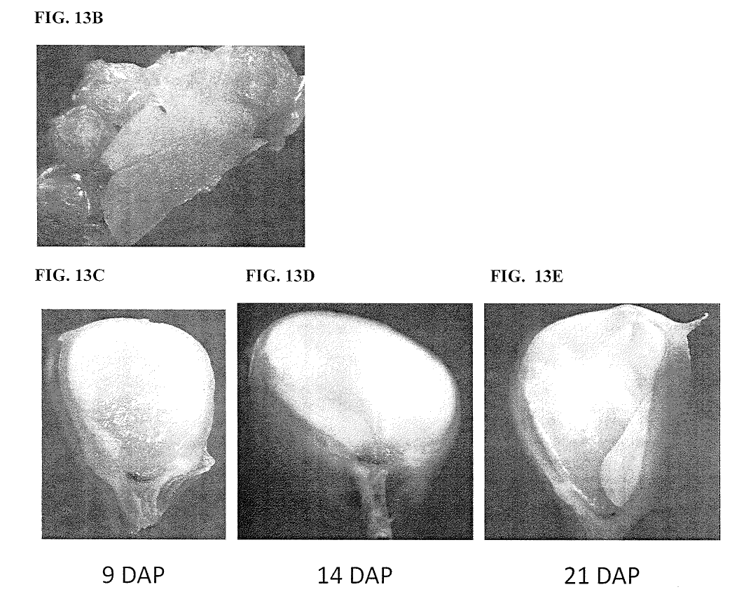

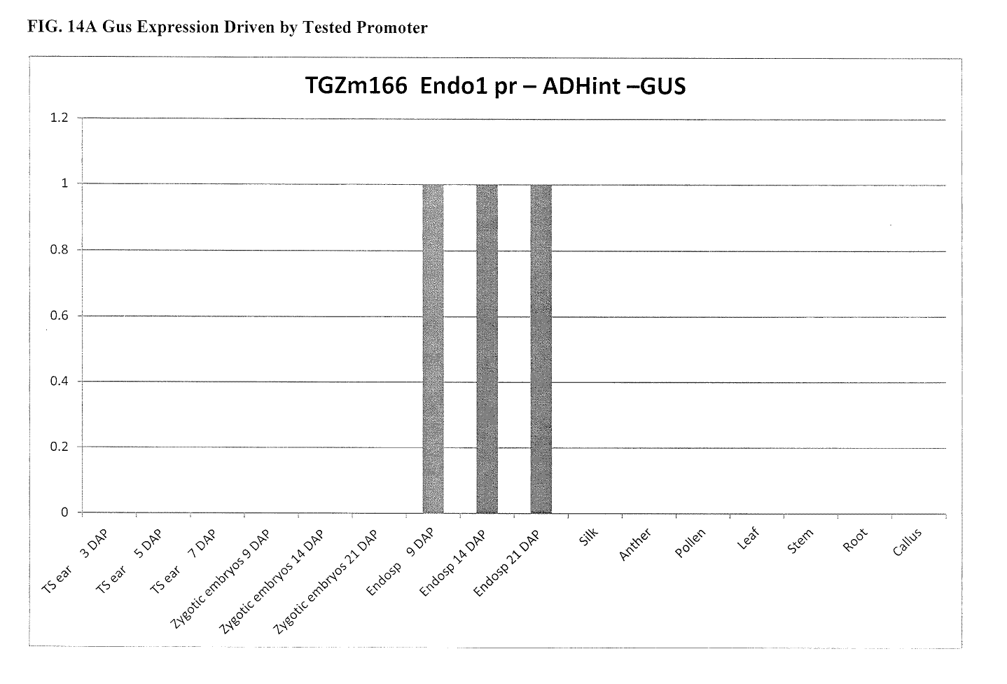

The expression pattern of GUS gene under the control of maize Emb2v1 promoter is shown in FIG. 13A. Emb2v1 promoter shows weak gene expression in basal endosperm transfer layer and/or endosperm at 9-21 DAP but no expression at 3-7 DAP or in other tissue types. There was no expression in any part of the transverse section (TS) of corn ear at 5 DAP (FIG. 13B). Weak GUS expression could be seen in the basal endosperm transfer layer at 9, 14 and 21 DAP. In addition, GUS expression could also be detected in the endosperm at 14 DAP (FIG. 13C).

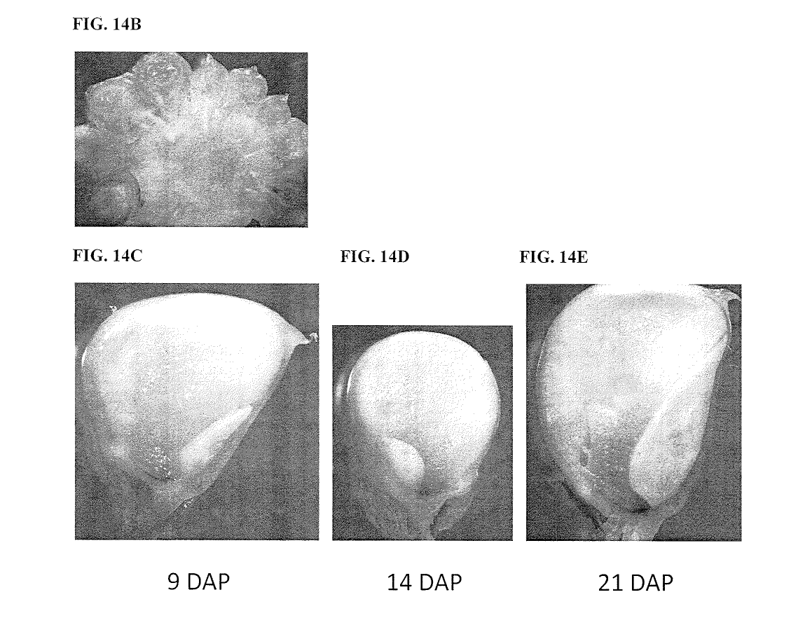

The expression pattern of GUS gene under the control of maize Endosperm1v1 promoter is shown in FIG. 14A. Endosperm1v1 promoter shows weak gene expression in basal endosperm transfer layer at 9-21 DAP but no expression at 3-7 DAP or in other tissue types. There was no expression in any part of the transverse section (TS) of corn ear at 5 DAP (FIG. 14B). Weak GUS expression is specific to the basal endosperm transfer layer at 9, 14 and 21 DAP (FIG. 14C).

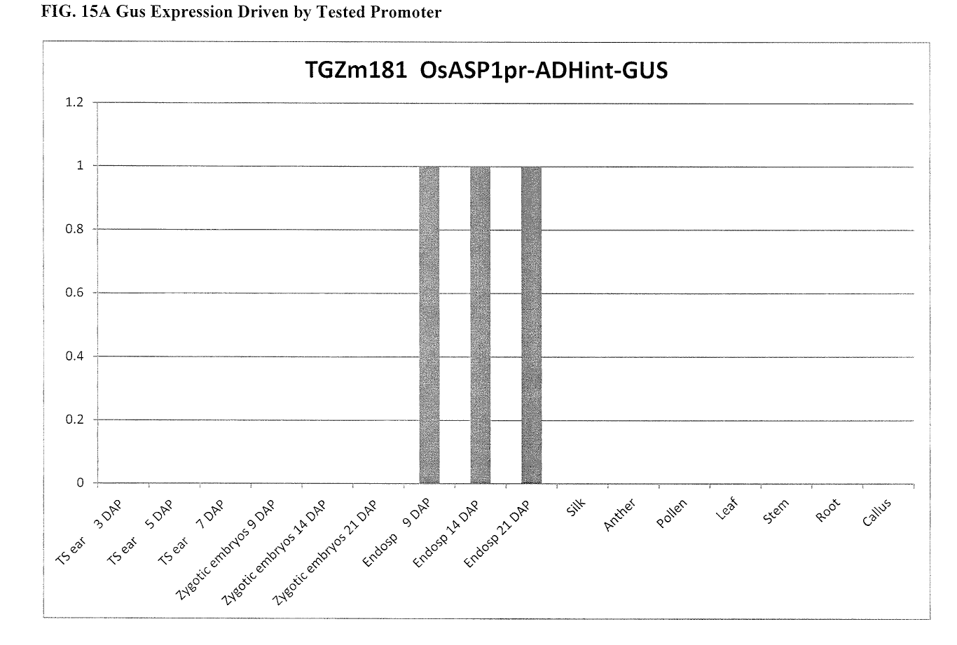

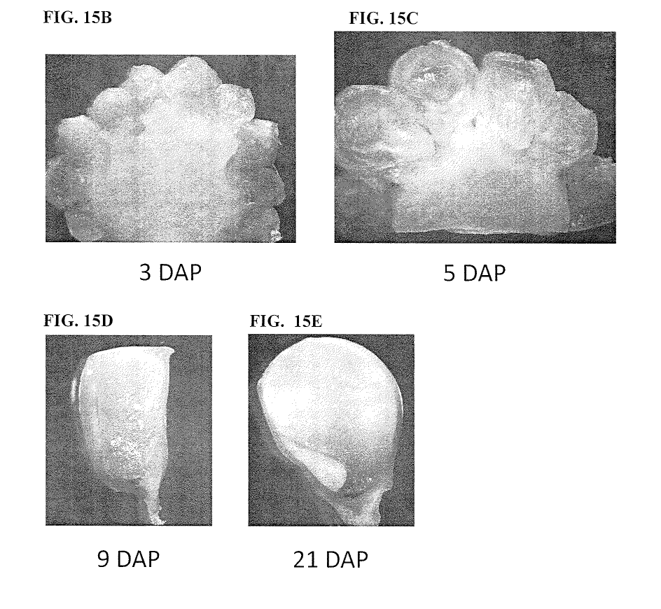

The expression pattern of GUS gene under the control of rice Asp1v2 promoter is shown in FIG. 15A. Asp1v2 promoter shows weak gene expression in basal endosperm transfer layer at 9-21 DAP but no expression at 3-7 DAP or in other tissue types. There was no expression in any part of the transverse section (TS) of corn ear at 3 or 5 DAP (FIG. 15B). Weak GUS expression is specific to the basal endosperm transfer layer at 9, 14 and 21 DAP (FIG. 15C).

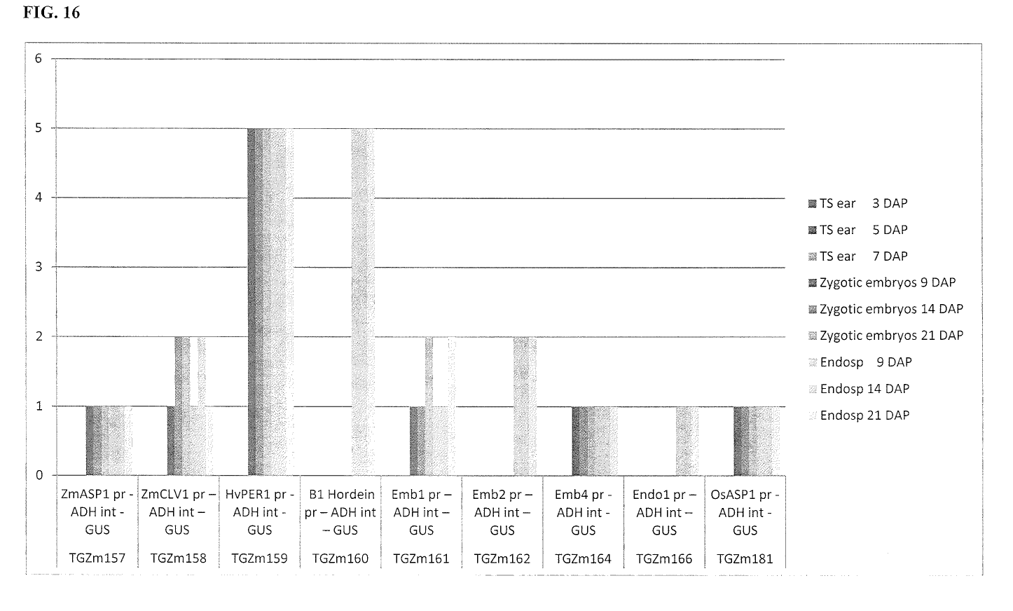

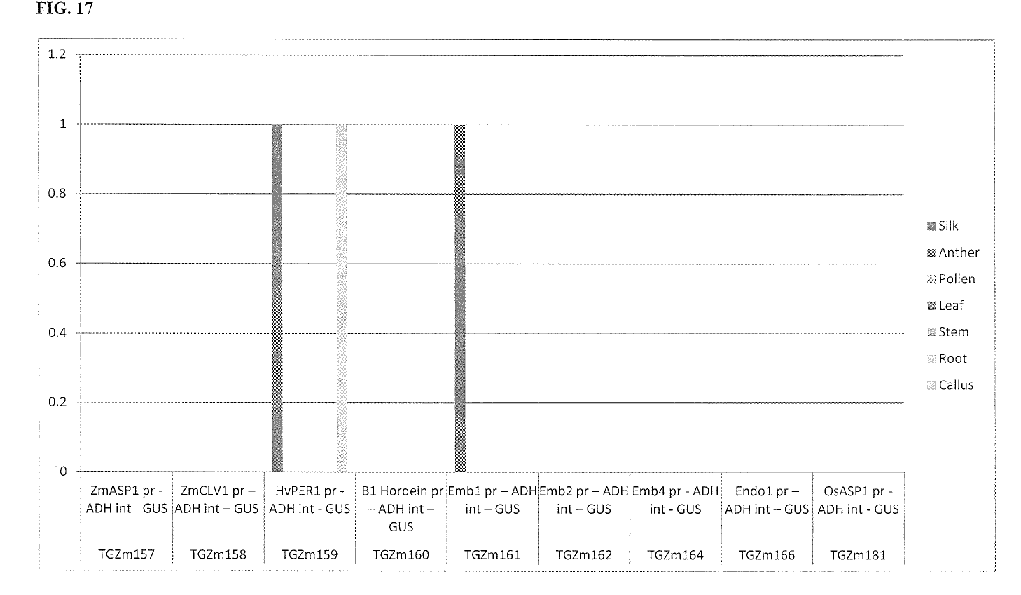

FIG. 16 shows comparison of histochemical GUS expression for 9 promoters in T1 zygotic embryo and endosperm. Rating of 5 equates to strong GUS expression, while rating of 1 equates to weak expression. Non-transformed control tissues showed no GUS expression. FIG. 17 shows comparison of gene expression for 9 promoters in other tissue types. HvPER1 has weak expression in silk and callus; Emb1v1 has weak expression in silk.

In summary:
HvPER1 promoter (TGZm159) showed strong GUS gene expression in zygotic embryos and endosperm at 9, 14 and 21 DAP but none in developing ears at 3, 5 and 7 DAP. Weak expression in callus and silk was observed. No expression in stem, leaf, roots, anthers and pollen was detected.
Hv B1 Hordein promoter (TGZm160) gave strong gene expression in endosperm but none in zygotic embryos at 9, 14 and 21 DAP. No expression was detected in developing ears at 3-7 DAP. No expression was detected in vegetative tissues (stem, leaves, roots) or in silk, anthers, pollen, or callus.
ZmAsp1 (TGZm157), ZmCLV1 (TGZm158), Emb1v1 (TGZm161) and Emb4v1 (TGZm164) promoters gave weak expression in 9, 14, 21 DAP zygotic embryos and endosperm but no expression in developing ears at 3, 5 and 7 DAP. The Emb1v1 promoter shows more amorphous, widely distributed weak GUS expression, while the Emb4v1 promoter gave highly tissue-specific gene expression in the basal endosperm transfer layer. No expression was observed in other tissue types with the exception of weak Emb1v1 expression in silk.
ZmEmb2v1 (TGZm162), ZmEndosperm1y1 (TGZm166), and OsASP1v2 (TGZm181) promoters gave weak expression in 9, 14, and 21 DAP basal endosperm transfer layer. ZmEmb2v1 promoter also gave weak expression in endosperm at 14 DAP. For these promoters, no expression was seen in developing ears at 3, 5 and 7 DAP or in other tissue types.
The embryo and/or endosperm promoters show a range of strengths and tissue-specificities by GUS assay. The identification of promoters of varying strengths and tissue-specificities is valuable for expression of genes whose efficacies depend upon the appropriate level of expression and/or temporal and spatial expression.

Based on the strongly positive results with Hv B1 Hordein (TGZm160), and HvPER (TGZm159) promoters, these promoters are used to drive tissue-specific gene expression in embryo and/or endosperm.

Meanwhile, expression of ZmCLV1 promoter (TGZm158) in inflorescence meristem tissues (developing ears before pollination) is being determined. Transformations are initiated with new constructs containing additional candidate promoters driving the GUS reporter gene.

### Example 4

### Further Corn Promoter Characterization

More constructs are made with candidate promoters driving genes of interest and the efficacy of the promoter-gene expression cassettes are evaluated in field trials (Table 5).

TABLE 5
Promoter/Gene

promoter associated with GRMZM2G124663/REV or dominant negative KRP
promoter associated with GRMZM2G176390/REV or dominant negative KRP
promoter associated with GRMZM2G040517/REV or dominant negative KRP
promoter associated with GRMZM2G066546/REV or dominant negative KRP
promoter associated with GRMZM2G046532/REV
promoter associated with GRMZM2G087413/REV or dominant negative KRP
promoter associated with GRMZM2G091054/REV or dominant negative KRP
promoter associated with GRMZM2G120008/REV or dominant negative KRP
promoter associated with GRMZM2G158407/REV or dominant negative KRP
promoter associated with GRMZM2G132162/REV or dominant negative KRP
promoter associated with GRMZM2G006585/REV or dominant negative KRP
promoter associated with GRMZM2G700896/REV or dominant negative KRP
promoter associated with GRMZM2G091445/REV or dominant negative KRP
promoter associated with GRMZM2G059620/REV or dominant negative KRP
promoter associated with GRMZM2G088896/REV or dominant negative KRP
promoter associated with GRMZM2G175976/REV or dominant negative KRP
promoter associated with GRMZM2G046086/REV or dominant negative KRP
promoter associated with GRMZM2G175912/REV or dominant negative KRP
promoter associated with GRMZM2G152655/REV or dominant negative KRP
promoter associated with GRMZM2G138727/REV or dominant negative KRP
promoter associated with GRMZM2G025763/REV or dominant negative KRP
promoter associated with GRMZM2G090264/REV or dominant negative KRP
promoter associated with GRMZM2G157806/REV or dominant negative KRP
promoter associated with GRMZM2G472234/REV or dominant negative KRP
promoter associated with GRMZM2G409372/REV or dominant negative KRP
promoter associated with GRMZM2G065157/REV or dominant negative KRP
promoter associated with GRMZM2G169149/REV or dominant negative KRP
promoter associated with GRMZM2G701926/REV or dominant negative KRP
promoter associated with GRMZM2G174883/REV or dominant negative KRP
promoter associated with GRMZM2G369799/REV or dominant negative KRP
promoter associated with GRMZM2G140302/REV or dominant negative KRP

The constructs are transformed into maize, and expression of the REV gene or the dominant negative KRP gene is analyzed.

### Example 5

### Promoter Variants Operably Linked to a Gene of Interest in Stably Transformed Maize

To assess the activity of representative functional variants of the embryo- and/or endosperm specific or preferred promoters, the functional variants of the promoters in Examples 3 and 4 are operably linked to a gene of interest and stably transformed into maize plants.

A non-limiting list of constructs comprising sequences that are tested is shown in Table 6 below.

TABLE 6
Constructs with Promoter Variants Operably Linked to a Gene of Interest

| Construct No. | Promoter Sequence |
|---|---|
| 1. | at least 90% identical to the promoter associated with GRMZM2G124663 |
| 2. | at least 90% identical to the promoter associated with GRMZM2G176390 |
| 3. | at least 90% identical to the promoter associated with GRMZM2G040517 |
| 4. | at least 90% identical to the promoter associated with GRMZM2G066546 |
| 5. | at least 90% identical to the promoter associated with GRMZM2G046532 |
| 6. | at least 90% identical to the promoter associated with GRMZM2G087413 |
| 7. | at least 90% identical to the promoter associated with GRMZM2G091054 |
| 8. | at least 90% identical to the promoter associated with GRMZM2G120008 |
| 9. | at least 90% identical to the promoter associated with GRMZM2G158407 |
| 10. | at least 90% identical to the promoter associated with GRMZM2G132162 |
| 11. | at least 90% identical to the promoter associated with GRMZM2G006585 |
| 12. | at least 90% identical to the promoter associated with GRMZM2G700896 |
| 13. | at least 90% identical to the promoter associated with GRMZM2G091445 |
| 14. | at least 90% identical to the promoter associated with GRMZM2G059620 |
| 15. | at least 90% identical to the promoter associated with GRMZM2G088896 |
| 16. | at least 90% identical to the promoter associated with GRMZM2G175976 |
| 17. | at least 90% identical to the promoter associated with GRMZM2G046086 |
| 18. | at least 90% identical to the promoter associated with GRMZM2G175912 |
| 19. | at least 90% identical to the promoter associated with GRMZM2G152655 |
| 20. | at least 90% identical to the promoter associated with GRMZM2G138727 |
| 21. | at least 90% identical to the promoter associated with GRMZM2G025763 |
| 22. | at least 90% identical to the promoter associated with GRMZM2G090264 |
| 23. | at least 90% identical to the promoter associated with GRMZM2G157806 |
| 24. | at least 90% identical to the promoter associated with GRMZM2G472234 |
| 25. | at least 90% identical to the promoter associated with GRMZM2G409372 |
| 26. | at least 90% identical to the promoter associated with GRMZM2G065157 |
| 27. | at least 90% identical to the promoter associated with GRMZM2G169149 |
| 28. | at least 90% identical to the promoter associated with GRMZM2G701926 |
| 29. | at least 90% identical to the promoter associated with GRMZM2G174883 |
| 30. | at least 90% identical to the promoter associated with GRMZM2G369799 |
| 31. | at least 90% identical to the promoter associated with GRMZM2G140302 |

q-PCR is used to measure the relative expression levels of the gene of interest in maize plants transformed with each of the expression vectors. The results indicate that one or more functional variants of the promoters of Examples 3 and 4 also lead to embryo- and/or endosperm-specific or preferred expression profiles.

### Example 6

### Promoter Fragments Operably Linked to a Gene of Interest in Stably Transformed Maize

To assess the activity of fragments of the embryo- and/or endosperm specific or preferred promoters characterized in Examples 3 and 4, certain fragments of the promoters in Examples 3 and 4 are operably linked to a gene of interest and stably transformed into maize plants.

A non-limiting list of constructs comprising sequences that are tested is shown in Table 7 below.

TABLE 7
Constructs with Promoter Fragment Operably Linked to a Gene of Interest

| Construct No. | Promoter Sequence |
|---|---|
| 32. | at least 500 bp, 1.0 kb, 1.5 kb, 2.0 kb, or 3 kb of promoter associated with GRMZM2G124663 |
| 33. | at least 500 bp, 1.0 kb, 1.5 kb, 2.0 kb, or 3 kb of promoter associated with GRMZM2G176390 |
| 34. | at least 500 bp, 1.0 kb, 1.5 kb, 2.0 kb, or 3 kb of promoter associated with GRMZM2G040517 |
| 35. | at least 500 bp, 1.0 kb, 1.5 kb, 2.0 kb, or 3 kb of promoter associated with GRMZM2G066546 |
| 36. | at least 500 bp, 1.0 kb, 1.5 kb, 2.0 kb, or 3 kb of promoter associated with GRMZM2G046532 |
| 37. | at least 500 bp, 1.0 kb, 1.5 kb, 2.0 kb, or 3 kb of promoter associated with GRMZM2G087413 |
| 38. | at least 500 bp, 1.0 kb, 1.5 kb, 2.0 kb, or 3 kb of promoter associated with GRMZM2G091054 |
| 39. | at least 500 bp, 1.0 kb, 1.5 kb, 2.0 kb, or 3 kb of promoter associated with GRMZM2G120008 |
| 40. | at least 500 bp, 1.0 kb, 1.5 kb, 2.0 kb, or 3 kb of promoter associated with GRMZM2G158407 |
| 41. | at least 500 bp, 1.0 kb, 1.5 kb, 2.0 kb, or 3 kb of promoter associated with GRMZM2G132162 |
| 42. | at least 500 bp, 1.0 kb, 1.5 kb, 2.0 kb, or 3 kb of promoter associated with GRMZM2G006585 |
| 43. | at least 500 bp, 1.0 kb, 1.5 kb, 2.0 kb, or 3 kb of promoter associated with GRMZM2G700896 |
| 44. | at least 500 bp, 1.0 kb, 1.5 kb, 2.0 kb, or 3 kb of promoter associated with GRMZM2G091445 |
| 45. | at least 500 bp, 1.0 kb, 1.5 kb, 2.0 kb, or 3 kb of promoter associated with GRMZM2G059620 |
| 46. | at least 500 bp, 1.0 kb, 1.5 kb, 2.0 kb, or 3 kb of promoter associated with GRMZM2G088896 |
| 47. | at least 500 bp, 1.0 kb, 1.5 kb, 2.0 kb, or 3 kb of promoter associated with GRMZM2G175976 |
| 48. | at least 500 bp, 1.0 kb, 1.5 kb, 2.0 kb, or 3 kb of promoter associated with GRMZM2G046086 |
| 49. | at least 500 bp, 1.0 kb, 1.5 kb, 2.0 kb, or 3 kb of promoter associated with GRMZM2G175912 |
| 50. | at least 500 bp, 1.0 kb, 1.5 kb, 2.0 kb, or 3 kb of promoter associated with GRMZM2G152655 |
| 51. | at least 500 bp, 1.0 kb, 1.5 kb, 2.0 kb, or 3 kb of promoter associated with GRMZM2G138727 |
| 52. | at least 500 bp, 1.0 kb, 1.5 kb, 2.0 kb, or 3 kb of promoter associated with GRMZM2G025763 |
| 53. | at least 500 bp, 1.0 kb, 1.5 kb, 2.0 kb, or 3 kb of promoter associated with GRMZM2G090264 |
| 54. | at least 500 bp, 1.0 kb, 1.5 kb, 2.0 kb, or 3 kb of promoter associated with GRMZM2G157806 |
| 55. | at least 500 bp, 1.0 kb, 1.5 kb, 2.0 kb, or 3 kb of promoter associated with GRMZM2G472234 |
| 56. | at least 500 bp, 1.0 kb, 1.5 kb, 2.0 kb, or 3 kb of promoter associated with GRMZM2G409372 |
| 57. | at least 500 bp, 1.0 kb, 1.5 kb, 2.0 kb, or 3 kb of promoter associated with GRMZM2G065157 |
| 58. | at least 500 bp, 1.0 kb, 1.5 kb, 2.0 kb, or 3 kb of promoter associated with GRMZM2G169149 |
| 59. | at least 500 bp, 1.0 kb, 1.5 kb, 2.0 kb, or 3 kb of promoter associated with GRMZM2G701926 |
| 60. | at least 500 bp, 1.0 kb, 1.5 kb, 2.0 kb, or 3 kb of promoter associated with GRMZM2G174883 |
| 61. | at least 500 bp, 1.0 kb, 1.5 kb, 2.0 kb, or 3 kb of promoter associated with GRMZM2G369799 |
| 62. | at least 500 bp, 1.0 kb, 1.5 kb, 2.0 kb, or 3 kb of promoter associated with GRMZM2G140302 |

q-PCR is used to measure the relative expression levels of the gene of interest in maize plants transformed with each of the expression vectors. The results indicate that one or more fragments of the promoters of Examples 3 and 4 also lead to embryo- and/or endosperm-specific or preferred expression profiles.

### Example 7

### Expressing a Gene of Interest in an Embryo- and/or Endosperm-Specific or Preferred Manner in Transformed Maize

Each of the embryo- and/or endosperm-specific or preferred promoters listed in Table 8 is operably linked to a gene of interest. Each of the references cited in Table 8 is incorporated herein by reference in its entirety.

In some embodiments, the gene of interest is a reporter gene (e.g., GUS or GFP), a plant REVOLUTA gene or a dominant negative KRP gene as described in WO 2007/016319 or WO 2007/079353 to make expression cassettes.

Each of the expression cassettes is then inserted into an expression vector suitable for maize transformation to make maize expression vectors.

Each of the maize expression vectors is then transformed into maize plants by using the methods described herein, followed by proper selection process to identify transformation events.

For each transformation, at least 3 transformation events are generated and selfed to produce T2 populations, and either heterozygous or homozygous transformed maize plants are selected and tested to confirm the gene of interest is expressed in an embryo- and/or endosperm-specific or preferred manner. q-PCR and/or other methods are used to test the expression pattern of the gene of interest (e.g., REVOLUTA or dominant negative KRP). The result suggests that each of the expression cassettes successfully give an embryo- and/or endosperm-specific or preferred expression of the gene of interest.

TABLE 8

| TG GUS | | | |
|---|---|---|---|
| construct | promoter | from | Description |
| TG_Zm 156 | Os aspartic protease 1v1 | Oryza | promoter from rice aspartic protease gene ASP |
| | | sativa | that drives early embryo-specific expression of the |
| | | | transgene |
| | | | (Bi et al., 2005, Plant Cell Physiol. 46(1): 87-98) |
| TG_Zm 157 | Zm aspartic protease 1 | Zea mays | promoter from corn aspartic protease gene ASP |
| | (ZmASP1, | | that drives early embryo-specific expression of the |
| | GRMZM2G128268, | | transgene |
| | ortholog of OsASP1) | | |
| TG_Zm 158 | Zm Clavata 1 (ZmCLV1, | Zea mays | Drives inflorescence meristem expression of the |
| | GRMZM2G300135, | | transgene |
| | ortholog of AtCLV1) | | (Bommert et al., Development 132 (6), 1235-1245) |
| TG_Zm 159 | Hv Peroxiredoxin 1 | Hordeum | Drives embryo specific expression of the transgene |
| | (a.k.a. B15C, homologue | vulgare | (WO 2005/007829; Aalen et al, 1994, Plant J 5: |
| | to AtPer1) | | 385-396; Stacy et al, 1999, Plant J. 19: 1-8; Stacy |
| | | | et al. 1996, Plant Mol Biol 31: 1205-1216) |
| TG_Zm 160 | Hv B1 hordein | Hordeum | Drives endosperm specific expression of the |
| | | vulgare | transgene |
| | | | (Cho et al., 2002, Physiol Plant. 2002 |
| | | | May; 115(1): 144-154.) |
| TG_Zm 161 | ZmEmbryo1v1 | Zea mays | promoter sequence obtained from deep sequencing |
| | (ZmEMB1, | | of cRNAs from early embryos (7-9 DAP) of corn |
| | GRMZM2G120008) | | and then finding the correlating promoter sequence |
| | | | from a public corn genome database. The |
| | | | sequence identifier for the promoter in the |
| | | | database is GRMZM2G120008. Drives early |
| | | | embryo-specific expression of the transgene. |
| | | | Version 1 is GRMZM2G120008; −2031 to +15 from ATG. |
| TG_Zm 162 | Zm Embryo2v1 | Zea mays | promoter sequence obtained from deep sequencing |
| | (ZmEMB2, | | of cRN As from early embryos (7-9 DAP) of corn |
| | GRMZM2G124663, | | and then finding the correlating promoter sequence |
| | ortholog of AtLEC1, also | | from a public corn genome database. The |
| | called TGI ZmLEC1 | | sequence identifier for the promoter in the |
| | herein) | | database is GRMZM2G124663. Drives early |
| | | | embryo-specific expression of the transgene. |
| | | | Version 1 is GRMZM2G124663; −866 to -42 from ATG. |
| TG_Zm 163 | ZmEmbryo3 (ZmEMB3, | Zea mays | promoter sequence obtained from deep sequencing |
| | GRMZM2G157806, | | of cRN As from early embryos (7-9 DAP) of corn |
| | ortholog of AtTPD1) | | and then finding the correlating promoter sequence |
| | | | from a public corn genome database. The |
| | | | sequence identifier for the promoter in the |
| | | | database is GRMZM2G157806. Drives early |
| | | | embryo-specific expression of the transgene. |
| TG_Zm 164 | Zm Embryo4v1 | Zea mays | promoter sequence obtained from deep |
| | (ZmEMB4, | | sequencing of cRNAs from early embryos (7-9 |
| | GRMZM2G176390) | | DAP) of corn and then finding the correlating |
| | | | promoter sequence from a public corn genome |
| | | | database. The sequence identifier for the promoter |
| | | | in the database is GRMZM2G176390. Drives |
| | | | early embryo-specific expression of the transgene. |
| | | | Version 1 is GRMZM2G176390; −1527 to −52 from ATG. |
| TG_Zm 165 | ZmEmbryo5 (ZmEMB5, | Zea mays | promoter sequence obtained from deep sequencing |
| | GRMZM2G472234; −1965 | | of cRN As from early embryo (7-9 DAP) of corn |
| | to −399 from ATG, | | and then finding the correlating promoter sequence |
| | Ortholog of Arabidopsis | | from a public corn genome database. The |
| | hydroxyproline rich | | sequence identifier for the promoter in the |
| | glycoprotein) | | database is GRMZM2G472234. Drives early |
| | | | embryo-specific expression of the transgene. |
| TG_Zm 166 | ZmEndosperm1v1 | Zea mays | promoter sequence obtained from deep sequencing |
| | (ZmEND1, | | of cRNAs from early endosperm (7-9 DAP) of |
| | GRMZM2G006585, | | corn and then finding the correlating promoter |
| | ortholog of rice zinc- | | sequence from a public corn genome database. |
| | binding protein) | | The sequence identifier for the promoter in the |
| | | | database is GRMZM2G006585. Drives early |
| | | | endosperm-specific expression of the transgene. |
| | | | Version 1 is GRMZM2G006585; −1902 to −43 from ATG. |
| TG_Zm 167 | ZmEndosperm2 | Zea mays | promoter sequence obtained from deep sequencing |
| | (ZmEND2, | | of cRNAs from early endosperm (7-9 DAP) of |
| | GRMZM2G138727) | | corn and then finding the correlating promoter |
| | | | sequence from a public corn genome database. |
| | | | The sequence identifier for the promoter in the |
| | | | database is GRMZM2G138727. Drives early |
| | | | endosperm-specific expression of the transgene. |
| TG_Zm 181 | Os aspartic protease 1v2 | Oryza | promoter from rice aspartic protease gene ASP |
| | (internal hairpin 568 bp- | sativa | that drives early embryo-specific expression of the |
| | 639 bp missing) | | transgene. While cloning, a hairpin caused a |
| | | | deletion of base pairs 568 to 639. |
| TG_Zm 212 | ZmEmb2v2 | Zea mays | promoter sequence obtained from deep sequencing |
| | | | of cRNAs from early embryos (7-9 DAP) of corn |
| | | | and then finding the correlating promoter sequence |
| | | | from a public corn genome database. The |
| | | | sequence identifier for the promoter in the |
| | | | database is GRMZM2G124663. Drives early |
| | | | embryo-specific expression of the transgene. |
| | | | Version 2 is GRMZM2G124663; −866 to 1 from ATG. |
| TG_Zm 213 | ZmEmb2v3 | Zea mays | promoter sequence obtained from deep sequencing |
| | | | of cRNAs from early embryos (7-9 DAP) of corn |
| | | | and then finding the correlating promoter sequence |
| | | | from a public corn genome database. The |
| | | | sequence identifier for the promoter in the |
| | | | database is GRMZM2G124663. Drives early |
| | | | embryo-specific expression of the transgene. |
| | | | Version 3 is GRMZM2G124663; −600 to −1 from ATG. |
| TG_Zm 214 | ZmEmb4v2 | Zea mays | promoter sequence obtained from deep sequencing |
| | | | of cRNAs from early embryos (7-9 DAP) of corn |
| | | | and then finding the correlating promoter sequence |
| | | | from a public corn genome database. The |
| | | | sequence identifier for the promoter in the |
| | | | database is GRMZM2G176390. Drives early |
| | | | embryo-specific expression of the transgene. |
| | | | Version 2 is GRMZM2G176390; −1527 to −1 from ATG. |
| TG_Zm 215 | ZmEmb4v3 | Zea mays | promoter sequence obtained from deep sequencing |
| | | | of cRNAs from early embryos (7-9 DAP) of corn |
| | | | and then finding the correlating promoter sequence |
| | | | from a public corn genome database. The |
| | | | sequence identifier for the promoter in the |
| | | | database is GRMZM2G176390. Drives early |
| | | | embryo-specific expression of the transgene. |
| | | | Version 3 is GRMZM2G176390; −900 to −1 from ATG. |
| TG_Zm 216 | ZmEmb4v4 | Zea mays | promoter sequence obtained from deep sequencing |
| | | | of cRNAs from early embryos (7-9 DAP) of corn |
| | | | and then finding the correlating promoter sequence |
| | | | from a public corn genome database. The |
| | | | sequence identifier for the promoter in the |
| | | | database is GRMZM2G176390. Drives early |
| | | | embryo-specific expression of the transgene. |
| | | | Version 4 is GRMZM2G176390; −818 to −1 from ATG. |
| | ZmBETL-3 | Zea mays | Drives expression of gene of interest in a BETL-, |
| | (GRMZM2G175976) | | endosperm-, or embryo-specific manner. |
| | ZmMRP-1 | Zea mays | Drives expression of gene of interest in a BETL- |
| | | | specific manner. |
| | | | (Barrerro et al, 2009, Planta 229: 235-247; Gomez |
| | | | et al, 2009, The Plant Cell 21: 2022-2035; Gomez |
| | | | et al, 2002, The Plant Cell 14: 599-610) |
| | Unnamed | Zea mays | Drives expression of gene of interest in a BETL-, |
| | (GRMZM2G066546) | | endosperm-, or embryo-specific manner. |
| | ZmBETL-9 | Zea mays | Drives expression of gene of interest in a BETL-, |
| | (GRMZM2G087413) | | endosperm-, or embryo-specific manner. |
| | ZmSh-2 | Zea mays | Drives expression of gene of interest in an |
| | (GRMZM2G158407) | | endosperm-, or embryo-specific manner. |
| | ZmBETL-10 | Zea mays | Drives expression of gene of interest in a BETL-, |
| | (GRMZM2G091445) | | endosperm-, or embryo-specific manner. |
| | Unnamed | Zea mays | Drives expression of gene of interest in an |
| | (GRMZM2G700896) | | endosperm-, or embryo-specific manner. |
| | Unnamed | Zea mays | Drives expression of gene of interest in an |
| | (GRMZM2G175912) | | endosperm-, or embryo-specific manner. |
| | ZmBETL-2 | Zea mays | Drives expression of gene of interest in a BETL-, |
| | (GRMZM2G152655) | | endosperm-, or embryo-specific manner. |

BETL stands for basal endosperm transfer layer. According to Royo et al., (Plant Cell Monogr (8): 73-89, 2007), it is differentiated endosperm

### Example 8

### Corn Breeding Program Using the Transgenic Plants Comprising the Embryo- and/or Endosperm-Specific or Preferred Promoters

Non-limiting methods for corn breeding and agriculturally important traits are described in, for example, Allard, Principles of Plant Breeding, 1960; Simmonds, Principles of Crop Improvement, 1979; Fehr, “Breeding Methods for Cultivar Development”, Production and Uses, 2nd ed., Wilcox editor, 1987, Carena et al., 2010 (Quantitative Genetics in Maize Breeding, Springer, 2010 ISBN 1441907653, 9781441907653); and Kriz and Larkins, 2008 (Molecular Genetic Approaches to Maize Improvement, Springer, 2008, ISBN 3540689192, 9783540689195).

A corn plant comprising one or more embryo- and/or endosperm-specific or preferred promoters of the present invention can be self-crossed to produce offspring comprising the same transgene.

A corn plant comprising one or more embryo- and/or endosperm-specific or preferred promoters of the present invention (“donor plant”, a.k.a. as the non-recurrent parent) can also be crossed with another plant (“recipient plant”, a.k.a. as the recurrent parent) to produce a F1 hybrid plant.

Some of the F1 hybrid plants can be back-crossed to the recipient (i.e., the recurrent parent) plant for 1, 2, 3, 4, 5, 6, 7, or more times. After each backcross, seeds are harvested and can be planted to select plants that comprise the embryo- and/or endosperm-specific or preferred promoters, and preferred traits inherited from the recipient plant. Such selected plants can be used as either male or female plants to backcross with the recipient plant.

As a result, a new corn plant can be produced which comprises all preferred traits inherited from the recipient plant and the embryo- and/or endosperm-specific or preferred promoters inherited from the donor plant.

### Example 9

### Identification of Conserved Motifs in Embryo- and/or Endosperm-Specific or Preferred Promoters

Embryo and/or endosperm promoter sequences from Table 2 were entered into the Multiple Em for Motif Elicitation (MEME) Suite (Timothy L. Bailey and Charles Elkan, “Fitting a mixture model by expectation maximization to discover motifs in biopolymers”, Proceedings of the Second International Conference on Intelligent Systems for Molecular Biology, pp. 28-36, AAAI Press, Menlo Park, Calif., 1994) to discover any pattern similarities amongst the sequences. The following sets of sequences were submitted: 1) all 31 embryo and/or endosperm promoter sequences, 2) only embryo-specific and embryo+endosperm promoter sequences, or 3) only endosperm-specific and embryo+endosperm sequences. As used herein, “Embryo-specific”, “Endosperm-specific”, and “Embryo+Endosperm” categories are based on qPCR data (FIGS. 2-5). All default settings were used.

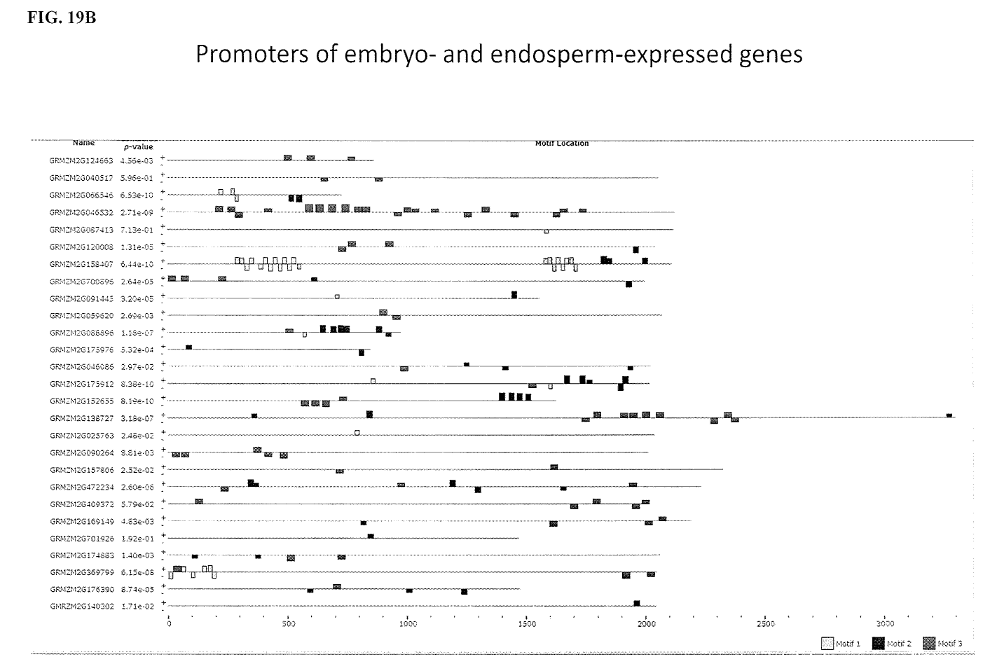

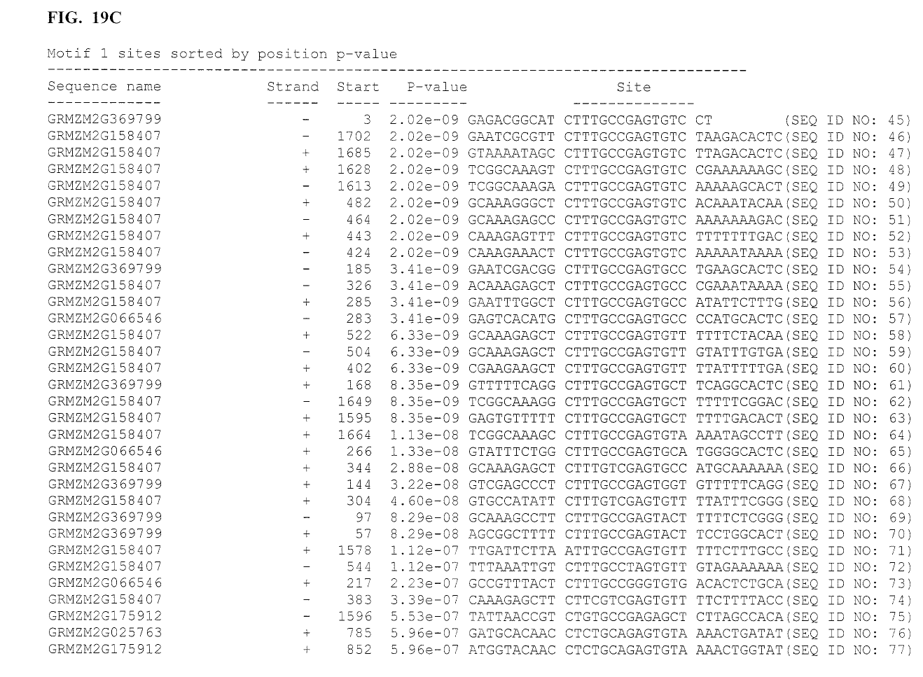

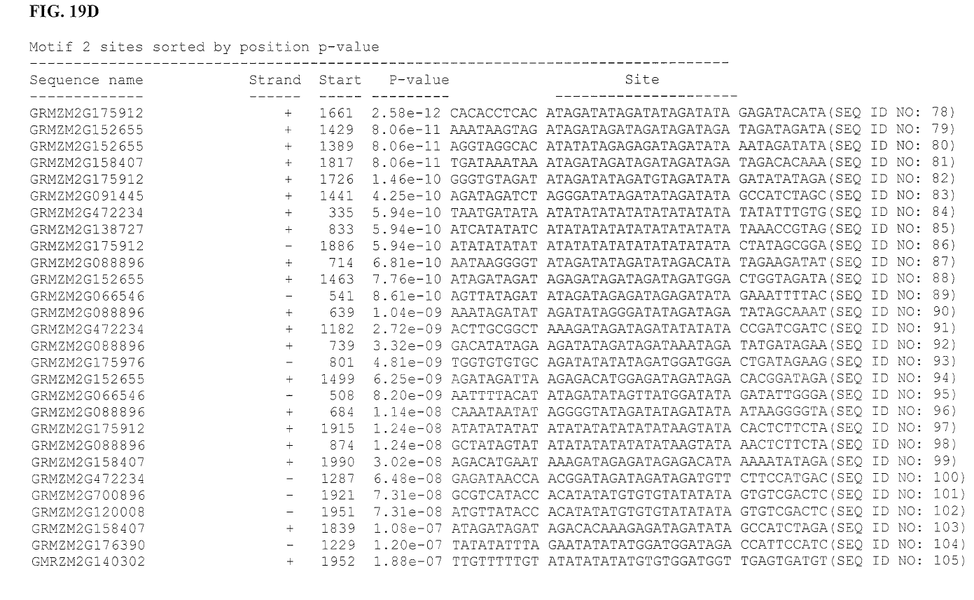

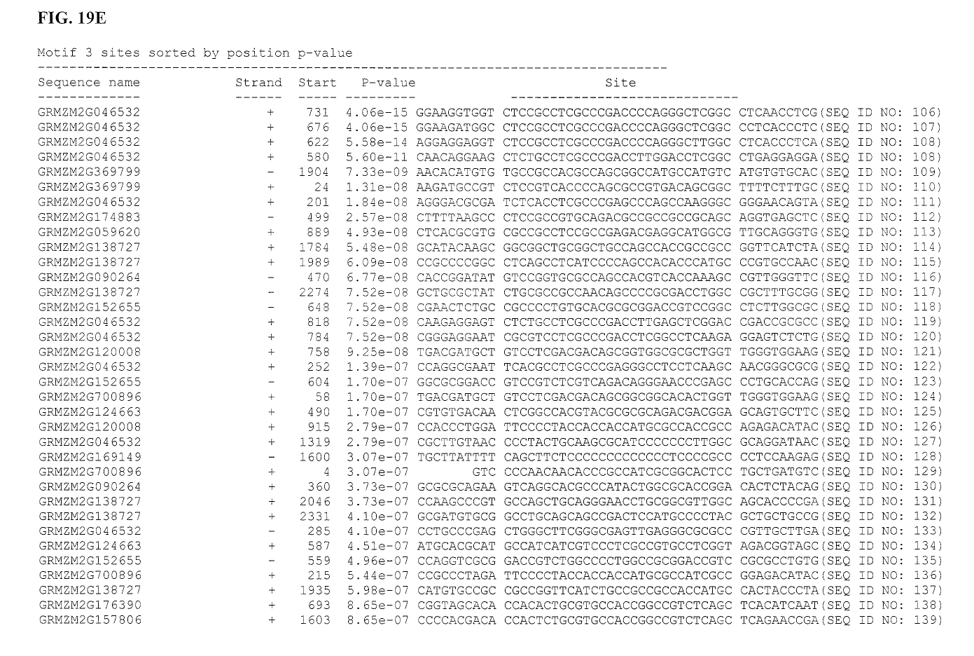

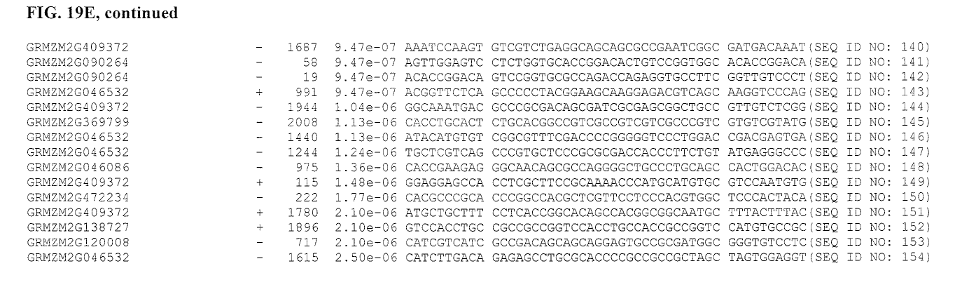

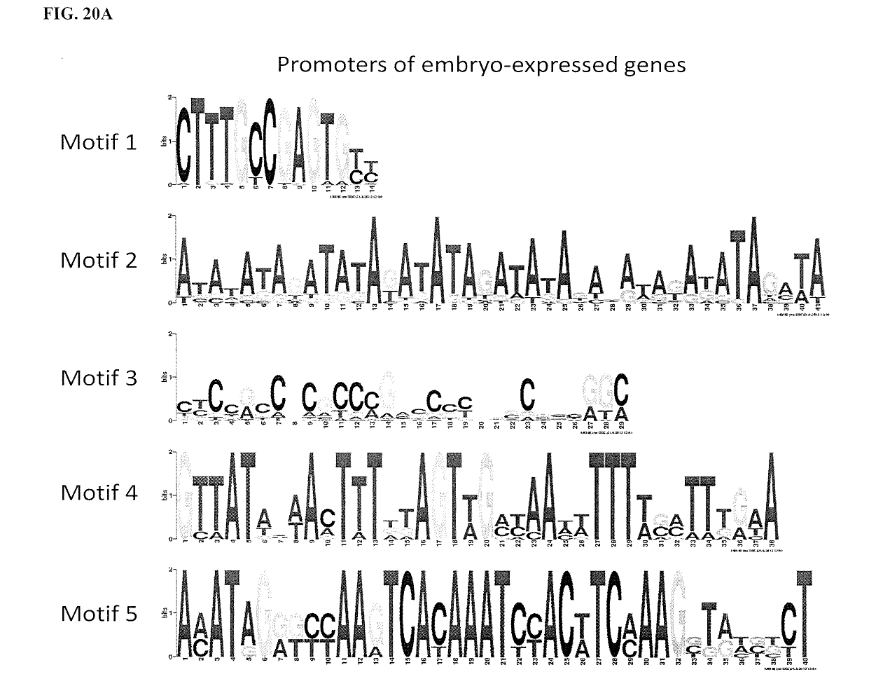

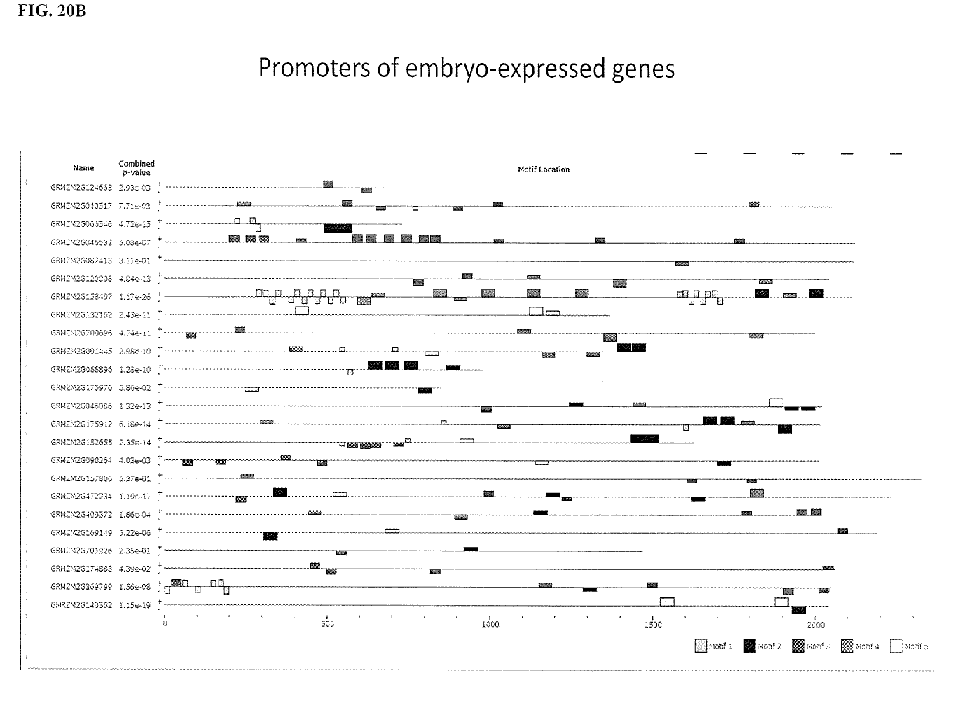

For all 31 embryo and/or endosperm promoter sequences, the top three motifs are shown in FIG. 19A. FIG. 19B shows a combined block diagram of the three motifs across 27 of the 31 promoter sequences. Blocks indicate non-overlapping sites with a p-value better than 0.0001. The height of the motif block is proportional to −log(p-value), truncated at the height for a motif with a p-value of 1e-10. FIG. 19C-E give the nucleotide start positions and DNA strand (+=sense strand, −=antisense strand) on which each motif occurs for motifs 1, 2 and 3, respectively, for relevant embryo and/or endosperm promoter sequences, sorted according to their p-values.

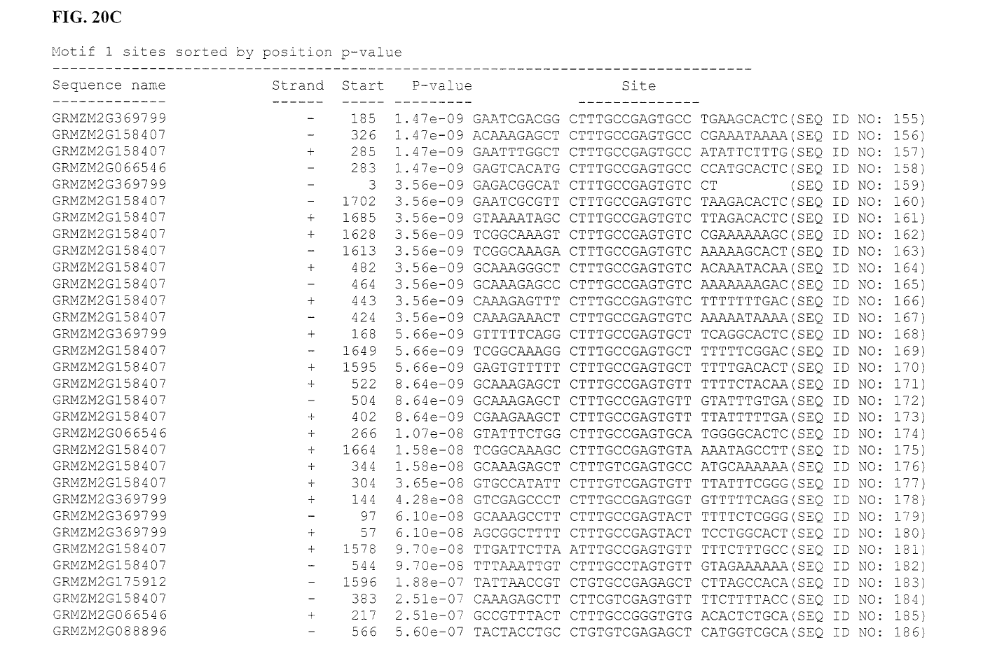

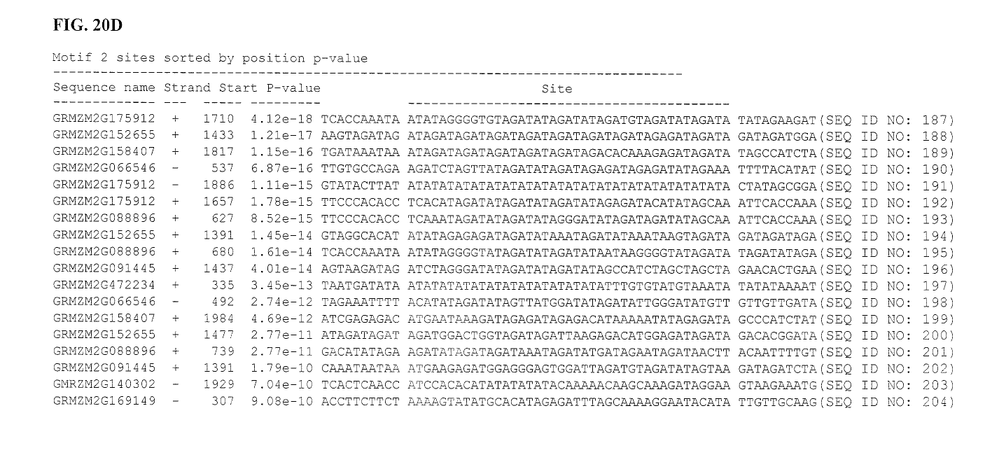

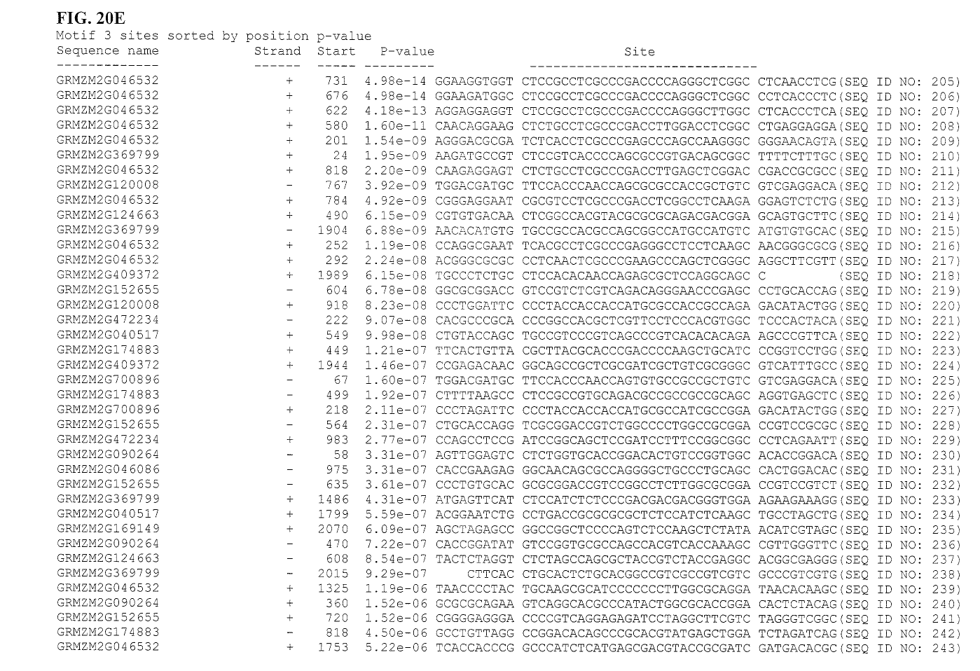

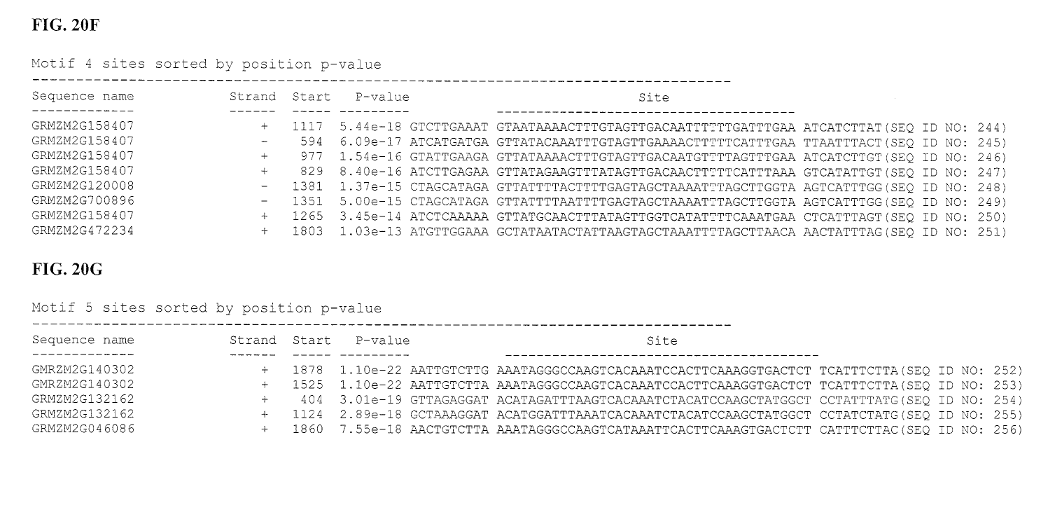

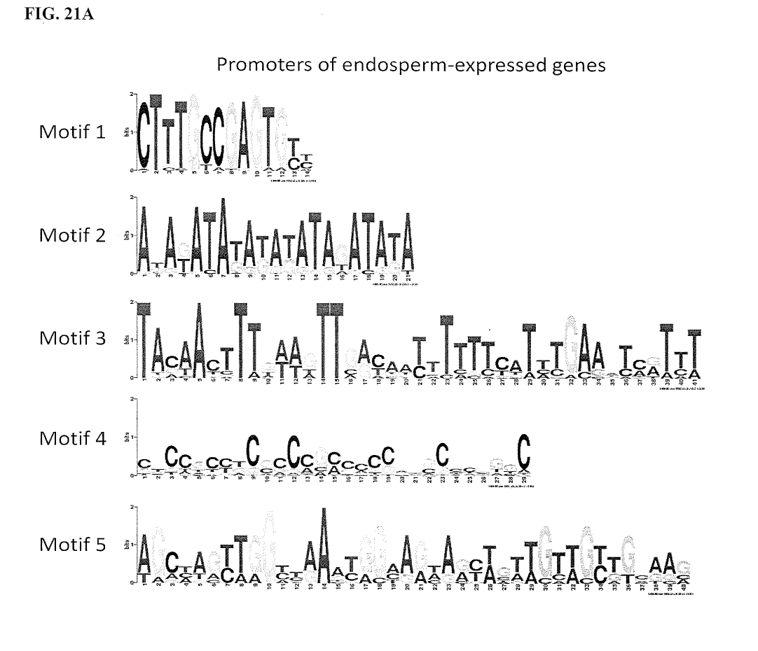

For only embryo-specific and embryo+endosperm promoter sequences, the top five motifs are shown in FIG. 20A. FIG. 20B shows a combined block diagram of the five motifs across 24 of the 26 promoter sequences. FIG. 20C-G give the nucleotide start positions and DNA strand (+=sense strand, −=antisense strand) on which each motif occurs for motifs 1, 2, 3, 4 and 5, respectively, for relevant embryo and/or endosperm promoter sequences, sorted according to their p-values.

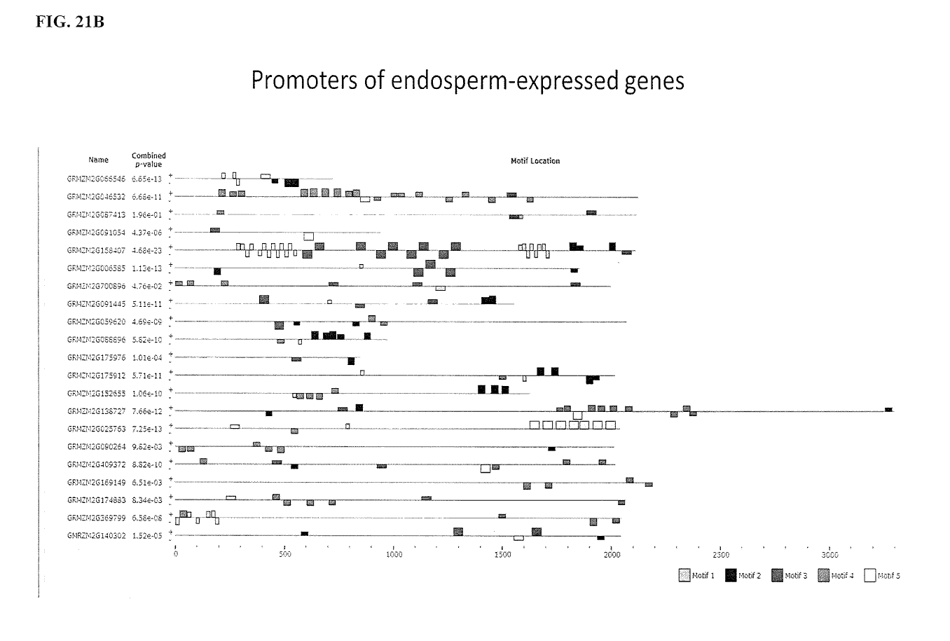

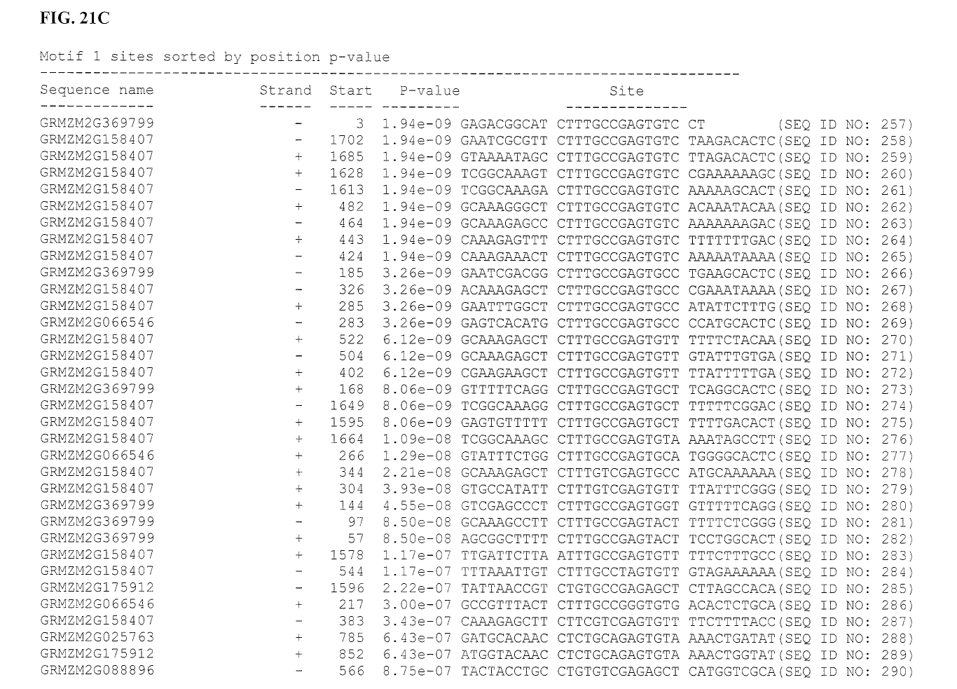

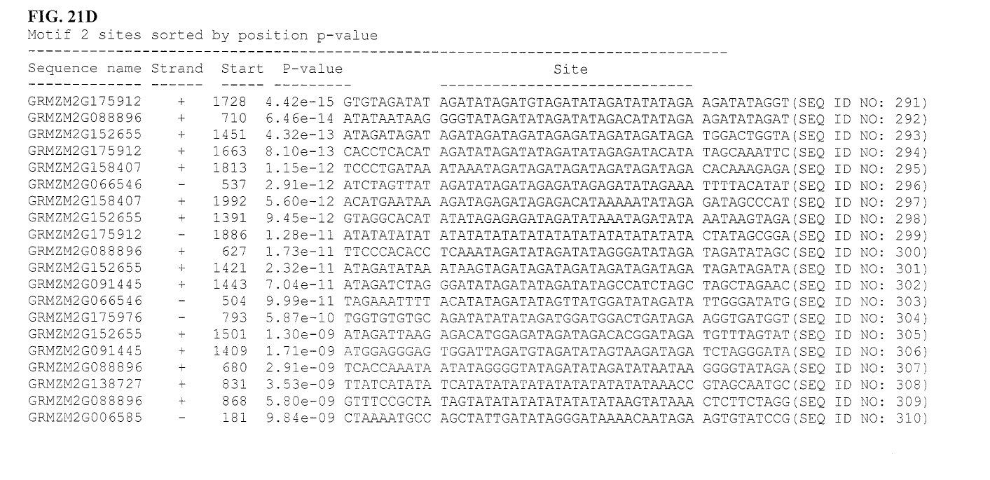

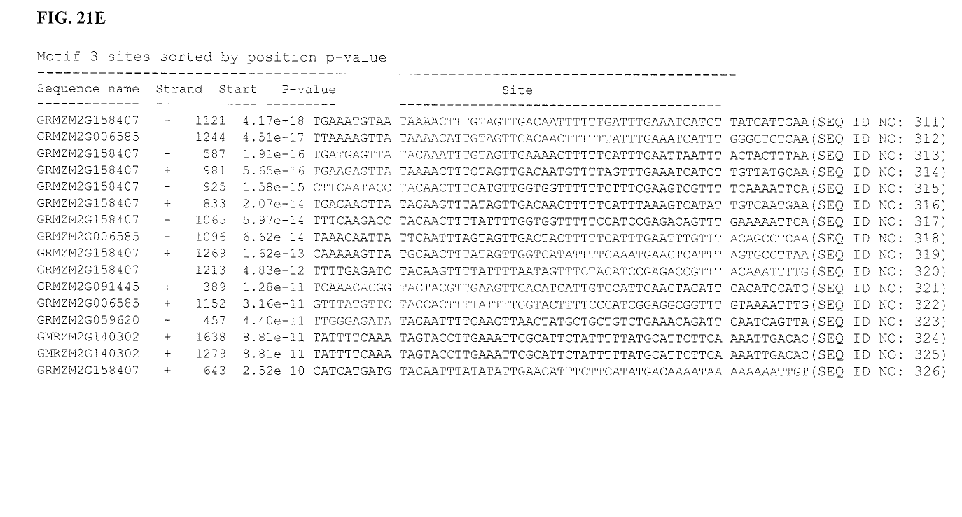

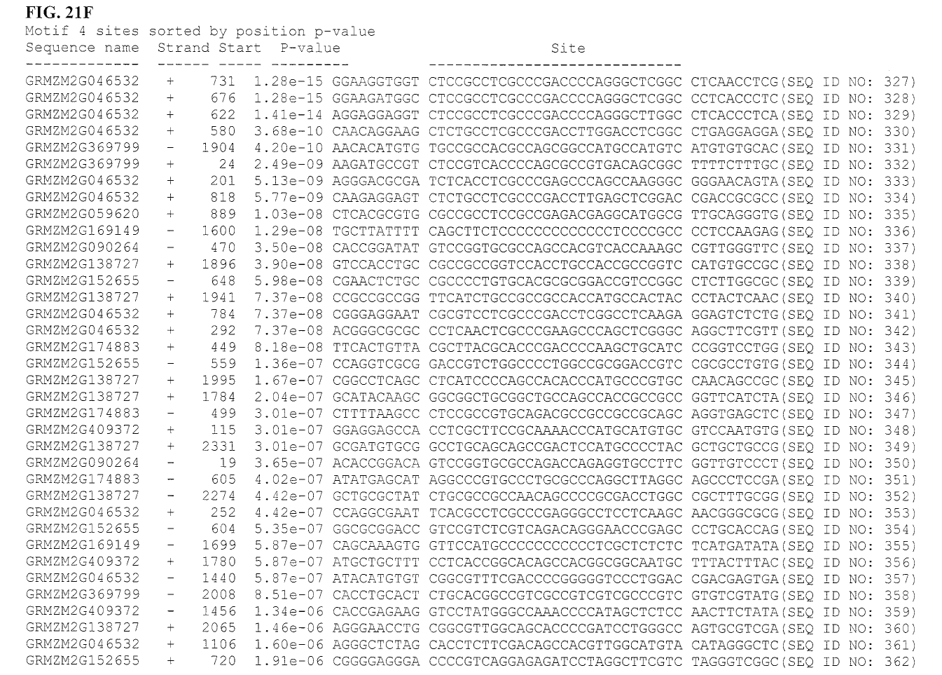

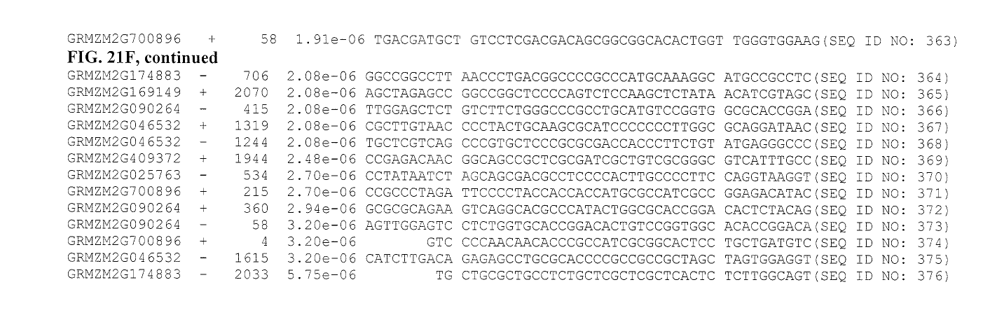

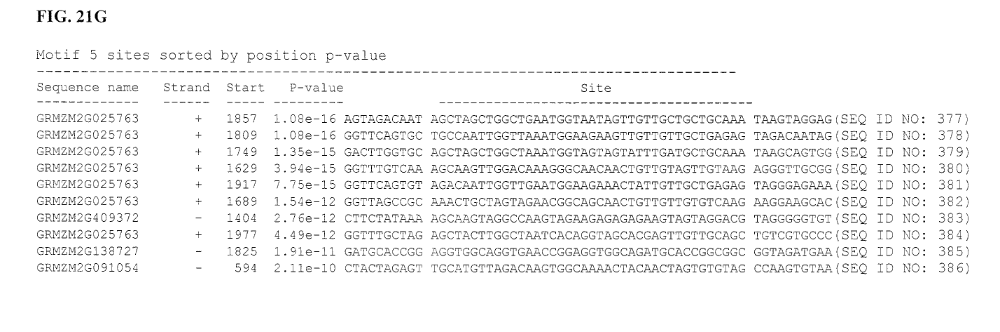

For only endosperm-specific and embryo+endosperm promoter sequences, the top five motifs are shown in FIG. 21A. FIG. 21B shows a combined block diagram of the five motifs across 21 of the 23 promoter sequences. FIG. 21C-G give the nucleotide start positions and DNA strand (+=sense strand, −=antisense strand) on which each motif occurs for motifs 1, 2, 3, 4 and 5, respectively, for relevant embryo and/or endosperm promoter sequences, sorted according to their p-values.

Motif 1 in the promoter sequences of embryo and/or endosperm-expressed genes, of embryo-expressed genes and of endosperm-expressed genes are identical. Motif 2 in the promoter sequences of the embryo and/or endosperm-expressed genes and the endosperm-expressed genes are identical. Motif 3 in the promoter sequences of embryo and/or endosperm-expressed genes is identical to Motif 3 in the promoter sequences of the embryo-expressed genes and to Motif 4 in the promoter sequences of the endosperm-expressed genes. The remaining motifs appear to be unique to each analysis.

### Example 10

### Expression of a Gene of Interest in an Embryo- and/or Endosperm-Specific or Preferred Manner at a Specific Expression Level

Each of the embryo- and/or endosperm-specific or preferred promoters of the present invention is operably linked to a reporter gene, such as GUS or GFP, or other gene of interest to make a group of expression constructs. Each construct is introduced into a maize plant cell, plant part, or plant by methods described herein.

The expression level of the reporter gene or other gene of interest in each construct is analyzed and ranked from the highest to the lowest. The expression construct that provides a desired expression level is selected. The selected expression construct is then transformed into maize plants by using the methods described herein, followed by proper selection process to identify transformation events.

In some embodiments, the gene of interest is toxic to the plant cell, plant part or plant when expressed at high level under other commonly used promoters. A desired expression level can be achieved by selecting a suitable embryo- and/or endosperm-specific or preferred promoters of the present invention, so that the gene of interest is expressed at a desired level which leads to a desired phenotype without causing toxicity or only causing bearable toxicity to the plant cell, plant part, or plant.

Unless defined otherwise, all technical and scientific terms herein have the same meaning as commonly understood by one of ordinary skill in the art to which this invention belongs. Although any methods and materials, similar or equivalent to those described herein, can be used in the practice or testing of the present invention, the non-limiting exemplary methods and materials are described herein.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference. Nothing herein is to be construed as an admission that the present invention is not entitled to antedate such publication by virtue of prior invention.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

1. An expression cassette for embryo- and/or endosperm-specific or preferred gene expression of a gene of interest comprising a promoter sequence comprising a nucleotide sequence selected from the group consisting of:
(a) the nucleotide sequence set forth in SEQ ID NOs: 1-37;
(b) a nucleotide sequence comprising at least 90% nucleotide sequence identity to the nucleotide sequence set forth in SEQ ID NOs: 1-37;
(c) nucleotide sequences that hybridize to a sequence of SEQ ID NOs: 1-37 under stringent conditions, wherein said stringent conditions are hybridization in 0.25 M Na₂HPO₄ buffer (pH 7.2) containing 1 mM Na₂EDTA, 0.5-20% sodium dodecyl sulfate at 45° C., followed by a wash in 5×SSC, containing 0.1% (w/v) sodium dodecyl sulfate, at 55° C. to 65° C.;
(d) nucleotide sequences that comprise at least 1000-mers of a fragment of SEQ ID NOs: 1-37;
(e) nucleotide sequences that comprise at least 500-mers of a fragment of SEQ ID NOs: 1-37;
(f) nucleotide sequences that comprise at least 200-mers of a fragment of SEQ ID NOs: 1-37; and
(g) nucleotide sequences that comprise one or more conserved consensus fragment of a sequence of SEQ ID NOs: 1-37,
wherein the nucleotide molecule is capable of driving the expression of the operably linked gene of interest in an embryo- and/or endosperm-specific or preferred manner in a plant.

2. The expression cassette of claim 1, wherein the expression cassette further comprises at least one enhancer sequence.

3. The expression cassette of claim 2, wherein the enhancer sequence is a transcriptional enhancer sequence and/or a translation enhancer sequence.

4. The expression cassette of claim 3, wherein the enhancer sequence is derived from a virus or a plant.

5.-6. (canceled)

7. The expression cassette of claim 1, wherein the gene of interest is selected from the group consisting of genes encoding REVOLUTA proteins, dominant negative Kinase Inhibitor Protein (KIP) Related Proteins (KRP), acyl-acyl carrier protein (acyl-ACP) thioesterases, acyl-acyl carrier protein (acyl-ACP) desaturases, fatty acid elongases, fatty acid desaturases, acyl-acyl carrier protein synthase III, leafy cotyledon proteins, glycerol-3-phosphate dehydrogenases, diacylglycerol acyltransferases, lysophosphatidic acid acyltransferases, seed storage proteins, aspartate kinases, dihydrodipicolinic acid synthases, phosphoenolpyruvate carboxylases, puroindolines, phytoene synthases, abscisic acid biosynthesis proteins, isopentenyl transferases, sucrose symporters, starch branching enzymes, levansucrases, invertases, sucrose synthases, sucrose isomerases, phytases, DREB1A, ABI3, ADP-glucose pyrophosphorylases, and recombinant proteins for molecular farming applications.

8. An expression vector for expressing a gene of interest in an embryo- and/or endosperm-specific or preferred manner in a maize plant comprising the expression cassette of claim 1.

9.-11. (canceled)

12. A non-human transgenic cell comprising the expression cassette of claim 1.

13. (canceled)

14. An organism comprising a transgenic cell of claim 12.

15. A transformed maize plant, plant part or plant cell comprising the expression cassette of claim 1.

16.-20. (canceled)

21. A seed of the transformed maize plant of claim 15, wherein said seed comprises said expression cassette.

22. A method of expressing a gene of interest in a maize plant or plant cell, said method comprising incorporating into a plant cell a polynucleotide construct comprising a nucleic acid molecule, wherein the nucleic acid molecule comprises at least one expression cassette of claim 1.

23.-25. (canceled)

26. Progeny plants of the plant of claim 1, wherein the progeny plants have the nucleic acid molecule comprising the expression cassette of claim 1.

27. A method of producing hybrid maize seed comprising crossing the plant of claim 15 with a different plant of the same species, and harvesting the resultant seed.

28. A method of expressing a gene of interest in an embryo- and/or endosperm-specific or preferred manner in a maize plant, plant part, or plant cell, comprising utilizing at least one expression cassette of claim 1.

29. A method for producing a plant having a recombinant gene under the control of an embryo- and/or endosperm-specific or preferred promoter comprising: (a) transforming a plant cell with an expression cassette of claim 1; and (b) cultivating the transgenic cell under conditions conducive to regeneration and mature plant growth of a plant having the expression cassette.

30. A method for modifying a phenotype of a target organism, comprising stably incorporating into the genome of the target organism an expression cassette of claim 1.

31. A process of determining the presence or absence of an expression cassette of claim 1, and fragments and variations thereof in a plant, wherein the process comprises at least one of:
(a) isolating nucleic acid molecules from said plant and amplifying sequences homologous to the expression cassette;
(b) isolating nucleic acid molecules from said plant and performing a Southern hybridization to detect the expression cassette;
(c) isolating proteins from said plant and performing a Western Blot using antibodies to a protein encoded by the expression cassette; and/or
(d) demonstrating the presence of mRNA sequences derived from a polynucleotide mRNA transcript and unique to the expression cassette.

32. A method of breeding plants to produce a plant having an expression cassette of claim 1 comprising:
i) making a cross between a plant with an expression cassette of any one of claims 1 to 7 with a second plant to produce a F1 plant;
ii) backcrossing the F1 plant to the second plant; and
iii) repeating the backcrossing step to generate a near isogenic or isogenic line, wherein the expression cassette of claim 1 is integrated into the genome of the second plant and the near isogenic or isogenic line derived from the second plant with the expression cassette.

33. A method of expressing a gene of interest in an embryo- and/or endosperm-specific or preferred manner in a maize plant, plant part, or plant cell at a preferred expression level, comprising utilizing at least one expression cassette of claim 1.

34.-35. (canceled)

36. The expression cassette of claim 1, wherein the gene of interest is an interference RNA.


SEQUENCE LISTING (386 sequences)

SEQ ID NO: 1 - DNA, 865 bp, Zea mays
aactgttgtccagcatggttggtgagatcaattcctgtggccagactcaggcaagatcaacgctttccagtggtgtctgttttgcttagtgaagttgtggccggtcgcgttgtcgatcaaatcaatcaccacacagacaacgcaggacatggtgtattggagcaaacgtgcctccactgttcctcgttctccaatggcaagatctctctattctcgcgagcagtgctgataacgtgttagaactcagtgaaaacgagattcgagagaatagtgcaaatgacagttgtactttattattgcaggggagtcctgattatatacatgtgaaggggtgtctctcaaggggtatgtcccttagggagaatctgtcgcaccctttggattggtcacatatacaatatacaatattttgtaacagtttcgatctaccggattcatccgttttcacgtacgtcgttccatctttcgcgcgcgtactacgtgtgacaactcggccacgtacgcgcgcagacgacggagcagtgcttcacagaatcctccgccacttgtcaccaacacaggcgcgcgctggtgtgcatgcacgcatgccatcatcgtccctcgccgtgcctcggtagacggtagcgctggctagagacctagagtagtagtagcctgtgcttgcttgctagttgctagccggcctggtcaggtcaacacatcgtcgtagcctcctatttaacgtatcctcgcacatcacaagaaggacactctccaccagccagccagagccagtagccaccaccagatcggcctccccgttgctcatcaccagcaagagcaactcagcaacctagctagctataggtttaggtagcaagagcca

SEQ ID NO: 2 - DNA, 2058 bp, Zea mays
gatttatatcaactctagtggacaatgtaaatggctctctatcctttctaccgttcacggctaccactattcatctttccagtagaccgcgcctagtgacgttatcgtggcgagttctttctcccccctcacttcaacagattcctgaaaatttatatattttctttagttcaattgagaaccacattttccagattactgacatcgatgttataaggtgtcatgctcttattgaatctttagttgatagttgtttcaactaaaccaaatttcgtttttttccataattgctagattacggtagctttctcgtagctcgtagttagaattagattttcaaaaaactacacatgaaccattaaggagctgtttggttcgtggctaaatgtgacacactttgcttaatgttagtcattcgaatataaaaactaaccttaaacagaaaagttagacaaagtgtcacaaattagtcatccaaccaaaacagcccctaatccttttacttgatagtgtgtctctctcgcacgcacatacataactgtaccagctgccgtcccgtcagcccgtcacacacagaagcccgttcaaatttgggcggtcgagacatcgtgggctaggccctgtacgtgggcaattcctaaagtcacgtggccgcgtgggcctttatggcgtgctatgcctaggccattaggcccattcaggaataccacttcccgttgctcctctgcaggtccgtaaagaaaactgaaacaagagcgataccccagtaccccacgaagagaggacgggaaaaaaaaaaggaaaactgaaacgaaaagatcaatcaaggcaaaaatggattgctttcccccaaaacaagtaaacgaacgatccatggagacggacgcttccgaggccggaaatccagggagccatggcgaccggcttgcttgtcgtagcagtaacgccacacgcataccactgcggaaattaatcccatgtcgtttgtctggatgggtaacaacaacgatcgccgttccgcacaaatctccgagcgagacacggacaccgatgcaccccacgtccccaccccaaccaaaaaaaaaaaccatccgaatactagtactactgtactgtaaaagcaaaattgttacatgcacagcacatgggtgctaactgttaagagcttctttggaacaaaggttttttccctgtttccaggattttttatgtgaaaatgaactgatttctacgaaatttgtatatgacttatctaaaaattcctacattccaaagaagccctaaggttgaactcagacagataaatgtggccagagtaaggcctgttttaatctcacgggataaactttagttatatgaattcaagtgctaaagtttagctttaattaccaccattagctctactgtttagattacaaatggctaaaagtagctaaaaaaagctgctaaagtttatctcgcgagattggaacaccacctaagctttcattaccttttgtgtgtcgtttagcaacaagccgttgctgctcggatgccgcagggctgtgtcgttagatggcagcacatgcatgcatgcatggatccagctttaatttgatacgcatgcatgcagtcattgttccttaattcactgtaattctgcagcagtcgtcgaagacataattcagctcagaaattaacccgcagttccctgccctgcgttgatctttaacttttacaaacctagctgacgcactcatccgttgcggcctgcgggtaatgccagacaaaagattgcagtaacttgaaacggaatctgcctgaccgcgcgcgctctccatctcaagctgcctagctggttcagtttgtttgtttgtttgtttgatgagaaagcgcaaacgggagagaaaccctagctggctagctctcccgcctctctataaatcagcgcagcctgttcgtacagcgccacacaccacagttagcttcctttcaattattgtatttcagtcgagttgctaaacaacacgaactcctcagtcgtttgagttttgaggaggcaagcaagaggacccatcg

SEQ ID NO: 3 - DNA, 733 bp, Zea mays
taccggaactaggagattcgttcttccttcggtgtcatcacacatcgttggaccacattccagagtgaccaccagtatacaggaaagagagacaacggggtgaaccgttggacacggcctggaaacaatggtggcggctggaaacaacagcagcgattgtgacaacgactaaatagggttgaaccgcctttgccaatgatttttttgccgtttactctttgccgggtgtgacactctgcaaaaggtttactgtgtgtatttctggctttgccgagtgcatggggcactcggcaaagcatgtgactctggtagtgtctatgctatgtagacgcacctccaagcttaggtatgtttcgtctaagcatttatggattaatgtctaatatcttcttattagattgcaaaataattagtagtggaagaacaaccattgcaaatatgttggttgtctttgtatgactaaaccaatatcatcatattatatcaacaacaacatatcccaatatctatatccataactatatctatatgtaaaatttctatatctctatctctatctatatctataactagatcttctggcacaaatgagatgtgctagagatggattcgtcttctatataagtacaagtgagatcgaagatggagattagaacaaacaaatcatataatctctcttcgtcactagctcgtggtaataagggaagcttgatcatctttccttacggcggtg

SEQ ID NO: 4 - DNA, 2125 bp, Zea mays
gagccgtcctccaaagcgcgagagaacagctcttggatcgctcgaggcacgacgctgagcttattaccgtcagttccttcctgcaatgcaacccacctatcatcagaagcacacgcaccgcgggaaaaaaagccatagatttagctcgagttctgatgggcgcaattcaggtcaaggcttcgtctactcaagggacgcgatctcacctcgcccgagcccagccaagggcgggaacagtagtccaggcgaattcacgcctcgcccgagggcctcctcaagcaacgggcgcgccctcaactcgcccgaagcccagctcgggcaggcttcgttgtgaagcaaccttggccaaattgcctcaccaaccgaccgcatcgcaggcgcattcaatgcgaggatcgcctgacaccttatcctgacacacgcgcctcagtcggcaaggtcgaagtgaccgcagtcactttgcccttccactgaccgacctgacaggaaaataacgtcgctcgcccctctccgactgttgtgccactcgccagggtaaggttgacaactgctaagtctagcctcaggcgcaacaggaagctctgcctcgcccgaccttggacctcggcctgaggaggaggtctccgcctcgcccgaccccagggcttggcctcaccctcagcctcggaagatggcctccgcctcgcccgaccccagggctcggccctcaccctcggcctcggaaggtggtctccgcctcgcccgaccccagggctcggcctcaacctcgacctcgggaggaatcgcgtcctcgcccgacctcggcctcaagaggagtctctgcctcgcccgaccttgagctcggaccgaccgcgccacggaggatacatcattaccctacccctagctagctcaggctacgggggaacaagaccgacgtcccatccaggctcgccccggtaacaagtaatgatggctccccgcgtgcgtccatgacgacgacggttctcagccccctacggaagcaaggagacgtcagcaaggtcccagcagccccgacagctgtgcttctacagggctcaaatgctccttcgatggccacgacaccgcgtacacagggctctagcacctcttcgacagccacgttggcatgtacatagggctctggctcctctctaccggacacgttagcatattgctacacccctattgtacacctggaccctctccttgcatctataaaaggaagggccagggccctcatacagaagggtggtcgcgcgggagcacgggctgacgagcagactcgctctctctctccctcgcgagcgcttgtaacccctactgcaagcgcatcccccccttggcgcaggataacacaagccgcgttcttcccccttgtgttccatctcgtgccaacccatctgggctgggacatgcagcgacaatttcactcgtcggtccagggacccccggggtcgaaacgccgacacatgtattgctaaaagtgatataaaaaaatactacggttgtgttatgacattttgaaacgtagtatttcaaaatcatggttttgaaatagtttatatccaaaagataccctaaacatgaacggcacgacgagcacctccactagctagcggcggcggggtgcgcaggctctctgtcaagatgtgcaacgacgacggcacggcgagcatctcccctagctagcgacgattcaagaaaaaaaagacaaaagacagctcatacctcgtaagtactcaccacccggcccatctcatgagcgacgtaccgcgatcgatgacacgcattcggcatggcagtgacaacatcggtgcagcacattcatggctgccatatattgggtcaattgtactggctataggctatagccagccagtttgtaacggcataataataaataaataaataataaatctgtgtgctcgatcgcctctgtaaaaccatctaagaatttctgtgtgccgccgctacaaaccaaccaaccaacctatgtacaagcagtatataagaagggtacggtacggtgcccataccatagcttcgtctgtataccctatatctctatagccaacacacacacaaatactctccattccattccatctcaaaggatagaata

SEQ ID NO: 5 - DNA, 2121 bp, Zea mays
ctagaagaccggcgtacttgttggtcaaccccaatctagaaacctccaatcttggccacgtatgcttctagtgtcgtcatatgtcatgccatatgaccactttaggccatatgcgtcatgtctcatatgcccgaacccctagccgacacacgcccatgaaaccatgagggttggctctgatatcatttgtaacactcaaccactcccgaatcgatggtacttactcctgatggtcatctaggatcatagaccatccccacagaccaacatgagtcttttctacgcactttgttcactcgtgtgcatcaaagaaaacttcttggttggtcactcatccaaaaattgctctgagccaagcatgcttatcttagaggtttttttgagataggcttttgaaaaagaaggtgcaccttgtttgtatgagtattataccattcctattaagccttggacaaagatatcacaatccacctaggccaagatatcacatttcccaacttagtctatagaaggactagacaagacatctccttagaagagaagccctacctcttgtgcccataacaggcacctccaacttgagaactaatttcacaaagagtcacgctcttgggaactccatgtactcatatgtacacactacatctaatgcatagaaacaccaagatcacattgtactagcaaaatgtcatagaagactagttaaaaccttgtttggtccgctcaaccttaaaaatcacctaggagacatgctagaagtatctcaacagagaatgacaccatatagtagtggcaccaagtgcctaatctgcacacaaaaaatcgtacaatacatgacatcaaggcttaataatagagtgtatgttaaagcgagcatgcaacctatgagtggtatgtaggagttaggtttaaacaaggtaatggctcaagcaccacacatcctaccacaatgtcgtaataaatataaaagcactagcaatctatttagcatgcctaaatgggatactatgaggttgggtgggatgtggcacctttgtataatggcccagttccttagtgtagtcttgatcctccccgttaggttcagactcctctagggattttgtaggaatcatcaaattttcataagcaatttcttgtgcacaaagaaccaaatagattgaaaaagttccaaattcactcaaacacaaaaccatggcacatagcttatgtgacaaaatatttggacactagtttcatattttttgagatcatataagtttattatcaaactccaaggattaaattattttttgaaaaaaaaagaaaaagggaaaacatcataaggtgacacatggcaacctctgaatgactagacttttaccatctctcaggtgggtctggtcaacaatcactgttggtcggtccttaccttgcctagacgggtccttagtaggcctactgggttgagttatgggataaattgtggcctagaaacataccagtccaccaaccttgggaccacttaaaaaattgcatcttgcaccattatactatttagatgtttttaaaaaacaataataacttttacatcgaaatcaaaactagacaaattttatactttcacagagcagcagaaatttatacaatatgattgaatacaagatgtaggacccaatggagagaatttttttgtctcctatatgcttgaatacccaacataatatcttcgcagcatactatctatctaatagaaaaattataatatagttaaatacttaagtagtatctagtggatagaattcaatatctcatacatgcatgaggagtaatatctactagacatgcaacatatttttatctatctaatagaatatatataataaagttaaatattatatgcatcacctactatatataatttgatatcttttagatgtataagggactaagaataatatctctagcacacatgcaatgcattatctatctaaatatattatataatagttaaatattaattatacgtagtctaaacctacatataagcctacccatccccacttaaagatctcagtgtcacacatagaccatacatctcacttcgccaaaaaaatttcgtcaacagttgaagttataccc

SEQ ID NO: 6 - DNA, 945 bp, Zea mays
gatccagcttcgcttagtttttagtttttggcagaaaaaatgatcaatgtttcacaaaccaaatatttttataacttttgatgaaagaagatcaccacggtcatatctaggggtggtaacaaattgcgatctaaatgtttcttcataaaaaataaggcttcttaataaattttagttcaaaataaatacgaataaagtctgattctaatctgattcgatccttaaattttataatgcaaaatttagagctcattaccacctctagtcatatgtctagtctgaggtatatccaaaaagccctttctctaaattccacacccaactcagatgtttgcaaataaatactccgactccaaaatgtaggtgaagtgcaactttctccattttatatcaacatttgttattttttgtttaacatttcacactcaaaactaattaataaaatacgtggttgttgaacgtgcgcacatgtctcccttacattatgtttttttatttatgtattattgttgttttcctccgaacaacttgtcaacatatcatcattggtctttaatatttatgaatatggaagcctagttatttacacttggctacacactagttgtagttttgccacttgtctaacatgcaactctagtagttttgccacttgcctggcatgcaactctagtattgacacttgtatagcatataatgccaatacgacacctgccttacatgaaacattatttttgacacttgtataccatgcaacattaccattgacatttgtccatacacattatatcaaatatattgagcgcatgtcacaaactcgatacaaagctggatgaccctccctcaccacatctataaaaacccgagcgctactctaaatcactcacaacacaacacatatcttttagtaacctttcaataggcgtcccccaagaactagtaaac

SEQ ID NO: 7 - DNA, 2046 bp, Zea mays
atacccggaacggaggttcaaacattcaatatgacaggggctaaattttagtttggggtaaccaaacacccccttattcgtccctgtacttagctgtccatgaggatgtttgagacagctttattttttttttctaaagagcttttctgataatctaaatttctaaataatctaattatcatgataatctagatatcgttaaggttttaaatagccggctaagagatgcactatttagcgctaatcaagattagtcgctaatagatttatttagcccgataaatacataacattagcttgatttagctgataaactgcaatccttttactgggtcgatcctttgaaacattaagagacaaaaacaagttatgggtaggtccttaacttaatgtttatgattttgttagataatgattcaatgcctttcatatgtgtcattgaatttacatgcatgttcatttcttattggacatttggactactagtttgaactgtcatatttaaatgttgaacatttaaactatctatgaagttatatcatgctttttcctaaaaactgttaaatggtttagctcatagctatagccggctatagcctttatagcttatccaaaagcttgcccctaaggcccttacctagacatttaaaacattattggtacctgctctgctagctccgataatggcagcgcaaaaggcctccatgaggacacccgccatcgcggcactcctgctgctgtcggcgatgacgatgctgtcctcgacgacagcggtggcgcgctggttgggtggaagcatcgtccaacaagcccaaccctttcgtgaccctcctccgcggcattggggaccatggtcagttatcctctgccacagaacatggggcaagcgtcgtggtctcctctgccaccctggattcccctaccaccaccatgcgccaccgccagagacatactggccataggcgccgagtaattattgattgattcattggaatgttaggacaagatggccgatttagggggaaaatatgtgacgccattgatggactggcgtgctactgaattgttttacacatctaaatctggcccaataagcccatatgattggttgatgaggcagaatatatttttagttcattatgcttatgatgaaataaattaattatctcaaacatatatgattgttgtatatatctcattgctgtcaagaaatgaaataattcaaatctacatggtagttatcttagttgtgtttgcctactaaataaatgaaggtaggcatctttggatgccaaatgactcgtcaagctaaatgagacggacatgacttcctcttttgatactattaatgaaggtggacgtctttgatgccaaatgacttaccaagctaaattttagctactcaaaagtaaaataactctatgctagtggttgacctaagtattttacctatgtaaatttgtttacaaaggcgacatcgttatgccaataatgacccgctgcaatcatacgccaatggaacacataatttccataatctttcatgtcagcatttcacaaaaccacctatgaagatacaaaaactaccactagtaaaacctatttctatactagtggtggtcgaaacttgagaaagtttgatggccaagaatcataaagcgacttgtaattacaattagctaacatagataataaattattatagaaacagacggtagttcaaggcttgaagttggatcacaaagatagaaaatttttaatgaggttaaattgacaaacacgaaagagagcaaagtataggagcacattaaattcattccaatgttgattgtccatctatatttatgcaacataatatttgcacctctcttagagcaaaaattaaattaccacctagtttagatgacaaccatctatccatgctaaatttaagcactagagagtcgacactatatatacacacatatatgtggtataacatcggcaacgttctctaacaagaagcacagatagcaacaacaataacaacaatggtcgcgttcgagc

SEQ ID NO: 8 - DNA, 2114 bp, Zea mays
gtgagcatcttgtttctgcaataatctccagagtctgagccaatacgtccccggaaaagaacctgctaaatgatggtattggtttaaaattaaaggccacatcattgcggcacaaccaaatagaccaaaacattgccgccactccaattaaaagttttttccgatgagctattcccttgttagtagcccaattctcaattatatgattaatattgatgggtctctctatgttgaaagcaaagtaaataatactccaaatgttcctgcactaccggaatttggctctttgccgagtgccatattctttgtcgagtgttttatttcgggcactcggcaaagagctctttgtcgagtgccatgcaaaaaaccctcggtaaaagaaaacactcgacgaagaagctctttgccgagtgttttatttttgacactcggcaaagagtttctttgccgagtgtctttttttgacactcggcaaagggctctttgccgagtgtcacaaatacaacactcggcaaagagctctttgccgagtgttttttctacaacactaggcaaagacaatttaaaaatcacattttaaagtagtaaattaattcaaatgaaaaagttttcaactacaaatttgtataactcatcatgatgtacaatttatatattgaacatttcttcatatgacaaaataaaaaaaattgttcataaaacatatatctctctcgtagtttatgaaactacgagagagacatatagaatttgtgcatattgttagaaccatcatctgagataaacaaatgatcaaacaaccaaaataaactttgtagatcttgagaagttatagaagtttatagttgacaactttttcatttaaagtcatattgtcaatgaaaactacgtctgaatttaaaaaatttaaaatttgaattttgaaaacgacttcgaaagaaaaaaccaccaacatgaaagttgtaggtattgaagagttataaaactttgtagttgacaatgttttagtttgaaatcatcttgttatgcaaaactatgtttgaattttgaaatttgaatttttcaaactgtctcggatggaaaaaccaccaaaataaaagttgtaggtcttgaaatgtaataaaactttgtagttgacaattttttgatttgaaatcatcttatcattgaaaatttcgtgtgaagttttcagatttaaaattcaaaatttgtaaacggtctcggatgtagaaactattaaaataaaacttgtagatctcaaaaagttatgcaactttatagttggtcatattttcaaatgaactcatttagtgccttaaataatcaaattactcttgatttgttatagtacatggggaatgaaaacataatataaacataatttgtgtagtagtgtagtggtatatgagggtatgcgcgagggagaggttgcgagttcgaatctcaccatttacaaaacttttataagtttgattcaaaatgatagggcaacgggtaaggtaatagggtgggttggagagttgttcctagaatttaaaaaatgttttgctgtttttttttaatttttttgattcttaatttgccgagtgtttttctttgccgagtgctttttgacactcggcaaagtctttgccgagtgtccgaaaaaagcactcggcaaagcctttgccgagtgtaaaatagcctttgccgagtgtcttagacactcggcaaagaacgcgattccggtagtgctgataaggtaacaatcaaaaaaatatgttgaatagtttcattctgattacaaaagcagcatttcatactccctgataaataaatagatagatagatagatagatagacacaaagagatagatatagccatctagaacactaaaagtgtcttgaaggagaactaagtaaattttaaacaattgatgtagctatattttaagacggagggactcgattagatatattatagtgagatctagatcgagagacatgaataaagatagagatagagacataaaaatatagagatagcccatctatagaacatcgaaagtatcatgctggtggaagtataaatattgaacatctatatgttcttttcctcacccaaataattaacc

SEQ ID NO: 9 - DNA, 1368 bp, Zea mays
ttatattagaatgcaaatataatatatatcagtattttttatatggatcttggtggactggtcatccggtgggctggcttaggatatgccgtggctcacccggaccacccactgtttacgccactgaccacggctgctcccacttaccaccatttcttgtgcctataaatttggcatgtagaaggctctccgatccatggcgacaaatcaactctgattttcttcgacaagagatagcgctgtttttttttttttgtgtctggccactttatccgacggcagccaatctctgacactagttttaaatgcggttttttatatcaaaaccatatatatgtccaaacttcatttatttcgtagtgaccattgtgacgaggataccgcagcattaatgttagaggatacatagatttaagtcacaaatctacatccaagctatggctcctatttatggatattggtaaaagatatagtaccatttaaaaaaagtatctcagacattaatattcacactaaagatgtatttccatcggaggccaattgaaaattaataaagaagtgaattttaaaacgagatagcaaacgatttcaaatggaacgatcatgtgatctaaaaattgtaggccccttatttataaagttggtgttccacggaaattatttgcaaacgttataaatttatttttgttgatcctattttcggaggcgggtcaaattatccagtgacgactaatttttttgttgaagtataaaccgtatctataaattcatcagtagtatgtcgtcataccaatgcacaagtgccctaaaactagataccatttaaaagatggctacccaagcaccaaacgcccatagaaatcccaatctcatcaaggcagccaatctctgacactagtttttagatgcgtttttttatcaaaaccatatatatgtccaaacttaatttattttgtggggaccattgtgacgaggataccacaacattaagagctagtttggaaactcaaatccccttcgggattgaagaggattggagaggaaattagttcatttccacctcaatcctctccaatctcgaagaggatttgaggttcccaaagtagctctaaggctaaaggatacatggatttaaatcacaaatctacatccaagctatggctcctatctatggatattggtgcaagatataataccatttaaaaaaggtatctctcagacattaatattcacactaaagatgtgtctcagactctatataactacgagcaaaccaagatcatcagcaagcatactattagacataccggtatatattattgttcttagtcatcgcacaagactcttgtcaacaagaccaacaacagg

SEQ ID NO: 10 - DNA, 1859 bp, Zea mays
attcctcattgccaccgctagtcacatcacaatgcctattgtgtgggcaccaagatattatataataagctctataactgtgggcataatgtttctagctggcattcagtttcttaacattccaaattatgcgaatattcttattatgctagtggttttaaaaataaaaacggatacacttctattgttttatccctatatcaatagctggcattttagtatttagtggcaaaaaaattgcaaaacttcttatttcctagcggttttctagaaaaaggtgattgtacaattcaatacttttcctattatttagctggcagtcagttcttagtgttgcaagttatcacaagaaactgctacaatcattacaaacttcacaatgcatcaatattaggccttgtttggactccagaaaatttacgggagttggtggagcaggtcattaagtactataaaaaatcatgtagctgaagctgcaagtatttagaagacatttagataagttattttatttattatttagattaaaaaaatttaaaactatttaaattgatattataaactacagctccacactggagctagatcctggagtcattacaaacacccccttaatgggaaaagagaagataatgtatatctaattattgtttctgtgtcacctatagctattagttcaaaacttcataatcactggtacaaataagctctagagaggcggttcggaacccatttttattgttgtttttcaaaaccactagtgttagggaccgccagtggaaactgaaacgccattggaaattgattttcactgatggtgagctaagaaaaccgccattggtaatcctttgcagaaaacataaactaggttttaaaaatagtaaacaaatatttttattaggagaggccccacctagtcgcaccatttttcacgcattattcacgcgctacgcaaccaatggtaattgaacctcagagacttcactcttgtgtagcctcctttgccactccactaaacacttacttgtgtcttgattgcattttgttgcccacatattagaacaaacagagtgtaaattgattgtttgaggctgtaaacaaattcaaatgaaaaagtagtcaactactaaattgaataattgtttatgttctaccacttttattttggtacttttcccatcggaggcggtttgtaaaatttgcattttaagttttacaaatttcaatgaaattttgagagcccaaatgatttcaaataaaaaagttgtcaactacaatgttttataacttttaatttggtggttttttaaacaagctcatttgaaaaactaaaaagatcgattctacatgatttttaggtcgattttttaaggaatcgcctgtacaaatatttctactgacagtttttaagaaaccacctgtggaaatcatagatttgtactagcggtttttctcaagtaactgctagtagaaatatggtggttttcttaagaaaactgtttgtaggaatgcacgatttatataaatggatttgttaagaaaaccgctagtggaatgttctttcaactaacggttattgagtcgtgacagccaatttaatttccttgataactaaaagcggctgtaaaaattagaccatgatgtaggcacggagctgtttttgtactgaatgcgcccactgttttgttggaaaagtgcatgtacttattattcattctgtttatttctagctggcattcagttcttacagccacagattatgcaaaacgcctatttctgccagcaaatttacaggaaaagtcatggacttttccgggttattttcctataagtacagccattcctttcacttacaggcc

SEQ ID NO: 11 - DNA, 2000 bp, Zea mays
gtccccaacaacacccgccatcgcggcactcctgctgatgtcggcgatgacgatgctgtcctcgacgacagcggcggcacactggttgggtggaagcatcgtccaacaaccccaaccctttcgtgaccctcctccgcggcattggggtccatggtcagttatcctctaccacagaacatggggcaagtgtcgtggtctcctctgccgccctagattcccctaccaccaccatgcgccatcgccggagacatactggccatcggcgccaagtaattattgattggttcattggaatgttaggacaagatggctgatgttgggggccttcggcttccgaaggtcctcaaaaacatgatttaacaatgtttctggagtataatgtgtgaacaggtatcttcggacttgaatcagaaagggagaagctcaaaagatacgaaggttgacacagtgccgaagctgtgaagcaggaaagcttcggcatgttcgcagaaaagggaaaccgacttaaagatgaaaaggccatttagacctcgatagaatgctatagaattatcaccaaatgtaaagggcatgtatgtaatttttgtatgggttgtaccccgtgcctataaataggtgaacagtacccccgtactgctcacgctgacttggcatttgcttttgcgtcacacttgtattttcatctccttccaagccgaaggtacatttataattcaatattgtttctatttctttatgatgagttaataaggttaaataaataatgttatatgattattcacgctatcttttatgtttcatatgcttcgtcttttattaatgtatattgtagtgatgaaggttgatccttcatgaccttcgtccggagatcgttatatcctaagggaaataatgcttcgaaggacgaaggatcttaatgtttaacattttctgtgttgccttgttcttaactcatagcatttgagaacaagtccccaacagctgatttagggaaaaatatgcgacgtcattgatggaccggcgtgctactgaattgttttacacatctaaatctggcccaacaagcccatatgattggttgatgaggcagaatatatttttagttcattatgcttatgatgaaataaattaatcatctcaaacagatttgattgttgtatatatctcattgctgtcaagaaatgaaataattcaaatctacaaggtagttatcttagttgtgtttgcctactaattaaatgaaggtaggcatctttggatgccaaatgactcgtcaagctaaatgagacgaacatgacttcctctgttgatactattaatgaaggtggacgtctttgatgccaaatgacttaccaagctaaattttagctactcaaaattaaaataactctatgctagtggttgacctaagtattttacctatgtaaatttgtttacaaaggcgacatcgttatgtcaataatgacccgccgcaatcatacaccaatggagcacatcatttccataatctttcatgtgagcatttcacaaaaccacctatgaagatacaaaaactaccactagtaaaacctatttctatactagtggtggtcgaaacttgagaaagtttgacggccaagaatcataaagcgacttgtaattacaattagctaacatagataataaattattatagaaacagacggtagttcaaggcttgaagttggatcacaaagatagaaattttttaatgaggttaaattgacaaacacgaaagagagcaaagtataggagcacattaaattcattccaatgttgattgtccatctatatttatgcaacataatatttgcacctctcttagagcaaaaattaaattaccacctagtttagatgacaagcatctatccatgctaaatttaagcactagagagtcgacactatatatacacacatatatgtggtatgacgccggcaacgttctctaacaagaagcacagatagcaacaacaataacaaca

SEQ ID NO: 12 - DNA, 1556 bp, Zea mays
gggttaactagagtgtcatgtccttaatctagggtcttaaaatacccaatgttagaccttcatcacccaccttggctccaaggtgggcatcaatatctatgatgccgaagattattgtgacaaagtcctgcctatttaatgtcacgttagcttgccatgccacctggggctcaagggtcggtgccaaattctatgacattgagatgtgcaaccttggtgtcaaaagtcatagtgatgaaatatatgctctatattcgaaatgaagtcgtacaatgatgtatatgtcagtattcttcaaaatatgttatattatggggggttgggtgcgtcaacaccttctaggtcatgacccactcttcttgttctctagagaagcagtcaaacacggtactacgttgaagttcacatcattgtccattgaactagattcacatgcatgtcgaggagctggacgagcctgatgtcgccgtcaatcaagagcaaagtctatcgctaccaccactgcttgctagtgatttctttgcctgcctaccgattttttttgtctagagcacattcgctaggttcatgagaaggccaagccattaccccatggaaaggcattgccaatcctatcctatattgtgcacctctctttctagtactaacctctccttcaagggcatacctaccgggtatgccaggtatgccctggcataccctacttgcctagtgtgtgtaaagaataaaaaaataatatgataaggatgctccactaacagtcttatatcttgtacttcttttatgttcgggtctacgatcttaggacctatttgatgaaatcttaggataaagacagatggcacggtcacaaaaatcaatatttcaacgcttgatggctcaaattctttgatagacatgattcgcgagatgccgctggagactaatctgcggataaattaatctatgcaagtataatttgtttaattgtttgttattgtgtttgatattttgttttttaactagatgaagatgatataataaagatattgattaggtcccttataaggtcttagctacaaagtgagatgaatctctcttgcatgcctacattttataaactaacaatatggctttttcacctgtttatactataatttgtgaatattttaaagttaaattttaaatttaaaagttattatttatatttttaaattagtcttcagttttagtatttaatgtgtagtttagtgtatatgtactaaataatgttcaaaattttaaaacgttgctaaattgttaaacggtttgatcgaatatatatatgaaaattttagacggcataaaattatagatccgccgctgctcaccttcttccctgtttatcatctacgtacatctcaaataataaatgaagagatggagggagtggattagatgtagatatagtaagatagatctagggatatagatatagatatagccatctagctagctagaacactgaatgtaccagaaccctctaaggcctaagaaaccactataaatattaagcatgtgtatgctgttctcctcac

SEQ ID NO: 13 - DNA, 2072 bp, Zea mays
aattgtgttggactatgtccatacttctaagaatttggtatgccagtttatagaagatttgtcacataatgtgatagatggtgcatcgagaaagatggacttgagacccacttaatgccattccatagtagtagcattttctatgtgattggagatctcatgaagtaaaatggtgaaataagctagagaatgactgatggagagactccttaaataaaatctaggtaatttcatatgaacatttctccttcgatgtacgataggctaactaaaatcttaatatgatttgagtgacatatctaagcagggatgttatcttatagaacatcctataaggaacacacctatatgactttgactactaatcacagtctatgatatgtgggaaatatttttttactcatgaaaggcctcgaagtttgacttatatgcttcaaaccaatggataactgattgaatctgtttcagacagcagcatagttaacttcaaaattctatatctcccaaacttctcaaccaaaaattattaaattatgctgacagtaagaacttttatccctctacaaaagtctccatagttggaagagccaattcggccatttactagatgaaacctaccagttaacagacccgctcatttttgtcaggcaaacaagaacccttatatatcaaagagtagtggatttgaatttgtctaatatgaagtttatgggcttggatcatttaaagtattttataattctgaaagacaagattaaactaactatgtatgatcctttagcaccgtacagatggtcgatgggacagttacatggtttatatagatggacctatctatattccttcagtggttgagaacaaggtaaaggtgtgtcacactcacgcgtgcgccgcctccgccgagacgaggcatggcgttgcagggtgaggcatggcatggtgcagcacgatcgcagtcatggtcgtggcacagtgtagcactgttgtggatagatagataaactattgttgtagttagagtgcatgagtttttgaatgcaacactagaattttcacaatttaacttaattaagaagttatatcttgatgacacagagttacctgaccattgatctcacatgcccttttagctctctcgtactctagacttaccctgactatttaggtctcctgactcgccaatataaacaccatcctatcatttgctgtcgaatacttctgtgtcacaactcaactgtcagatgagtactccattacagcactgagcaggcctccgaaatgaatatctactcagatgaaggacgacaatcgtgttctagggaaatatcagcgacacaactccttacttcctccgcttcttcctagtgttttttgttgtgattgagtcgacatagcaacaacactgcactattacaaccagtacgactatatcaactagcaatgtcttccttatatgttactatttattttgctcatattcattatgtttaaatcacataggcgcctttatgttggcatcaaaaaatcagtatcaactttatagattaaaatgaaactaaaagtacctaaatttctatcggtggggatcgagtgattctttaaaccgattattacacaagttaaccacactaaaattaacaatggtgaatcgtgccatgatttttttctagtggaaaatagccaaaccaagcaacacatatgtggctatcgttacacatgtgtaaaggtattgcatcacaccattgtcacccatgtatgtggacaataccgagaggaaaaaccacttatttattgtattttatcaagtttgtcttgcttacgtataaattataacccaacaaagtaatcactaaatgtcaaaaccaactagataccatgtcatctctaccttatcttactaatattctttttgcaaaatccaaaattaatcttgcacaagcacaaggactgagatgtgtataaatatctcttaaattagtagctaatatatcgcacatattattgagaccaactagcaacatagaaagcacaatagtgtaccaacaatggcagccaaaatattttgcctcctt

SEQ ID NO: 14 - DNA, 977 bp, Zea mays
aggaaagaatcaagcttgaattcgttataggcacaggacgcagcctgtgatgaattacaagtatgccccttataaaagcttacaacaatgactcagacctttagggactaaaaggtcattctacctttaggtcggtttgtaataccgaagcttctcgaagaggaaccttcggccatcttatacaaacagcttcagccgaagaccgcttcttcctgtaagaccttcggcgacgaagcataggcccaacacctatggtgggttaatttacgttggctacgtttcaattctgtgtactttgatttatattatgtaaatgactctagtctttaatattattacttactctttattgttattcgaagcattgtgctatgatgagtcatttatgtaatcgctatgtacgtgagttctgatcctggcacgtacatggttcgcattcggtttaccttctaaaacctggggtgacaggtggcatagcaggagtggagggcagcgacggctgcacagctctgcgtgcaatggcttgcattttttgctcctcgttggcgatgcgtgtgcgaccatgagctctcgacacaggcaggtagtagtagagcaagaattgtaaccttgggttttcccacacctcaaatagatatagatatagggatatagatagatatagcaaattcaccaaataatataggggtatagatatagatataataaggggtatagatatagatatagacatatagaagatatagatagataaatagatatgatagaatagataacttacaattttgtctaaaagaaactaaatcactgctaagtttggagtagcatatctttggtgaatacttgctagtgaattggtttccgctatagtatatatatatatatataagtataaactcttctaggattatagtatatatataagtatacactcttctaggatcaatcgtgaggagttccttaaattgtcttgcgac

SEQ ID NO: 15 - DNA, 850 bp, Zea mays
gatccgcaaagaatttgagtccggtagtgctagcaccattgtgactctttgaaaaagccatggaaagtatttgacaagatttatggttagagatatttgctcgttaataattacgtgggaatcgacatatggccatgaactcaaggtatgtacaatcctgagactccgatttagttttcaaaaaatacaataaacagttaagagactgcatatacaacactgagattctggatcataaatataactaagagacaacatgtatggagagtcgtgaagaaacgtactattcgcgaagagcctgtcgcttgtatttttttcacttagcctcttagaaactcctagagaaaccctatattacatagggttgtatatgtcggaatacattccaatgttacaattaatgttcgtatttaccattaagcaagtagatacttaagcattaaatcattgtgcaattactttgaatactaatgtctatgatatctgtatctggatttagttgtttagataccatcgaaattaatagggttgttcataatgtgtttgaattaaaaaaacaatagctatttcgtgccataattaaattgtttgatacggatgatatcaacaaagatctagatagaaggttgggatatttttcacaactgcaaaatcattcacagcagaatatcttcgcattattataagacgaaaacatattttagtagaagattggacgatatcgaagtttcatctcgagatatcaagcaattaatgtaggacacatacaatagatttgcctaaccatcaccttctatcagtccatccatctatatatatctgcacacaccaccacagcaggttggtaaag

SEQ ID NO: 16 - DNA, 2023 bp, Zea mays
gctgatgggggttaccagctgagattaggagtcttgggggtacccctaattatggtccccgacacccctagtcatctcacgatacatgtaagcttccttccttctctggcgaaaaatcattgtcagctcgaatatactcatccatcttctggagaagcttctccagggtttatggtggcttccttgcaaaatattgtgttgtcggtcctagtcaaagccccttaatcatggcttcgatgatgatctcattaggcaccatgggcgcctgggctctgagtcgcaagaatcttcagacgtacgcctgcaagtactcttcatggtcttgtgtgcattggaatagagcttgagcagtcactggctttgtttggaagccctggaagctagtgactagcatatcattcagcttctgccacgatgtgattgttctcggccgaagagaagaataccaagtttgagccacacttctgactgccatgacgaaggactttgccatgacagcagtgttgccaccatatgatgatatggttgcttcgtagctcatgaggaattgtttcgggtcggatgcccgtcatacacggtgagttgtggcggtttgtatgaaggtggccatggggtagcctacaactctgctgctaggggagaagcatcatcaaaagcaaagttatcatggtaaaggtcatcataccatccatcatcgttggttggactttcttgatgaagctctctatgttgaggccttcgtccttgatcatcctgcgtgatgtggcgcatttcttcaatggatttgtcgatcttcctttgaagttcggctagccgaagcatcttctcccttttcctctgtacttgctgatgtatagcttctaggtctctgatttcctggtccaactcctcttcctatggtgttgagctggtagccttccttttctggctcctagcttctctcagtgagagtgtctcttgattggtgtccagtggctgcagggcagcccctggcgctgttgccctcttcggtggcatgacgaaggcgaacgcttgccaaaggttgtggaagtgagttcaccggaggtgggcgctaatgttggtcacttgttcttaaatgttgtgaattaagaacaaggcaacacaattgttaaaggtataaaaaccttcatcctccgaagcattgcttcctctaggattcaatgatcattggacgaaggtcttgaaggacatgccttcaacatgttatcaataaataaggatacacaaggatagatacaatgattataaatcataagtctattcatatcagcattccataaagtttatatgaacatgaatatttattacattgataccttcgacttgctcgaaggtgttgacacgaaagcaagattacaattcagcgtgaacagtacggtgctactgttcatctatttataggcacggggcgcagcctgggtaaaattacatttatgtccctgacatttacttataaccataacatggatcattaaggactaagcagtctttttccctttcggcttgcatcatctttagtcttcatcactattataagccgaagctattaccccttggctatagcttcggtgttcatctttattatcttcggactatgtcttcaccttgtatacctttgtcttgggggaaaaccttcatcctgaagccgaagctccctgtaataattcatatcatgctaaaaataaatggtcagtcctgtttttgaggaccttcggaagaggaaggccccccaacaagacgattaactagtattgtctcactgcattgtttttttggcacttcatcaataatgcctcaatagcatacttcattttaggaactttattaaaactgtcttaaaatagggccaagtcataaattcacttcaaagtgactcttcatttcttacttcctatctttggtggttttgtatatatatatgttcatggttgagtgatgttcctacaccactacaccacaccttagatatatatacagaaaatagcttcactatctagattcc

SEQ ID NO: 17 - DNA, 2018 bp, Zea mays
ttcataatatagtttaatatttaaatctcatagatcttccaaaaatcacaactccttaaccgtaactctgaatttagcgattctcgaacccacgatctcgtagcgatacatagattattattatgtagtttattcttatgtttggtgtaatgttaattttacttataccatgtttgtttgtattgctacgtttagcagtgaggtcatgagtcatctgaagatcatcctggtaccttgaatctcaagtcccaggcaagttgtgcccttgatcacttttgtttacccaataatgttctttataataatttattcatgcataggtttaattttgatgggacccaataggtcaccctagtctgattatctttataccttgtttacctctgaactatttgggtagttttgctattgctctatatgcttttgggattaatatcacattatgtccatgttccaattattatgttattttatttattgttcatgtcaagatcattattttaattggaacacggagcttaacttgagaaacatgtgccaccgcaagggtggaatgggacgcccttggctgactaattaggaaagctagtggaagactaccttacctgaaaggggcaagggcagtaggggagtaggagtgtagggaggttctcgggttgattttgctgcgatggtggtcagacgagggattcctacattgctcttcctagaaactgtagcaggttttcggaagctagtggaactttgtaaaggcctcgtagtggatccctagccattcacctcagaagtgtctaagggccttacaaaccctggcaacatgggatacatgacttgtgggtatatggtacaacctctgcagagtgtaaaactggtatactagccgtgctcgtggtcatgagcaactctggactctcgcatgattaaattatggaactaaattcaatttgtcatttgcattgcatgagatttattattaatgttgttctaatattattatggtttggtattgacttatatttagtaactgctaataaaatttgaccaacttattaaaagcaatgctcagctttaacccttattattgatcagccttacacttcacgtgaactcccacctttggtgagttcatgcacattattccccacaacttgttgagcgatgaatatatgtgagctcacctttgctgtcttacaccccccataggtcaagaacaggtaaaacaggatgaggcgcatggaggatgctgcaatgtgttcgtgagaggtctaggccatcgtctcctagtcaactttgggttgctggatcgttgtctccttacgatgtaattattaatttattttgtacagaactcctattgtataataaagttgtggcattcgtttctgtgcatgattcatcatatgtgtgagacttggtcccagcacacctggtgattatgttcacgcctaggtccctaaaacccgagtgtgacaggagtcggtggcatgatttgtgcatggcggtgttggaggagaggagggcagtggcggctgcacagtgctgcgtgtagtggcttgcagttgcagctcctcgttggggacaggtgtgtggctaagagctctcggcacagacggttaatagtagagccagaattttaaccttgggtattcccacacctcacatagatatagatatagatatagagatacatatagcaaattcaccaaataatataggggtgtagatatagatatagatgtagatatagatatatagaagatataggtagatatgatatgatagaatatataacttacaattttgtttaaaaggaactaaatcactgctaatactggagtagcatatctttggtgaatacttgccagtgaattggtttccgctatagtatatatatatatatatatatatatatatatatatatatatataagtatacactcttctaggattatagtatatagataagtatacactcttctaggatcaatcgtgaggagttctttagattttcttgcgtc

SEQ ID NO: 18 - DNA, 1628 bp, Zea mays
agggctgcgaattcccatttgttgccgatgtattagacatagatatcctgttactagtttcatatgatggaaaactaggagcaacagacttctccaacgtacgtgttaattttctaattgattcttctaaccctccaatttgttgtttcatttgattctgctgatgatctacatattgtttaatagattggatgtcgtcgggtgtacttacgttagggacttgaagcggagatagaagagatgtgatgtcggtctctccatgtttgacaactttctggtggcgatccaccgtgtattgtgataagaatttctccttcgcttgacacatgtagtcctcgtattgctgttgctcatcggccgttagtctttcaacagctggcttcaggatattgtctagggagatatcagtgtgatctttagaaccggccatttgagggcctgatttttagtagatcaagacacctgtccccagcggagtcgccaaaaagtgtgttggcgccggttcgggcaccaatcactgcgttgagaacctgcggcggtgctctctacacaggcgcggacggtccgcggccaggggccagacggtccgcgacctggtgcagggctcgggttccctgtctgacgagacggacggtccgcgccaagaggccggacggtccgcgcgtgcacaggggcggcagagttcgtcggcggcgtctgaatctcgctccggggagggaccccgtcaggagagatcctaggcttcgtctagggtcggcaggccaccctagacacctctaatcaacgtagagccgaagagaagcgaagaatttgggggtagagaaaggctaatctagagctaaactagaactattcctaatgcataagtaaaacgagaaatagacttgatttgatcgattgttgggggttcaatcggccgtagcccttcatctatataaaggggaggtctggatccgctataagttgtttcccgagctaatcccataattttaggtaacaaatcccgcgagaaactcataaccttaactgactctagccgacgtaaattatcgaatttccttgtagtatctgtccctaagaatgaaaatacctttataagatggcaaggaccaacattgttctagaaaagaaaaatcaggaaaagagagggcattgtattgcccacgggaacatgcaaccgaggtaatgatattcatgaggtgtttggttccattgaactaaagtttagtatgtgccacttcgaatgttccaatgataattgtgagtgttaaatatagtttaaatataaaaactaattatacagataagactaaaggcgagacaattttattaaacctaattagtttgtatttaattctcatagttgatataactagataggtaggcacatatatagagagatagatataaatagatataaataagtagatagatagatagatagatagatagatagatagatagagatagatagatagatggactggtagatagattaagagacatggagatagatagacacggatagatgtttagtatataaagcaggcgtggtgatatcaaacacatcaacacatttttggtattagactattgtagctcatatcatctgtcacccatggcgaagt

SEQ ID NO: 19 - DNA, 3300 bp, Zea mays
tagcttattggagattattttggcaaaagacccactacccatggttatgtaaatagaaattataatatatatcatcttttttctcaccttaaataaacaaataagggtattgttgtctttatgaataatctacatttgtataatctaaactaccaaacaactacatctagattataatctggattatataatttaaattataatctagattatataatttataagctgaaacaacccggccctaaagcactatcgtatcacctatctgaaataagtcacgggtttcgaacgtccacttgcgtcgcacggaattgcatgtttcttgttggaagcatattcacgcaatctccacacataaaggtttatgtataaacttacatttagctcagtttaattacagtcttatttggatgcatatgtatggttctcaatccatataagttagagtaaaaaataagtttaaattttatcttaattcactccaacatatatggattgagtacaatactcatgtgcatccaaacaaactacttatattgaggtgaatttggatagaaattaaactaacttacacactaagccaatctttactatattaaagcaccagtttcaacgatcgtcccgcgtcaatattattaaaaaactcctacatttctttataatcaacccgcactcttataatctcttctctactactataataagagagtttatgtacaaaataaggtgaaattatgtataagtgttctggatattggttgttggctccatattcacacaacctaatcaatagaaaacatatgttttattaaaacaaaatttatcatatatcatatatatatatatatatatataaaccgtagcaatgcacgggcatataactagtgcaacttaatacatgtgtgtattaagatgaataagagggtatccaaataaaaaacttgttcgcttacgtctggatcgaaaggggttggaaacgattaaatctcttcctagtcaaaattgaatagaaggagatttaatctctcccaatccctttcgatcatccaggtgcaaccgtataagtcctaaagtggtgaggaacacgaaacaaccatgcattggcatgtaaagctccaagaatttgttgtatccttaacaactcacagaacatcaaccaaaattgcacgtcaagggtattgggtaagaaacaatcaaacaaatcctctctgtgtgcaaagaaacacggtgagtcatgccgagatcatactcatctgatatacatgcttacagctcacaagacattacaaacaactcatattgcattacaaagatcgtttcatgaaaaataaaataggccggacaggacaaaaatccttgacgtgtaaagtaaatttacaacaaaaaaaagccatatgtcaagctaaatctaattcgttttacgtagatcaacaacctgtagaaggcaacaaaactgagccacgcagaagtacagaatgattccagatgaaccatcgacgtgctacgtaaagagagtgacgagtcatatacatttggcaagaaaccatgaagctgcctacagccgtctcggtggcataagaacacaagaaattgtgttaattaatcaaagctataaataacgctcgcatgcctgtgcacttctccatcaccaccactgggtcttcagaccattagctttatctactccagagcgcagaagaacccgatcgacaccatgagggtgttgctcgttgccctcgctctcctggctctcgctgcgagcgccacctccacgcatacaagcggcggctgcggctgccagccaccgccgccggttcatctaccgccgccggtgcatctgccacctccggttcacctgccacctccggtgcatctcccaccgccggtccacctgccgccgccggtccacctgccaccgccggtccatgtgccgccgccggttcatctgccgccgccaccatgccactaccctactcaaccgccccggcctcagcctcatccccagccacacccatgcccgtgccaacagccgcatccaagcccgtgccagctgcagggaacctgcggcgttggcagcaccccgatcctgggccagtgcgtcgagttcctgaggcatcagtgcagcccgacggcgacgccctactgctcgcctcagtgccagtcgttgcggcagcagtgttgccagcagctcaggcaggtggagccgcagcaccggtaccaggcgatcttcggcttggtcctccagtccatcctgcagcagcagccgcaaagcggccaggtcgcggggctgttggcggcgcagatagcgcagcaactgacggcgatgtgcggcctgcagcagccgactccatgcccctacgctgctgccggcggtgtcccccactgaagaaactatgtgctgtagtatagccgctggctagctagctagttgagtcatttagcggcgatgattgagtaataatgtgtcacgcatcaccatgggtggcagtgtcagtgtgagcaatgacctgaatgaacaattgaaatgaaaagaaaaaaagtactccatctgttccaaattaaaattggttttaaccttttaataggtttatacaataattgatatatgttttctgtatatgtctaatttgttatcatccatttagatatagacaaaaaaaatctaagaactaaaacaaatgctaatttgaaatgaagggagtatatattgggataatgtcgatgagatccctcgtaatatcaccgacatcacacgtgtccagttaatgtatcagtgatacgtgtattcacatttgttgcgcgtaggcgtacccaacaattttgatcgactatcagaaagtcaacggaagcgagtcgactcaaaaaaaatgggttgtggatgaggcgttaggcccaacttatggcagtccgatatccacaattcctggtctatatatagcttcgctgctatgtccttcgcctcccttagttaaagactcgaagctaagcttcatcgttgtatccgtcagtttaatgtagcatattaagcactttgtgttgggtcaacatcgtcggagctcgagtacatagtgagtgtgatttcatagagtaaaagaagagtgggatgagtaaaataaaagtggtgtaagcaaaatagagagtgataggtgcgaatgaatccaaacatcttctcaacttaaaggccagccatattctaaaaaattaaaaaaataaaaaatataatttgtagcctttaaacggttaaaaactagctaacatgatatatgtatatagattagaatatgtcatgtcgctaaagtc

SEQ ID NO: 20 - DNA, 2041 bp, Zea mays
cattaatattctaagaactactgtaaattactaattttggattttaataacatactcatggttatttcgtcataaatagatattctactaacttttagtgaataagcgatatgcgtatgaatacatatatcattactagcaaacatctatattttaacttgaccaagaagatgtggcctagtacctctttcaatttcttatatcagtaaaactatacttaaacaactactaacataatattgttttatgtgctaaaatgagaagttaatcgcaaacatttctagtactaaatcacaaaattaatgaatcgtgacataagttatttactttcgaaatataaaaaatacccatatccaaaatattgttgaggggctggttcacaattaagcctatacatcagggagtagcaagaaataaacagggatctatttttaatggtttctgaactagaaaatatctgaacaaaatttacacaattccaggggctaaaacataaaattgtctgtaaagctagggagagattaccttacctggaaggggcaagtggggaggcgtcgctgctagattatagggaggtcctcaggtcgaactatctgtaaagcttttcggacgagggattcctatgcttccttcttcttgaatccgtagcgggttttctcgaactagttgaactgtggaaaggcctcgtagtgctaccctgcctcgcttcctcggtagaggtgtatggggtccaatcaaccccgtggcaaatgggtaacatgacttatgggtaaagatgcacaacctctgcagagtgtaaaactgatatatcagccgtgctcacggtcatgagcggctcggacactcacatgattaacttatggaattaaactcaatttgtcatttgcattgcattgtgggtgttattataaatcttgatctcttacttaatggggttgatatctacttatacttagtaactgctaataaaattttgaccaactctaaaagcaatgctcagctttaaccatctcttttggtaagccttacacttcacatgatttcccacctttggtgagttcatgcacattattccccacaacttgttgagcgatgaacatatgtgagctcacccttgctgtactcacatccccctaggtcaagaatagataccacatgacgaggcgcatgaaggatgctgtgatgagttcatgggtggtctaggccgtcatctcccagtcaactttgggttgctggatcgttgtcttcgtatgatgtaattattaaactattttgtacagaactctgttatatattaataatgtgacattcgtttctgtaccatgagtcatcatatgtgtgagacttgatcccagcacacttggtgattattttgcgccccggttttggaccccctaaaatctgggtgtgacagatttaaagcttgttactcccaatcttaggaaaaataattatcgaaaacccttggagaccaatatcacattagaatatggaaatgtaacttcttatttctgggaggccacatacattagccaaaaaacttcattagtgaattacaactaataagcaatctaaaagagggcaccaccaatgatgtgttgttggtaagaggctgctgggtttgtcaaagcaagttggacaaagggcaacaactgttgtagttgtaagagggttgcggggttagccgcaaactgctagtagaacggcagcaactgttgttgtgtcaagaaggaagcacgacttggtgcagctagctggctaaatggtagtagtatttgatgctgcaaataagcagtggggttcagtgctgccaattggttaaatggaagaagttgttgttgctgagagtagacaatagctagctggctgaatggtaatagttgttgctgctgcaaataagtaggagggttcagtgtagacaattggttgaatggaagaaactattgttgctgagagtagggagaaaggtttgctagagctacttggctaatcacaggtagcacgagttgttgcagctgtcgtgccccgatggtttgcacca

SEQ ID NO: 21 - DNA, 2014 bp, Zea mays
aagagcaaagggacaaccgaaggcacctctggtctggcgcaccggactgtccggtgtgccaccggacagtgtccggtgcaccagaggactccaactcaaactcgtcaccttcgggaaagtccagaggcgctccgttataattcaccagactgtccggtgtacaccggacagtgtccggtgctccaaggaagagcggctctggaactcgccagcttcgggaattcgcaacggctagtccgctataattcaccggacatgtctggtgtacaccggactgtccggtgtgcaatcgaagcaacggatacttcggcgccaacggctacctgcagcgcattgaatgcgctccagcgcgcgcagaagtcaggcacgcccatactggcgcaccggacactctacagtacatgtccggtgcgccaccggacatgcaggcgggcccagaagacagagctccaacggtcggaacccaacggctttggtgacgtggctggcgcaccggacatatccggtgcaccatacgacagtccgccccaccaaacggctagtttggtggttggggctataaatacccccaaccacccaccattcattgcatccaagttttcacacttccaaccacttacaagagctaggcattcaattctatacacactcaagagatcaaatcctctcccaaattccacacaaagctttagtgactagtgagagagatttgcttgtgttctttcgagctcttgcgcttggattgctttcttctttcttgattctttcttgcgatcaaattcacttgtaattaaggcaagagacaccaatcttgtggtggtccttgtaggaactttgtgttccaagtgattgagaagagaaagctcactcggtccgaggggccgtttgagagagggaaagggttgaaagagacccggtctttgtgaccacctcaacggggagtaggtttgcgagaaccgaacctcggtaaaacaaatccgcgtgtcacactctttattcacttgcgatttgttttgcaccctctctcgcggactcgattatattcctaacgctaactcgacttgtagttgtgattatttttgagaatttcagtttcgccctattcacccccccctctaggcgactttcagctagtcataggcaaaaactttgggttggagtggagttttaaatacttgaaaaataccaaggtatctacgaaaccatagtatttataacaaactttttaccaggtagaaaaacatcttttggcaaaaatactatggtattgtccagtaccatggttttaccacaaaacctcaaaaatactttgcatccaaacaccccctaaatgttatgcctaaaatgtgcttacagttgtttctctatgaaataaggaaaaaatcggttccaaattaagcacaatttgtttgcccatagaaaatgaaatagtttgtcatgtaaggccccaagaaagccttaataaggagccaaacaaccatgtttctttggtttttaatctagcaaagttgtatagtacttaccaaaggaactaaacatatcttgaatgattcatattcctgaaagtgccaaatcatgcaaatgaaaggcttttatgattaaaaatatagctaggatcctcctgagatatgttctatatcttaaagatgtactcactcatgttaaaagatatgatagttatatcctactattgtttactcttttctctatattaaatatattatcaatatacctactcatttattccatatcttatggatatcatttgtataattgcaaacttgtcgtttatattaaacatatcatcaatatagctacaacttatgattttctatcttaaggctatagatagtagccagccagtatttgatactatatttaatataccattagtatagccagaaatttctatataaagaggtccactttagatccagtccatcataaacatcgcaatacaaacagtttcaaggtcatccttaaaggaaggaaaaggaagttgtgggagagtatc

SEQ ID NO: 22 - DNA, 2329 bp, Zea mays
taatgtttggagcaaggggttgtgggttcaagtgcttgctttgctgcattattttttctgtggtttggtagcacggagagtaaaaccgtggtagcatgagttgtctacattaggttcttaatagtatagatatagatttgctcactataacaagaaggtgaaaatattaggatttacttgtatccgagttttatatgtattatttgaggagatatataataaatttaaggtttagcttttattaatctttacaagctcaatgttaaattcaatgttaaaaataataagaagaattttgctaccatatatatccgtttttattctattcgtgagtgattggcatgaccaaaatgatcgccaaactgattaccaccagtaaaatttaatttttttgagctaagagattaagcagtcggtcgacacataaacaaggatccagtctcaaatcatgaacagtgaataatcaccgccatataacaaatagaactgtgtgggtaatcttgacaggacgatgaaacttttggctcatcagtcaaacatgggtagtacagctgtatgctggtgtgtactactttagctattgtctatacacctagcgtgaagcagctgccggccggtcaggctgcatcgagcagttagtactaactagtctttctcatatttggtacgtcgtctgcatgtgtatgtctgtcggataaacgcgtgcagcgtcttagcttgcggcgaggaagcaagatgcatcgtcctggcactggcacacatgcaagttgctcgatccctgtgagtgctacgtacatgttgttacacttttattttttttattatagtgctaattaatcacagctacccatctgtccttggtttgacggctaatataatataaccatcgacgcatgctagtagagtactagatagcactacatgcagggtttccatgtggtagggacgctagccacaaaggttacccatctttctgcatgcggagttgggtttcatataacctttaaaagctgcagctaaggctactcacaatggggatttcatgtctctgtttccaaaaatgccacatcagatttatggatgaatgaaataccctctctcaatagagagtttcatctcactatttcatatgtcaacatacttcttaaatgctgcatataaataaccaataggatttactcaattatatgaagatcaaacaaaacactctcaatggaggtttcacacagtttccaagatcttggaaacgagttaacatggtttcatccccatgaaactccatgaaactcttccttcttaaatgatgtgacatgtcatcaaaatagctgatgtggcaggctaattaatacatgaaacaccccatgaaacctccattgtgaatagcctaatggtgcgactttgtggaagattggtcgttagttaggcatatagtacgtactatactagagtagctggcattgggatgatgggaacatttaagcaaagtcttgtgagcttcgttccaatgctccggttaccgatgcatgactagttaatctgccgacaagtacgtacgatgtctacatgcatggacgacaatgcatgcagtgaacccccacgacaccactctgcgtgccaccggccgtctcagctcagaaccgacctactactagcttggtagagtagtcgtaatctctgctaggccggttgcagcagtattgaatggtcatcagcctcgagctgagctgctagaattaaacaacttgctgctttctcgactctcatgcatgcatgcgcgcgcgcgtgtgacgactgacgacgacgactcttggactggagcttttgtcgcggatgcatgtttgctctcgttgaatcttgatcggtcccccgtgtatcgtgcacaacgcatgcatgccgcagcagtacagctacataggtgtgtactactgatgatgagatgcatgcatatatggccggtgcagatggcagttcagacgacattgatagcgctacatatatatgtactaaaccctagtcccctggtatactactacagtacaggccactccagtttctctgacgcagacaatatttgtatattattcacgtattatttaccaaacattttctatttttcccttttcttataagaaaaataccggccagacggccagtgcatgctacaatgaagccaaaatcggtcggatgatgatagatgaatcaataccaaccatatgtactctagtagcactatctagctagcttcgctaggccggccgtgctgcacacaaaccaattcaaagttgcaaactgttcattaaataagtaagctgcccccttttataaagta

SEQ ID NO: 23 - DNA, 2233 bp, Zea mays
  misc_feature (816)..(816): n is a, c, g, or t
gatccactagtaacggccgccagtgtgctggaattcgcccttacacgggtgagcatgtgaaaggtaacttcctacaacttttataaaataccgtaggaagttatgtttatttcctagagctaccagttgcctctcgaaagttatttatttcctacagtttgttataaaagctgtaggaagttaaaaaaccgtaggaagtgtatgattttggtgtagtgggagccacgtgggaggaacgagcgtggccgggtgcgggcgtgtgggtcatgatggtccaggtgatagatcgtttgctctattgccctagataaagaatagaatatataatgatataatatatatatatatatatatatatatttgtgtatgtaaatatatataaaattcagttttgtttttaaaatttgaaaaaaaacgagaaaactaccactaacggccaaataaagaaaaccgtatgtgaaaagtgtatttccccttttggttggctagtagaaatgttatttacattggtggttccaaaataaacgtaaatgaaaatgttatttttcaatgacctctagcactggcagcacataaaaacgtcagtataaatatctctagaaccgccactataaagcttgtgtgtactagtggtaaatagataatttaacaaattaagttagacgccttattcatgatgtcctttctcaataattaataaagatgggtgttttactagagtgaggttgacgatttacggagagcagatgtctctattattattagtcagagagattatctccaaaaatcgtttttgtgatagtgggattctctcangatcttgttgatcgccaccgccggtgcttcatgcatgtctgggtgagcgagtaggaccacgtagtccgtgtacgactgcacttgcgagtagctataggacctcctccgtttccctgacgataacgcagctttcgtccgtgtttcttctcgctgcgaccagcctccgatccggcagctccgatcctttccggcggccctcagaattgatgcttgcaaagcttgcttgatcgttcgaccaaaatctgtacgactttcatatgcatgccagagagaagacgacgaccatgctctctatacgcgtagccaatcattctctatagcttactagggtacaaggagagatgggacggacagcacttgcggctaaagatagatagatatatataccgatcgatcgtgtacacgtatatattcaatgctctcgctcggccgggtcgtgttgatcccaatcgatccatcggtcatggaagaacatctatctatctatccgttggttatctctacactcttgtaggccgcctgcttatcttttatgtcactgattgcgattttatcgtcgatcaagtaagggttgtttgaatgcactagaaataatagttagtggctaaaattagttgagacatcaaaacaccctagttaatagttcagctattatctatttttggtaaattagttaatagttgggtagttatttgttagctagctaattctactaacaagttttagtcaactaactaatagttttagtgcatttaaacacccctaagagcgtctccaagagactttttatttttcactctctatttaactctttatttggctcttcataaatattaaactctttatatataacatttcattacaacagactctctaacatgcaagttagatcgactaacgagagttgctagccaaatttggctaatgagggagttaatttagagagtcggataacttggtaagttaaatagttagtatgttggaaagctataatactattaagtagctaaattttagcttaacaaactatttagctaattctgctaaaaaagtgcatgcatgcagcaacataacaacaacaacaacaaatcgagttgtccatatttgaattccacaccgtgcatgcatggccgtcgcccgtcggtcgattgcatgcatgcaataatgatgcaaccaacactcctagctagctagcttctagtctaatagtagtaataatattatacttgctgtgtttgttttgctttcttggcgtcgatcgatcgatttctaaataatttacgacgacgtccatcggcggcaaaaggaatatgaatccctgatcaactccttccaatggaatctatccaaaatcttgaccatcaaatccaagactattttgtatataaatccatcctctgtccctgagagaacgagg

SEQ ID NO: 24 - DNA, 2018 bp, Zea mays
ccaaagcatctagttttaaacaacttagtatatatggtgctgacatggatatcactctagggcacgctccaatgggggcaatataacctctatcaacgagcgacggaggagccacctcgcttccgcaaaacccatgcatgtgcgtccaatgtgggaggggggtcgccttgtcgggaagcatttcaccctttattggaaccaacctaagtgccctttagctcatgtttagattaaggttggttccaatagtggccgataccaccctctggtcaggcgatatctctccggcacggggtgataggtacaaattttaagggagcgggatggctctcacattgtccatcgtccgatgataggggacgatattacccccactagagcacgtgagcagcggtatcgcccccacattagcgccatatgtactggccatttaaaactagccgtttttataatttgaactccaatttttttttaaaaaaaaaaatctagtttcatccttagtccacctatataaaggaggtcggtttgagcttattttactccatcgctctctctcctgctttcttgcacaatggcaggttggacctttgattttcaaacactcatcgatcttttgcagcctgacggctctccagtcccaactcaagagaatttggacatggcttattcgttgcatcgacgcacagagaacacttaatcacccagtacatggtcactattttttctgacatattcataccattattctgagttttcataccttctacgacagtcatacaggtgtatcgtgcgcgtatatatacatgattcacttattatctaaaacggttactacggggtgttcgaatacattagagctaatagttaattggctaaaaattgttagtggaattatctagctaacaaataactactaactaatttaacaaaaaatagctaataactaaactattagcttgaatattttgatgtctcaactaattttaggccccgtttcaatctcacgggataaactttagcttcctgctaaattttagccatatgaatagaagtgctaaagtttagcttattggggtgtttggactactctaataaagtttagcatattggaacaaatagacactcatgtccctaatgcggagagaaagaatgagattagagggtatggatagaaaatagggttcgatggcccacgtttagctacttttagtactctctgagcagcttatgagataaggagtgctaaattttatcagaccaccattagctctcctgtttagatcgccaaatggctaaaaacagctaaaaagttgctaaagtttatctgttgagattgaaacaggtccttagtcactaactgttagctttattgtattcaaacacccttattcaaacaccccctacgtcctactacttctctctcttctacttggcctacttgcttttatagaagttggagagctatggggtttggcccataggaccttctcggtgcaggactgcaggccgaaacattgaccagagttatctaatgccagtgtgctatgctaggtttacggaagtgatgaaacagtaggattgtatagagaatgatcctagtggtaggattttgttgctgttacacaacgttccaagtcagttttaatttgtgagcctgcgcgcccaagtgattgccaatttgtcatcgccgattcggcgctgctgcctcagacgacacttggattttggaggtaaaatggcacgccgcggtgttctgatttctgaaagcaatgctgctttcctcaccggcacagccacggcggcaatgctttactttacacgtgccgtgcatgtagtagcatggctgccatgctctcattgaacagtgctcctacacttgtagggcagggcagctgaagaaagataaaaacacccaaaggatcaactctccaagccgagacaacggcagccgctcgcgatcgctgtcgcgggcgtcatttgccctctgcctccacacaaccagagcgctccaggcagcc

SEQ ID NO: 25 - DNA, 965 bp, Zea mays
ttcgataatagtataaatttttgggactatcatgttatattttattatgttatattttattaggtggattttccctcaaataaatcgtggaccgttccggccatcgaaaccaaacctcacgagaaatgactcccaaactgaatcatgaagtacgtgatcgctgtgaattgagtaggaatttctcccattcaaggtacatacagatacaaacatgaatggctttatttgagaacaaagtttttgaaaaccacagtatttaaaatactataacatactttaatcaagacaatactatagtttaaaatattaaagttttggaaactgaggtccagacctagatttagaatactttaaaacattacagtttttgcaatacttcagtttgaaaacatagattttagctagcttgtaaaacaccattttgtatgtaatactacagtatttggaaatactgaagcatttttccaaaactgtcaaacaaacttcactaccaaacacctgtatattgtactgtatgtatcgatcggtttgaaaaatctgtcgatgggtctcttgatcggctgtaccggccaacagtgagactgtctgctactcactcattaatcgatgtgagactagtagtgataggtcgtttaggctcggttgtcctacacagccacagctacggcttaaaaacaaaatactcgcatcaagccgtagtgaacaatagtatattggcgtctcaccaataatcctggtacaccaaaaagtcaatcaagccgtcaaactccccgcaactacacgcaaatttaccttccttgcatgccaagtctgtgctacataactagggccctcctctctatgtctctctatagtcgaactctatagatacatcgccttgacgctccatccttgcacaaacgcaactacgagagagaaggacaaggaaaaaaacaggggacaagatcgagtgagaaagaggagat

SEQ ID NO: 26 - DNA, 2191 bp, Zea mays
gacaaggtctctaatcgttaacaattgtgttgttctgttcttggtatttaacatccgagaacaaggaccaataggagtgaatgcttaatattgatcacaattgtgagacattgaatatcatattgtatgccttgaaggagttggcttcatgatcaatctatgaacctttataaaagtttctatcatatagatctagtcgatcgaacacgaccagtcaaagtggcttatttatgttgatacttgcacttaactcgtatattctaaaagtgagttgaggtgaagtttctgagataatccttgcaacaatatgtattccttttgctaaatctctatgtgcatatacttttagaagaaggtattttgccatactaagacagttttgcaaggactagggggagcacatttctaaagttagcccatatataagatcatatggaatctagatctagaagatcaaaatatgtcttgatgatagttgttatactctttttcccttggtgagttttcctgaagtttctcacaaaaggtttttaacgaggcaacaaagttcagttgcaatttgcgacaccatgcactctttctccatatttttcccactgggctttttgagagttttaatgaggcatgtgttggtcgtggtattcgcccaagggggagtgttaagaaaccctaacgaagggttatgtaggcaaataacgaatataccactgaggggtatcacgtctctatatatagagcatgtaccccctcatatagaatagaaaagagaaagagtccataggcccaacacctatatcatgtgtctcgtgtgtttccctctctgtgtgtgggttgctggggaaggaaacatgcttctactactgctcgcgcctaatctgttgatgggagggaatggagtggatctggtgatccgcggtaacgtagttattcaacacaaccgacctacaaacgttagtctctaaaatttagcaactaactgttggtgcttaataagggtcgctaaaactaaaccatggtgtagtgagaattatccttacatgtgcatgtacgaccgatattctaggcgtaagaattacaccacgctgctgttgagggggataagttggaccacagtttagattagaaaaagtacaaaaagttttttttgaaaaaaggtcgacgattgaaaccagagaaactggtgctttataggagtagagactaggagcggaccctatgaagttgcgggcctagagcgaccgctcatttaccgcacactcagggctagcttggaaactcaaatcctcttctgaattggaggggattgaggtagagatgaactattttttcactcaatctccttcaatactgaaggggattgagtttcatgtttgcttcaactttttcctgacaagtttttctgagaatccagccgtgagggaatctgagcattgtggagattatatgcggaggaaggtgaaagtatccattaggtttagaatctagaaagtgacggatttctacttttgtgacgactcgtccgattttgtgttcacgttgaatttgaacagctttcaccaaaacgatactcgcaaaagcggggtaaaaaactcttggaggggcggggagggggggggggggagaagctgaaaataagcaaaaaaaaaagggccctaagttaggtttggtgtgtacgcaacggctcggattatatcatgagagagagcgagggggggggggcatggaaccactttgctggctggcgtacgtgcttaattcatcatcgtttgccgcatccatggttgcggctagtttagatgacgttactctgaatacgtcagggggaccggcggagtacctcgtcatcagcataggagtcagtaataacccgtcgttctcggggaccatttgtctcatttcgcgcgatccggctccgaaagacagaagccccagtttgataagctacgacttgactccaccggaaaacttgtgagttgtgacgctagcgggagcacgtcaggaagtgacgcagcacggatgtggcgaggcccaactgcccaaagcaaaggtcaagtcgccaagctagagccggccggctccccagtctccaagctctataacatcgtagcttagctctgctattacaattgttacgtagtagtagtacttaacaacaacacgtggcgccgccgtggcgctgatagatgcggcc

SEQ ID NO: 27 - DNA, 1471 bp, Zea mays
tgctctcctggagcaggtgtggcattaaacttcaagtccatacctggatatataatcggtctgaaaacaatgacacgggcaatattaagaaagaccaaaacaaaagaaaaacaagtagtgaaagctcaaaaaaagacaacaagaagcgtgggacaaagagaacaaaaacttggactgagaaagtacgtaaaacaaaaaatgagcagtcatccgaaggataagattcaccctcctctatttgttggaatatgcttgacaagctttgcaagatcatatgacttgagccgacatacctgaacaacagttgcgctaaggaaggaattcgacagcactccaggcctaccatccacaagttatacaagcaagattgctgatatgttcaagcaggtagaaaacagaggagggggtaggagcgtcgtttatcttgtgaatgaggcagatgcactcggccctatctggaaagaaaacataaccaaacataagcggaacgcgaagaaaagacatgacagacgggaacaaactgaggctagctagctgtggcttactgacgaatcgggatgtgcctccagcagcaaacgccattataacatatgatctggcgtgtagcagggtagaagggaagaaaataattggactgtagcaatttgtgaaaatcccatcctcaatatcctaatacgcaatccctagaccactacacaatcatagataaaaggttgagataattcagttcaaaaacagaacagaacatactaagcggtgatcataccatccattagactatgagcaccagagaatgaatacccaagcaatcagaatgtacaataggtcatcatcattgtatacatgtatgggagtgtaattgtgttaacttgaacttaactgatgtgcttattagaggacgattagatacacaacactttaagaaatattgatagcaagtggtttagacaacaaaacagtagcaatctttgcaaaggtcgatagagtaggcaacaagtaattatgatacagtagtaggcatattcgtaatttgtccagataatacagcgataaaataataaaatacagtgatcaaataaccataatacgatgatcaaataatgtagtgtacctggtgaaataaaacctcctcaaaggagacataatacaagtctcaaaataccaatcgattcatcaacatcagcataaccacttgcatattaggagtacttaccagtgttaggtgttctacgagacctagacgtattattttgcctagaaaataaaacaatcagtttgctggcatacatacacaaaagaataggcaagcaaaataggggtgcccaaaattcaccagcaagggtgccacttcgctgatgtgttggatctaaatctcccctgttttacatgtgttgatgctaggaaaagaaatccaccgcgcaaactcaacggttggagcaacgattagcaagcagtgacaaatctaggtaaa

SEQ ID NO: 28 - DNA, 2063 bp, Zea mays
ttaggcaagtttgtgttcctatcctttttctcttctctcctccaaacattacagtatatatggtctcctgctaggtcgattatacaaccatttgaatgtatacgcacaccactacatgtattatgtacaacacagaccattctggcgattttgacccatgaatcgtcccaactcctgtctctgtttctccagaaccgactgctgaatgcgtaccaataaggctgctctatatcgacagttgctggcaaatgagattgaacacatgtccctgcaactccggcttcagaccctttatgaaatgcgccacaaacattacagtatatatggtctcctgctaggtcgattatacgaccaactgaatgtatacgcacaccactacatgtattatgtacatatactaccagtatatgagtactgaatactacttcctttattatattcactgttacgcttacgcacccgaccccaagctgcatcccggtcctggggagctcacctgctgcggcggcggcgtctgcacggcggagggcttaaaagtgaggaacagcgggcggcgacaccaaggttagcttctttgcatcgctgtatcgtggatcggagggctgcctaagcctgggcgcagggcacgggcctatgctcatattcatatcgctgtcgtatactcgcaggctgagtcgctgatggactgaacgagtgaggcggcatgcctttgcatgggcggggccgtcagggttaaggccggcctcggttgcgtgtgtataattgtgccaatttcatgttttacggggtttttctttttctgtcgagctgatctagatccagctcatacgtgcgggctgtgtccggcctaacaggccaatatttaagattcggtctaggcctgtcttcgttcgtgttagcctggtctaaattactaagtgttaggctttgctttggctactgattttagatcgtggttgtgcttgtattaacccaaaaatagggctagatatcgagtcaggtcaacttggccaaacttaaactgtctcggctcgatatattaaggaaccgaataagagaaccaactcgtctcatttgtgagttcgagctctcccgagctaaaaaaacaatatacacatatataaaatagtataaccaattattagttaattctagacctatttaacactaaaaaagagtaacaatactcacactttcacatatcatgtcaatataacaccaaattaacaaatcacttattaattcatccaacacaagtgcgagatttgtttttctgacaaatggttgctcattcaagctaaagagctgactcgaacacagctcgagctggcttgttaacaaatcttgctgagatactagctcagctcgtgacaaaatcaaaatgagctgagctgaattgagtcgagctaaccatgaaccgagcgatctcacgagccacgagtattttgtctagtcctaccaaaaagaccggtccattcttctagtactagtccgaaccccgaaaactttatgatttccatagcatttgtcaaggctgcctcattaatcattttgttgacgatctagagtactctagcgaaaacatgcaagcaaccaaaccgtagagaagtgtagtaggcaaggctggtcgctaatgcgtgcacctggacagtcgtaatcggactgtgcgtgaacgaactaaaaaggcgcaacaaactgttggagtcgctagtcagtacaaaactgaagcggcctttgcccttttgtgactgtcagcacaagcaacaagtccaaactgttgagcacacgatccatccaagtcgacatcctactgctgatcgatcgcgagcttgtcaggttcttcccatccaacgtgcacagctcctatcacggcaaagcaaagcaccagcagcgtacgaggacaaggcctgaatttgttcccaggtgcaacaaacactttttgttctttttagctttgcatccttctcgttccacttacttaatggcacaccatcagcaatgcacaccacggcaacagcattcactgccaagagagtgagcgagcgagcagaggcagcgcagca

SEQ ID NO: 29 - DNA, 2049 bp, Zea mays
aggacactcggcaaagatgccgtctccgtcaccccagcgccgtgacagcggcttttctttgccgagtacttcctggcactcggcagcccgagaaaaagtactcggcaaagaaggctttgccgatgaaatgtgtgtcgagccctctttgccgagtggtgtttttcaggctttgccgagtgcttcaggcactcggcaaagccgtcgattccggtagtgttggggttctcaaaactatcaaataacaaatattcttatgccactattaagggaactatgatgtacctaggctgagacgatatttttctagcaactttcttctcaaaatgagttctttgaatcttctacacaggtggggagctcaaatatctgtcatgagttttctcgctattttaggtttgtgactcctagactatcgagcatcaggcattcttgcaccattattaagccaactaggatgtatctgggttgagataataaaaaactctagctattttctgctcataagcaacgagttctttcgattttctatatgagtgaggagctcaaatacctcagggcatgagttttttttgtggttttgattttgcaatttctatgttttttgttggttccaacatgacaaacatgtcaatcttagcattcaccacatgtttttttcatatgtctaatttattgtgtagattcttgaaattattccatgccgtgtcgcatcagttttcttgttttcatcatttttgtgagctatagctgattggtttcaggtgctttgagtgtaccagaaccaacaaatctcagacattcttaggccattattaagtcaacttggatgtaatttgcctgagacaacaattttttttctagccagtttctgctcgcaaacaacaagttctttcggtttaattttttacacccatgagaagctcaaatactagaggtaacgagttttgttttgttgctatatattttcgatttctggtttgtatgtttttgttagttctcgcagtggcacggtcaaacatcgcaatctagacatttagcctgatattatttttcttgtgtatatttctgaagtaccatggtctcggaccgattttaatgtcttcatcattttttgtcactagttttcgtctgtgtgcttgtatttttagaatagccattttaacttagggttgaatatagttagggtgtaaaactctttatttacacattagccactcggattaggatttaattattgcaagaaagtttagagaagtaaaattcagttctcttagagatggtggttctttcgataccatctgctgaataaatatatcccatatctcgtataactaactgatggataggaaagtagtggtcgaataaacaaaggaaacgtaaaacaaattaaaacgacggagccttccgccgcttggcatcatgcatctccaatcaataataatcccctccttgcagctgttgtctttgtgtgatggatgagttcatctccatctctcccgacgacgacgggtggaagaagaaaggcaggcaggctggctgcacttggcgtgtgcatgactgcatggcctggcgccctggcaaacgacagacacgatgactagcagtctagcacagcatgcatgtgtgctttgcgtggctacccgtccacgcacgtatgcgagagcccctagctgtagcgggtactacatacaaatgaaaataccctcacgcgtacacactactagcctagtagatcatgtatatcatctgctcatcgcagtgaatgcaagctcatacatggaaagttagctccgcgcgcaacaaacaaaaagtgaaaaggaggccggggtagtacataccaaaacatgcagctggctttggtgtgcatgcagacatggaagcgtgcaacagatcgtgcacacatgacatggcatggccgctggcgtggcggcacacatgtgttgtacactgttcgttcaattctccgattgaaaaaagcgcacgtaacgtcgcatatacatacgacacgacgggcgacgacggcgacggccgtgcagagtgcaggtgaag

SEQ ID NO: 30 - DNA, 2045 bp, Zea mays
tactccatagagtccccaacatcctagacacatgtcctaccagtcctcaaccgcatcaacaacacggtatactctgtcatgtcctcgctcctactaatgaccctaggtgttagccactgcatgtggtcacctggcttctctagtccctcagtcaagaactagcgctcatccttcatcatttctgatccatcgacataaactcacatgaactttacctacactgttgatcatttcctttagactcccacacatgtgcatcataagcctagaggcacgttgcacacacacacacacacacacacgccccggttagtccaaacaactcaacccaagattctactccgttgataatcacaaacacgaaccacaagggcacatatcaaccttgtgttcgtaactttaatactatcctctaaagcctcattgaaatatagaggacattccttcaccctctctatatagagactctctcttctcttctccatcgatttttatactttatacaacagagtagtcaagcctaaaatatgatcagacatttgagggtatgaccaatatgtagatacatctctaatatgggtctttgacatatagagaacaatatatagaggacactattaaagggaactgagaaataaagaagagaatcttttagaagagtttgtaaaggacaaatatagaagatatcgctggagatagcatacgagtgacaagagaatatatgtttatattttttaataggaaatatggcatgcactacaatggaatgaaacccaacaataagtatttcatgacaataacataacatcgctactttattatggtatggttatcggatgcttgacctgcgaactattgtagctaatattgtagcttcatgagggcggtaaaggcaaaggaaaaaagtgatagctcctaagctcttctttgaagaaacaactactagatgaagtttctccaaaagtacgtccattgaatggtggagtaaagagtcatttgacctctcagaataaaattaaaatgagaataagtaagaataaaacatctctgttctcaaatctaggccatacaaacattgggtattactaaaaaatagctaatgccatctttcaacatttggaagttaaaaccaaccaatcctcactcattcccaagaaatattggatcatatttaacattttgtgtcacttacaaaattggcttaatcttttatgcggcaatgccaaccttttttagcagggtgccacttgtaacatgaaaactataactattttcaaatagtaccttgaaattcgcattctatttttatgcattcttcaaaattgacacaaattatactaggagaattcaattcattcttgccataacatattctaatgcaaatattaagtagattgctcaacatcggtatacatcttttggacgattaattagtattgtctcactacattctttgttttagcagttcattaataatgcctcaatagcatacttcattttaggaactttatgaaaattgtcttaaaatagggccaagtcacaaatccacttcaaaggtgactcttcatttcttacttcctatcttcctattttagcagggtgccacttgtaacatgaaaactataactattttcaaatagtaccttgaaattcgcattctatttttatgcattcttcaaaattgacacaaattatactaggagaattcaatacattcttgccataacatattctaatgcaaatattaagtagattgctcaacatcggtatacatcttttggacgattaattagtattgtctcactacattctttgttttagcagttcattaataatgcctcaatagcatacttcattttaggatgaaaattgtcttgaaatagggccaagtcacaaatccacttcaaaggtgactcttcatttcttacttcctatctttgcttgtttttgtatatatatatgtgtggatggttgagtgatgttcctacaccactacaccactccttagacacatatatggaaaatagcttcactgtctagattcc

SEQ ID NO: 31 - DNA, 1475 bp, Zea mays
aatctgccgaccagagacgagtttcacatgcatgcatgcttgaagaagatcgagtatagggcattgtaaaatctaattcaatggaaggttttttaacgggtctttacagtgctaagggaaaaaaattaaaagacaaactcttcatgtgagtccgtctctacccgactttttatatgtattgtgttttaattgtatttataatctatagggtcatagttgtatcatacagtatattagttatctcttgtatttagaaatccgatccaaaggctcaggaccgtacatacccttaggggcacgtacaacggcattgaataccccgtctctaaggagccacgtaagcaacaattaaatgaaaaaaattcaacgtgaacgctaattaaggaggagatagaggcatgggtcgtcctttcgtaagaagacggcttcggcccatgttccaatgccaaaagacggctttggcaagtcaaccatggtatgattgcttcgatccgtgtttcacatatgaattaaaaactttataaacagtgtgagcagacaattcaccgtataacttgttatttatattttctatacgtgttttttcaatacatatatatacatcagttgtctatgagatatacatgcccttaccataagattgtcatggacgacaatggatgcagacattgcagtgaacccacggtagcacaccacactgcgtgccaccggccgtctcagctcacatcaatatcaatagaactagctagctactactagctgcgtggtagagtagggtacgcggtcggttgcggtattgaatgatcatcgtcagccctcgagctgctaaagcgacgtgcagaggatggacttgtttgcgcgtgtgacgatcagatgtagtatcaatcaatgcggtggtgtagcacgttaaatgcacacacacttggacggggtttcttttggtcgtggatgatgcatcatgtacatgcttgctcctcgcgctcgctcgttgaatcttgatcgatccccctccctatcttgtgtgcacacgcatatgctgcagcaacaggacgacgaattgaacgtatgcatggccaagtggagatggcagtccagacattgaaatattggctacgtacgcgacgcgacatgcaggcaagtcgttgggctacgtactgtacactcctaatcctagtcctagccatggtacatactactactacacagctacttaattaagatggaatggtctatccatccatatatattctaaatatatagcctataggatgtcagtcgtctgtgctacagcagttctgtctagctagcttagctaagcggtgcacacgaaacaaagcaaagtcgttgcaaactgctcattactaagtagctgatgcttatcttattttatatataggggctgagaaggagaggagattctcgtaccatgcatgtgttcaaaacggaaagtctgatctgaagccagcagacgtgaa

SEQ ID NO: 32 - DNA, 992 bp, Zea mays
aattcaggagtcagcttttttttatttttcattacgaaaaaaatatgaaatcaaaaatggtttacatatttaaaggaaaaaatgttcatcgatttaaaaaaaattcatacctttcaaaaacgttcatcaattttttttaaaagttcactaaaaatgaaaaaaagttcatccaatttaaaaaaagttcattaattttttataaatttcaataaatttgaaaaaacttcataaaaattcaacaaaagttcattgaagtgaaaaaagctcataaatctttaaaaagtgcatccattttcaaaaagatgttcatcaaaattcaatatagttcaccaatattcaaaaaagttcattaatcttaaaaaatattcgctaaaatttaaaaaatgtttatcaatatttaaacggcgtctagatgagccggtctatttacaaacaccataggcgccaattaacaaaaatgcacgttagatcacgtctacggcgtcaaataggaaatgcccatcggccttactattaagagttgttttggttatcctttaggatttatgctgtgggctggacttaacacaaaacccacagccatggtaggccggaatctattattcagctcacaaacgatgttctactcaaaagaagaaaaaaatctgttgtcagaaaaggagaacaaaaaaggctcacgaacatgcggcggctcgcacaggtggccgtgagcttctgaatgacttggccacccggcatgtccactgcccccctagacggtgtgggtgggtggacaggtcaagcgcattgaacaaggtcaccctgcgttctgccacgaggccaactgcgtggccctcatgcaacgcgccttgctgccacttctacacacgccctcgccggccgaccgctgctataaaagcagctccccgttgcgtcctcgacggcatccatcgagagacgttcgcagcagcaaagagcacgcagcaactagctttcagttgtttcaacccctg

SEQ ID NO: 33 - DNA, 567 bp, Zea mays
gtgcaggtgtatgagtcattgttatgatctataagtgtcagttcatcttatcatctcggagaacaatacaaagctagttttataaaaaacagtctagtctagaagaacagtccacatgtaaggctttaaaaatcgagcatatcttaacaacccacacacgattgcaacttagtcctacaaaagttttgcctttcttgtttctcgctagcaacctatacaaggttccaaaatcgtttgcagaagtgatactagcttgataagtgcgtgacatgtaaagtgaagaaggtgagtcatgtataccaaacctcgggatttctatactttgtgtatgatcatatgcacaactaaaaagcaactttgatgatcaatccaaaagtacgtttgtagcttgtgcaacctaacacaatgtaccaaaaatccatttccaaacatccaaacacaattgttaaatttgatgcaaagaaggaaagagatgaagccatggctactataaataggcatgtagtatagagatcatcacaagcacaagtatcaaaaccaagaaacactagttaacaccaatccact

SEQ ID NO: 34 - DNA, 2983 bp, Zea mays
accttccacatgactcttcaggtgagcagcaaggagaacctgcggccacattcattcatctatgctcttcttcgctcaactaaaccctaaccctttttccccaattcctcctttcagatggctcgcaagaggcacccacccaggcacccagcgctgaggaggtcgtcggagccgcagcccggcgggaagcatctcaggcgacgggctcgtaccttgcacctccgctgtgtattggtactgacataatcggctgcaaagtggaacattcaggtatattgtgagatagccttgtacagttctacaattgttcaatttccttgtgaccctgcaagacaatgtcacaaactcacaatgcatgtatgatacttagtttcaagggtgggtagtgtcaagtactacacttcagtaacattcttgtgcacaggattcagtaacatttcaagacgaaacggattccgtgttaagtatgagcaaaccttagaggcgctgctcattttttgagacatgtatactgtcatgtactccctccgtctcaaaatatagcaaccttgtactggatgggacacattctagtacaacgaatgtggattctgctgtaaccttgggtctgtccacattcgttgtactaggatgtgtcccatccagtacaaggttgctatatttagggaccgagggagtatttatatgaatttatgcacgcttggaagtctgcatttaactgggttcatagagaattttcagatcgaacacaaaggtatttgttacacattctgaacttttcagatttgggattaattctcaccgcgtattcacatcgtgtcatgggttctattatgataacttaaaaggcccattatgatcacttaaaaggctttgctttgctgtgtaaacagatggatatacaaggtatattgaaagttgtagatactattgtgatattgctatttctggtatcacatcttcagaacaaaaatgctgtcaattaatgtgtcctagctaggattggatcatcggatttacagctattgtccaatcttgtcaaatgccacttacaaaaccttgtgaacagtttactctaattctcaccactatagagatcagtcttatatatgtaagaaagatctataccaaatgagcaaaatcaagtcaagatagtatatagggtcttaccgattctgaaggaagtggggctacattctgttcaatctttatgcaagagttctaccaagttgcacgcaagtctgttctgctgtaaccgtgtattagaaacaaacttgtgtgttgtgatagctcataactaattatagctagttagcatattatccttttaataacaatatgcttctctagttagttttcctctatgtacattgggatgcaacagacttggattccagatctggcagttggttttaacatacacaccaacctttacctttagcacaggtcctcccttgatattataaatttataatcatattgttaattgttctaagatcacttaaaattattaataacaattaaacaaataagtataagatgatgggtgacagtttacacaaactacattgttttgtctgtttctagtatagatataacataacagaagagctgtagttaagtagatgcctaccagctatgatcatgagtaaccattatttactaatctcagttattgacccttacaaaaaatctgtgaatttttaacagcacgcacactgataaaagtgctagcctgctagagctgtaacaagacacagagacaacttccaaaaacttgttggttgatggagattgaaaccgaattttattctcatcagctattaaggcaaggactcattcctataaatttcgcaacaaattgatatacatgaattgcaaataatatttttgaagaaaaaaatgtgaaggaaaaaacgacaactattttagctgtagcatgaatgattagcgatgagattactaaagtagaatttttttctctgtgatgcagttatattactggaaacggacactttctccctatggagcttactgtttatgtacacatgaaagattgctattcacactgattagaatattctgcagaaaataatgtgtacaaatgtggcagttacagagcagttatggatatcatatatagctaagaagaacaagagctgggcttttctgaagttgacaaatccaaatatggagctaaaaaaaatgctgactagtctatagaatgctgcctcgtcatatcatttgaaagtatcaactggcatgcatatcctaaaatattatcatttgctactaagtacatcccaattcagtctgcatctctgttctgcaaattttcaaaatcgactaccagtagatgtttactccctgtttacaagtcaatcataatactcgtgtgagcaatgtaactgcaaactagagaaagatgtacagaaaatcaaagattatcttagctttgtacagtgcctgaaaatcaaaacatgttcatatttcaccgagcctggtcttgttagccaccttagtctcaatgatcactatcactagcagtctgtttgttatgaatcacatgcgcagctaatatctgttttgctaaaaagatttttattgtaaatttatctatgtacagatgttctaatgcatcctaatacccatggaagctggggtgttacgcatgatgatagaggtgtaccacattctgaagctatcatacacgaaactccaaacagaaaggtaacacctgtcaccctgtcttgccactagagtacaaattcaatattggaacccagaattacgaaaattaaaaacaaaataataaaaatgaaatggaaaaacagaatgtacacaagacagagagaagacatgccgaggcctataaatacttcacgagttcacatccccttctgcactcaaaccatcccaatgaacagagtttcaaaagagaaagaagtatagcccttcaggctgtcacagcacttgtgtctaa

SEQ ID NO: 35 - DNA, 3346 bp, Zea mays
taactctcagactctcagtggttattcctctagcaaggcatgaaatttgtgtattgcccagattcatcttcccaaaattactgactttacttgctatcccagtggttattcttctagcctcttttttatacagaaacagaacatacagcttactgaacattcagaagcaaaattcagactttgtagcaaaatacagaggcaacattcaggatacattcagaagcaaaattcagactttggtagcaaaccacatcaaattaatctaaagaggtgcctccatgtctttcctcgcattctttcctttgcgagacttcggtagttctttagccctcttgattcttttactcctaccctttgatcgaacatcatttggagggtgtatttgtatgtctgctggaattttgcaaccaataaaattctcatattcttcttgtgtaggctgcaccatgctagatacaatgtgtcccaaagattcacctatgttcagcacacttgatactaagaaatccatggcttcatttgaactttttgctctttgaatcgcctcttctatcttgtttcttaccactgttatctttttccttttttccgcttcattagcgtcttcgggcttctcttccaataggttgccatcatcatcatacacacactctctaaagtaacatgtaaaaaaagatcgagtaagagggtgtaaaataggatgtgaaaattattacttttaccttttgcatcgtgtttcccatctcttcaaaatataagatgaaggtagctcatatagcttttcacctcgcattgccaaaatgatatggcaacatggaattcctatcttttcaaacagtttgcacgaacaacgtccaaaaatatttgatgtgcaccactgaaccactctatccctcatagacccatcactaactacaacaaacttaacagcatcttgttgtgtagttcctagaacagagcaatggtctctagcagcaatcacctcttcctgaaatattttgaacactttatgtgtgtacaatatgcttgcttgtttctctaccacccatggggtacgcaagacaggtgtgctatgtatactaatatgatcagccttcaactcctcatgtcgttggcattccaaggccgtatcaaatctaagccaaaactcaacaaaacataatttgcggtgtataaagcgattgaagaatgaatttgcgctttcagatcttgaagtagttctaaggactcctgcaagaggtgtatccataaagaacgctggaatccatgactcacgaattttatatctattagctaaccattcattgctctcaagaccataagcatccatgagagcattccagcgcatctcaaattcttctggagtttctgagccccatacacaagtgttcaaagcatcccaaaactccttgtcatgacttgttggatgacccaccttctcagaaaccttctccataatgtgccacatgcagagcctatgaattgtatctgggagagtggttctaattgctgattttatactagcatcttcatctgttatgataagtcttggggctttcccccccattgccaccaggaaagtccgaaacaaccactcataggactcaatcttctcatttgctaggaaagcagctccgaaaaaaacactttgcatatgatggttcacaccggtaaaaggtgcaaatttcatattgtattggtttgtgctatatgttgcatcaaccgatactaaatcaccaaagtgagtataattctttctacatgtagcatcagcccagaatatatacactaactttccatgctcatccacagcaaaatcatagaagaaggccgaatttgcctctttcttcctctccatctgtgccacaaataactgagcatctgcattctttattttctccctaagtccccgatagtagttctgaagatcccgcttcatacatccaatattatcgaaaccatcgcttacttgtaaaagtctatatgcttgtgatgtaccaatacttgctttatggcacgtgaataaagtagtcttggctctttgacagattgttcggtttgatcggagaaaaggaattttatctggtgagactagaccatggttatgctcctctacaaatgaagcgatataatacctcttgtcctgacccaacctcacatatacacgtgcattgcatccacatcttgtttcaaagtgcttcttttggttttttgcttcagcacatctccttgtgaatccttccctagaacacacaaacctcttatgctcaaccacatcatttttcttgccttgagctcctatacgaaccgaaaacccacattcatgtgcatatgtcttgtagaatccttctaattcatcaagactatcaaatgacattccgatcactggtttcaaactatgctcgcaatcaggtacaaacgatgacgacgactgccatgaaaaaaataaatgacttatcatgtctaaatacgcagctcataataaacaaaaagtacacaaaattgatcatgaaaaataaatgactgatcacgaaaaaaacaaattcctcaccgatgtacaatggaattgatcctttttgggtgtactaaatccatatgaaggatcaacacgttctgctgctagaacatgatcatgtacaggagcttccgcttgaacagaattatggataggagcatccgcttcaacaggatcatgggcaggttgtacgtgagcgtcggctttaagaccatcgaattcaacgtgagcatcgtcttgaagaccactagagccaggtatccctcgctcgtccatggcagtgaggagtgccatcaagctgccactagggcagagtggcggtggctcctccatgcccctacggacacgattttgccacggcggcggcggtacgcgatgaggccgagcgacggcgcgcgacgactgcggcggtggcggcacacgatggggctgaacaggggcacgcgatggggccgagcaggggcgcgcgacgactgtgacggtggcggtaggcgaggagtcgctggcgtacaagagaagcctcgatggcgcacgggtctgtttttgcaaaaaggaaggcgagcacgggatggttgcgcggggggttctttgaaaaaagaccagctgccgttggttgccggatgtgtggacttgatgcgctttgtgcatttgtgcacgggccgtcgtttgtgcacctgatacggagccaagtttttttctcgttcctctcttctcctgctttctaagtccagtctcccgggcagaagaggcacgagaggaaggtggggaaggaaaccacagccgcttctggcgggcgctcaccctgccccttccacctcgcctcgcctcgca

SEQ ID NO: 36 - DNA, 2198 bp, Zea mays
tgagcataagaaaaattattaagaaaatatttgtagggactttaaaaatgattctgaaggtatttctcaggaggaaaatatttggagaaatattggtagaatatttagactgtatttctggaaagaatttgagagggagtattgaggaaatatttgtaggatattttaagtaggattttggagagattatggaccactatattttgatttgttgagatcaacttgcaaataaacatgttgaaacgaaattttaaatttatttttaatttagagcgtgtttgagaaattttcaaaaatttgaattttcgtgatgctacatgaacccgcagccgcaggcgcgggcacggtggcactcacaaacaaacattttgaaacaaaatttaaaattcattttgaatttagagcgagtttgaaaaattttcaagatttgaatttttgtggtgctacacgaacccgccagcgcaggcgcggcgacgcttcggctcgaccaaggtggcttgagccccggttcgggcgccggtacggtggcccggccatggcatggcagcgccgcgggcacgaccttggtgcatggcggccacggcgctgcacgggcgcacgacctagcctagcacgggcgcggtagccccgacgcggcgcgggcgcctcaacctggcgtaggtgcgacgatctcgacggtcatctcctccggccgcgccccgacagtcatgatgcgggcgcacagccggtggcgcggcctggcgcgaccgtggctcaggtccgtgcgcgcgacctcgacgttcggacgcgtagccccaacctatgctggtgtggcctcggcctgagcgaccagcgtggctctgacgcgtgggcgcgcaaccccggtggctcgacgtcgtcgacaacacgaagatggcgcatatgacgagtgtgaagagaaactgtacataaacctttctgttattatagaggtcgagacttcctctcttcttctttctccaacaacttctttttgttcccgccatctgttgttaacatgttggattgcaaatgtaatgagatacagagactagccacaattaaatgaatccatagttctttattaacataatgattacttatatagagatacaagggatgtgacatacggatgcattcgtacgggtgcgacgaacggatgtgtccatttgtcgataacccttccacaaaagatgtcggttgagatcatccttcaactgctttgactcatacatacggcagtggggctgtggggggcacacagtgcagtgctactccaaatctccaatacgaacggatcaacggagcagtaaccgcagcagtacatgcatgcatgcatgcaggtatggtgccctggcctgtgcgtggcgtggtgccgtggtgacgtttgagctgtgggctgtggcgctgagagagtccgagcaggtgactgtcaggaagtgggaccagtcctctgtctgctcatcgcactcgaggtgtaccgtggagatttcctctgatcatcagatcatgatccttcgttccttttgtgagggaaaaaagaacggcggaaatgcgctgccaattccgggaaactacacatccttcttctttcttcttgcacgaccacacaacacaaatggtggccggtgcccttaacctgttacagctttcggacgatcttcgaatgcacttcaactgtgggaagaaatcagaccatcattttcaccgatgagacatatactcacacacttctacttgaatggttgtttatcttttgagcaaaaagtcaccgccagaaatggcgccgatgggtgtgcctgtgctgccgagggttaacgatggaagctactagcgtagcccctagtgctggagcaaattgatggcgctcgtcccgtcccgtcccgtccctccctctgctgcgcgcgcctacgccccgcccctctttctctctctctgacaccgcacgccgtgcacccagccgtccactgatccaccaccaccacccctcccgtctcccgccatgccacctgcctttgacctgcctatcatctcgcccgcgccgtccccgtccccgtccccgccgccgccgattccattccatatgcccattccctcctccaccaccaccaccgctaagcccctcactccgctcgcttcccttttattccatccgccacccccacgcccccctcgccaccacaccacaacgccacaatcac

SEQ ID NO: 37 - DNA, 922 bp, Zea mays
gatccagcgaatctcggcatcagtttccttctatcctgtgtgctcaagaatagtggcatggcctatgcgcatccataggtatatattatggatgtttgcatgcatgtattaattccttgacaccttatcagtatgccaagcacactatcaacaaattgctgggctgcaccaagatttgtcctcgcgtcgtactctaagtcaacgctagcgcgtctgcatgtagctatactatgtattattttattttatcacggtttagtttaaaaataaactaatcagacgataaatatttaaaaaaatgtcttaacatcatatgtcctgcaggagtgcatcctttgtgtacccaagttgaccagacagatgtgaacgtttacgcaattgctgccttaattctatcataaaattgtgttcttatgacaaagatatactcttgttggaagtatggccctgggatcaacccggctagtccaaccaaccggcctaaattgcactcctagctagtattgtcttgtgtcaaatggtctttttatattgtggaataggccctcttcagttaaaatggacattatatcactgtgaacgatatgtggtctcatttgtccttagccattgatatctcaatcttttcaatcgttgacctaagccattgaagcatgcctcactacaagtataaaattatagcatcaccaagatatggacacacatgactagatatatatatcattaatattatgattaaaataaattgtttgactaaatatatataagttaaataatgtagaataaattgaaactagaactaaaagatttgccacaaggcctataaataccgggccttgtccttgacatccactccacatccaaacactgtaacaacttgtgtagtacttaatccctcgcacctcatgaatcccaacttcag

SEQ ID NO: 38 - DNA, 463 bp, Zea mays
aaggtgcaaggattgctggagcgtcaaggatcattggtgtcgacctgaaccccagcagattcgaagaaggtacagtacacacacatgtatatatgtatgatgtatcccttcgatcgaaggcatgccttggtataatcactgagtagtcattttattactttgttttgacaagtcagtagttcatccatttgtcccattttttcagcttggaagtttggttgcactggccttggtctaataactgagtagtcattttattacgttgtttcgacaagtcagtagctcatccatctgtcccattttttcagctaggaagtttggttgcactggccttggactaataactgattagtcattttattacattgtttcgacaagtcagtagctcatccatctgtcccatttttcagctaggaagttcggttgcactgaatttgtgaacccaaaagaccacaacaagccg

SEQ ID NO: 39 - DNA, 1433 bp, Zea mays
tcctaatcttcaaataaccatctcaaaagttttttaaaacatcttttgaggatatgtatcccatagccctagagcgctaaattgactacttttagtcgattaaaaggtattagacatccttacaagtcctaagtatcaaatcaccttctatcggctatacacaactaacggaagttatctctagtcacactaacttatgtcggtttccgcatggcagatcaaaattagctaacttttgttggctaataagagcaattccaaaagaacgtgtaaactaatctcaaaacagatattagttaagaatagtaatttttcttactccaacagttccctcagtcttccccaaaaaattaagcgttccgcatccacagcctcctctcggtcgtattttggtgtgtttcatccctccccaatccatttctcaacgtatcagatcatccaccgcctacgacgactgtacagtttgcgtcacatatcacatttaaaggaactgttggagtacccatcataattcactcttaaaaaattttagcctgctctcaataatcaattgggggggtaaaatttttaacatcctttcggatctaatccaacttatggaagttagctagctctggtcgcgctaacttctgtcgatcgcctattagctaatactccatctgtcccattatataaggtataaccaactctgattcaaagaccaaaaatatacttaattgtgtctataccacttcatcgatgtacgtatgcatagaaagagcacatcttatattgtggaacaagaacaaaaatatggttacgccttatattataagacgtagaaatcaatggtttacaatagccaagaatagatgtttttatttatttcctatatagatgtttttatttatttcctatatgtttcacaatagccttatattgtgccgaaaatttaggcacacgtgccacgaacgtctgaaatgtactccgcgcgtattaccatgcactacgacgtacgtaggagtatgtacgttgaaccaagcacacatatatctctgacacagtacaatgatatactacaacaacaacagtactgcccaattcatccattttcacgttccatcttccgcgtgtgacaactcgatcggccacgcacgcagacgacgacggagcagtacttcacagaatcctccgccactcgtcacaccaacaggcgcgcgctggtgcgcatgcatcatgtgcatgccatcgtccgtcccttggcgtgcctcggtagacggtagctagagtagtagcctgtgcttgctacccctggtcaacacatcgtagcctcctatatttaacgtatcctcacacatcacaagaacgacacacagaaaccagtagccactactccatccaccacgagcgagcgagcgataaccctagctagcttcaggatccagcgagagccc

SEQ ID NO: 40 - DNA, 1998 bp, Zea mays
  misc_feature (762)..(762): n is a, c, g, or t
agatccaaaacaggtcatggactgggccgtaaactctatccaaaattcttcatgtttttccatctttcaaaaatctttatccaccattccattactagggtgttggttttattttatttgttgattaattatgtattagaaaatgtaaagcaatattcaattgtaacatgcatcatctaacaccaatatcttgtactaaccttttgtaattttcctataaacattttaaaaggctaatttaaataaaaattacaataaacgtgataactcactttcgtaacgcatatttattcaaatataccaaaatttaccattttaagtaagagaatctttttaaaattaattttcaatttcattaattaagaaacaaagaatttactgaaacctatattttattaaattttaataaaatatatgactaaaataacgtcacgtgaatctttctcagccgttcgataatcgaatactttattgactaagtatttatttagaaaattttaaacaacacttaatttctagaaacaaagagagcctcatatgtataaaaatcttcttcttatctttctttctttcttaatagtctttatttttacttaattactttggtaatttgtgaaaaacacaaccaatgagagaagagcagtttgactggccacatagccaatgagacaagccaatgggaaagagatatagagacctcgtaagaaccgctcctttgccatttgtatcatctctctataaaaccactcaaccatcaacctntctttgcatgcaacaaatcactcaaataattattttataaagaacaaaaaaaaaaagacggcagagaaacaatggaacgtggagctcccttctctcactatcagctacccaaatccatctctggtaatctaagtggctatttgtatacagtatatacttgcctccatgtatatttatattctcgtgaaaaattggagacatgctttatgaattttatgagactttgcaacaacgaacgagatgctttctctctagaaatttaaatttagatttgtgaaggttttgggaatggcccggagaagacgattttatatatacatgcatgcaagagtttgatatgtatattgtttcatcatggctgagtcaaagttttatccaaatatttccatggtgtggtattagttaaacaaatctctcgtatgtgtcattgaatatacccgtgcatgtaccaggaatgtttttgattctaaaaacgtttttttctttgttgtaacggttgagtttttttcttcgtttcaaaacgagattctcgtttgtctcttcccttgtctaaaaacatctacggttcatgtgattcaaaaacactaaaaaaatataaactcatttttttttaatacttaacatttaaactatatatatatatatatatatatatatcttatactagtcccaagttttagtgtgaggtttttttattcaaaatctatcagtacattttttggaaaagaactaagtgaaattttctccaaattttccttttactattgattttttaattactggatgtcattaactttaatcttttgattctttcaacgtttaccattgggaaccttcacatgaaataaatgtctactttattgagtcataccttcgtcaacataaattaattgatgttcttctccaaattttgagtttttggtttttctaataatcttaacgaaagctttttggtatacatgtaaaacgtaacggcaagaatctgaacagtctactcaacggggtccataagtctagaatgtagaccccacaaacttactcttatcttattggtccgtaactaagaacgtgtccctctgattctcttgttttcttctaattaattcgtatcctacaaatttaattatcatttctacttcaactaatctttttttatttcctaaagatttcaatttctctctgtattttctatgaacagaattgaacttggaccagcacagcaacaacccaacccca

SEQ ID NO: 41 - DNA, 4427 bp, Zea mays
  misc_feature (5)..(5): n is a, c, g, or t
aattnaccctcactaaagggaacaaaagctgggtaccgggccccccctcgaggtcgacggtatcgataagcttgatatcgaattcgtggccattagacccataactatatgacgatgttaaagagaaaataaatcataaataaaataagagtccttatcaataaacctaattggctaatttcaacctcaaagagtagtaggaacaggtaaggtgaagccaaacagctccttttacagttggaccactagagctgatctggcatacaaagtatgcttattgggctgtcacggcccatccgcaaaatgtcgttggttacgaagcatccacgacatagacggtgccacatgttagaaaagtgtttcggcgatcaagattgtgtccacatcattagacgtctgaactgtccacgtgtctatcaaagctggcgtcaaacattacgttttcgtcgtttgcgcctcctagttcacacgtgcaacgaacgcgtgcgacgtatcaaaattgttaattttagccatgtataaagaatatctacaaaattaacctcaggaatatttttgttttttcaattgaggccataatatacntnccgatngaaaaattttncancatatcnctaatatcaaaaaattatgatgttagtaaacgtaaaaaatttacacaaaataantttcacaaaacttannggggaaattggaacaaanaaaagactggtgagtgataagcgatgatggccggtgaatcaggtagccgtcctacaacgtggttgattttgagcaaactcctatctactcttcacactattggaaatcccaaaatgtcgtcacaccataataatgtgaattttgttatggaatttgagggaaacagtagatatatgtttcaaccagtgaaagttaccctcctttggacatatctacganagtagaaagtagaaacattcactaaacgtgacaactttataaattttctttttgtaacttttctttagatttatttacganaagagaaatataaacgtcatgctaataaaaaatgcattattttctaccatctagctagaatattgatcaagtcttcacgttttttgtttatctcttctctcataggcatgtccacaaaagggtaagttttactggttcaaaatattgcatgagtactactaagctcgtatagtttgatcttactatcattgcgatgagggttgttagtttggaagaaataaggatttatgcaaatggtaatcattatgtctgctatttaagaagtaaattatgatgcttgttgcgtgaacatattaaatttgcgaaaaataagcaaggatacacgagagaagctcagatattcacgtaacgatgtttcatctcttctcattgaggaaacatatggccatgatatagctaataagcctacgggattgtcntttcaacgccgaatctaccaaactgttccatctcttattatatatagtttggttatttaagtaattagatgcatcataatctttttttctgccagttgtaatgcagataaaaatatattggttgttctaaggattgttcaaacgtgcatgtgtacaagttattatttatatactttcatctacatgcgatgcgttatttataatgataaaactaagatttttagttaaatttaataaagagcttacgagctacaattaattagaaatggttgctcagaaatcagaatactatatatgaaaaaagaagttggtatacttgaaaaaagaaaaaactacttgaaaagatggtaaaagatatagaacgagtatatatcttactcaagcacgatagaagtttgtatcaaaacattgcgttccaaaccaatgtttgaagatggtcaaaggtgctactcatgatgtggtgcgaagaagcttacgaaaaattctgcaatgagagataactttatgggctgcttgttcaatatattgaaaatcatggtagacaacaccaaactctcctttaccagaagtcatatttccttaacctcagaataagtaaatcttctagtttattatttgaaagttgagcgtataattgcaatgaaacttttaccaattcaccgcctcctaactgagttgttgtattatcctatctctttagctatcctttccttgctcttgctccacctgcatgtggcctctttatttataatctctctagattctgctaaagatgtntgttcaaaatggtttatctttaagggaagcaaagtgaatggaaacatttaaagaaaaaaaaaacttttagcagagttccatgagatttcatactgatgataactaaaataatcttatatgcgtaagattattttagttctaaacttcattttgaaatgagaggtcattggccaggaaagattcaatattggttctttgttaattctcgttggtttgtttttagtatgggctagatccaaaacaggtcatggactgggccgtaaactctatccaaaattcttcatgtttttccatctttcaaaaatctttatccaccattccattactagggtgttggttttattttatttgttgattaattatgtattagaaaatgtaaagcaatattcaattgtaacatgcatcatctaacaccaatatcttgtactaaccttttgtaattttcctataaacattttaaaaggctaatttaaataaaaattacaataaacgtgataactcactttcgtaacgcatatttattcaaatataccaaaatttaccattttaagtaagagaatctttttaaaattaattttcaatttcattaattaagaaacaaagaatttactgaaacctatattttattaaattttaataaaatatatgactaaaataacgtcacgtgaatctttctcagccgttcgataatcgaatactttattgactaagtatttatttagaaaattttaaacaacacttaatttctagaaacaaagagagcctcatatgtataaaaatcttcttcttatctttctttctttcttaatagtctttatttttacttaattactttggtaatttgtgaaaaacacaaccaatgagagaagagcagtttgactggccacatagccaatgagacaagccaatgggaaagagatatagagacctcgtaagaaccgctcctttgccatttgtatcatctctctataaaaccactcaaccatcaacctntctttgcatgcaacaaatcactcaaataattattttataaagaacaaaaaaaaaaagacggcagagaaacaatggaacgtggagctcccttctctcactatcagctacccaaatccatctctggtaatctaagtggctatttgtatacagtatatacttgcctccatgtatatttatattctcgtgaaaaattggagacatgctttatgaattttatgagactttgcaacaacgaacgagatgctttctctctagaaatttaaatttagatttgtgaaggttttgggaatggcccggagaagacgattttatatatacatgcatgcaagagtttgatatgtatattgtttcatcatggctgagtcaaagttttatccaaatatttccatggtgtggtattagttaaacaaatctctcgtatgtgtcattgaatatacccgtgcatgtaccaggaatgtttttgattctaaaaacgtttttttctttgttgtaacggttgagtttttttcttcgtttcaaaacgagattctcgtttgtctcttcccttgtctaaaaacatctacggttcatgtgattcaaaaacactaaaaaaatataaactcatttttttttaatacttaacatttaaactatatatatatatatatatatatatatcttatactagtcccaagttttagtgtgaggtttttttattcaaaatctatcagtacattttttggaaaagaactaagtgaaattttctccaaattttccttttactattgattttttaattactggatgtcattaactttaatcttttgattctttcaacgtttaccattgggaaccttcacatgaaataaatgtctactttattgagtcataccttcgtcaacataaattaattgatgttcttctccaaattttgagtttttggtttttctaataatcttaacgaaagctttttggtatacatgtaaaacgtaacggcaagaatctgaacagtctactcaacggggtccataagtctagaatgtagaccccacaaacttactcttatcttattggtccgtaactaagaacgtgtccctctgattctcttgttttcttctaattaattcgtatcctacaaatttaattatcatttctacttcaactaatctttttttatttcctaaagatttcaatttctctctgtattttctatgaacagaattgaacttggaccagcacagcaacaacccaacccca

SEQ ID NO: 42 - DNA, 161 bp, Zea mays
ctgcagaaggtacgtacgtacattatataatgcattattgcttgcttgcttgcttgtttattcatgcatgcacgtgtagttggcagcacgtacatacagaaaggcaagctagctaactgagcaatgcaaaccttctgattgcattgcatgtcgatctgcag

SEQ ID NO: 43 - DNA, 534 bp, Zea mays
gtccgccttgtttctcctctgtctcttgatctgactaatcttggtttatgattcgttgagtaattttggggaaagcttcgtccacagtttttttttcgatgaacagtgccgcagtggcgctgatcttgtatgctatcctgcaatcgtggtgaacttatgtcttttatatccttcactaccatgaaaagactagtaatctttctcgatgtaacatcgtccagcactgctattaccgtgtggtccatccgacagtctggctgaacacatcatacgatattgagcaaagatctatcttccctgttctttaatgaaagacgtcattttcatcagtatgatctaagaatgttgcaacttgcaaggaggcgtttctttctttgaatttaactaactcgttgagtggccctgtttctcggacgtaaggcctttgctgctccacacatgtccattcgaattttaccgtgtttagcaagggcgaaaagtttgcatcttgatgatttagcttgactatgcgattgctttcctggacccgtgcag

SEQ ID NO: 44 - DNA, 97 bp, Zea mays
gtatctaatcagccatcccatttgtgatctttgtcagtagatatgatacaacaactcgcggttgacttgcgccttcttggcggcttatctgtctcag

SEQ ID NO: 45 - DNA, 14 bp, Zea mays
ctttgccgagtgtc

SEQ ID NO: 46 - DNA, 14 bp, Zea mays
ctttgccgagtgtc

SEQ ID NO: 47 - DNA, 14 bp, Zea mays
ctttgccgagtgtc

SEQ ID NO: 48 - DNA, 14 bp, Zea mays
ctttgccgagtgtc

SEQ ID NO: 49 - DNA, 14 bp, Zea mays
ctttgccgagtgtc

SEQ ID NO: 50 - DNA, 14 bp, Zea mays
ctttgccgagtgtc

SEQ ID NO: 51 - DNA, 14 bp, Zea mays
ctttgccgagtgtc

SEQ ID NO: 52 - DNA, 14 bp, Zea mays
ctttgccgagtgtc

SEQ ID NO: 53 - DNA, 14 bp, Zea mays
ctttgccgagtgtc

SEQ ID NO: 54 - DNA, 14 bp, Zea mays
ctttgccgagtgcc

SEQ ID NO: 55 - DNA, 14 bp, Zea mays
ctttgccgagtgcc

SEQ ID NO: 56 - DNA, 14 bp, Zea mays
ctttgccgagtgcc

SEQ ID NO: 57 - DNA, 14 bp, Zea mays
ctttgccgagtgcc

SEQ ID NO: 58 - DNA, 14 bp, Zea mays
ctttgccgagtgtt

SEQ ID NO: 59 - DNA, 14 bp, Zea mays
ctttgccgagtgtt

SEQ ID NO: 60 - DNA, 14 bp, Zea mays
ctttgccgagtgtt

SEQ ID NO: 61 - DNA, 14 bp, Zea mays
ctttgccgagtgct

SEQ ID NO: 62 - DNA, 14 bp, Zea mays
ctttgccgagtgct

SEQ ID NO: 63 - DNA, 14 bp, Zea mays
ctttgccgagtgct

SEQ ID NO: 64 - DNA, 14 bp, Zea mays
ctttgccgagtgta

SEQ ID NO: 65 - DNA, 14 bp, Zea mays
ctttgccgagtgca

SEQ ID NO: 66 - DNA, 14 bp, Zea mays
ctttgtcgagtgcc

SEQ ID NO: 67 - DNA, 14 bp, Zea mays
ctttgccgagtggt

SEQ ID NO: 68 - DNA, 14 bp, Zea mays
ctttgtcgagtgtt

SEQ ID NO: 69 - DNA, 14 bp, Zea mays
ctttgccgagtact

SEQ ID NO: 70 - DNA, 14 bp, Zea mays
ctttgccgagtact

SEQ ID NO: 71 - DNA, 14 bp, Zea mays
atttgccgagtgtt

SEQ ID NO: 72 - DNA, 14 bp, Zea mays
ctttgcctagtgtt

SEQ ID NO: 73 - DNA, 14 bp, Zea mays
ctttgccgggtgtg

SEQ ID NO: 74 - DNA, 14 bp, Zea mays
cttcgtcgagtgtt

SEQ ID NO: 75 - DNA, 14 bp, Zea mays
ctgtgccgagagct

SEQ ID NO: 76 - DNA, 14 bp, Zea mays
ctctgcagagtgta

SEQ ID NO: 77 - DNA, 14 bp, Zea mays
ctctgcagagtgta

SEQ ID NO: 78 - DNA, 21 bp, Zea mays
atagatatagatatagatata

SEQ ID NO: 79 - DNA, 21 bp, Zea mays
atagatagatagatagataga

SEQ ID NO: 80 - DNA, 21 bp, Zea mays
atatatagagagatagatata

SEQ ID NO: 81 - DNA, 21 bp, Zea mays
atagatagatagatagataga

SEQ ID NO: 82 - DNA, 21 bp, Zea mays
atagatatagatgtagatata

SEQ ID NO: 83 - DNA, 21 bp, Zea mays
agggatatagatatagatata

SEQ ID NO: 84 - DNA, 21 bp, Zea mays
atatatatatatatatatata

SEQ ID NO: 85 - DNA, 21 bp, Zea mays
atatatatatatatatatata

SEQ ID NO: 86 - DNA, 21 bp, Zea mays
atatatatatatatatatata

SEQ ID NO: 87 - DNA, 21 bp, Zea mays
atagatatagatatagacata

SEQ ID NO: 88 - DNA, 21 bp, Zea mays
agagatagatagatagatgga

SEQ ID NO: 89 - DNA, 21 bp, Zea mays
atagatagagatagagatata

SEQ ID NO: 90 - DNA, 21 bp, Zea mays
agatatagggatatagataga

SEQ ID NO: 91 - DNA, 21 bp, Zea mays
aaagatagatagatatatata

SEQ ID NO: 92 - DNA, 21 bp, Zea mays
agatatagatagataaataga

SEQ ID NO: 93 - DNA, 21 bp, Zea mays
agatatatatagatggatgga

SEQ ID NO: 94 - DNA, 21 bp, Zea mays
agagacatggagatagataga

SEQ ID NO: 95 - DNA, 21 bp, Zea mays
atagatatagttatggatata

SEQ ID NO: 96 - DNA, 21 bp, Zea mays
aggggtatagatatagatata

SEQ ID NO: 97 - DNA, 21 bp, Zea mays
atatatatatatataagtata

SEQ ID NO: 98 - DNA, 21 bp, Zea mays
atatatatatatataagtata

SEQ ID NO: 99 - DNA, 21 bp, Zea mays
aaagatagagatagagacata

SEQ ID NO: 100 - DNA, 21 bp, Zea mays
acggatagatagatagatgtt

SEQ ID NO: 101 - DNA, 21 bp, Zea mays
acatatatgtgtgtatatata

SEQ ID NO: 102 - DNA, 21 bp, Zea mays
acatatatgtgtgtatatata

SEQ ID NO: 103 - DNA, 21 bp, Zea mays
agacacaaagagatagatata

SEQ ID NO: 104 - DNA, 21 bp, Zea mays
gaatatatatggatggataga

SEQ ID NO: 105 - DNA, 21 bp, Zea mays
atatatatatgtgtggatggt

SEQ ID NO: 106 - DNA, 29 bp, Zea mays
ctccgcctcgcccgaccccagggctcggc

SEQ ID NO: 107 - DNA, 29 bp, Zea mays
ctccgcctcgcccgaccccagggctcggc

SEQ ID NO: 108 - DNA, 29 bp, Zea mays
ctccgcctcgcccgaccccagggcttggc

SEQ ID NO: 109 - DNA, 29 bp, Zea mays
tgccgccacgccagcggccatgccatgtc

SEQ ID NO: 110 - DNA, 29 bp, Zea mays
ctccgtcaccccagcgccgtgacagcggc

SEQ ID NO: 111 - DNA, 29 bp, Zea mays
tctcacctcgcccgagcccagccaagggc

SEQ ID NO: 112 - DNA, 29 bp, Zea mays
ctccgccgtgcagacgccgccgccgcagc

SEQ ID NO: 113 - DNA, 29 bp, Zea mays
cgccgcctccgccgagacgaggcatggcg

SEQ ID NO: 114 - DNA, 29 bp, Zea mays
ggcggctgcggctgccagccaccgccgcc

SEQ ID NO: 115 - DNA, 29 bp, Zea mays
ctcagcctcatccccagccacacccatgc

SEQ ID NO: 116 - DNA, 29 bp, Zea mays
gtccggtgcgccagccacgtcaccaaagc

SEQ ID NO: 117 - DNA, 29 bp, Zea mays
ctgcgccgccaacagccccgcgacctggc

SEQ ID NO: 118 - DNA, 29 bp, Zea mays
cgcccctgtgcacgcgcggaccgtccggc

SEQ ID NO: 119 - DNA, 29 bp, Zea mays
ctctgcctcgcccgaccttgagctcggac

SEQ ID NO: 120 - DNA, 29 bp, Zea mays
cgcgtcctcgcccgacctcggcctcaaga

SEQ ID NO: 121 - DNA, 29 bp, Zea mays
gtcctcgacgacagcggtggcgcgctggt

SEQ ID NO: 122 - DNA, 29 bp, Zea mays
tcacgcctcgcccgagggcctcctcaagc

SEQ ID NO: 123 - DNA, 29 bp, Zea mays
gtccgtctcgtcagacagggaacccgagc

SEQ ID NO: 124 - DNA, 29 bp, Zea mays
gtcctcgacgacagcggcggcacactggt

SEQ ID NO: 125 - DNA, 29 bp, Zea mays
ctcggccacgtacgcgcgcagacgacgga

SEQ ID NO: 126 - DNA, 29 bp, Zea mays
ttcccctaccaccaccatgcgccaccgcc

SEQ ID NO: 127 - DNA, 29 bp, Zea mays
ccctactgcaagcgcatcccccccttggc

SEQ ID NO: 128 - DNA, 29 bp, Zea mays
cagcttctccccccccccccctccccgcc

SEQ ID NO: 129 - DNA, 29 bp, Zea mays
cccaacaacacccgccatcgcggcactcc

SEQ ID NO: 130 - DNA, 29 bp, Zea mays
gtcaggcacgcccatactggcgcaccgga

SEQ ID NO: 131 - DNA, 29 bp, Zea mays
gccagctgcagggaacctgcggcgttggc

SEQ ID NO: 132 - DNA, 29 bp, Zea mays
gcctgcagcagccgactccatgcccctac

SEQ ID NO: 133 - DNA, 29 bp, Zea mays
ctgggcttcgggcgagttgagggcgcgcc

SEQ ID NO: 134 - DNA, 29 bp, Zea mays
gccatcatcgtccctcgccgtgcctcggt

SEQ ID NO: 135 - DNA, 29 bp, Zea mays
gaccgtctggcccctggccgcggaccgtc

SEQ ID NO: 136 - DNA, 29 bp, Zea mays
ttcccctaccaccaccatgcgccatcgcc

SEQ ID NO: 137 - DNA, 29 bp, Zea mays
cgccggttcatctgccgccgccaccatgc

SEQ ID NO: 138 - DNA, 29 bp, Zea mays
ccacactgcgtgccaccggccgtctcagc

SEQ ID NO: 139 - DNA, 29 bp, Zea mays
ccactctgcgtgccaccggccgtctcagc

SEQ ID NO: 140 - DNA, 29 bp, Zea mays
gtcgtctgaggcagcagcgccgaatcggc

SEQ ID NO: 141 - DNA, 29 bp, Zea mays
ctctggtgcaccggacactgtccggtggc

SEQ ID NO: 142 - DNA, 29 bp, Zea mays
gtccggtgcgccagaccagaggtgccttc

SEQ ID NO: 143 - DNA, 29 bp, Zea mays
gccccctacggaagcaaggagacgtcagc

SEQ ID NO: 144 - DNA, 29 bp, Zea mays
gcccgcgacagcgatcgcgagcggctgcc

SEQ ID NO: 145 - DNA, 29 bp, Zea mays
ctgcacggccgtcgccgtcgtcgcccgtc

SEQ ID NO: 146 - DNA, 29 bp, Zea mays
cggcgtttcgaccccgggggtccctggac

SEQ ID NO: 147 - DNA, 29 bp, Zea mays
cccgtgctcccgcgcgaccacccttctgt

SEQ ID NO: 148 - DNA, 29 bp, Zea mays
ggcaacagcgccaggggctgccctgcagc

SEQ ID NO: 149 - DNA, 29 bp, Zea mays
cctcgcttccgcaaaacccatgcatgtgc

SEQ ID NO: 150 - DNA, 29 bp, Zea mays
cccggccacgctcgttcctcccacgtggc

SEQ ID NO: 151 - DNA, 29 bp, Zea mays
cctcaccggcacagccacggcggcaatgc

SEQ ID NO: 152 - DNA, 29 bp, Zea mays
cgccgccggtccacctgccaccgccggtc

SEQ ID NO: 153 - DNA, 29 bp, Zea mays
gccgacagcagcaggagtgccgcgatggc

SEQ ID NO: 154 - DNA, 29 bp, Zea mays
gagagcctgcgcaccccgccgccgctagc

SEQ ID NO: 155 - DNA, 14 bp, Zea mays
ctttgccgagtgcc

SEQ ID NO: 156 - DNA, 14 bp, Zea mays
ctttgccgagtgcc

SEQ ID NO: 157 - DNA, 14 bp, Zea mays
ctttgccgagtgcc

SEQ ID NO: 158 - DNA, 14 bp, Zea mays
ctttgccgagtgcc

SEQ ID NO: 159 - DNA, 14 bp, Zea mays
ctttgccgagtgtc

SEQ ID NO: 160 - DNA, 14 bp, Zea mays
ctttgccgagtgtc

SEQ ID NO: 161 - DNA, 14 bp, Zea mays
ctttgccgagtgtc

SEQ ID NO: 162 - DNA, 14 bp, Zea mays
ctttgccgagtgtc

SEQ ID NO: 163 - DNA, 14 bp, Zea mays
ctttgccgagtgtc

SEQ ID NO: 164 - DNA, 14 bp, Zea mays
ctttgccgagtgtc

SEQ ID NO: 165 - DNA, 14 bp, Zea mays
ctttgccgagtgtc

SEQ ID NO: 166 - DNA, 14 bp, Zea mays
ctttgccgagtgtc

SEQ ID NO: 167 - DNA, 14 bp, Zea mays
ctttgccgagtgtc

SEQ ID NO: 168 - DNA, 14 bp, Zea mays
ctttgccgagtgct

SEQ ID NO: 169 - DNA, 14 bp, Zea mays
ctttgccgagtgct

SEQ ID NO: 170 - DNA, 14 bp, Zea mays
ctttgccgagtgct

SEQ ID NO: 171 - DNA, 14 bp, Zea mays
ctttgccgagtgtt

SEQ ID NO: 172 - DNA, 14 bp, Zea mays
ctttgccgagtgtt

SEQ ID NO: 173 - DNA, 14 bp, Zea mays
ctttgccgagtgtt

SEQ ID NO: 174 - DNA, 14 bp, Zea mays
ctttgccgagtgca

SEQ ID NO: 175 - DNA, 14 bp, Zea mays
ctttgccgagtgta

SEQ ID NO: 176 - DNA, 14 bp, Zea mays
ctttgtcgagtgcc

SEQ ID NO: 177 - DNA, 14 bp, Zea mays
ctttgtcgagtgtt

SEQ ID NO: 178 - DNA, 14 bp, Zea mays
ctttgccgagtggt

SEQ ID NO: 179 - DNA, 14 bp, Zea mays
ctttgccgagtact

SEQ ID NO: 180 - DNA, 14 bp, Zea mays
ctttgccgagtact

SEQ ID NO: 181 - DNA, 14 bp, Zea mays
atttgccgagtgtt

SEQ ID NO: 182 - DNA, 14 bp, Zea mays
ctttgcctagtgtt

SEQ ID NO: 183 - DNA, 14 bp, Zea mays
ctgtgccgagagct

SEQ ID NO: 184 - DNA, 14 bp, Zea mays
cttcgtcgagtgtt

SEQ ID NO: 185 - DNA, 14 bp, Zea mays
ctttgccgggtgtg

SEQ ID NO: 186 - DNA, 14 bp, Zea mays
ctgtgtcgagagct

SEQ ID NO: 187 - DNA, 41 bp, Zea mays
atataggggtgtagatatagatatagatgtagatatagata

SEQ ID NO: 188 - DNA, 41 bp, Zea mays
atagatagatagatagatagatagatagatagagatagata

SEQ ID NO: 189 - DNA, 41 bp, Zea mays
atagatagatagatagatagatagacacaaagagatagata

SEQ ID NO: 190 - DNA, 41 bp, Zea mays
agatctagttatagatatagatagagatagagatatagaaa

SEQ ID NO: 191 - DNA, 41 bp, Zea mays
atatatatatatatatatatatatatatatatatatatata

SEQ ID NO: 192 - DNA, 41 bp, Zea mays
tcacatagatatagatatagatatagagatacatatagcaa

SEQ ID NO: 193 - DNA, 41 bp, Zea mays
tcaaatagatatagatatagggatatagatagatatagcaa

SEQ ID NO: 194 - DNA, 41 bp, Zea mays
atatagagagatagatataaatagatataaataagtagata

SEQ ID NO: 195 - DNA, 41 bp, Zea mays
atataggggtatagatatagatataataaggggtatagata

SEQ ID NO: 196 - DNA, 41 bp, Zea mays
atctagggatatagatatagatatagccatctagctagcta

SEQ ID NO: 197 - DNA, 41 bp, Zea mays
atatatatatatatatatatatatatttgtgtatgtaaata

SEQ ID NO: 198 - DNA, 41 bp, Zea mays
acatatagatatagttatggatatagatattgggatatgtt

SEQ ID NO: 199 - DNA, 41 bp, Zea mays
atgaataaagatagagatagagacataaaaatatagagata

SEQ ID NO: 200 - DNA, 41 bp, Zea mays
agatggactggtagatagattaagagacatggagatagata

SEQ ID NO: 201 - DNA, 41 bp, Zea mays
agatatagatagataaatagatatgatagaatagataactt

SEQ ID NO: 202 - DNA, 41 bp, Zea mays
atgaagagatggagggagtggattagatgtagatatagtaa

SEQ ID NO: 203 - DNA, 41 bp, Zea mays
atccacacatatatatatacaaaaacaagcaaagataggaa

SEQ ID NO: 204 - DNA, 41 bp, Zea mays
aaaagtatatgcacatagagatttagcaaaaggaatacata

SEQ ID NO: 205 - DNA, 29 bp, Zea mays
ctccgcctcgcccgaccccagggctcggc

SEQ ID NO: 206 - DNA, 29 bp, Zea mays
ctccgcctcgcccgaccccagggctcggc

SEQ ID NO: 207 - DNA, 29 bp, Zea mays
ctccgcctcgcccgaccccagggcttggc

SEQ ID NO: 208 - DNA, 29 bp, Zea mays
ctctgcctcgcccgaccttggacctcggc

SEQ ID NO: 209 - DNA, 29 bp, Zea mays
tctcacctcgcccgagcccagccaagggc

SEQ ID NO: 210 - DNA, 29 bp, Zea mays
ctccgtcaccccagcgccgtgacagcggc

SEQ ID NO: 211 - DNA, 29 bp, Zea mays
ctctgcctcgcccgaccttgagctcggac

SEQ ID NO: 212 - DNA, 29 bp, Zea mays
ttccacccaaccagcgcgccaccgctgtc

SEQ ID NO: 213 - DNA, 29 bp, Zea mays
cgcgtcctcgcccgacctcggcctcaaga

SEQ ID NO: 214 - DNA, 29 bp, Zea mays
ctcggccacgtacgcgcgcagacgacgga

SEQ ID NO: 215 - DNA, 29 bp, Zea mays
tgccgccacgccagcggccatgccatgtc

SEQ ID NO: 216 - DNA, 29 bp, Zea mays
tcacgcctcgcccgagggcctcctcaagc

SEQ ID NO: 217 - DNA, 29 bp, Zea mays
cctcaactcgcccgaagcccagctcgggc

SEQ ID NO: 218 - DNA, 29 bp, Zea mays
ctccacacaaccagagcgctccaggcagc

SEQ ID NO: 219 - DNA, 29 bp, Zea mays
gtccgtctcgtcagacagggaacccgagc

SEQ ID NO: 220 - DNA, 29 bp, Zea mays
ccctaccaccaccatgcgccaccgccaga

SEQ ID NO: 221 - DNA, 29 bp, Zea mays
cccggccacgctcgttcctcccacgtggc

SEQ ID NO: 222 - DNA, 29 bp, Zea mays
tgccgtcccgtcagcccgtcacacacaga

SEQ ID NO: 223 - DNA, 29 bp, Zea mays
cgcttacgcacccgaccccaagctgcatc

SEQ ID NO: 224 - DNA, 29 bp, Zea mays
ggcagccgctcgcgatcgctgtcgcgggc

SEQ ID NO: 225 - DNA, 29 bp, Zea mays
ttccacccaaccagtgtgccgccgctgtc

SEQ ID NO: 226 - DNA, 29 bp, Zea mays
ctccgccgtgcagacgccgccgccgcagc

SEQ ID NO: 227 - DNA, 29 bp, Zea mays
ccctaccaccaccatgcgccatcgccgga

SEQ ID NO: 228 - DNA, 29 bp, Zea mays
tcgcggaccgtctggcccctggccgcgga

SEQ ID NO: 229 - DNA, 29 bp, Zea mays
atccggcagctccgatcctttccggcggc

SEQ ID NO: 230 - DNA, 29 bp, Zea mays
ctctggtgcaccggacactgtccggtggc

SEQ ID NO: 231 - DNA, 29 bp, Zea mays
ggcaacagcgccaggggctgccctgcagc

SEQ ID NO: 232 - DNA, 29 bp, Zea mays
gcgcggaccgtccggcctcttggcgcgga

SEQ ID NO: 233 - DNA, 29 bp, Zea mays
ctccatctctcccgacgacgacgggtgga

SEQ ID NO: 234 - DNA, 29 bp, Zea mays
cctgaccgcgcgcgctctccatctcaagc

SEQ ID NO: 235 - DNA, 29 bp, Zea mays
ggccggctccccagtctccaagctctata

SEQ ID NO: 236 - DNA, 29 bp, Zea mays
gtccggtgcgccagccacgtcaccaaagc

SEQ ID NO: 237 - DNA, 29 bp, Zea mays
ctctagccagcgctaccgtctaccgaggc

SEQ ID NO: 238 - DNA, 29 bp, Zea mays
ctgcactctgcacggccgtcgccgtcgtc

SEQ ID NO: 239 - DNA, 29 bp, Zea mays
tgcaagcgcatcccccccttggcgcagga

SEQ ID NO: 240 - DNA, 29 bp, Zea mays
gtcaggcacgcccatactggcgcaccgga

SEQ ID NO: 241 - DNA, 29 bp, Zea mays
ccccgtcaggagagatcctaggcttcgtc

SEQ ID NO: 242 - DNA, 29 bp, Zea mays
ccggacacagcccgcacgtatgagctgga

SEQ ID NO: 243 - DNA, 29 bp, Zea mays
gcccatctcatgagcgacgtaccgcgatc

SEQ ID NO: 244 - DNA, 38 bp, Zea mays
gtaataaaactttgtagttgacaattttttgatttgaa

SEQ ID NO: 245 - DNA, 38 bp, Zea mays
gttatacaaatttgtagttgaaaactttttcatttgaa

SEQ ID NO: 246 - DNA, 38 bp, Zea mays
gttataaaactttgtagttgacaatgttttagtttgaa

SEQ ID NO: 247 - DNA, 38 bp, Zea mays
gttatagaagtttatagttgacaactttttcatttaaa

SEQ ID NO: 248 - DNA, 38 bp, Zea mays
gttattttacttttgagtagctaaaatttagcttggta

SEQ ID NO: 249 - DNA, 38 bp, Zea mays
gttattttaattttgagtagctaaaatttagcttggta

SEQ ID NO: 250 - DNA, 38 bp, Zea mays
gttatgcaactttatagttggtcatattttcaaatgaa

SEQ ID NO: 251 - DNA, 38 bp, Zea mays
gctataatactattaagtagctaaattttagcttaaca

SEQ ID NO: 252 - DNA, 40 bp, Zea mays
aaatagggccaagtcacaaatccacttcaaaggtgactct

SEQ ID NO: 253 - DNA, 40 bp, Zea mays; identical to SEQ ID NO: 252

SEQ ID NO: 254 - DNA, 40 bp, Zea mays
acatagatttaagtcacaaatctacatccaagctatggct

SEQ ID NO: 255 - DNA, 40 bp, Zea mays
acatggatttaaatcacaaatctacatccaagctatggct

SEQ ID NO: 256 - DNA, 40 bp, Zea mays
aaatagggccaagtcataaattcacttcaaagtgactctt

SEQ ID NO: 257 - DNA, 14 bp, Zea mays
ctttgccgagtgtc

SEQ ID NO: 258 - DNA, 14 bp, Zea mays
ctttgccgagtgtc

SEQ ID NO: 259 - DNA, 14 bp, Zea mays
ctttgccgagtgtc

SEQ ID NO: 260 - DNA, 14 bp, Zea mays
ctttgccgagtgtc

SEQ ID NO: 261 - DNA, 14 bp, Zea mays
ctttgccgagtgtc

SEQ ID NO: 262 - DNA, 14 bp, Zea mays
ctttgccgagtgtc

SEQ ID NO: 263 - DNA, 14 bp, Zea mays
ctttgccgagtgtc

SEQ ID NO: 264 - DNA, 14 bp, Zea mays
ctttgccgagtgtc

SEQ ID NO: 265 - DNA, 14 bp, Zea mays
ctttgccgagtgtc

SEQ ID NO: 266 - DNA, 14 bp, Zea mays
ctttgccgagtgcc

SEQ ID NO: 267 - DNA, 14 bp, Zea mays
ctttgccgagtgcc

SEQ ID NO: 268 - DNA, 14 bp, Zea mays
ctttgccgagtgcc

SEQ ID NO: 269 - DNA, 14 bp, Zea mays
ctttgccgagtgcc

SEQ ID NO: 270 - DNA, 14 bp, Zea mays
ctttgccgagtgtt

SEQ ID NO: 271 - DNA, 14 bp, Zea mays
ctttgccgagtgtt

SEQ ID NO: 272 - DNA, 14 bp, Zea mays
ctttgccgagtgtt

SEQ ID NO: 273 - DNA, 14 bp, Zea mays
ctttgccgagtgct

SEQ ID NO: 274 - DNA, 14 bp, Zea mays
ctttgccgagtgct

SEQ ID NO: 275 - DNA, 14 bp, Zea mays
ctttgccgagtgct

SEQ ID NO: 276 - DNA, 14 bp, Zea mays
ctttgccgagtgta

SEQ ID NO: 277 - DNA, 14 bp, Zea mays
ctttgccgagtgca

SEQ ID NO: 278 - DNA, 14 bp, Zea mays
ctttgtcgagtgcc

SEQ ID NO: 279 - DNA, 14 bp, Zea mays
ctttgtcgagtgtt

SEQ ID NO: 280 - DNA, 14 bp, Zea mays
ctttgccgagtggt

SEQ ID NO: 281 - DNA, 14 bp, Zea mays
ctttgccgagtact

SEQ ID NO: 282 - DNA, 14 bp, Zea mays
ctttgccgagtact

SEQ ID NO: 283 - DNA, 14 bp, Zea mays
atttgccgagtgtt

SEQ ID NO: 284 - DNA, 14 bp, Zea mays
ctttgcctagtgtt

SEQ ID NO: 285 - DNA, 14 bp, Zea mays
ctgtgccgagagct

SEQ ID NO: 286 - DNA, 14 bp, Zea mays
ctttgccgggtgtg

SEQ ID NO: 287 - DNA, 14 bp, Zea mays
cttcgtcgagtgtt

SEQ ID NO: 288 - DNA, 14 bp, Zea mays
ctctgcagagtgta

SEQ ID NO: 289 - DNA, 14 bp, Zea mays
ctctgcagagtgta

SEQ ID NO: 290 - DNA, 14 bp, Zea mays
ctgtgtcgagagct

SEQ ID NO: 291 - DNA, 29 bp, Zea mays
agatatagatgtagatatagatatataga

SEQ ID NO: 292 - DNA, 29 bp, Zea mays
gggtatagatatagatatagacatataga

SEQ ID NO: 293 - DNA, 29 bp, Zea mays
agatagatagatagagatagatagataga

SEQ ID NO: 294 - DNA, 29 bp, Zea mays
agatatagatatagatatagagatacata

SEQ ID NO: 295 - DNA, 29 bp, Zea mays
ataaatagatagatagatagatagataga

SEQ ID NO: 296 - DNA, 29 bp, Zea mays
agatatagatagagatagagatatagaaa

SEQ ID NO: 297 - DNA, 29 bp, Zea mays
agatagagatagagacataaaaatataga

SEQ ID NO: 298 - DNA, 29 bp, Zea mays
atatagagagatagatataaatagatata

SEQ ID NO: 299 - DNA, 29 bp, Zea mays
atatatatatatatatatatatatatata

SEQ ID NO: 300 - DNA, 29 bp, Zea mays
tcaaatagatatagatatagggatataga

SEQ ID NO: 301 - DNA, 29 bp, Zea mays
ataagtagatagatagatagatagataga

SEQ ID NO: 302 - DNA, 29 bp, Zea mays
ggatatagatatagatatagccatctagc

SEQ ID NO: 303 - DNA, 29 bp, Zea mays
acatatagatatagttatggatatagata

SEQ ID NO: 304 - DNA, 29 bp, Zea mays
agatatatatagatggatggactgataga

SEQ ID NO: 305 - DNA, 29 bp, Zea mays
agacatggagatagatagacacggataga

SEQ ID NO: 306 - DNA, 29 bp, Zea mays
tggattagatgtagatatagtaagataga

SEQ ID NO: 307 - DNA, 29 bp, Zea mays
atataggggtatagatatagatataataa

SEQ ID NO: 308 - DNA, 29 bp, Zea mays
tcatatatatatatatatatatataaacc

SEQ ID NO: 309 - DNA, 29 bp, Zea mays
tagtatatatatatatatataagtataaa

SEQ ID NO: 310 - DNA, 29 bp, Zea mays
agctattgatatagggataaaacaataga

SEQ ID NO: 311 - DNA, 41 bp, Zea mays
taaaactttgtagttgacaattttttgatttgaaatcatct

SEQ ID NO: 312 - DNA, 41 bp, Zea mays
taaaacattgtagttgacaacttttttatttgaaatcattt

SEQ ID NO: 313 - DNA, 41 bp, Zea mays
tacaaatttgtagttgaaaactttttcatttgaattaattt

SEQ ID NO: 314 - DNA, 41 bp, Zea mays
taaaactttgtagttgacaatgttttagtttgaaatcatct

SEQ ID NO: 315 - DNA, 41 bp, Zea mays
tacaactttcatgttggtggttttttctttcgaagtcgttt

SEQ ID NO: 316 - DNA, 41 bp, Zea mays
tagaagtttatagttgacaactttttcatttaaagtcatat

SEQ ID NO: 317 - DNA, 41 bp, Zea mays
tacaacttttattttggtggtttttccatccgagacagttt

SEQ ID NO: 318 - DNA, 41 bp, Zea mays
ttcaatttagtagttgactactttttcatttgaatttgttt

SEQ ID NO: 319 - DNA, 41 bp, Zea mays
tgcaactttatagttggtcatattttcaaatgaactcattt

SEQ ID NO: 320 - DNA, 41 bp, Zea mays
tacaagttttattttaatagtttctacatccgagaccgttt

SEQ ID NO: 321 - DNA, 41 bp, Zea mays
tactacgttgaagttcacatcattgtccattgaactagatt

SEQ ID NO: 322 - DNA, 41 bp, Zea mays
taccacttttattttggtacttttcccatcggaggcggttt

SEQ ID NO: 323 - DNA, 41 bp, Zea mays
tagaattttgaagttaactatgctgctgtctgaaacagatt

SEQ ID NO: 324 - DNA, 41 bp, Zea mays
tagtaccttgaaattcgcattctatttttatgcattcttca

SEQ ID NO: 325 - DNA, 41 bp, Zea mays; identical to SEQ ID NO: 324

SEQ ID NO: 326 - DNA, 41 bp, Zea mays
tacaatttatatattgaacatttcttcatatgacaaaataa

SEQ ID NO: 327 - DNA, 29 bp, Zea mays
ctccgcctcgcccgaccccagggctcggc

SEQ ID NO: 328 - DNA, 29 bp, Zea mays
ctccgcctcgcccgaccccagggctcggc

SEQ ID NO: 329 - DNA, 29 bp, Zea mays
ctccgcctcgcccgaccccagggcttggc

SEQ ID NO: 330 - DNA, 29 bp, Zea mays
ctctgcctcgcccgaccttggacctcggc

SEQ ID NO: 331 - DNA, 29 bp, Zea mays
tgccgccacgccagcggccatgccatgtc

SEQ ID NO: 332 - DNA, 29 bp, Zea mays
ctccgtcaccccagcgccgtgacagcggc

SEQ ID NO: 333 - DNA, 29 bp, Zea mays
tctcacctcgcccgagcccagccaagggc

SEQ ID NO: 334 - DNA, 29 bp, Zea mays
ctctgcctcgcccgaccttgagctcggac

SEQ ID NO: 335 - DNA, 29 bp, Zea mays
cgccgcctccgccgagacgaggcatggcg

SEQ ID NO: 336 - DNA, 29 bp, Zea mays
cagcttctccccccccccccctccccgcc

SEQ ID NO: 337 - DNA, 29 bp, Zea mays
gtccggtgcgccagccacgtcaccaaagc

SEQ ID NO: 338 - DNA, 29 bp, Zea mays
cgccgccggtccacctgccaccgccggtc

SEQ ID NO: 339 - DNA, 29 bp, Zea mays
cgcccctgtgcacgcgcggaccgtccggc

SEQ ID NO: 340 - DNA, 29 bp, Zea mays
ttcatctgccgccgccaccatgccactac

SEQ ID NO: 341 - DNA, 29 bp, Zea mays
cgcgtcctcgcccgacctcggcctcaaga

SEQ ID NO: 342 - DNA, 29 bp, Zea mays
cctcaactcgcccgaagcccagctcgggc

SEQ ID NO: 343 - DNA, 29 bp, Zea mays
cgcttacgcacccgaccccaagctgcatc

SEQ ID NO: 344 - DNA, 29 bp, Zea mays
gaccgtctggcccctggccgcggaccgtc

SEQ ID NO: 345 - DNA, 29 bp, Zea mays
ctcatccccagccacacccatgcccgtgc

SEQ ID NO: 346 - DNA, 29 bp, Zea mays
ggcggctgcggctgccagccaccgccgcc

SEQ ID NO: 347 - DNA, 29 bp, Zea mays
ctccgccgtgcagacgccgccgccgcagc

SEQ ID NO: 348 - DNA, 29 bp, Zea mays
cctcgcttccgcaaaacccatgcatgtgc

SEQ ID NO: 349 - DNA, 29 bp, Zea mays
gcctgcagcagccgactccatgcccctac

SEQ ID NO: 350 - DNA, 29 bp, Zea mays
gtccggtgcgccagaccagaggtgccttc

SEQ ID NO: 351 - DNA, 29 bp, Zea mays
aggcccgtgccctgcgcccaggcttaggc

SEQ ID NO: 352 - DNA, 29 bp, Zea mays
ctgcgccgccaacagccccgcgacctggc

SEQ ID NO: 353 - DNA, 29 bp, Zea mays
tcacgcctcgcccgagggcctcctcaagc

SEQ ID NO: 354 - DNA, 29 bp, Zea mays
gtccgtctcgtcagacagggaacccgagc

SEQ ID NO: 355 - DNA, 29 bp, Zea mays
gttccatgccccccccccctcgctctctc

SEQ ID NO: 356 - DNA, 29 bp, Zea mays
cctcaccggcacagccacggcggcaatgc

SEQ ID NO: 357 - DNA, 29 bp, Zea mays
cggcgtttcgaccccgggggtccctggac

SEQ ID NO: 358 - DNA, 29 bp, Zea mays
ctgcacggccgtcgccgtcgtcgcccgtc

SEQ ID NO: 359 - DNA, 29 bp, Zea mays
gtcctatgggccaaaccccatagctctcc

SEQ ID NO: 360 - DNA, 29 bp, Zea mays
cggcgttggcagcaccccgatcctgggcc

SEQ ID NO: 361 - DNA, 29 bp, Zea mays
cacctcttcgacagccacgttggcatgta

SEQ ID NO: 362 - DNA, 29 bp, Zea mays
ccccgtcaggagagatcctaggcttcgtc

SEQ ID NO: 363 - DNA, 29 bp, Zea mays
gtcctcgacgacagcggcggcacactggt

SEQ ID NO: 364 - DNA, 29 bp, Zea mays
aaccctgacggccccgcccatgcaaaggc

SEQ ID NO: 365 - DNA, 29 bp, Zea mays
ggccggctccccagtctccaagctctata

SEQ ID NO: 366 - DNA, 29 bp, Zea mays
gtcttctgggcccgcctgcatgtccggtg

SEQ ID NO: 367 - DNA, 29 bp, Zea mays
ccctactgcaagcgcatcccccccttggc

SEQ ID NO: 368 - DNA, 29 bp, Zea mays
cccgtgctcccgcgcgaccacccttctgt

SEQ ID NO: 369 - DNA, 29 bp, Zea mays
ggcagccgctcgcgatcgctgtcgcgggc

SEQ ID NO: 370 - DNA, 29 bp, Zea mays
agcagcgacgcctccccacttgccccttc

SEQ ID NO: 371 - DNA, 29 bp, Zea mays
ttcccctaccaccaccatgcgccatcgcc

SEQ ID NO: 372 - DNA, 29 bp, Zea mays
gtcaggcacgcccatactggcgcaccgga

SEQ ID NO: 373 - DNA, 29 bp, Zea mays
ctctggtgcaccggacactgtccggtggc

SEQ ID NO: 374 - DNA, 29 bp, Zea mays
cccaacaacacccgccatcgcggcactcc

SEQ ID NO: 375 - DNA, 29 bp, Zea mays
gagagcctgcgcaccccgccgccgctagc

SEQ ID NO: 376 - DNA, 29 bp, Zea mays
ctgcgctgcctctgctcgctcgctcactc

SEQ ID NO: 377 - DNA, 40 bp, Zea mays
agctagctggctgaatggtaatagttgttgctgctgcaaa

SEQ ID NO: 378 - DNA, 40 bp, Zea mays
tgccaattggttaaatggaagaagttgttgttgctgagag

SEQ ID NO: 379 - DNA, 40 bp, Zea mays
agctagctggctaaatggtagtagtatttgatgctgcaaa

SEQ ID NO: 380 - DNA, 40 bp, Zea mays
agcaagttggacaaagggcaacaactgttgtagttgtaag

SEQ ID NO: 381 - DNA, 40 bp, Zea mays
agacaattggttgaatggaagaaactattgttgctgagag

SEQ ID NO: 382 - DNA, 40 bp, Zea mays
aaactgctagtagaacggcagcaactgttgttgtgtcaag

SEQ ID NO: 383 - DNA, 40 bp, Zea mays
agcaagtaggccaagtagaagagagagaagtagtaggacg

SEQ ID NO: 384 - DNA, 40 bp, Zea mays
agctacttggctaatcacaggtagcacgagttgttgcagc

SEQ ID NO: 385 - DNA, 40 bp, Zea mays
aggtggcaggtgaaccggaggtggcagatgcaccggcggc

SEQ ID NO: 386 - DNA, 40 bp, Zea mays
tgcatgttagacaagtggcaaaactacaactagtgtgtag